(19) United States
(12) Patent Application Publication
(10) Pub. No.: US 2023/0114801 A1
(43) Pub. Date: Apr. 13, 2023

(54) MINIATURE GUIDANCE AND NAVIGATION CONTROL (miniGNC) ANTIBODY-LIKE PROTEINS AND METHODS OF MAKING AND USING THEREOF

(71) Applicants: SYSTIMMUNE, INC., Reamond, WA (US); BAILI-BIO (CHENGDU) PHARMACEUTICAL CO., LTD., Chengdu, Sichuan (CN)

(72) Inventors: Yi Zhu, Redmond, WA (US); Soumili Chatterjee, Bothell, WA (US); Dennis R. Goulet, Redmond, WA (US); Tsung-I Tsai, Redmond, WA (US); Blair Renshaw, Renton, WA (US); Christopher G. Vincent, Lynnwood, WA (US); Andrew Waight, Kenmore, WA (US); Nga Sze Amanda Mak, Mukilteo, WA (US)

(21) Appl. No.: 17/909,360

(22) PCT Filed: Mar. 17, 2021
(86) PCT No.: PCT/US21/22847

§ 371 Date: Sep. 3, 2022

Related U.S. Application Data
(60) Provisional application No. 62/991,042, filed on Mar. 17, 2020.

(51) Int. Cl. C07K 16/30; C07K 16/28; C07K 16/32

(52) U.S. Cl. CPC C07K 16/30; C07K 16/2803; C07K 16/2809; C07K 16/2851; C07K 16/2863; C07K 16/3007; C07K 16/32; A61K 2039/507

(57) ABSTRACT

A multi-specific antibody-like protein having a first monomer comprising a first binding monomer, a CH1 domain, a first hinge, a first CH2 domain, and a first CH3 domain, a second monomer comprising a second binding monomer, a CL domain, a second hinge, a second CH2 domain, and a second CH3 domain, wherein the first binding monomer and a second binding monomer are configured to form a dimer, wherein the first monomer and the second monomer are covalently paired through at least one disulfide bond between the CH1 domain and the CL domain and at least one disulfide bond between the first hinge and the second hinge, wherein the first monomer and second monomer may comprise optionally a first binding domain (D1) and a second binding domain (D2), and a fourth binding domain (D4) and a fifth binding domain (D5), and wherein the multi-specific antibody-like protein is at least bi-specific.

### CROSS REFERENCE TO RELATED APPLICATIONS

This application claims the benefit of the filing date of U.S. Provisional Application Ser. No. 62/991,042 filed Mar. 17, 2020 under 35 U.S.C. 119(e), the entire disclosures of which are incorporated by reference herein.

### TECHNICAL FIELD

The present application generally relates to the technical field of multi-specific antibody for cancer immunotherapy and more particularly relates to making and using miniature Guidance and Navigation Control (miniGNC) antibodies with multiple binding activities against surface molecules of both immune cells and tumor cells.

### BACKGROUND

Therapeutic antibodies have become a mainstay in treatment of several diseases, including but not limited to cancer, infection, and autoimmunity. While monoclonal, monospecific antibodies provide a straightforward mechanism to treat disease, e.g., via inhibition or activation of specific signalling pathways, multi-specific antibodies allow more complex therapeutic mechanisms to be explored. By targeting multiple distinct antigens, or multiple epitopes within a single antigen, biological responses such as cell colocalization and activation can be elicited, which enables, for example, the redirection of T cells and other immune cells to the site of specific antigen-expressing tumor cells. In this way, bispecific and multi-specific antibodies have become important platforms in the field of immunooncology.

Due to the advantages of multi-specific antibody platforms, many such frameworks have been developed. One such platform is known as Guidance and Navigation Control (GNC). The GNC proteins include the proteins linking multiple functionally independent binding moieties into a single entity that is capable of bringing both effector cells and target cells together (see Applicant's applications, WO/2019/005641, WO2019191120, and PCT/US20/59230, incorporated herein in its entirety). In general, these platforms are restricted to at least two specificities, preventing the exploration of complex therapeutic mechanisms that require binding to more than two antigens. For antibody platforms with more than two specificities, the increased number of binding domains usually necessitates formation of large molecules which often have undesirable physical and biological properties. The increase in molecular weight can reduce developability, as larger and more complex molecules are more likely to have problems with aggregation and solubility. Furthermore, the ability of large macromolecules to penetrate into solid tumors may be hindered compared to proteins of smaller size. Thus, there is a need for multi-specific antibody platforms with more than two specificities that are not significantly larger than monoclonal IgG antibodies.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides proteins with binding specificities such as multi-specific protein like antibodies including multi-specific antibodies, and the fragments of these binding proteins including without limitation scFv domain, Fab region, Fc domain, VH, VL, light chains, heavy chains, variable regions, and complementary determining region (CDR). The application further provides method of making and using the antibody-like proteins disclosed herein.

In one aspect, the application provides multi-specific antibody-like proteins. In one embodiment, the multi-specific antibody-like protein having a N-terminus and a C-terminus, including, a first monomer, including, from the N-terminus to the C-terminus, a first binding monomer, a CH1 domain, a first hinge, a first CH2 domain, and a first CH3 domain, wherein the first monomer may comprise optionally a first binding domain (D1) linked to the N-terminus, a fourth binding domain (D4) linked to the C-terminus, or both, a second monomer, including, from the N-terminus to the C-terminus, a second binding monomer, a CL domain, a second hinge, a second CH2 domain, and a second CH3 domain, wherein the second monomer may comprise optionally a second binding domain (D2) linked to the N-terminus, a fifth binding domain (D5) linked to the C-terminus, or both, wherein the first binding monomer and a second binding monomer are configured to form a dimer, wherein the first monomer and the second monomer are covalently paired through at least one disulfide bond between the CH1 domain and the CL domain and at least one disulfide bond between the first hinge and the second hinge, and wherein the multi-specific antibody-like protein is at least bi-specific.

In one embodiment, the multi-specific antibody-like protein may be tri-specific, tetra-specific, or penta-specific. In one embodiment, the multi-specific antibody-like protein may be monoclonal antibodies. In one embodiment, the multi-specific antibody-like protein may be purified monoclonal antibody. In one embodiment, the multi-specific antibody-like protein may be humanized antibodies.

In one embodiment, the multi-specific antibody-like protein may further comprise a disulfide bond between the first CH3 domain and a second CH3 domain.

In one embodiment, the multi-specific antibody-like protein may further comprise a second disulfide bond between the first hinge and the second hinge.

In one embodiment, connecting linkers are used between various domains. In one embodiment, the connecting linker may comprise a (GₓS_{y}), linker, wherein n, x, and y each independently is an integer from 1 to 10. In one embodiment, D1, D2, D4 or D5 are liked to N- or C-terminus through a linker. In one embodiment, the linker may comprise a (GₓS_{y}), linker. n, x, and y each independently may be an integer from 1 to 10. In one embodiment, n is 1, 2, 3, 4, 5, 6, 7, 8, 9 and 10. In one embodiment, x is 1, 2, 3, 4, 5, 6, 7, 8, 9 and 10. In one embodiment, y is 1, 2, 3, 4, 5, 6, 7, 8, 9 and 10.

In one embodiment, the first CH3 domain is configured to form a hole structure, wherein the second CH3 domain is configured to form a knob structure, and wherein the first CH2 and CH3 domains and the second CH2 and CH3 domains are configured to heterodimerize to form a complementary Fc domain.

In one embodiment, the first CH3 domain may comprise at least one ‘hole’ mutation at T366S, L368A or Y407V and the second CH3 domain may comprise a ‘knob’ mutation at T366W.

In one embodiment, the Fc domain may comprise mutations at H435R/Y436F.

In one embodiment, the Fc domain is engineered to eliminate effector cell functions selected from ADCC, ADCP, or CDC.

In one embodiment, the Fc domain may comprise at least one mutation at L234A, L235A, G237A, K322A (Eu numbering). In one embodiment, the Fc region comprise mutations at L234A/L235A/G237A/K322A. In one embodiment, the Fc region comprise mutations at L234A/L235A/K322A (Eu numbering). In one embodiment, the Fc domain comprise null mutation. In one embodiment, the IgG4 Fc domain comprises the mutation S228P (Eu numbering). In one embodiment, the IgG4 Fc domain comprises the mutations S228P/F234A/L234A (Eu numbering).

In one embodiment, the heavy chain constant sequence may be derived from IgG1 or IgG4.

In one embodiment, the Fc domain may include an amino acid sequence having at least 50%, 60%, 70%, 75%, 80%, 85%, 90%, 95%, 97%, 98%, 99%, or 100% identity to SEQ ID NO. 313 or 314.

In one embodiment, the first binding monomer may comprise a VH domain, a second binding monomer may comprise a VL domain, and the VH, CH1, VL, CL domains form a Fab region as a third binding domain (D3). In one embodiment, the Fab region may comprise a disulfide bond between VH-44C and VL 100C.

In one embodiment, the first binding monomer and the second binding monomer form a NKG2D receptor as a third binding domain (D3).

In one embodiment, the D1, D2, D4, and D5 is independently a scFv domain, a VHH domain, a receptor, or a ligand.

In one embodiment, the scFv domain may have a VH domain linked to a VL domain. In one embodiment, the scFv domain have may have VH-VL orientation. In one embodiment, the scFv domain have a VL-VH orientation.

In one embodiment, the scFv domain may comprise a disulphide bond between VL and VH. In one embodiment, the disulfide bond is between VL100 and VH44 of the scFv domain. In one embodiment, the scFv domain may comprise the mutation R19S in the VH.

In one embodiment, the VHH domain may comprise the mutation R19S in the VH or VHH.

In one embodiment, at least one, two, or three of D1, D2, D4, and D5 may be scFv. In one embodiment, all of D1, D2, D4, and D5 may be scFv.

In one embodiment, at least one, two, or three of D1, D2, D4, and D5 may be a VHH domain. In one embodiment, all of D1, D2, D4, and D5 may be VHH domains.

In one embodiment, at least one, two, or three of D1, D2, D4, and D5 may be a receptor. In one embodiment, all of D1, D2, D4, and D5 may be receptors.

In one embodiment, at least one, two, or three of D1, D2, D4, and D5 may be a ligand. In one embodiment, all of D1, D2, D4, and D5 may be ligands.

In one embodiment, the D1, D2, D3, D4, and D5 each independently may have a binding specificity against an antigen selected from a T cell activating receptor, an immune cell binding receptor, an immune checkpoint molecule, a co-stimulation factor, a receptor of a leukocyte, a tumor antigen, a tumor associated antigen (TAA), a receptor of a tissue cell, a receptor of a cancer cell, or a combination thereof.

In one embodiment, the binding domain for T cell activating receptor is adjacent to the binding domain for the tumor associated antigen (TAA).

In one embodiment, T cell activating receptor may comprise CD3.

In one embodiment, an immune checkpoint receptor may comprise PD-L1, PD-1, TIGIT, TIM-3, LAG-3, CTLA4, BTLA, VISTA, PD-L2, CD160, LOX-1, siglec-15, CD47, HVEM SIRPα CSF1R, CD73, Siglec-15, CD47 or a combination thereof.

In one embodiment, a co-stimulating receptor may comprise 4-1BB, CD28, OX40, GITR, CD40L, CD40, ICOS, LIGHT, CD27, CD30, or a combination thereof.

In one embodiment, a tumor associated antigen may comprise EGFR, HER2, HER3, EGRFVIII, CD19, BCMA, CD20, CD33, CD123, CD22, CD30, ROR1, CEA, LMP1, LMP2A, Mesothelin, PSMA, EpCAM, glypican-3, gpA33, GD2, TROP2, NKG2D ligand, CD39, CLDN18.2, DLL3, HLA-G, FcRH5, GPRC5D, LIV-1, MUC1, CD138, CD70, uPAR, CD38 or a combination thereof.

In one embodiment, the D1, D2, D3, D4, and D5 each independently may have a binding specificity against an antigen selected, EGFR, HER2, HER3, EGFRvIII, ROR1, CD3, CD28, CEA, LMP1, LMP2A, Mesothelin, PSMA, EpCAM, glypican-3, gpA33, GD2, TROP2, NKG2D, NKG2D ligand, BCMA, CD19, CD20, CD33, CD123, CD22, CD30, PD-L1, PD1, OX40, 4-1BB, GITR, TIGIT, TIM-3, LAG-3, CTLA4, CD40, CD40L, VISTA, ICOS, BTLA, LIGHT, HVEM, CSF1R, CD73, CD39, CLDN18.2, DLL3, HLA-G, FcRH5, GPRC5D, LIV-1, MUC1, CD138, CD70, CD16, uPAR, Siglec-15, CD47, CD38, NKp46, PD-L2, CD160, LOX-1, SIRPα CD27 and the Fc domain may comprise a human IgG Fc domain.

In one embodiment, D1 may have a binding specificity to CD3, CD20, CEA, HER2, EGFR or NKG2D ligand. In one embodiment, D2 may have a binding specificity to HER3, EGFR, CD3, or CD19. In one embodiment, D3 may have a binding specificity to HER3, EGFR, CD3, or NKG2D ligands. In one embodiment, D4 may have a binding specificity to 4-1BB, or EGFR. In one embodiment, D5 may have a binding specificity to PD-L1 or HER3.

In one embodiment, D1 may have a binding specificity to CD3, D2 may have a binding specificity to HER3, D3 may have a binding specificity to EGFR, D4 may have a binding specificity to 4-1BB, and D5 may have a binding specificity to PD-L1.

In one embodiment, D1 may have a binding specificity to EGFR, D2 may have a binding specificity to HER3, D3 may have a binding specificity to CD3, D4 may have a binding specificity to 4-1BB, and D5 may have a binding specificity to PD-L1.

In one embodiment, D1 may have a binding specificity to EGFR, D2 may have a binding specificity to CD3, D3 may have a binding specificity to HER3, D4 may have a binding specificity to 4-1BB, and D5 may have a binding specificity to PD-L1.

In one embodiment, D1 may have a binding specificity to CD3 or EGFR, D2 may have a binding specificity to CD19, D3 may have a binding specificity to CD3 or EGFR, D4 may have a binding specificity to 4-1BB, and D5 may have a binding specificity to PD-L1.

In one embodiment, D1 and D4 each may have a binding specificity to EGFR, D2 and D5 each may have a binding specificity to HER3, and D3 may have a binding specificity to CD3.

In one embodiment, D1 may have a binding specificity to CD20, D2 may have a binding specificity to CD19, D3 may have a binding specificity to CD3, D4 may have a binding specificity to 4-1BB, and D5 may have a binding specificity to PD-L1.

In one embodiment, D1 may have a binding specificity to NKG2D ligand, D2 may have a binding specificity to CD19, D3 may have a binding specificity to CD3, D4 may have a binding specificity to 4-1BB, and D5 may have a binding specificity to PD-L1.

In one embodiment, D1 may have a binding specificity to CD3, and D3 may comprise a NKG2D receptor. In one embodiment, the protein may further comprise D2 having a binding specificity to CD19. In one embodiment, the protein may further comprise D5 having a binding specificity to PD-L1. In one embodiment, the protein may further comprise D4 having a binding specificity to 4-1BB.

In one embodiment, the multi-specific antibody-like protein is bi-specific. In one embodiment, D2 may have a binding specificity to HER3, D3 may have a binding specificity to CD3. In one embodiment, D1 may have a binding specificity to HER2, D3 may have a binding specificity to CD3. In one embodiment, D1 may have a binding specificity to EGFR, D3 may have a binding specificity to CD3. In one embodiment, D3 may have a binding specificity to CD3, D5 may have a binding specificity to HER3. In one embodiment, D3 may have a binding specificity to CD3, D4 may have a binding specificity to EGFR.

In one embodiment, the bi-specific antibody-like protein may include an amino acid sequence having at least 50%, 60%, 70%, 75%, 80%, 85%, 90%, 95%, 97%, 98%, 99%, or 100% identity to SEQ ID NO. 1 and 3; 5 and 7; 9 and 11; 13 and 15; 53 and 55; 57 and 59; 113 and 115; 117 and 119; 121 and 123; 125 and 127; 157 and 159; 297 and 299; or 199 and 201.

In one embodiment, the multi-specific antibody-like protein is tri-specific. In one embodiment, D1 may have a binding specificity to EGFR, D2 may have a binding specificity to HER3, D3 may have a binding specificity to CD3. In one embodiment, D1 may have a binding specificity to CEA, D2 may have a binding specificity to EGFR, D3 may have a binding specificity to CD3. In one embodiment, D3 may have a binding specificity to CD3, D4 may have a binding specificity to EGFR, D5 may have a binding specificity to HER3. In one embodiment, the antibody is a tri-specific antibody having the binding specificity to EGFR, HER3, and CD3. In one embodiment, the tri-specific antibody-like protein may include an amino acid sequence having at least 50%, 60%, 70%, 75%, 80%, 85%, 90%, 95%, 97%, 98%, 99%, or 100% identity to SEQ ID NO. 41 and 43; 45 and 47; 49 and 51; 101 and 103; 105 and 107; 109 and 111; 195 and 197; 137 and 139; or 161 and 163.

In one embodiment, the multi-specific antibody-like protein is tetra-specific. In one embodiment, D1 may have a binding specificity to EGFR, D2 may have a binding specificity to HER3, D3 may have a binding specificity to CD3, D4 may have a binding specificity to 4-1BB. In one embodiment, D1 may have a binding specificity to EGFR, D2 may have a binding specificity to HER3, D3 may have a binding specificity to CD3, D5 may have a binding specificity to PD-L1. the antibody is a tetra-specific antibody having the binding specificity to EGFR, HER3, CD3, and 4-1BB. the antibody is a tetra-specific antibody having the binding specificity to EGFR, HER3, CD3, and PD-L1. In one embodiment, the tetra-specific antibody-like protein may include an amino acid sequence having at least 50%, 60%, 70%, 75%, 80%, 85%, 90%, 95%, 97%, 98%, 99%, or 100% identity to SEQ ID NO. 33 and 35; 37 and 39; 141 and 143; 145 and 147; or 165 and 167.

In one embodiment, the antibody is penta-specific. In one embodiment, the antibody-like protein may have the binding specificity to EGFR, HER3, CD3, 4-1BB and PD-L1. In one embodiment, the penta-specific antibody-like protein may include an amino acid sequence having at least 50%, 60%, 70%, 75%, 80%, 85%, 90%, 95%, 97%, 98%, 99%, or 100% identity to SEQ ID NO. 17 and 19; 21 and 23; 25 and 27; 29 and 31; 69 and 71; 73 and 75; 77 and 79; 81 and 83; 85 and 87; 89 and 91; 93 and 95; 97 and 99; 129 and 131; 133 and 135; 149 and 151; 169 and 171; 173 and 175; or 177 and 179.

In a second aspect, the application provides novel sequences for complimentary determining regions (CDRs). In one embodiment, the application provides proteins, antibody-like proteins or antibodies comprising the CDRs as disclosed herein.

In one embodiment, the CDRs may have an affinity to CEA. In one embodiment, the CDRs may have an equilibrium dissociation constant (KD) to CEA, wherein the KD is not more than 0.1 nM, 2 nM, 5 nM, 10 nM, 20 nM, 30 nM or 50 nM. In one embodiment, the CDR may include an amino acid sequence having at least 80% sequence identity to SEQ ID NO. 301, 302, 303, 304, 305, or 306.

In one embodiment, the application provides a protein having an affinity to CEA. In one embodiment, the protein may have a CDR H1 having an amino acid sequence having at least 80% sequence identity to SEQ ID NO. 301. In one embodiment, the protein may have a CDR H2 having an amino acid sequence having at least 80% sequence identity to SEQ ID NO. 302. In one embodiment, the protein may have a CDR H3 having an amino acid sequence having at least 80% sequence identity to SEQ ID NO. 303. In one embodiment, the protein may have a CDR L1 having an amino acid sequence having at least 80% sequence identity to SEQ ID NO. 314. In one embodiment, the protein may have a CDR L2 having an amino acid sequence having at least 80% sequence identity to SEQ ID NO. 305. In one embodiment, the protein may have a CDR L3 having an amino acid sequence having at least 80% sequence identity to SEQ ID NO. 306. In one embodiment, the protein may include an amino acid sequence having at least 50%, 60%, 70%, 75%, 80%, 85%, 90%, 95%, 97%, 98%, 99%, or 100% sequence identity to an amino acid sequence selected from SEQ ID NO. 279, 280, 281 or 282. In one embodiment, the protein may include an amino acid sequence having at least 50%, 60%, 70%, 75%, 80%, 85%, 90%, 95%, 97%, 98%, 99%, or 100% sequence identity to an amino acid sequence selected from SEQ ID NO. 279, 280, 281 or 282.

In one embodiment, the application provides a multi-specific antibody-like protein having a variable region, wherein the variable region may comprise an amino acid sequence selected from SEQ ID NO. 301, 302, 303, 304, 305, or 306.

In one embodiment, the CDRs may have an affinity to CD3. In one embodiment, the CDR may have an equilibrium dissociation constant (KD) to CD3, wherein the KD is not more than 10 nM, 20 nM, 30 nM or 50 nM, 100 nM, 200 nM, 300 nM, 400 nM or 500 nM. In one embodiment, the CDR may include an amino acid sequence having at least 80% sequence identity to SEQ ID NO. 307, 308, 309, 310, 311 or 312.

In one embodiment, the protein having an affinity to CD3 includes CDR H1 having an amino acid sequence having at least 80% sequence identity to SEQ ID NO. 307. In one embodiment, the protein having an affinity to CD3 includes CDR H2 having an amino acid sequence having at least 80% sequence identity to SEQ ID NO. 308. In one embodiment, the protein having an affinity to CD3 includes CDR H3 having an amino acid sequence having at least 80% sequence identity to SEQ ID NO. 309. In one embodiment, the protein having an affinity to CD3 includes CDR L1 having an amino acid sequence having at least 80% sequence identity to SEQ ID NO. 310. In one embodiment, the protein having an affinity to CD3 includes CDR L2 having an amino acid sequence having at least 80% sequence identity to SEQ ID NO. 311. In one embodiment, the protein having an affinity to CD3 includes CDR L3 having an amino acid sequence having at least 80% sequence identity to SEQ ID NO. 312.

In one embodiment, the protein may comprise an amino acid sequence having at least 50%, 60%, 70%, 75%, 80%, 85%, 90%, 95%, 97%, 98%, 99%, or 100% sequence identity to an amino acid sequence selected from SEQ ID NO. 227-230, 231-234, 235-238, 239-242 and 291-294. In one embodiment, the multi-specific antibody-like protein may comprise an amino acid sequence having at least 50%, 60%, 70%, 75%, 80%, 85%, 90%, 95%, 97%, 98%, 99%, or 100% sequence identity to an amino acid sequence selected from SEQ ID NO. 227-230, 231-234, 235-238, 239-242 and 291-294.

In one embodiment, the multi-specific antibody-like protein may have a variable region wherein the variable region may comprise an amino acid sequence selected from SEQ ID 307, 308, 309, 310, 311 or 312.

In a further aspect, the application provides isolated nucleic acid sequences encoding the multi-specific antibody-like proteins, fragments, domains, regions as disclosed herein.

In a further aspect, the application provides expression vectors including the isolated nucleic acid sequences as disclosed herein.

In a further aspect, the application provides host cells including the isolated nucleic acid sequences as disclosed herein. In one embodiment, the host cell includes the expression vector as disclosed herein. In one embodiment, the host cell may be a prokaryotic cell or a eukaryotic cell.

In a further aspect, the application provides methods for producing the multi-specific antibody like proteins as disclosed herein. In one embodiment, the method includes the steps of culturing a host cell such that the DNA sequence encoding the multi-specific antibody-like protein is expressed, and purifying said multi-specific antibody. In one embodiment, the method includes the steps of culturing a host cell under conditions wherein said multi-specific antibody-like proteins are produced and recovering said antibody-like protein.

In a further aspect, the application provides immunoconjugates. In one embodiment, the immunoconjugate may include the multi-specific antibody-like proteins linked to a cytotoxic agent, an imaging agent, or both.

In a further aspect, the application provides pharmaceutical compositions. In one embodiment, the pharmaceutical composition may include the multi-specific antibody-like protein and a pharmaceutically acceptable carrier. In one embodiment, the pharmaceutical composition may further include radioisotope, radionuclide, a toxin, a therapeutic agent, a chemotherapeutic agent or a combination thereof. In one embodiment, the pharmaceutical composition may include the immunoconjugate as disclosed thereof and a pharmaceutically acceptable carrier.

In a further aspect, the application provides methods for treating or preventing a cancer, an autoimmune disease, or an infectious disease in a subject. In one embodiment, the method may include the steps of administering to the subject a pharmaceutical composition including a purified multi-specific antibody-like protein, an immunoconjugate, or a pharmaceutical composition as disclosed herein. In one embodiment, the method may further include co-administering an effective amount of a therapeutic agent. In one embodiment, the therapeutic agent may comprise an antibody, a chemotherapy agent, an enzyme, or a combination thereof.

In one embodiment, the subject is a human. In one embodiment, the subject is a mammal. In one embodiment, the subject is a chimpanzee. In one embodiment, the subject is a pet animal.

In a further aspect, the application provides a solution including an effective concentration of the multi-specific antibody-like protein, immunoconjugate, or pharmaceutical composition as disclosed herein. In one embodiment, the solution is blood plasma in a subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 depicts the heterodimeric configuration of miniGNC molecules with five binding domains (D1-D5), of which the two monomers are encoded by Chain A (N-D1-D3/VH-CH1-CH2-CH3-D4-C) and Chain B (N-D2-D3/VL-CL-CH2-CH3-D5-C);

FIG. 2 shows the dimerization of engineered miniGNC Chain A and Chain B with knockout mutations (KO) on either VH or Fc: (2A) by Protein-A purification to fractionate the mixture into the homodimer (a), the desired heterodimer (b), and the Chain B monomer (c); and (2B) by SDS-PAGE analysis of Protein-A fractionated homodimer (a), heterodimer (b), Chain B monomer (c), and Chain A monomer (d), at the expected sizes under non-reducing and reducing conditions;

FIG. 3 shows the comparative potency of multi-specific miniGNC molecules mediated TDCC on BXPC3 tumor cells by using penta-miniGNC (SI-75P6), tetra-miniGNC (SI-75E2), tri-miniGNC (SI-75X3), bi-miniGNC (SI-75X2), and mono-miniGNC (SI-7502) molecules;

FIG. 4 shows the comparative potency of miniGNC molecules mediated TDCC on BXPC3 tumor cell line with EC50 values in the range of 0.1 to 5.5 pM;

FIG. 5 shows the survival curves of TDCC assays measuring comparative potency of (A) multi-specific miniGNC molecules with stapled domains, including penta-miniGNC (SI-68P1), tetra-miniGNC (SI-68E2 and SI-68E1), and tri-miniGNC (SI-68x2), for killing pancreatic cancer cells (HPAFII) in TDCC assay, and (B) SI-68X1 against MCF-7 breast cancer cells; and

FIG. 6 shows the functionality of multi-specific miniGNC molecules possessing a dimeric NKG2D receptor, including tri-specific (SI-49R25), tetra-specific (SI-49P_X), penta-specific (SI-49PM1 and SI-49P8) in killing breast cancer cells (MDA-MB-231) in TDCC assay.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibodies, methods of making such antibodies, bispecific or multi-specific molecules, antibody-drug conjugates and/or immuno-conjugates composed from such antibodies or antigen binding fragments, pharmaceutical compositions containing the antibodies, bispecific or multi-specific molecules, antibody-drug conjugates and/or immuno-conjugates, the methods for making the molecules and compositions, and the methods for treating cancer using the molecules and compositions disclosed herein.

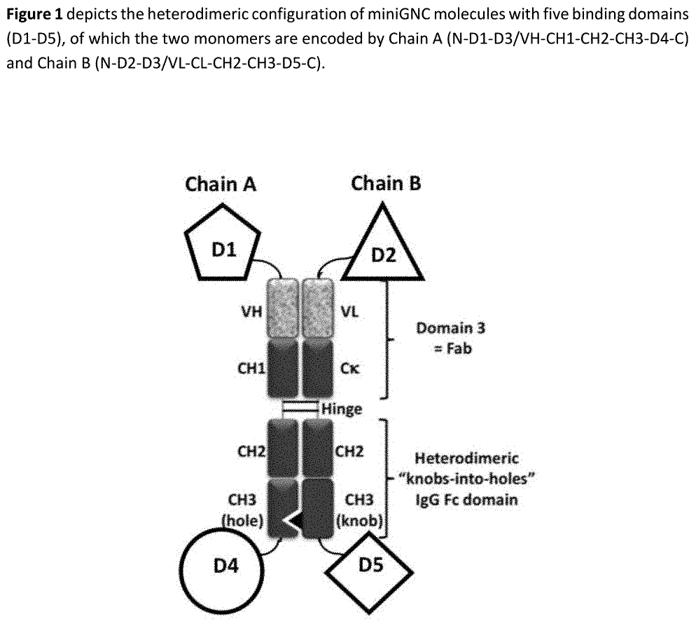

The present application discloses multi-specific Guidance and Navigation Control (GNC) antibodies in miniature configurations (miniGNC). A miniGNC platform molecule is an assembled heterodimer of two polypeptide chains characterized by the formation of a Fab-Hinge-Fc region core structure with one, two, three, or four additional binding domains (from D1 to D5), as elucidated in FIG. 1. The two polypeptides may be configured, for Chain A (or Chain 1): N-D1-D3(VH)-CH1-Hinge-CH2-CH3-D4-C; and for Chain B (or chain 2): N-D2-D3(VL)-CL-Hinge-CH2-CH3-D5-C. The Fc domains of the two chains are engineered to contain complementary mutations, also known as “knobs-into-holes”, to enhance the formation of the heterodimer. A miniGNC platform molecule may have variable components. For example, the VH and VL of the Fab region may be replaced by a non-Fab dimer with or without binding specificity. An additional binding domain may be an antibody fragment, such as scFv or VHH, or a non-antibody structure, such as a ligand or a receptor. A miniGNC platform antibody may be bivalent, trivalent, tetravalent, or pentavalent with up to five distinct specificities, and retains the approximate size of a normal bivalent IgG antibody (tri-miniGNC Ab at 150 kD) or slightly larger (tetra-miniGNC Ab at 175 kD, and penta-miniGNC Ab at 200 kD).

The present application relates to methods of making and using miniGNC antibodies, in particular, penta-specific miniGNC antibodies (penta-miniGNC Ab). In general, GNC proteins, such as GNC antibodies, are characterized by comprising two moieties for engaging immune cells, such as activating T cells, while targeting tumor cells. Similar to GNC antibodies, miniGNC antibodies retain multiple antigen binding domains for engaging immune cells, such as anti-CD3 for T cell activation, anti-4-1BB for co-stimulation, and anti-PD-L1 for inhibiting immune checkpoint. To improve the efficacy of antibody therapy for treating cancer, miniGNC antibodies are designed to be structurally stable and compact while retaining the characteristic feature of two moieties in GNC antibodies. This improvement allows an additional binding specificity to a second tumor associated antigen on the same or different tumor cell. As compared to GNC antibodies, miniGNC contains an Fc domain that allows for FcRn-mediated recycling and half-life extension, as well as facile protein A-based purification. The Fc receptor-mediated immunity may be incorporated if desired. An Fc-containing miniGNC antibody is quite compact, which allows for better developability and increased tumor penetration. GNC antibodies are usually larger than an IgG antibody due to increased number of antigen binding domains (AgBD), which provides spatial flexibility for binding to both a T cell and a tumor cell. On the other hand, miniGNC antibodies retains the same approximate size as human IgG antibodies calculated at approximately 110-130 kD for bi-specific; 120-160 for tri-specific; 130-190 kD for tetra-specific; 140-220 kD for penta-specific; as compared to 150 kD for human IgG. The incorporation of one or two scFvs onto each chain may reduce chain-pairing complications and improve stability while navigating through tumor microenvironment. With these characteristic features, GNC and miniGNC antibodies may be alterative of an efficacious antibody therapy for treating the same cancer, since the moiety for targeting tumor associated antigens remains the same, including but not limited to, EGFR, HER2, HER3, EGRFVIII, CD19, BCMA, CD20, CD33, CD123, CD22, CD30, ROR1, CEA, LMP1, LMP2A, Mesothelin, PSMA, EpCAM, glypican-3, gpA33, GD2, TROP2, NKG2D ligand, CD39, CLDN18.2, DLL3, HLA-G, FcRH5, GPRC5D, LIV-1, MUC1, CD138, CD70, uPAR, CD38.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, human and humanized antibodies as well as active fragments thereof. Examples of active fragments of molecules that bind to known antigens include Fab, F(ab′)₂, scFv and Fv fragments, including the products of aFab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the present disclosure antibodies were created by methods of immunizing rabbits, mice, or llama in combination with subsequent strategies like hybridoma or display. Rabbits are known to create antibodies of high affinity, diversity and specificity (Weber et al. Exp. Mol. Med. 49:e305). Besides immunization of rabbits followed by B cell culture, other common strategies for antibody generation and discovery include immunization of other animals (e.g., mice, llamas) followed by hybridoma and/or display on phage, yeast, or mammalian cells; or display using synthetic variable gene libraries. This general method of antibody discovery is similar to that described in Seeber et al. PLOS One. 9:e86184 (2014).

The term “antigen- or epitope-binding portion or fragment” refers to fragments of an antibody that are capable of binding to an antigen. These fragments may be capable of the antigen-binding function and additional functions of the intact antibody. Examples of binding fragments include, but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen- or epitope-binding fragments” can be derived from an antibody of the present disclosure by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, delta, epsilon, γ, and μ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic a binding specificity.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “affinity” refers to a measure of the attraction between two polypeptides, such as antibody/antigen, receptor/ligand, etc. The intrinsic attraction between two polypeptides can be expressed as the binding affinity equilibrium dissociation constant (KD) of a particular interaction. A KD binding affinity constant can be measured, e.g., by Bio-Layer Interferometry, where KD is the ratio of kdis (the dissociation rate constant) to kon (the association rate constant), as KD=kdis/kon.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to the equilibrium dissociation constant of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion or recombination.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1: Configuration of Penta-Specific miniGNC Antibodies

The design of miniGNC antibodies relies on the co-expression and assembling of two polypeptide chains of a heterodimer between Chain A and Chain B (FIG. 1). While Chain A may be a native heavy chain (HC), Chain B is a recombinant fusion peptide between the light chain and the Fc domain of HC. At the core of this antibody structure, there is a Fab region (VH-CH1/VL-CL) followed by the hinge and Fc region (CH2-CH3/CH2-CH3) of human IgG1. “Knobs-into-holes” mutations or other complementary sets of mutations may be introduced into the CH3 domains to destabilize homodimers and favor heterodimer formation (Ridgway J B, Presta L G, Carter P., Protein Eng 1996; 9:617-21). The “hole” mutations in the CH3 domain of Chain A consist of T366S, L368A, and Y470V, while the “knob” mutation in the CH3 domain of Chain B is T366W. The “knob” created by the T366W mutation on Chain B preferentially interacts with the “hole” created by the three mutations on Chain A. This preferential pairing may further stabilize the heterodimer while the non-covalent pairing of the Fab domain components (i.e. between VH-CH1 on Chain A and VL-Ck on Chain B) creates a functional Fab region. In other words, a functional D3 binding domain, such as a functional Fab region or a non-Fab receptor dimer, can only be created in Chain A-Chain B heterodimers. As compared to GNC antibodies, miniGNC antibodies also contain antibody fragments from native IgG (Fab, Fc) and other variable-sequence based structures, such as scFv and VHH, but are engineered to preferentially configure a Fab region-Hinge-Fc region structure after the formation of a heterodimer. The characteristic feature of miniGNC antibodies is the compactness of this engineered structure.

One or more structurally diversified antigen binding domain may be added to the two N-terminals and the two C-terminals of a miniGNC molecule as D1, D2 D4, or D5, thereby creating up to five binding specificities. Each of these added binding domains may be variable-sequence-based structures, such as scFv and VHH, or non-variable sequence-based structures, such as ligand and receptor. As elucidated in FIGS. 1, D1 and D2 refer to the binding domains linked to the N-terminals of Chain A and Chain B, respectively, whereas D4 and D5 refer to the binding domains linked to the C-terminals of Chain A and Chain B, respectively. Thus, the two polypeptides of a penta-specific miniGNC (penta-miniGNC) antibody may be configured as

Chain A: N-D1-D3(VH)-CH1-Hinge-CH2-CH3-D4-C; and

Chain B: N-D2-D3(VL)-CL-Hinge-CH2-CH3-D5-C;

where VL may be either Vλ or Vκ and CL may be either Cλ or Cκ as shown in FIG. 1.

Depending on the number of binding domains and their binding specificities, the structural configuration of the penta-miniGNC antibody in FIG. 1 may be simplified by reducing one or more binding domains and becoming tetra-, tri-, and bi-miniGNC. For incidence, Table 1 depicts the generation and characterization of a family of anti-CD3 miniGNC antibodies for optimizing their efficacy of targeting EGFR and HER3. In this case, each binding domain exerts one binding specificity. When two of its five binding domains target the same epitope of an antigen, i.e. the same binding specificity, the penta-miniGNC antibody becomes a tetra-specific penta-miniGNC antibody.

### Example 2: Engineered Core Structure of miniGNC Antibodies

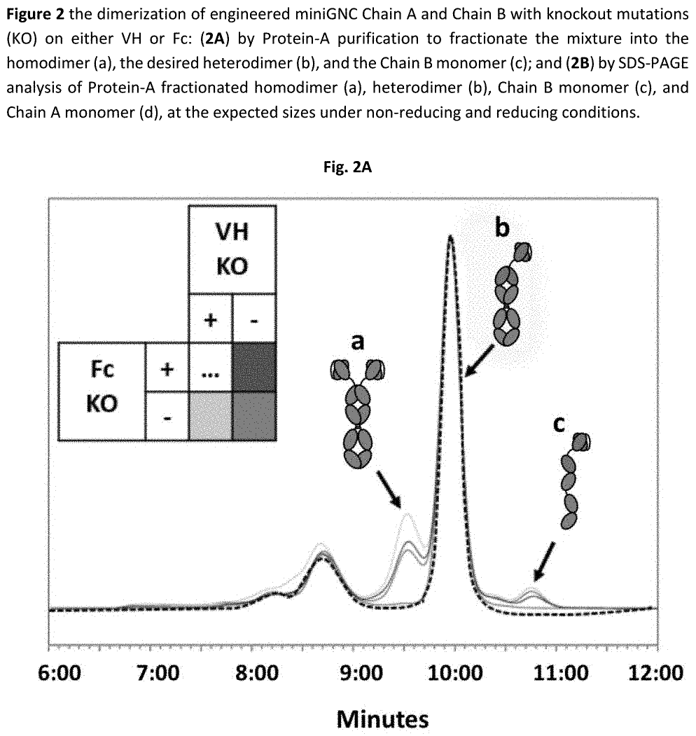

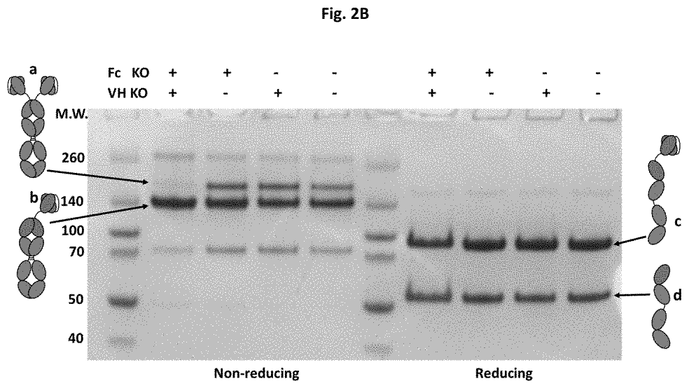

To demonstrate that the configuration of miniGNC may be translated into stable and functional heterodimeric protein, a simplified asymmetric format was designed for evaluating the effect of configuration and mutations to the heterodimer formation of miniGNC molecules. Briefly, a single scFv (D1) is fused to the N-terminus of Chain A, whereas the Chain B does not have any added binding domain. In this way, the proportion of each species of dimerization between Chain A and Chain B may be distinguished by SDS-PAGE and SEC, as the predicted sizes for a Chain A homodimer (not observed), a Chain B homodimer, and a Chain A and Chain B heterodimer are 100 kDa, 150 kDa, and 125 kDa, respectively) (FIG. 2).

To engineer a strategy for selectively purifying the heterodimeric miniGNC molecule from a mixture, two amino acid residues in the CH3 domain (H435 and Y436) of the Chain B are substituted. In particular, the histidine (H435) is critical for the Chain B to bind to protein-A resins during the purification of therapeutic antibodies (Tustian et al., 2016), and the substitution of this amino acid or a ProA knockout mutation (ProAKO) terminates the protein-A binding. This strategy may allow separation of the heterodimer via selective elution from a protein-A column. As the protein-A column retains the heterodimer through its parental heavy chain binding site, the Chain B homodimer flows through as the Chain B lacks protein-A binding. The design of a ProAKO on the Chain B minimizes any possible issue of altering immunogenicity by amino acid substitutions of the protein-A binding site with residues taken from the homologous region in the IgG3 subclass. Although most of miniGNC molecules will predominantly incorporate the structure and sequence of IgG1 subtype, IgG3 has an overall similar structure to other IgG subclasses with some specific structural perturbations in binding to protein-A, protein-G and FcRn (neonatal Fc receptor). This difference is based on Fc sequence difference as revealed earlier where the presence of R435 (H435 in other subtypes) in IgG3 ablates its Fc interaction with protein-A. Hence, in representative miniGNC molecules, amino acids critical for protein-A binding on miniGNC Chain B (H435 and F436) have been mutated to the IgG3 counterpart sequences (R435 and Y436).

In the class of multi-specific miniGNC antibodies with an addition of scFv fused to the N terminal of Chain B, the interaction to protein-A binding is still possible if the scFv comes from VH3 family. VH3 encoded antibodies are known to interact with Staphylococcal Protein A (SPA) and residues in FR1, CDR2 and FR3 have been identified to be involved in SPA binding (Roben, et al., 1995), and structural data indicated that the replacement of a single involved region with the corresponding region from the nonbinding immunoglobulin ablates protein-A binding by VH3 encoded antibodies. Substitution of the amino acid R19 in FR1 was identified to be critical in binding to protein-A and was undertaken with the amino acid sequence (S19) from equivalent region in the VH4 family.

Four sets of miniGNC molecules (SEQ ID 1-16) with four different versions of chain B mutations (SEQ ID 3, 7, 11 and 15) embracing different combinations of ProAKO: H435R/Y436F (Fc) and R19S (VH) were purified and analyzed with analytical size exclusion chromatography. Only protein containing Chain B with both sets of mutations allowed complete removal of Chain B contaminants (FIG. 2A). This VH3 protein A knockout mutation (R19S) together with Fc ProteinA knockout mutations (H435R, F436Y) on Chain B was incorporated in most of the multi-specific antibody formats and used to produce bi-, tri-, tetra- and penta-miniGNC molecules.

To demonstrate the feasibility of generating functional miniGNC proteins with alternative antibody isotypes, an example miniGNC containing IgG4 constant regions was generated. SI-75XM9 (SEQ ID 297-300) is analogous to SI-75X5 (SEQ ID 1-4) bispecific miniGNC, except that the IgG1 CH1, hinge, and Fc were replaced with IgG4 CH1, hinge, and Fc. Notably, both proteins have the same binding domains (anti-HER3 domain in D2 and anti-CD3* domain in D3) and contain the same core structure and mutations (Knobs-into-Holes Fc mutation; R19S in anti-HER3 scFv; and H435R/Y436F mutations in Fc). As shown in Table 8, SI-75XM9 had comparable titer to SI-77X5, and had a significant 19% increase in the percent protein of interest (85.6% POI vs. 72.2% POI) after protein A purification, as assessed by analytical SEC. The reduction in high molecular weight species could be related to the shorter hinge length of IgG4 (12 amino acids instead of 15), which could reduce the propensity for association of higher-order oligomers. Thus, generation of miniGNC proteins with alternative antibody isotypes is not only possible, but can lead to more favorable properties such as decreased aggregation.

### Example 3: Configuration of Multi-Specific miniGNC Antibodies

To generate and characterize the first group of multi-specific miniGNC antibodies, a trio of moiety 1 binding domains, i.e. αCD3, αPD-L1, and α4-1BB, and two moiety 2 binding domains against EGFR and HER3, respectively, were selected for comparison. As shown in Table 1, the group was subdivided into penta-miniGNC, tetra-miniGNC, tri-miniGNC, and bi-miniGNC groups. Of each subgroup, there was at least one miniGNC antibody having the CD3 binding from D3. The penta-miniGNC group includes SI-75P6 (SEQ ID 17, 19, with αEGFR from D3 position), SI-75P4 (SEQ ID 21, 23, with 41BBL, the ligand to 4-1BB receptor, at D4), SI-75P3 (SEQ ID 25, 27), and SI-75P9 (SEQ ID 29, 31, with αHER3 from D3). The tetra-miniGNC group have two antibodies, SI-75E1 (SEQ ID 33, 35) and SI-75E2 (SEQ ID 37, 39) comparing the effect in the presence and absence of either α4-1BB or αPD-L1 domain. The tri-miniGNC group have three antibodies, SI-75X3 (SEQ ID 41, 43), SI-75X16 (SEQ ID 45, 47), and SI-75X18 (SEQ ID 49, 51) for comparing the two moiety 2 binding domains on either N- or C-terminus of the heterodimer. The bi-miniGNC group have three antibodies, SI-75X1 (SEQ ID 53, 55), SI-75X2 (SEQ ID 57, 59), and SI-75X5 (SEQ ID 1, 3), each of which has one moiety 1 binding domain and one moiety 2 binding domain.

The DNA encoding each member of the first group of miniGNC antibodies were cloned into vector pTT5, which were expressed with acceptable titers using ExpiCHO expression systems for 9 days. The miniGNC antibodies were purified with 5 mL MabSelect protein-A columns followed by Size Exclusion using a highload 16/600 200 pg preparative SEC column on either an Akta Avant or Purifier system. SEC aggregates were analyzed using a waters HPLC linked to multi angle light scattering (MALS, Wyatt systems) to identify correct molecular weight by do/dc calculated methods.

### Example 4: Octet Analysis of Binding Affinity

To assess the functionality of miniGNC antibodies, the binding affinity of each domain was determined by using Biolayer Interferometry (Octet 384 system). Octet analysis was used to ensure that the selected miniGNC antibody retains the binding specificity and affinity to all their cognate antigens. Each miniGNC antibody was loaded onto AHC sensors for 180 seconds at 10 ug/ml, followed by a 60-second baseline step, a 180-second association step with 100 nM of commercially purchased human antigen, and a 360-second dissociation step. Samples for all steps were in Octet buffer (PBS containing 0.1% Tween 20 and 1% BSA). Fits were performed using a 1:1 binding model to extract affinity KD values. As shown in Table 1, the binding affinity for each binding domain, as measured by its KD, varies within an acceptable range. For moiety 1 binding, the KD for αCD3 domain may vary between 17.4 and 36.2 nM, for αPD-L1 between 1 and 2.72 nM, and for α4-1BB between 7.51 and 37.3 nM; and for moiety 2 binding, the KD for αEGFR varies between 5.56 and 11.1, and for αHER3 between 112.8 and 185 nM.

### Example 5: Comparative Potency of Multi-Specific miniGNC Antibodies

The first group of multi-specific miniGNC antibodies was subjected to T-cell dependent cellular cytotoxicity (TDCC) assay for measuring the potency for killing cancer cells. With an αCD3 binding domain in all molecules, this group of miniGNC antibodies was equipped to engage T cells, redirect T-cell mediated cytolysis, and ultimately kill target cells.

A luminescence-based T-cell dependent cellular cytotoxicity (TDCC) assay was used to measure the extent of antibody-induced cellular cytotoxicity by quantification of cell viability via constitutive expression of luciferase. Luciferised BXPC3 human pancreatic cancer cell lines (ATCC, Manassas, Va.) were cultured at 37° C., 5% CO2 in RPMI (ATCC, Manassas, Va.) medium with 10% heat-inactivated fetal bovine serum (Invitrogen, Waltham, Mass.). Cell viability was monitored with a Vi-CELL automated cell counter (Beckman Coulter, Pasadena, Calif.). Target cell surface expression was measured by flow cytometry. Human pancreatic cancer cells, BXPC3 cells, were co-cultured with human pan T-cells at an effector-to-target (E:T) ratio of 5:1 and antibodies were added with a 5-fold dilution series (0-30 nM). Using a Multidrop bulk liquid dispenser (BIOTEK, Winooski, Vt.), 500 cells per well of target cells (20 μL per well) and 25,00 cells per well of T-cells (20 μL/well) were plated sequentially into 384-well, white, flat-bottom, polystyrene TC-treated microplates (Corning, Corning, N.Y.). Antibody dilutions were added (10 μL/well) and plates were incubated for 72 hrs at 37° C., 5% CO2 before luminescence-based cell viability quantification. 20 ul of Bright-Glo (Promega) was added to wells, and luminescence corresponding to viability of tumor cells was determined using a CLARIOstar plate reader.

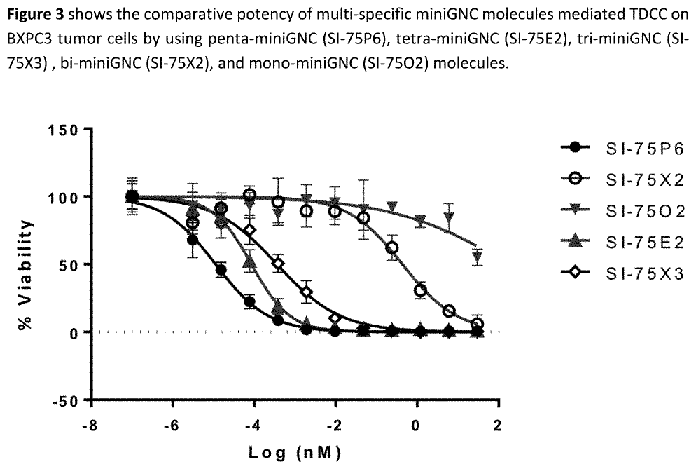

Data were fit to a sigmoidal function to calculate and list EC50 values in Table 1 for comparison. FIG. 3 shows the dose-dependent cell viability curves of miniGNC antibodies selected from a penta-miniGNC (SI-75P6), a tetra-miniGNC (SI-75E2), a tri-miniGNC (SI-75X3), a bi-miniGNC (SI-75X2), and a mono-miniGNC (SI-7502) molecule. While all multi-specific miniGNC antibodies were capable of killing BXPC3 cells completely, both pent- and tetra-miniGNC antibodies steadily displayed high potency in the range of EC50 in pM. In this regard, SI-75X1 was exceptionally potent. The data demonstrate the important roles of moiety 1 binding domains, in particular, the αPD-L1 domain as an immune checkpoint inhibitor when targeting BXPC3 cancer cells.

### Example 6: Configuration of CD3 Binding Domain in miniGNC Antibodies

By definition, a miniGNC antibody is capable of interfacing with at least one immune effector cell and one target cancer cell through the binding of its moiety 1 and 2 binding domains, respectively. Using the general scheme of miniGNC antibody configuration (FIG. 1), it is possible that assigning two moiety 2 binding domains to D1 and D2 may lead to different efficacy from assigning them to D1 and D4 or D2 and D5 because the relative position of the targets on the cell surface is different, either on the top and bottom or on both sides. In this context, a second group of penta-miniGNC antibodies were configured and listed in Table 2. This group of minGNC antibodies share the same binding specificities to CD3, CD19, EGFR, 4-1BB, and PD-L1, and the same binding domains at D2, D4, and D5. SI-68P13 (SEQ ID 69, 71) has αCD3 D3 domain, which is comparable to SI-75P3 of the first group except the difference in D2, i.e. αCD19 vs. αHER3, and to SI-68P17 (SEQ ID 73, 75) of the second group except the switch of αCD3 domain to D1. The rest of the second group of miniGNC antibodies share the same binding specificities from D1 (αCD3) and D3 (αEGFR). SI-68P17 (with the binding specificity to CD19, 4-1BB and PD-L1, respectively.

Anti-CD3 antibody plays a central role in T cell activation based immune therapy. Humanized antibody is desirable for the development of a therapeutic antibody. CD3 domain was incorporated into positions D1 and D3 to generate SI-68P17 (SEQ ID 73, 75) and SI-68P13 (SEQ ID 69, 71) for testing positional effect. The anti-CD3 sequences in humanized framework 1 (CD3**), 2 (CD3***) 3 (CD****) and 4 (CD*****) were cloned into an expression cassette for producing SI-68P15 (SEQ ID NO. 77, 79), SI-68P18 (SEQ ID 81, 83), SI-68P19 (SEQ ID 85, 87), and SI-68P16 (SEQ ID 89, 91) (Table 2). Each expression cassette was transfected into 25 mL of ExpiCHO and expressed for 8 days followed by protein-A affinity chromatography for harvesting and purifying each penta-miniGNC antibody. The antibodies were produced with good titer (Table 2). Octet was used to verify that the penta-miniGNC antibodies containing different CD3 domains, can bind to human CD3, respectively (Table 2). Each penta-miniGNC antibody was loaded via AHC sensors at 10 μg/ml and bound to a serial dilution (highest 200 nM, 1:2.5 dilutions) or a single 100-nM concentration of His-tagged human CD3. The resulting global fit to a 1:1 binding model demonstrated that the penta-GNC antibodies bind to CD3 with affinities in the low nanomolar range (Table 2).

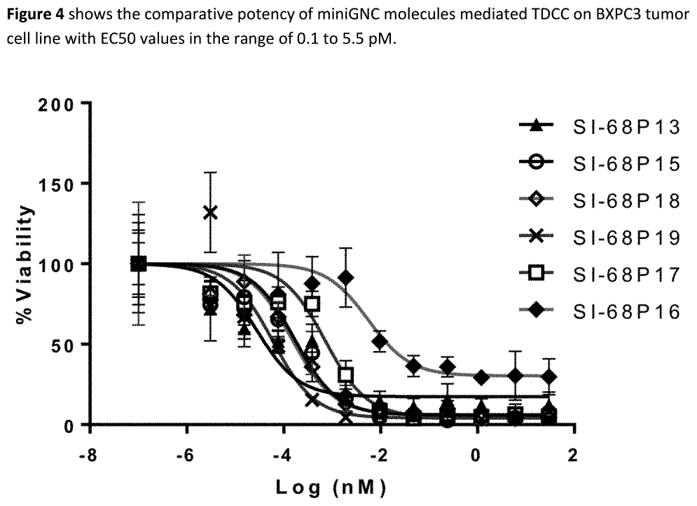

To evaluate the effect of CD3-mediated T cell directed cytotoxicity on cancer cells, BXPC3 tumor cell line was used as target. Serial dilutions (0 to 30 nM; 1 to 5 dilution factor) of antibodies were added to a white 384-well plate containing luciferized target cells and activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. Data were fit to a sigmoidal function to calculate EC50 values, which were in the range of 0.1 to 5.5 pM as shown by FIG. 4 and listed in Table 2. The data demonstrate that there are certain degrees of flexibility in anti-CD3 positioning, configuration, and binding affinity.

### Example 7. Multi-Specific miniGNC Antibodies Having VHH as Binding Domains

To multi-specific antibody platforms, such as GNC and miniGNC antibodies that contain multiple scFv domains, a potential issue is related to chain mispairing due to multiple VH and VL domains in the same molecule. Different VH and VL domains have different propensities to interact with each other. If binding domains of different specificity have superior interaction energy, then the wrong pairings may form when the protein assembles. As a result, none of the binding domains will bind their targeted antigens.

VHH refers to “antibodies” with single Ig domains, i.e. “heavy chain only”, also known as a single-domain antibody (sdAb) or a nanobody. The first single-domain antibodies were engineered from heavy-chain antibodies found in camelids, also known as V_{H}H fragment (VHH).

VHH domains have been increasingly incorporated into antibody-based therapeutics due to several favorable properties. VHH domains may have increased stability and solubility compared to more traditionally used scFv domains. Whereas the VH/VL interface of traditional antibody variable regions is hydrophobic, the transient exposure of these surfaces can result in significant aggregation or precipitation. On the other hand, VHH domains have a naturally more hydrophilic surface and therefore can have superior solubility and stability properties. An obvious benefit of VHH domains over Fab or scFv domains is their small size. Where Fab domains are about 50 kDa and scFv domains are about 25 kDa, VHH domains are a very compact 12-15 kDa. The penta-miniGNC antibodies contain five binding domains (FIG. 1). Incorporation of VHH domains instead of scFv domains may significantly decrease molecular size, and increase tumor penetration.

A third group of multi-specific miniGNC antibodies were created and configured to have VHH binding domains against EGFR and HER3 (Gottlin et al., 2009; Eliseev et al., 2018) (Table 3). Penta, Tri, and bi specific single domain variable regions (VHH)-incorporated miniGNC molecules were constructed and purified with reasonable titer. Binding activity of each domain of multi-specific miniGNCs: SI-75P5 (SEQ ID 93, 95), SI-75P8 (SEQ ID 97, 99), SI-75X4 (SEQ ID 101,103), SI-75X17 (SEQ ID 105, 107), SI-75X19 (SEQ ID 109, 111), SI-75X9 (SEQ ID 113-115), SI-75X11 (SEQ ID 117,119), SI-75X15 (SEQ ID 121,123) and SI-75X13 (SEQ ID 125, 127) were verified using Octet. AHC sensors were used to capture the GNC antibody at a concentration of 10 ug/ml for 180 seconds. After a 60-second baseline step, a single concentration of antigen was used for a 180-second association step. Antigens tested include 200 nM human EGFR (purified in-house), 100 nM human CD3 (Acro CDD-H52W1), 20 nM human PD-L1 (Acro PD1-H5229), 400 nM human 4-1BB (purified in-house), 200 nM human HER3 (Acro ER3-H5223). After association, a 420-second dissociation step was used. The tabulated K_{D} values were calculated using a one-to-one binding model in ForteBio Data Analysis software version 11. The data demonstrated that all domains retained high affinity in the VHH-incorporated miniGNC platform.

T cell directed cytotoxicity on cancer cells, BXPC3 tumor Cell line was used as target. Serial dilutions (0 to 30 nM; 1 to 5 dilution factor) of antibodies were added to a white 384-well plate containing luciferized target cells and activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. EC50 values are tabulated in Table 3.

### Example 8. Engineered Fab (D3) Domain for Improved Expression and Protein Quality

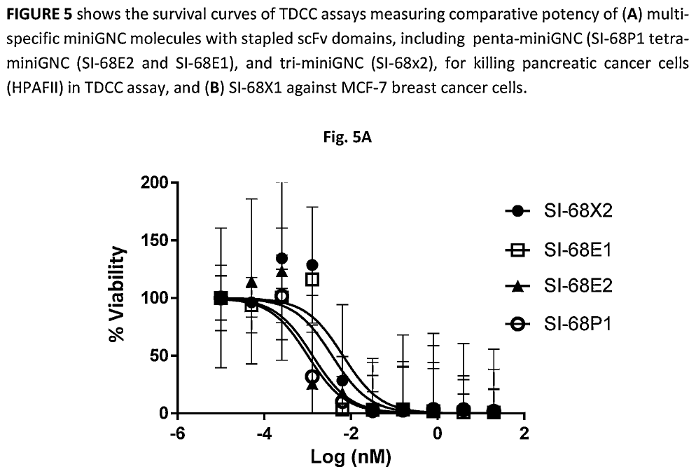

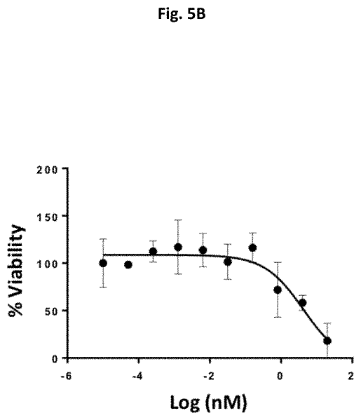

A disulphide staple was optionally introduced in the VH-VL interface of the fab region (FIG. 5). The purpose of this engineering was improving heterodimer pairing and stabilize the overall structure by the formation of a covalent disulphide bond (Weatherill et al., 2012) at the core-fab interface. Cysteines pair was optionally introduced to form disulphide bond at V_{L}A100 on Chain B and its corresponding V_{H}A44 on Chain A fab region.

A pair of penta-miniGNC antibodies (SI-38P11 and SI-76PM1) (SEQ ID NO. 129 and 131; 133 and 135) with identical binding specificities were created for analysing the effect of stapled variable region in the Fab. The Chain A of the two antibodies comprises αCD20 scFv at D1, αCD3 VH at D3 (VH Fab-CH1-Fc), and α4-1BB at D4, whereas Chain B comprises αCD19 at D2, αCD3V_{L} at D3 (VKappa Fab CL-Fc) and αPD-L1 scFv at D5 (Table 4) according to the naming system in FIG. 1. SI-76PM1 was constructed with an additional disulphide in the D3 (VH/VL Fab-CH1/CL-Fc) and SI-38P11 was constructed without any additional disulphide pairing in the VH/VL fab.

Penta-miniGNC antibody constructs with and without the disulphide staple at position D3 were expressed in ExpiCHO system. Construct with stapled D3 (SI-76PM1) was produced with significantly higher titer than SI-38P11. Both proteins were purified with 5 mL MabSelect protein A columns followed by Size Exclusion using a hiload 16/600 200 pg preparative SEC column on either an Akta Avant or Akta Pure Purifier system. SEC aggregates were analyzed using a waters HPLC linked to multi angle light scattering (MALS, Wyatt systems) to identify correct molecular weight by do/dc calculated methods. With all of the analyses conducted as shown in Table 4, the disulfide bonded, i.e. “D3 stapled Fab”, penta-miniGNC antibodies displayed 5% higher protein of interest production. Both molecules exhibited comparable antigen binding kinetics (Table 4).

### Example 9. Multi-Specific miniGNC Antibodies with Stapled scFv Binding Domains

The core structure of multi-specific miniGNC molecules were engineered to acquire several features in order to stabilize the heterodimer, including the covalently linked hinge and non-covalent and preferential interaction of “knobs-into-holes” by the CH3 domains in Fc region. While the stability and compactness are desirable, it remains unclear whether the closer proximity of D1 and D2 or D3 and D4 may affect their stability and independent function. To maintain the stability and independence of each added binding domains, one option is to introduce a disulfide bond at V_{L}100 and V_{H}44 in each scFv domain, i.e. to staple each scFv domain. A disulfide bond between V_{L} and V_{H} may be used for all scFv domains to stabilize the overall structure. Alternatively, a disulphide bond may be introduced into at least one selected scFv domain at any position.

Four miniGNC antibodies have their D1 and D2 targeting EGFR and HER3 were grouped for measuring comparative potency of TDCC to the pancreatic cancer cells (HPAF-II), including tri-miniGNC (SI-68X2, SEQ ID NO. 137,139), tetra-miniGNC (SI-68E1, SEQ ID NO, 141, 143, and SI-68E2, SEQ ID NO. 145, 147), and penta-miniGNC (SI-68P1, SEQ ID NO. 149, 151) molecules. The moiety 1 binding specificities to 4-1BB and PD-L1 were the variable, whereas CD3 was the constant as D3 in the group interms of multispecificity. The expression construct encoding each of these four miniGNC antibodies was modified such that all scFv domains carrying a disulfide bond. The constructs were expressed individually in ExpiCHO system and each antibody was purified with reasonable titer. The binding kinetics to respective antigens were verified using Octet. AHC sensors were used to capture the miniGNC antibody at a concentration of 10 ug/ml for 180 seconds. After a 60-second baseline step, a single concentration of antigen was used for a 180-second association step. Antigens tested included 200 nM human EGFR (purified in-house), 100 nM human CD3 (Acro CDD-H52W1), 20 nM human PD-L1 (Acro PD1-H5229), 400 nM human 4-1BB (purified in-house), 200 nM human HER3 (Acro ER3-H5223). After association, a 420-second dissociation step was used. The tabulated K_{D} values were calculated using a one-to-one binding model in ForteBio Data Analysis software version 11. Analysed KD values for each miniGNC molecule with all stapled scFv domains, as shown in Table 5, were compared with KDs of monoclonal antibody controls for respective antigens as shown in Table 6. The binding kinetics data shows comparable binding affinity of each stapled scFv domain with its respective mAb or Fc-scFv control SI-1C3 (SEQ ID 181, 183) SI-1C7 (SEQ ID 185), SI-9C21 (SEQ ID 187, 189), SI-35SF11 (SEQ ID 191), SI-3SF11 (SEQ ID 193), SI-20C14 (SEQ ID 287 and 289) (Table 6), suggesting that stapling one or more scFv domains seems to have little effect to the binding affinity.

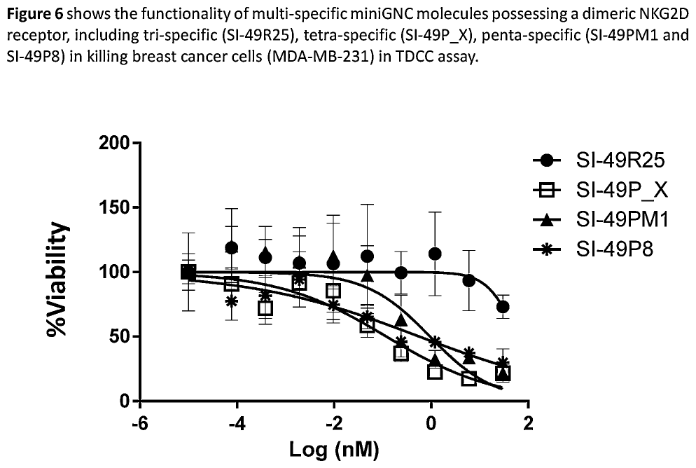

To evaluate the effect of stapled scFv domains to the potency of the miniGNC antibody, T cell directed cytotoxicity (TDCC) assay was used and the target cells were HPAF-II, human a human pancreatic cancer cell line (ATCC, Manassas, Va.). The surface expression of the target cells was validated by flow cytometry. Serial dilutions (0 to 30 nM; 1 to 5 dilution factor) of antibodies were added to a white 384-well plate containing target cells and activated T cells (plated immediately before drug; effector:target=5:1) in a total volume of 50 ul. After an additional 72 hours, 20 ul of Bright-Glo (Promega) was added to wells, and luminescence corresponding to viability of luciferised tumor cells was determined using a CLARIOstar plate reader. Data were fit to a sigmoidal function to calculate EC50 values. As shown in FIG. 6 and Table 5, the survival curves revealed that the potency of all four miniGNC antibodies was be within the dose range of pM and with a tendency of the penta-miniGNC antibody exerting higher TDCC potency than that of tetra-miniGNC and tri-miniGNC antibodies. This observation again shows little effect by using stapled scFv domains.

### Example 10. miniGNC Having NKG2D Receptor Dimer as a Binding Domain

An increased number of binding specificities allows the GNC antibodies to bind not only T cells but also subsets of T cells, natural killer cells, and other types of immune cells, collectively known as moiety 1 binding domains/specificities (see Applicant's applications, WO/2019/005641 and WO2019191120, incorporated herein in its entirety). Some of moiety 1 binding specificities may replace the cellular response to or recognition of targeted cells. For example, NKG2D is a major recognition receptor for the detection and elimination of transformed and infected cells, as its ligands are induced during cellular stress, either as a result of viral infection or genomic stress, such as in cancer. In humans, NKG2D is expressed by NK cells, cells, and CD8+ T cells. The addition of NKG2D as a binding domain/specificity to the class of miniGNC antibodies may improve the cytotoxicity and efficacy of the antibody as a single multi-functional therapeutic agent.

In NK cells, NKG2D serves as an activating receptor, which itself can trigger cytotoxicity, whereas on CD8⁺ T cells the function of NKG2D is to send co-stimulatory signals to activate them. NKG2D forms a homodimer whose ectodomains serve for ligand binding. This feature qualifies NKG2D as a non-variable-sequence-based binding domain in a miniGNC format and other binding domains can be added to create a class of multi-specific NKG2D-miniGNC protein. In one miniGNC format, individual NKG2D monomer was incorporated in the D3 position on chain A and chain B which formed a dimeric NKG2D receptor on Fc dimerization. Thus, NKG2D can act as a receptor for the multi-specific miniGNC molecule to bind its ligand. In other miniGNC format, a NKG2D tandem repeat was designed by adding a (GxSy)n spacer/linker between individual NKG2D monomers which homodimerizes and forms a functional dimeric receptor. This NKG2D tandem dimeric structure can be positioned in D1, D2, D4 or D5.

As listed in Table 7, this class includes mono-NKG2D-miniGNC (SI-49R26, SEQ ID NO. 153, 155), bi-miniGNC (SI-49R27, SEQ ID NO. 157, 159), tri-miniGNC (SI-49R25, SEQ ID NO. 161, 163), tetra-miniGNC (SI-49P_X, SEQ ID NO. 165, 167), and penta-mini-GNC molecules (SI49P8, SI-49P9, SEQ ID. NO. 169, 171, 177, and 179). A control penta-miniGNC, SI-49PM1 (SEQ ID NO. 173, 175), was created by switching the positions of D1 (αCD3) and D3 of SI-49P8, and the Octet binding affinity for either NKG2D or CD3 was not affected by this switch. While the binding affinity of other moiety 1 binding domains remained stable in this class of NKG2D-miniGNC molecules, the binding affinity of NKG2D was within 2-fold. Thus, the penta-miniGNC molecule possesses the binding function of NKG2D receptor for binding to NKG2D ligand.

To assess and compare the potency of TDCC of NKG2D-miniGNC molecules, tri-, tetra-, and penta-miniGNC molecules, SI-49R25, SI-49P_X, and SI-49P8 and SI-49PM1 were used to target MDA-MB-231 breast cancer cell line. Serial dilutions (0 to 30 nM; 1 to 5 dilution factor) of the miniGNC protein were added to a white 384-well plate containing luciferised MDA-MB-231 cells 24 hours prior and grown at 37° C. Activated T cells were plated immediately before miniGNC molecules (effector:target=15:1) in a total volume of 50 ul. After incubated for additional 72 hours, 20 ul of Bright-Glo (Promega) was added to wells, and the luminescence corresponding to viability of luciferised tumor cells was determined using a CLARIOstar plate reader. Data were fit to a sigmoidal function to calculate EC50 values (FIG. 7). Based on the dose-viability curves, there were two groups, the tetra- and penta-miniGNC molecules were more potent than the tri-miniGNC molecule, SI-49R25. As listed in Table 7, the difference might result from the addition of αPD-L1. However, the EC50 value (59.3 nM) of SI-49R25 should be considered as relatively potent. To the breast cancer cell line MDA-MB-231, CD19, which is a pan-B cell marker, is a non-participating tumor antigen. In this regard, SI-49R25 may be viewed as having two binding specificities to CD3 and NKG2D ligand. Thus, the data indicates that the incorporation of NKG2D receptor to different miniGNC antibody format contributes to the potency of TDCC. In a broader sense, the miniGNC antibody molecules can accommodate multiple binding specificities to modulate, cooperate, and direct an optimized immune response to targeted cells, such as cancer.

### TABLES

TABLE 1
The configuration, production, binding affinity, and potency
of multi-specific minGNC molecules when targeting EGFR
and/or HER3-expressing pancreatic cancer cells (BXPC3).

| | | | | | | | |
|---|---|---|---|---|---|---|---|
| Protein | D1/KD | D2/KD | D3/KD | D4/KD | D5/KD | Titer | EC50 |
| ID | (nM) | (nM) | (nM) | (nM) | (nM) | (μg/ml | (pM) |

Penta-miniGNC

| | | | | | | | |
|---|---|---|---|---|---|---|---|
| SI-75P6 | αCD3 | αHER3 | αEGFR | α4-1BB | αPDL-1 | 96.5 | 0.011 |
| | 36.2 | 138 | 5.56 | 37.3 | 1.45 | | |
| SI-75P4 | αEGFR | αHER3 | αCD3 | α4-1BBL | αPDL-1 | 48.4 | 0.102 |
| | 10.4 | 150 | 22.7 | 115 | 2.72 | | |
| SI-75P3 | αEGFR | αHER3 | αCD3 | α4-1BB | αPDL-1 | 39.4 | 1.300 |
| | 8.91 | 158 | 17.4 | 7.51 | 1 | | |
| SI-75P9 | αEGFR | αCD3 | αHER3 | α4-1BB | αPDL-1 | 30 | 0.160 |
| | 11.1 | 20.1 | 140 | 21.2 | 2.7 | | |

Tetra-miniGNC

| | | | | | | | |
|---|---|---|---|---|---|---|---|
| SI-75E1 | αEGFR | αHER3 | αCD3 | α4-1BB | — | 30.2 | 5.800 |
| | 8.9 | 185 | 20.3 | 17.8 | — | | |
| SI-75E2 | αEGFR | αHER3 | αCD3 | — | αPDL-1 | 172.3 | 0.085 |
| | 8.24 | 114 | 22.1 | — | 1.36 | | |

Tri-miniGNC

| | | | | | | | |
|---|---|---|---|---|---|---|---|
| SI-75X3 | αEGFR | αHER3 | αCD3 | | | 193.5 | 5417 |
| | 8.47 | 146 | 18.2 | — | — | | |
| SI-75X16 | αEGFR | αHER3 | αCD3* | | | 183.5 | 95540 |
| | 6.8 | 112.8 | 33.4 | — | — | | |
| SI-75X18 | | | αCD3* | αEGFR | αHER3 | 38.2 | 6090 |
| | | | 47.3 | 2.72 | 93.75 | | |

Bi-miniGNC

| | | | | | | | |
|---|---|---|---|---|---|---|---|
| SI-75X1 | αEGFR | | αCD3 | | | 246.1 | 0.71 |
| | 8.11 | | 24 | — | — | | |
| SI-75X2 | | αHER3 | αCD3 | | | 237.7 | 433.8 |
| | | 162 | 27.8 | — | — | | |
| SI-75X5 | | αHER3 | αCD3* | | | 192.9 | 9.095 |
| | | 139.2 | 44.4 | — | — | | |

TABLE 2
The effect of the position and binding affinity of the CD3
binding domain on the potency of penta-minGNC molecules when
targeting EGFR-expressing pancreatic cancer cells (BXPC3).

| Protein | D1/KD | D2/KD | D3/KD | D4/KD | D5/KD | Titer | EC50 |
|---|---|---|---|---|---|---|---|
| ID | (nM) | (nM) | (nM) | (nM) | (nM) | (μg/ml) | (pM) |
| SI-68P13 | αEGFR | αCD19 | αCD3 | α4-1BB | αPD-L1 | 35.1 | 0.09 |
| | 5.64 | 4.33 | 25.9 | 14.8 | 1.04 | | |
| SI-68P17 | αCD3 | αCD19 | αEGFR | α4-1BB | αPD-L1 | 40 | 0.6 |
| | 15.82 | 5.05 | 5.38 | 26.8 | 2.72 | | |
| SI-68P15 | αCD3** | αCD19 | αEGFR | α4-1BB | αPD-L1 | 58.1 | 0.16 |
| | 22.4 | 2.85 | 2.42 | 17.1 | 0.4 | | |
| SI-68P18 | αCD3*** | αCD19 | αEGFR | α4-1BB | αPD-L1 | 26 | 0.19 |
| | 14.46 | 1.35 | 8.18 | 7.51 | 1.00 | | |
| SI-68P19 | αCD3**** | αCD19 | αEGFR | α4-1BB | αPD-L1 | 28 | 0.12 |
| | 16.74 | 3.44 | 4.43 | 21.2 | 0.7 | | |
| SI-68P16 | αCD3***** | αCD19 | αEGFR | α4-1BB | αPD-L1 | 72 | 5.5 |
| | 60.6 | 2.55 | 2.41 | 23.3 | 0.5 | | |

TABLE 3
The effect of VHH single binding domain(s) on the potency of multi-
specific minGNC molecules when targeting BXPC3 tumor cells

| Protein | D1/KD | D2/KD | D3/KD | D4/KD | D5/KD | Titer | EC50 |
|---|---|---|---|---|---|---|---|
| ID | (nM) | (nM) | (nM) | (nM) | (nM) | (μg/ml) | (pM) |
| SI-75P5 | αEGFR VHH | αHER3 VHH | αCD3 | α4-1BB | αPD-L1 | 200.5 | 443 |
| | 23.5 | 4.68 | 26.1 | 23.3 | 3.25 | | |
| SI-75P8 | αEGFR VHH | αHER3 VHH | αCD3* | αEGFR | αHER3 | 295 | 967468000 |
| | 6.22 | 0.786 | 44.1 | 6.22 | 0.786 | | |
| SI-75X4 | αEGFR VHH | αHER3 VHH | αCD3 | — | — | 65.8 | 5766 |
| | 51.5 | 7.68 | 26.3 | — | — | | |
| SI-75X17 | αEGFR VHH | αHER3 VHH | αCD3* | — | — | 330 | No killing |
| | 14.8 | 4.58 | 68 | — | — | | |
| SI-75X19 | — | — | αCD3* | αEGFR VHH | αHER3 VHH | 223.4 | 1873 |
| | | | 64.4 | 20.6 | 10.52 | | |
| SI-75X9 | — | αHER3 VHH | αCD3* | — | — | 234 | 2584 |
| | — | 3.86 | 84.2 | — | — | | |
| SI-75X11 | αEGFR VHH | — | αCD3* | — | — | 214 | 564 |
| | 21.6 | — | 65.7 | — | — | | |
| SI-75X15 | — | — | αCD3* | — | αHER3 VHH | 161.3 | 27400 |
| | — | | 38.08 | — | 13.7 | | |
| SI-75X13 | — | — | αCD3* | αEGFR VHH | — | 165.6 | 4858 |
| | | — | 56 | 34− | — | | |

TABLE 4
The effect of “stapled” CD3 binding domain at D3 position
on the production of multi-specific miniGNC molecules.

| | | | D3 | | | | |
|---|---|---|---|---|---|---|---|
| | D1/KD | D2/KD | (Stapled)/ | D4/KD | D5/KD | | |
| Protein | (nM) | (nM) | KD (nM) | (nM) | (nM) | Titer | |
| ID | αCD20 | αCD19 | αCD3 | α4-1BB | αPD-L1 | (μg/ml) | % POI |
| SI-38P11 | 7.33 | 1.89 | 5.23 (No) | 14.8 | 1.1 | 80 | 69 |
| SI-76PM1 | 6.09 | 3.30 | 3.22 (Yes) | 22.43 | 1.47 | 129 | 75 |

TABLE 5
Comparative potency of multi-specific miniGNC molecules with “stapled”
scFv at all positions when targeting tumor cells.

| | D1 | D2 | | D4 | D5 | | |
|---|---|---|---|---|---|---|---|
| | (Stapled)/ | (Stapled)/ | D3/KD | (Stapled)/ | (Stapled)/ | | EC50 |
| Protein | KD (nM) | KD (nM) | (nM) | KD (nM) | KD (nM) | Titer | (pM) |
| ID | αEGFR* | αHER3 | αCD3 | α4-1BB | αPD-L1 | (μg/ml) | HPAF-II |
| SI-68X2 | <0.001 | 114.4 | 24.3 | — | — | 88.9 | 6.6 |
| SI-68E1 | <0.001 | 138.8 | 26.2 | 12.7 | — | 114.8 | 3.6 |
| SI-68E2 | <0.001 | 120 | 28.4 | — | 0.4 | 67 | 1.3 |
| SI-68P1 | <0.001 | 93.4 | 24.3 | 9.8 | 0.6 | 87.1 | 1.02 |
| | αCEA | αEGFR* | αCD3 | | | | MCF-7 |
| SI-68X1 | <0.001 | <0.001 | 14.3 | — | — | 69 | 4.2 |
| | αHER2 | — | αCD3 | | | | |
| SI-68X3 | NA | | NA | | | 57 | NA |

TABLE 6
Characterization of the unstapled versions of each domain
(D1-D5) in mAb or scFv format to respective antigens.

| Protein | Unstapled | Domain | Titer | KD |
|---|---|---|---|---|
| ID | Controls | Structure | (μg/ml) | (nM) |
| SI-1C3 | D1 = αEGFR* | mAb | 44.9 | <0.001 |
| SI-1C7 | D2 = αHER3 | Fc-scFv | 290.7 | 136.5 |
| SI-9C21 | D3 = αCD3 | mAb | 252.1 | 26.3 |
| SI-35FS11 | D4 = α4-1BB | Fc-ScFv | 7.67 | 14.9 |
| SI-3FS11 | D5 = αPD-L1 | Fc-ScFv | 18 | 0.94 |
| SI-20C14 | D1 = αCEA | mAb | NA | <0.001 |

TABLE 7
The characterization of multi-specific minGNC molecules with NKG2D
receptor dimer incorporated in miniGNC format and potency of tri,
tetra and penta molecules in killing MDA-MB-231 tumor cells

| Protein | D1/KD | D2/KD | D3/KD | D4/KD | D5/KD | Titer | EC50 |
|---|---|---|---|---|---|---|---|
| ID | (nM) | (nM) | (nM) | (nM) | (nM) | (μg/ml) | (pM) |
| SI-49R26 | — | — | NKG2D | — | — | 28 | n.a. |
| | — | — | 17.7− | — | — | | |
| SI-49R27 | αCD3 | — | NKG2D | — | — | 38.6 | n.a. |
| | 32.2 | — | 16 | — | — | | |
| SI-49R25 | αCD3 | αCD19 | NKG2D | — | — | 50 | 59330 |
| | 34.50 | 7.62 | 12.80 | — | — | | |
| SI-49P_X | αCD3 | αCD19 | NKG2D | — | αPD-L1 | 8.8 | 119.5 |
| | 30.45 | 1.31 | 10.4 | — | 0.2 | | |
| SI-49P8 | αCD3 | αCD19 | NKG2D | α4-1BB | αPD-L1 | 62.3 | 570.3 |
| | 36.3 | 2.98 | 24.3 | 7.71 | 1.08 | | |
| SI-49PM1 | NKG2D | αCD19 | αCD3 | α4-1BB | αPD-L1 | 30.3 | 946.5 |
| | 24.49 | 5.5 | 30.2 | 2.74 | 2.23 | | |
| SI-49P9 | αCD3 | αCD19 | NKG2D | 41BBL | αPD-L1 | 28.9 | n.a. |
| | 29.80 | 6.4 | 27.2 | 112 | 2.01 | | |

TABLE 8
The characterization of bispecific miniGNC molecules
with different IgG subtypes in the Fc domain.

| | IgG | | | | | | Titer | |
|---|---|---|---|---|---|---|---|---|
| Protein | subtype | D1 | D2 | D3 | D4 | D5 | (μg/ml) | % POI |
| SI-75X5 | IgG1 | — | αHER3 | αCD3* | — | — | 192.9 | 72.2 |
| SI-75XM9 | IgG4 | — | αHER3 | αCD3* | — | — | 150.5 | 85.6 |

TABLE 9
Annotation of binding domains for their binding specificity, domain
structure, origin, and sequence identification number (SEQ ID).

| | Binding | Structure in | | SEQ |
|---|---|---|---|---|
| Target | Domain | miniGNC | Origin | ID NO. |
| CD20 | Ritu | scFv | Rituximab | 259-262 |
| EGFR | EGFR | scFv and Fab | αEGFR H7 | 203-206 |
| EGFR | EGFR* | scFv | Panitumumab | 207-210 |
| HER3 | MM-111 | scFv and Fab | MM-111 | 255-258 |
| CD3 | αCD3 | scFv and Fab | 284A10 | 219-222 |
| CD3 | αCD3* | scFv and Fab | 283E3BSM | 227-230 |
| CD3 | αCD3** | scFv | 284A10 FR 1 | 231-234 |
| CD3 | αCD3*** | scFv | 284A10 FR 2 | 235-238 |
| CD3 | αCD3**** | scFv | 284A10 FR 3 | 239-242 |
| CD3 | αCD3***** | scFv | 283E3FRS | 291-294 |
| PD-L1 | αPD-L1 | scFv | PL221G5 | 243-246 |
| 4-1BB | α4-1BB | scFv | 466F6 | 247-250 |
| NKG2D | NKG2D | Receptor | | 253-254 |
| Ligand | | | | |
| 4-1BB | 41BBL | Ligand | | 251-252 |
| EGFR | αEGFR VHH | VHH | VHH-122 | 263-264 |
| HER3 | αHER3 VHH | VHH | VHH BCD090-M2 | 265-266 |
| CD19 | huBU12 | scFv | SI-huBU12-H4, | 215-218 |
| CEA | αCEA-H1 | scFv | CEA-656E11 | 279-282 |
| HER2 | Tras | scFv | Trastuzumab | 284-285 |
| | | Human IgG1 | | 313 |
| | | heavy chain | | |
| | | Human IgG4 | | 314 |
| | | light chain | | |

### SEQUENCE LISTING

| Sample | | SEQ ID NO. | | Fc ProA KO | VH3 ProA KO |
|---|---|---|---|---|---|
| ID | Chain | Protein | DNA | (H435R/Y436F) | (R19S) |
| SI-75X5 | A | 1 | 2 | + | + |
| | B | 3 | 4 | | |
| SI-75X6 | A | 5 | 6 | + | − |
| | B | 7 | 8 | | |
| SI-75X7 | A | 9 | 10 | − | + |
| | B | 11 | 12 | | |
| SI-75X8 | A | 13 | 14 | − | − |
| | B | 15 | 16 | | |
| SI-75P6 | A | 17 | 18 | + | + |
| | B | 19 | 20 | | |
| SI-75P4 | A | 21 | 22 | + | + |
| | B | 23 | 24 | | |
| SI-75P3 | A | 25 | 26 | + | + |
| | B | 27 | 28 | | |
| SI-75P9 | A | 29 | 30 | + | + |
| | B | 31 | 32 | | |
| SI-75E1 | A | 33 | 34 | + | + |
| | B | 35 | 36 | | |
| SI-75E2 | A | 37 | 38 | + | + |
| | B | 39 | 40 | | |
| SI-75X3 | A | 41 | 42 | + | + |
| | B | 43 | 44 | | |
| SI-75X16 | A | 45 | 46 | + | + |
| | B | 47 | 48 | | |
| SI-75X18 | A | 49 | 50 | + | + |
| | B | 51 | 52 | | |
| SI-75X1 | A | 53 | 54 | + | NA |
| | B | 55 | 56 | | |
| SI-75X2 | A | 57 | 58 | + | + |
| | B | 59 | 60 | | |
| SI-75O2 | A | 61 | 62 | + | NA |
| | B | 63 | 64 | | |
| SI-75O7 | A | 65 | 66 | + | NA |
| | B | 67 | 68 | | |
| SI-68P13 | A | 69 | 70 | + | + |
| | B | 71 | 72 | | |
| SI-68P17 | A | 73 | 74 | + | + |
| | B | 75 | 76 | | |
| SI-68P15 | A | 77 | 78 | + | + |
| | B | 79 | 80 | | |
| SI-68P18 | A | 81 | 82 | + | + |
| | B | 83 | 84 | | |
| SI-68P19 | A | 85 | 86 | + | + |
| | B | 87 | 88 | | |
| SI-68P16 | A | 89 | 90 | + | + |
| | B | 91 | 92 | | |
| SI-75P5 | A | 93 | 94 | + | + |
| | B | 95 | 96 | | |
| SI-75P8 | A | 97 | 98 | + | + |
| | B | 99 | 100 | | |
| SI-75X4 | A | 101 | 102 | + | NA |
| | B | 103 | 104 | | |
| SI-75X17 | A | 105 | 106 | + | NA |
| | B | 107 | 108 | | |
| SI-75X19 | A | 109 | 110 | + | NA |
| | B | 111 | 112 | | |
| SI-75X9 | A | 113 | 114 | + | NA |
| | B | 115 | 116 | | |
| SI-75X11 | A | 117 | 118 | + | NA |
| | B | 119 | 120 | | |
| SI-75X15 | A | 121 | 122 | + | NA |
| | B | 123 | 124 | | |
| SI-75X13 | A | 125 | 126 | + | NA |
| | B | 127 | 128 | | |
| SI-38P11 | A | 129 | 130 | + | + |
| | B | 131 | 132 | | |
| SI-76PM1 | A | 133 | 134 | + | + |
| | B | 135 | 136 | | |
| SI-68X1 | A | 195 | 196 | − | − |
| | B | 197 | 198 | | |
| SI-68X3 | A | 199 | 200 | − | − |
| | B | 201 | 202 | | |
| SI-68X2 | A | 137 | 138 | − | − |
| | B | 139 | 140 | | |
| SI-68E1 | A | 141 | 142 | − | − |
| | B | 143 | 144 | | |
| SI-68E2 | A | 145 | 146 | − | − |
| | B | 147 | 148 | | |
| SI-68P1 | A | 149 | 150 | − | − |
| | B | 151 | 152 | | |
| SI-49R26 | A | 153 | 154 | + | NA |
| | B | 155 | 156 | | |
| SI-49R27 | A | 157 | 158 | + | NA |
| | B | 159 | 160 | | |
| SI-49R25 | A | 161 | 162 | − | − |
| | B | 163 | 164 | | |
| SI-49P_X | A | 165 | 166 | − | − |
| | B | 167 | 168 | | |
| SI-49P8 | A | 169 | 170 | − | − |
| | B | 171 | 172 | | |
| SI-49PM1 | A | 173 | 174 | + | + |
| | B | 175 | 176 | | |
| SI-49P9 | A | 177 | 178 | − | − |
| | B | 179 | 180 | | |
| SI-75XM9 | A | 297 | 298 | + | + |
| | B | 299 | 300 | | |

SEQ ID NO.

| Protein ID | Format | Protein | Protein |
|---|---|---|---|
| SI-1C3 HC | αEGFR mAb | 181 | 182 |
| SI-1C3 LC | αEGFR mAb | 183 | 184 |
| SI-1C7 | αHER3 Fc-scFv | 185 | 186 |
| SI-9C21 HC | αCD3 mAb | 187 | 188 |
| SI-9C21 LC | αCD3 mAb | 189 | 190 |
| SI-35FS11 | α4-1BB Fc-scFv | 191 | 192 |
| SI-3FS11 | αPD-L1 Fc-SCFv | 193 | 194 |
| SI-20C14 HC | αCEA mAb 656E11 | 287 | 288 |
| SI-20C14 LC | αCEA mAb 656E11 | 289 | 290 |

| | |
|---|---|
| Variable | SEQ ID |

| Domains | Chains | Protein | DNA |
|---|---|---|---|
| αEGFR | VH | 203 | 204 |
| | VL | 205 | 206 |
| αEGFR * | VH | 207 | 208 |
| | VL | 209 | 210 |
| αEGFR * Stapled | VH | 211 | 212 |
| | VL | 213 | 214 |
| αCD19 | VH | 215 | 216 |
| | VL | 217 | 218 |
| αCD3 | VH | 219 | 220 |
| | VL | 221 | 222 |
| αCD3 Stapled | VH | 223 | 224 |
| | VL | 225 | 226 |
| αCD3 * | VH | 227 | 228 |
| | VL | 229 | 230 |
| αCD3 ** | VH | 231 | 232 |
| | VL | 233 | 234 |
| αCD3 *** | VH | 235 | 236 |
| | VL | 237 | 238 |
| αCD3 **** | VH | 239 | 240 |
| | VL | 241 | 242 |
| αPDL1 | VH | 243 | 244 |
| | VL | 245 | 246 |
| α41BB | VH | 247 | 248 |
| | VL | 249 | 250 |
| 41BBL | NA | 251 | 252 |
| NKG2D dimer | NA | 253 | 254 |
| αHER3 | VH | 255 | 256 |
| | VL | 257 | 258 |
| αCD20 | VH | 259 | 260 |
| | VL | 261 | 262 |
| EGFR VHH | VHH | 263 | 264 |
| HER3 VHH | VHH | 265 | 266 |
| αPDL1 Stapled | VH | 267 | 268 |
| | VL | 269 | 270 |
| α41BB stapled | VH | 271 | 272 |
| | VL | 273 | 274 |
| αHER3 stapled | VH | 275 | 276 |
| | VL | 277 | 278 |
| αCEA | VH | 279 | 280 |
| | VL | 281 | 282 |
| αHER2 stapled | VH | 283 | 284 |
| | VL | 285 | 286 |
| αCD3 ***** | VH | 291 | 292 |
| | VL | 293 | 294 |
| NKG2D monomer | NA | 295 | 296 |

| Antibody | Kabat CDR | AA SEQ ID |
|---|---|---|
| αCEA 656E11 | CDR-H1 | 301 |
| | CDR-H2 | 302 |
| | CDR-H3 | 303 |
| | CDR-L1 | 304 |
| | CDR-L2 | 305 |
| | CDR-L3 | 306 |
| αCD3 283E3 | CDR-H1 | 307 |
| (BSM/FRS) | CDR-H2 | 308 |
| | CDR-H3 | 309 |
| | CDR-L1 | 310 |
| | CDR-L2 | 311 |
| | CDR-L3 | 312 |

>Sequence ID 1: SI-75X5 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 2: SI-75X5 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGA
>Sequence ID 3: SI-75X5 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLWCLVKGFYPSDIAVEWESNGQPENNYKTTPPVLDSDGSFFLYSKLTVDKSRWQQGNVFSCSVMHEALHNR
FTQKSLSLSPG
>Sequence ID 4: SI-75X5 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCCCCCTCTGTGTTCATCTTCCCACCCAGTGATGAG
CAGCTGAAGAGTGGCACAGCCTCTGTGGTGTGCCTGCTCAACAACTTCTACCCCAGAGAAGCCAAGGTGCAGTGGAA
GGTGGACAATGCCCTGCAGAGTGGCAACAGCCAGGAGAGTGTGACAGAGCAGGACAGCAAGGACAGCACCTACAGCC
TGTCCTCCACCCTGACCCTGTCCAAGGCTGACTATGAGAAGCACAAGGTGTATGCCTGTGAGGTGACCCACCAGGGC
CTGAGCAGCCCTGTGACCAAGAGCTTCAACCGAGGAGAGTGTGGAGGAGACAAGACCCACACCTGCCCTCCCTGCCC
AGCCCCAGAAGCTGCTGGAGGCCCCTCTGTGTTCCTCTTCCCTCCCAAGCCCAAGGACACCCTGATGATCTCCAGGA
CCCCAGAAGTCACCTGTGTGGTGGTGGATGTGTCCCATGAAGACCCAGAAGTGAAGTTCAACTGGTATGTGGATGGA
GTGGAAGTGCACAATGCCAAGACCAAGCCCAGAGAAGAGCAGTACAACAGCACCTACCGAGTGGTGTCTGTGCTGAC
TGTGCTGCACCAGGACTGGCTGAATGGGAAGGAGTACAAGTGTGCTGTGTCCAACAAGGCCCTGCCTGCCCCCATTG
AGAAGACCATCTCCAAGGCCAAGGGCCAGCCCAGAGAGCCCCAGGTCTACACCCTGCCTCCCTCCAGAGATGAGCTG
ACCAAGAACCAGGTGTCCCTGTGGTGCCTGGTGAAAGGCTTCTACCCCAGTGACATTGCTGTGGAGTGGGAGAGCAA
TGGCCAGCCAGAGAACAACTACAAGACCACCCCTCCTGTGCTGGACTCTGATGGCTCCTTCTTCCTGTACAGCAAGC
TGACTGTGGACAAGAGCCGCTGGCAGCAGGGCAATGTGTTCTCCTGCTCTGTCATGCATGAGGCCCTGCACAACCGC
TTCACCCAGAAGAGCCTGAGCCTGAGCCCTGGA
>Sequence ID 5: SI-75X6 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 6: SI-75X6 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGA
>Sequence ID 7: SI-75X6 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLRLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLWCLVKGFYPSDIAVEWESNGQPENNYKTTPPVLDSDGSFFLYSKLTVDKSRWQQGNVFSCSVMHEALHNR
FTQKSLSLSPG
>Sequence ID 8: SI-75X6 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCCCCCTCTGTGTTCATCTTCCCACCCAGTGATGAG
CAGCTGAAGAGTGGCACAGCCTCTGTGGTGTGCCTGCTCAACAACTTCTACCCCAGAGAAGCCAAGGTGCAGTGGAA
GGTGGACAATGCCCTGCAGAGTGGCAACAGCCAGGAGAGTGTGACAGAGCAGGACAGCAAGGACAGCACCTACAGCC
TGTCCTCCACCCTGACCCTGTCCAAGGCTGACTATGAGAAGCACAAGGTGTATGCCTGTGAGGTGACCCACCAGGGC
CTGAGCAGCCCTGTGACCAAGAGCTTCAACCGAGGAGAGTGTGGAGGAGACAAGACCCACACCTGCCCTCCCTGCCC
AGCCCCAGAAGCTGCTGGAGGCCCCTCTGTGTTCCTCTTCCCTCCCAAGCCCAAGGACACCCTGATGATCTCCAGGA
CCCCAGAAGTCACCTGTGTGGTGGTGGATGTGTCCCATGAAGACCCAGAAGTGAAGTTCAACTGGTATGTGGATGGA
GTGGAAGTGCACAATGCCAAGACCAAGCCCAGAGAAGAGCAGTACAACAGCACCTACCGAGTGGTGTCTGTGCTGAC
TGTGCTGCACCAGGACTGGCTGAATGGGAAGGAGTACAAGTGTGCTGTGTCCAACAAGGCCCTGCCTGCCCCCATTG
AGAAGACCATCTCCAAGGCCAAGGGCCAGCCCAGAGAGCCCCAGGTCTACACCCTGCCTCCCTCCAGAGATGAGCTG
ACCAAGAACCAGGTGTCCCTGTGGTGCCTGGTGAAAGGCTTCTACCCCAGTGACATTGCTGTGGAGTGGGAGAGCAA
TGGCCAGCCAGAGAACAACTACAAGACCACCCCTCCTGTGCTGGACTCTGATGGCTCCTTCTTCCTGTACAGCAAGC
TGACTGTGGACAAGAGCCGCTGGCAGCAGGGCAATGTGTTCTCCTGCTCTGTCATGCATGAGGCCCTGCACAACCGC
TTCACCCAGAAGAGCCTGAGCCTGAGCCCTGGA
>Sequence ID 9: SI-75X7 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 10: SI-75X7 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGA
>Sequence ID 11: SI-75X7 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLWCLVKGFYPSDIAVEWESNGQPENNYKTTPPVLDSDGSFFLYSKLTVDKSRWQQGNVFSCSVMHEALHNH
YTQKSLSLSPG
>Sequence ID 12: SI-75X7 Chain B Amino Acid Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCCCCCTCTGTGTTCATCTTCCCACCCAGTGATGAG
CAGCTGAAGAGTGGCACAGCCTCTGTGGTGTGCCTGCTCAACAACTTCTACCCCAGAGAAGCCAAGGTGCAGTGGAA
GGTGGACAATGCCCTGCAGAGTGGCAACAGCCAGGAGAGTGTGACAGAGCAGGACAGCAAGGACAGCACCTACAGCC
TGTCCTCCACCCTGACCCTGTCCAAGGCTGACTATGAGAAGCACAAGGTGTATGCCTGTGAGGTGACCCACCAGGGC
CTGAGCAGCCCTGTGACCAAGAGCTTCAACCGAGGAGAGTGTGGAGGAGACAAGACCCACACCTGCCCTCCCTGCCC
AGCCCCAGAAGCTGCTGGAGGCCCCTCTGTGTTCCTCTTCCCTCCCAAGCCCAAGGACACCCTGATGATCTCCAGGA
CCCCAGAAGTCACCTGTGTGGTGGTGGATGTGTCCCATGAAGACCCAGAAGTGAAGTTCAACTGGTATGTGGATGGA
GTGGAAGTGCACAATGCCAAGACCAAGCCCAGAGAAGAGCAGTACAACAGCACCTACCGAGTGGTGTCTGTGCTGAC
TGTGCTGCACCAGGACTGGCTGAATGGGAAGGAGTACAAGTGTGCTGTGTCCAACAAGGCCCTGCCTGCCCCCATTG
AGAAGACCATCTCCAAGGCCAAGGGCCAGCCCAGAGAGCCCCAGGTCTACACCCTGCCTCCCTCCAGAGATGAGCTG
ACCAAGAACCAGGTGTCCCTGTGGTGCCTGGTGAAAGGCTTCTACCCCAGTGACATTGCTGTGGAGTGGGAGAGCAA
TGGCCAGCCAGAGAACAACTACAAGACCACCCCTCCTGTGCTGGACTCTGATGGCTCCTTCTTCCTGTACAGCAAGC
TGACTGTGGACAAGAGCCGCTGGCAGCAGGGCAATGTGTTCTCCTGCTCTGTCATGCATGAGGCCCTGCACAACCAC
TACACCCAGAAGAGCCTGAGCCTGAGCCCTGGA
>Sequence ID 13: SI-75X8 Chain A Amino Acid Sequence
QVQLQESGGRLVQPGEPLSLTCKTSGIDLSSNAIGWVRQAPGKGLEWIGVIFGSGNTYYASWAKGRETISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 14: SI-75X8 Chain A Nucleotide Acid Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGA
>Sequence ID 15: SI-75X8 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLRLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLWCLVKGFYPSDIAVEWESNGQPENNYKTTPPVLDSDGSFFLYSKLTVDKSRWQQGNVFSCSVMHEALHNH
YTQKSLSLSPG
>Sequence ID 16: SI-75X8 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCCCCCTCTGTGTTCATCTTCCCACCCAGTGATGAG
CAGCTGAAGAGTGGCACAGCCTCTGTGGTGTGCCTGCTCAACAACTTCTACCCCAGAGAAGCCAAGGTGCAGTGGAA
GGTGGACAATGCCCTGCAGAGTGGCAACAGCCAGGAGAGTGTGACAGAGCAGGACAGCAAGGACAGCACCTACAGCC
TGTCCTCCACCCTGACCCTGTCCAAGGCTGACTATGAGAAGCACAAGGTGTATGCCTGTGAGGTGACCCACCAGGGC
CTGAGCAGCCCTGTGACCAAGAGCTTCAACCGAGGAGAGTGTGGAGGAGACAAGACCCACACCTGCCCTCCCTGCCC
AGCCCCAGAAGCTGCTGGAGGCCCCTCTGTGTTCCTCTTCCCTCCCAAGCCCAAGGACACCCTGATGATCTCCAGGA
CCCCAGAAGTCACCTGTGTGGTGGTGGATGTGTCCCATGAAGACCCAGAAGTGAAGTTCAACTGGTATGTGGATGGA
GTGGAAGTGCACAATGCCAAGACCAAGCCCAGAGAAGAGCAGTACAACAGCACCTACCGAGTGGTGTCTGTGCTGAC
TGTGCTGCACCAGGACTGGCTGAATGGGAAGGAGTACAAGTGTGCTGTGTCCAACAAGGCCCTGCCTGCCCCCATTG
AGAAGACCATCTCCAAGGCCAAGGGCCAGCCCAGAGAGCCCCAGGTCTACACCCTGCCTCCCTCCAGAGATGAGCTG
ACCAAGAACCAGGTGTCCCTGTGGTGCCTGGTGAAAGGCTTCTACCCCAGTGACATTGCTGTGGAGTGGGAGAGCAA
TGGCCAGCCAGAGAACAACTACAAGACCACCCCTCCTGTGCTGGACTCTGATGGCTCCTTCTTCCTGTACAGCAAGC
TGACTGTGGACAAGAGCCGCTGGCAGCAGGGCAATGTGTTCTCCTGCTCTGTCATGCATGAGGCCCTGCACAACCAC
TACACCCAGAAGAGCCTGAGCCTGAGCCCTGGA
>Sequence ID 17: SI-75P6 Chain A Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSSGGGGSGGGGSQVQLQQSGPGLVKPSETLSITCTVSGFSLTNYGVHWIRQAPGKGLE
WLGVIWSGGNTDYNTPFTSRFTITKDNSKNQVYFKLRSVRADDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRWQQGNVFSCSVMH
EALHNHYTQKSLSLSPGGGGGSGGGGSDVVMTQSPSSVSASVGDRVTITCQASQNIRTYLSWYQQKPGKAPKLLIYA
AANLASGVPSRFSGSGSGTDFTLTISDLEPGDAATYYCQSTYLGTDYVGGAFGGGTKLTVLGGGGSGGGGSGGGGSG
GGGSEVQLVESGGGLVQPGGSLRLSCTASGFTISSYHMQWVRQAPGKGLEYIGTISSGGNVYYASSARGRFTISRPS
SKNTVDLQMNSLRAEDTAVYYCARDSGYSDPMWGQGTLVTVSS
>Sequence ID 18: SI-75P6 Chain A Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTCTCGAGTGGCGGTGGAGGGTC
CGGCGGTGGTGGATCACAAGTACAGTTGCAGCAATCCGGTCCCGGTCTCGTCAAACCGAGTGAGACGCTTAGTATAA
CGTGTACTGTTTCAGGCTTTAGCCTTACGAACTATGGAGTTCACTGGATTCGGCAGGCACCCGGCAAAGGTTTGGAA
TGGCTGGGTGTTATTTGGTCAGGTGGAAATACAGACTATAACACCCCCTTTACAAGTCGGTTCACAATTACGAAAGA
TAATTCCAAAAATCAAGTTTATTTCAAGTTGAGATCCGTCCGCGCGGACGACACTGCGATCTACTATTGTGCGAGGG
CACTGACCTACTACGATTACGAATTTGCGTATTGGGGGCAAGGGACTCTTGTAACAGTCTCCAGTGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTATACCCTGCCC
CCATCCCGGGATGAGCTGACCAAGAACCAGGTCAGCCTGTCCTGCGCCGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCGTGAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGCGGCGGTTCAGGTGGTGGGGG
ATCCGACGTTGTGATGACGCAGTCTCCTAGTTCAGTATCAGCGTCTGTTGGGGATCGAGTCACGATTACATGCCAGG
CGAGCCAAAATATTAGGACGTATCTGTCTTGGTATCAACAAAAGCCTGGTAAGGCTCCAAAACTCCTCATTTACGCC
GCTGCCAACTTGGCTAGTGGAGTACCTTCACGCTTTAGTGGGTCAGGTTCTGGAACAGATTTTACGTTGACGATCTC
CGATTTGGAACCAGGGGACGCTGCCACGTATTATTGCCAGTCCACCTACCTCGGGACTGACTACGTTGGTGGGGCGT
TTGGCGGAGGAACAAAACTCACTGTACTTGGTGGTGGTGGTTCAGGCGGGGGGGGTTCCGGCGGAGGCGGTTCTGGG
GGTGGAGGTTCCGAGGTTCAGTTGGTTGAAAGTGGGGGGGGACTCGTTCAGCCTGGAGGCTCTTTGCGACTTTCTTG
TACCGCTTCTGGGTTCACTATAAGTTCATATCATATGCAATGGGTAAGACAGGCGCCTGGTAAGGGACTTGAATACA
TAGGAACAATAAGCTCTGGTGGTAACGTCTATTACGCCTCATCCGCGCGGGGGAGGTTTACAATTTCCAGGCCTTCT
AGCAAAAACACGGTAGATCTGCAAATGAACTCTCTTCGCGCTGAAGATACAGCAGTCTACTACTGCGCCAGGGATTC
AGGGTATTCTGACCCCATGTGGGGGCAGGGCACATTGGTTACCGTGTCCTCT
>Sequence ID 19: SI-75P6 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSEIVLTQSPSTLSVSPGERATFSCRASQSIGTNIHWYQQKPGKPPRLLIK
YASESISGIPDRFSGSGSGTEFTLTISSVQSEDFAVYYCQQNNNWPTTFGPGTKLTVLRTVAAPSVFIFPPSDEQLK
SGTASVVCLLNNFYPREAKVQWKVDNALQSGNSQESVTEQDSKDSTYSLSSTLTLSKADYEKHKVYACEVTHQGLSS
PVTKSFNRGECGGDKTHTCPPCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCAVSNKALPAPIEKTISKAKGQPREPQVYTLPPSRDELTKN
QVSLWCLVKGFYPSDIAVEWESNGQPENNYKTTPPVLDSDGSFFLYSKLTVDKSRWQQGNVFSCSVMHEALHNRFTQ
KSLSLSPGGGGGSGGGGSDIQMTQSPSTLSASVGDRVTITCQASQSISSHLNWYQQKPGKAPKLLIYKASTLASGVP
SRFSGSGSGTEFTLTISSLQPDDFATYYCQQGYSWGNVDNVFGGGTKVTVLGGGGSGGGGSGGGGSGGGGSEVQLLE
SGGGLVQPGGSLSLSCAASGFSFSSGYDMCWVRQAPGKGLEWIACIAAGSAGITYDANWAKGRFTISRDNSKNTLYL
QMNSLRAEDTAVYYCARSAFSFDYAMDLWGQGTLVTVSS
>Sequence ID 20: SI-75P6 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGAAATCGTCCTTACACAATCTCCTAGCACACTGAGTGTGAGCCCCGGCGAACGCGCGACTTTCTCTTGCA
GGGCAAGTCAATCCATAGGGACTAATATACATTGGTATCAACAAAAGCCAGGTAAACCACCCAGGCTTTTGATTAAG
TATGCAAGTGAGTCTATTTCCGGTATCCCTGACCGCTTCTCTGGATCAGGCAGTGGCACAGAGTTCACACTCACCAT
ATCTAGTGTGCAATCAGAGGACTTCGCCGTGTATTACTGCCAACAGAATAATAACTGGCCGACTACCTTCGGACCCG
GTACAAAGCTGACCGTTTTACGTACGGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCAGCA
CCCTGACGCTGAGCAAAGCAGACTACGAGAAACACAAAGTCTACGCCTGCGAAGTCACCCATCAGGGCCTGAGCTCG
CCCGTCACAAAGAGCTTCAACAGGGGAGAGTGTGGAGGCGACAAAACTCACACATGCCCACCGTGCCCAGCACCTGA
AGCCGCGGGGGGACCGTCAGTCTTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCGCGGTCTCCAACAAAGCCCTCCCAGCCCCCATCGAGAAAACCA
TCTCCAAAGCCAAAGGGCAGCCCCGAGAACCACAGGTGTACACCCTGCCCCCAAGCCGGGATGAGCTGACCAAGAAC
CAGGTCAGCCTGTGGTGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTATAGCAAGCTCACCGTGG
ACAAGAGCAGGTGGCAGCAGGGGAACGTCTTCTCATGCTCCGTGATGCATGAGGCTCTGCACAACAGATTCACGCAG
AAGAGCCTCTCCCTGTCTCCGGGTGGCGGTGGAGGGTCCGGCGGTGGTGGATCCGACATTCAGATGACACAGTCACC
GTCTACGCTCTCAGCCTCAGTAGGCGATAGGGTGACTATCACCTGCCAAGCCTCCCAATCTATAAGTTCACACCTCA
ACTGGTATCAACAGAAACCCGGTAAAGCACCGAAGCTGCTCATCTACAAGGCATCAACTCTTGCCTCAGGTGTGCCT
AGTCGCTTCTCCGGGAGCGGCTCCGGTACTGAGTTTACTTTGACGATATCTTCCCTCCAACCCGACGACTTCGCCAC
ATACTATTGTCAGCAGGGTTATAGTTGGGGAAATGTTGACAATGTGTTTGGAGGGGGAACGAAAGTGACTGTGTTGG
GAGGGGGAGGAAGTGGCGGCGGCGGAAGCGGAGGTGGTGGCTCCGGCGGGGGTGGAAGCGAAGTGCAATTGCTCGAA
AGTGGTGGCGGACTTGTGCAACCTGGTGGAAGCCTCTCACTCTCTTGCGCGGCGAGTGGTTTTAGCTTCAGTAGCGG
CTACGATATGTGCTGGGTAAGACAGGCTCCTGGTAAAGGCCTTGAGTGGATTGCGTGTATCGCCGCCGGGTCTGCTG
GTATAACATACGATGCAAACTGGGCGAAAGGGCGCTTCACAATCTCCAGGGACAACTCAAAAAATACATTGTACCTC
CAGATGAATAGCCTGAGAGCCGAAGACACCGCAGTCTACTATTGCGCAAGGTCAGCATTTAGCTTCGATTATGCAAT
GGATCTTTGGGGCCAGGGGACGCTCGTCACCGTCTCATCTTGA
>Sequence ID 21: SI-75P4 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSREGPELSPDDPAGLLDLRQGMFAQLVAQNVLLIDGPLSWYSDPGLAGVSLTGGLS
YKEDTKELVVAKAGVYYVFFQLELRRVVAGEGSGSVSLALHLQPLRSAAGAAALALTVDLPPASSEARNSAFGFQGR
LLHLSAGQRLGVHLHTEARARHAWQLTQGATVLGLFRVTPEIPAGLGSTGSGSKPGSGEGSTKGREGPELSPDDPAG
LLDLRQGMFAQLVAQNVLLIDGPLSWYSDPGLAGVSLTGGLSYKEDTKELVVAKAGVYYVFFQLELRRVVAGEGSGS
VSLALHLQPLRSAAGAAALALTVDLPPASSEARNSAFGFQGRLLHLSAGQRLGVHLHTEARARHAWQLTQGATVLGL
FRVTPEIPAGLGGGGSGGGGSREGPELSPDDPAGLLDLRQGMFAQLVAQNVLLIDGPLSWYSDPGLAGVSLTGGLSY
KEDTKELVVAKAGVYYVFFQLELRRVVAGEGSGSVSLALHLQPLRSAAGAAALALTVDLPPASSEARNSAFGFQGRL
LHLSAGQRLGVHLHTEARARHAWQLTQGATVLGLFRVTPEIPAGL
>Sequence ID 22: SI-75P4 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTATACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGCGGCGGTTCAGGTGGTGGGGGATCCCGAGAGGGCCC
CGAGCTGTCTCCTGATGACCCAGCAGGCCTCTTGGACTTGCGGCAGGGTATGTTCGCTCAACTTGTGGCTCAGAATG
TTCTGCTCATTGATGGACCACTCTCTTGGTATAGTGACCCCGGTCTGGCCGGGGTGAGTCTGACCGGCGGGCTCTCT
TATAAAGAGGATACTAAGGAACTGGTCGTAGCAAAAGCGGGCGTTTATTACGTTTTTTTTCAGCTGGAGCTCAGGCG
CGTGGTGGCCGGCGAGGGCAGTGGCTCTGTGTCCCTGGCCCTGCACTTACAGCCCTTGAGAAGCGCTGCAGGTGCTG
CCGCACTGGCTTTAACTGTTGACCTCCCTCCGGCCTCTTCTGAAGCTAGAAACAGCGCTTTCGGCTTCCAAGGGCGC
CTGCTGCACCTGAGCGCAGGCCAGCGCTTAGGTGTGCACCTTCATACAGAGGCCAGGGCCCGACACGCTTGGCAGCT
CACACAGGGTGCCACGGTTCTCGGACTTTTCCGCGTTACTCCCGAGATCCCCGCTGGCCTCGGAAGTACTGGTTCTG
GGTCTAAACCCGGTTCCGGCGAAGGTAGTACTAAAGGACGAGAAGGGCCAGAGTTAAGTCCAGATGACCCTGCTGGG
CTTTTGGACCTGCGGCAGGGCATGTTCGCTCAACTGGTGGCTCAGAACGTGCTGCTGATCGATGGCCCCCTGAGTTG
GTACAGCGATCCCGGGCTGGCAGGCGTGTCACTTACAGGGGGCCTCTCTTACAAGGAAGACACCAAGGAGTTAGTGG
TCGCTAAGGCTGGCGTGTATTACGTGTTCTTCCAACTGGAGCTGAGAAGGGTTGTGGCAGGAGAGGGTAGCGGCAGC
GTGTCTTTAGCCCTTCACTTGCAGCCCCTGAGGTCTGCTGCAGGTGCAGCCGCTCTCGCGCTCACCGTGGATCTCCC
CCCAGCCTCATCTGAAGCTAGGAACAGTGCATTTGGCTTTCAGGGACGCTTGCTGCACCTCTCCGCTGGACAGAGGC
TGGGCGTGCACCTTCACACAGAGGCCCGTGCCAGGCATGCATGGCAGCTCACTCAGGGGGCAACAGTGCTGGGTCTC
TTCCGCGTGACTCCTGAAATACCAGCTGGACTTGGCGGTGGAGGCAGCGGCGGAGGAGGATCTCGTGAGGGGCCAGA
ACTGTCCCCCGATGACCCAGCCGGACTGCTCGATCTCAGACAGGGCATGTTCGCTCAGCTTGTAGCCCAAAATGTCC
TCCTGATTGACGGCCCTTTGAGCTGGTATAGTGATCCCGGCTTGGCCGGGGTATCTCTGACCGGAGGCCTCTCCTAC
AAGGAAGACACCAAAGAGCTGGTGGTGGCAAAAGCGGGGGTGTATTATGTGTTCTTTCAGCTCGAGCTGCGGAGAGT
TGTGGCCGGGGAAGGGTCTGGGAGCGTATCTCTTGCACTTCACCTGCAGCCCCTGCGCAGCGCCGCTGGAGCCGCCG
CCCTTGCTCTTACTGTGGATCTGCCTCCTGCTTCCTCAGAAGCACGCAACAGCGCCTTCGGCTTTCAAGGACGTCTC
CTGCACTTGTCCGCAGGACAGAGGTTGGGCGTCCATTTACACACTGAGGCACGGGCACGGCACGCTTGGCAGCTTAC
CCAGGGAGCCACCGTGCTGGGACTCTTTAGAGTGACACCCGAGATCCCCGCTGGCTTGTGA
>Sequence ID 23: SI-75P4 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPGGGGGSGGGGSDIQMTQSPSTLSASVGDRVTITCQASQSISSHLNWYQQKPGKAPKLLIYKASTL
ASGVPSRFSGSGSGTEFTLTISSLQPDDFATYYCQQGYSWGNVDNVFGGGTKVTVLGGGGSGGGGSGGGGSGGGGSE
VQLLESGGGLVQPGGSLSLSCAASGFSFSSGYDMCWVRQAPGKGLEWIACIAAGSAGITYDANWAKGRFTISRDNSK
NTLYLQMNSLRAEDTAVYYCARSAFSFDYAMDLWGQGTLVTVSS
>Sequence ID 24: SI-75P4 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCAAGCCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACAGATTCACGCAGAAGAGCCTCTCCCTGTCTCCGGGTGGCGGTGGAGGGTCCGGCGGTGGTGGATCCGACATTCA
GATGACACAGTCACCGTCTACGCTCTCAGCCTCAGTAGGCGATAGGGTGACTATCACCTGCCAAGCCTCCCAATCTA
TAAGTTCACACCTCAACTGGTATCAACAGAAACCCGGTAAAGCACCGAAGCTGCTCATCTACAAGGCATCAACTCTT
GCCTCAGGTGTGCCTAGTCGCTTCTCCGGGAGCGGCTCCGGTACTGAGTTTACTTTGACGATATCTTCCCTCCAACC
CGACGACTTCGCCACATACTATTGTCAGCAGGGTTATAGTTGGGGAAATGTTGACAATGTGTTTGGAGGGGGAACGA
AAGTGACTGTGTTGGGAGGGGGAGGAAGTGGCGGCGGCGGAAGCGGAGGTGGTGGCTCCGGCGGGGGTGGAAGCGAA
GTGCAATTGCTCGAAAGTGGTGGCGGACTTGTGCAACCTGGTGGAAGCCTCTCACTCTCTTGCGCGGCGAGTGGTTT
TAGCTTCAGTAGCGGCTACGATATGTGCTGGGTAAGACAGGCTCCTGGTAAAGGCCTTGAGTGGATTGCGTGTATCG
CCGCCGGGTCTGCTGGTATAACATACGATGCAAACTGGGCGAAAGGGCGCTTCACAATCTCCAGGGACAACTCAAAA
AATACATTGTACCTCCAGATGAATAGCCTGAGAGCCGAAGACACCGCAGTCTACTATTGCGCAAGGTCAGCATTTAG
CTTCGATTATGCAATGGATCTTTGGGGCCAGGGGACGCTCGTCACCGTCTCATCTTGA
>Sequence ID 25: SI-75P3 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSDVVMTQSPSSVSASVGDRVTITCQASQNIRTYLSWYQQKPGKAPKLLIYAAANLA
SGVPSRFSGSGSGTDFTLTISDLEPGDAATYYCQSTYLGTDYVGGAFGGGTKLTVLGGGGSGGGGSGGGGSGGGGSE
VQLVESGGGLVQPGGSLRLSCTASGFTISSYHMQWVRQAPGKGLEYIGTISSGGNVYYASSARGRFTISRPSSKNTV
DLQMNSLRAEDTAVYYCARDSGYSDPMWGQGTLVTVSS
>Sequence ID 26: SI-75P3 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTATACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGCGGCGGTTCAGGTGGTGGGGGATCCGACGTTGTGAT
GACGCAGTCTCCTAGTTCAGTATCAGCGTCTGTTGGGGATCGAGTCACGATTACATGCCAGGCGAGCCAAAATATTA
GGACGTATCTGTCTTGGTATCAACAAAAGCCTGGTAAGGCTCCAAAACTCCTCATTTACGCCGCTGCCAACTTGGCT
AGTGGAGTACCTTCACGCTTTAGTGGGTCAGGTTCTGGAACAGATTTTACGTTGACGATCTCCGATTTGGAACCAGG
GGACGCTGCCACGTATTATTGCCAGTCCACCTACCTCGGGACTGACTACGTTGGTGGGGCGTTTGGCGGAGGAACAA
AACTCACTGTACTTGGTGGTGGTGGTTCAGGCGGGGGGGGTTCCGGCGGAGGCGGTTCTGGGGGTGGAGGTTCCGAG
GTTCAGTTGGTTGAAAGTGGGGGGGGACTCGTTCAGCCTGGAGGCTCTTTGCGACTTTCTTGTACCGCTTCTGGGTT
CACTATAAGTTCATATCATATGCAATGGGTAAGACAGGCGCCTGGTAAGGGACTTGAATACATAGGAACAATAAGCT
CTGGTGGTAACGTCTATTACGCCTCATCCGCGCGGGGGAGGTTTACAATTTCCAGGCCTTCTAGCAAAAACACGGTA
GATCTGCAAATGAACTCTCTTCGCGCTGAAGATACAGCAGTCTACTACTGCGCCAGGGATTCAGGGTATTCTGACCC
CATGTGGGGGCAGGGCACATTGGTTACCGTGTCCTCT
>Sequence ID 27: SI-75P3 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPGGGGGSGGGGSDIQMTQSPSTLSASVGDRVTITCQASQSISSHLNWYQQKPGKAPKLLIYKASTL
ASGVPSRFSGSGSGTEFTLTISSLQPDDFATYYCQQGYSWGNVDNVFGGGTKVTVLGGGGSGGGGSGGGGSGGGGSE
VQLLESGGGLVQPGGSLSLSCAASGFSFSSGYDMCWVRQAPGKGLEWIACIAAGSAGITYDANWAKGRFTISRDNSK
NTLYLQMNSLRAEDTAVYYCARSAFSFDYAMDLWGQGTLVTVSS
>Sequence ID 28: SI-75P3 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCAAGCCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACAGATTCACGCAGAAGAGCCTCTCCCTGTCTCCGGGTGGCGGTGGAGGGTCCGGCGGTGGTGGATCCGACATTCA
GATGACACAGTCACCGTCTACGCTCTCAGCCTCAGTAGGCGATAGGGTGACTATCACCTGCCAAGCCTCCCAATCTA
TAAGTTCACACCTCAACTGGTATCAACAGAAACCCGGTAAAGCACCGAAGCTGCTCATCTACAAGGCATCAACTCTT
GCCTCAGGTGTGCCTAGTCGCTTCTCCGGGAGCGGCTCCGGTACTGAGTTTACTTTGACGATATCTTCCCTCCAACC
CGACGACTTCGCCACATACTATTGTCAGCAGGGTTATAGTTGGGGAAATGTTGACAATGTGTTTGGAGGGGGAACGA
AAGTGACTGTGTTGGGAGGGGGAGGAAGTGGCGGCGGCGGAAGCGGAGGTGGTGGCTCCGGCGGGGGTGGAAGCGAA
GTGCAATTGCTCGAAAGTGGTGGCGGACTTGTGCAACCTGGTGGAAGCCTCTCACTCTCTTGCGCGGCGAGTGGTTT
TAGCTTCAGTAGCGGCTACGATATGTGCTGGGTAAGACAGGCTCCTGGTAAAGGCCTTGAGTGGATTGCGTGTATCG
CCGCCGGGTCTGCTGGTATAACATACGATGCAAACTGGGCGAAAGGGCGCTTCACAATCTCCAGGGACAACTCAAAA
AATACATTGTACCTCCAGATGAATAGCCTGAGAGCCGAAGACACCGCAGTCTACTATTGCGCAAGGTCAGCATTTAG
CTTCGATTATGCAATGGATCTTTGGGGCCAGGGGACGCTCGTCACCGTCTCATCTTGA
>Sequence ID 29: SI-75P9 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSQVQLQESGGGLVKPGGSLRLSCAASGFTFSSYWMSWVRQAPGKGLEWVANIN
RDGSASYYVDSVKGRFTISRDDAKNSLYLQMNSLRAEDTAVYYCARDRGVGYFDLWGRGTLVTVSSASTKGPSVFPL
APSSKSTSGGTAALGCLVKDYFPEPVTVSWNSGALTSGVHTFPAVLQSSGLYSLSSVVTVPSSSLGTQTYICNVNHK
PSNTKVDKRVEPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLPPSRDELT
KNQVSLSCAVKGFYPSDIAVEWESNGQPENNYKTTPPVLDSDGSFFLVSKLTVDKSRWQQGNVFSCSVMHEALHNHY
TQKSLSLSPGGGGGSGGGGSDVVMTQSPSSVSASVGDRVTITCQASQNIRTYLSWYQQKPGKAPKLLIYAAANLASG
VPSRFSGSGSGTDFTLTISDLEPGDAATYYCQSTYLGTDYVGGAFGGGTKLTVLGGGGSGGGGSGGGGSGGGGSEVQ
LVESGGGLVQPGGSLRLSCTASGFTISSYHMQWVRQAPGKGLEYIGTISSGGNVYYASSARGRFTISRPSSKNTVDL
QMNSLRAEDTAVYYCARDSGYSDPMWGQGTLVTVSS
>Sequence ID 30: SI-75P9 Chain A Nucleotide
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCACA
GGTGCAGCTGCAGGAGAGTGGAGGAGGCCTGGTGAAGCCTGGAGGCAGCCTGCGGCTGTCCTGTGCTGCCTCTGGCT
TCACCTTCTCCTCCTACTGGATGAGCTGGGTGCGCCAGGCCCCAGGCAAGGGCCTGGAGTGGGTGGCCAACATCAAC
AGAGATGGCTCTGCCTCCTACTATGTGGACTCTGTGAAAGGCCGCTTCACCATCTCCAGAGATGATGCCAAGAACAG
CCTGTACCTGCAGATGAACAGCCTGAGAGCTGAGGACACAGCTGTGTACTACTGTGCCAGAGACCGAGGTGTGGGCT
ACTTTGACCTGTGGGGCAGAGGCACCCTGGTGACTGTGTCCTCTGCTAGCACCAAGGGCCCATCGGTCTTCCCCCTG
GCACCCTCCTCCAAGAGCACCTCTGGGGGCACAGCGGCCCTGGGCTGCCTGGTCAAGGACTACTTCCCCGAACCGGT
GACGGTGTCGTGGAACTCAGGCGCCCTGACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAGACCTACATCTGCAACGTGAATCACAAG
CCCAGCAACACCAAGGTGGACAAGAGAGTTGAGCCCAAATCTTGTGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTATACCCTGCCCCCATCCCGGGATGAGCTGACC
AAGAACCAGGTCAGCCTGTCCTGCGCCGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCGTGAGCAAGCTCA
CCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGCGGCGGTTCAGGTGGTGGGGGATCCGACGTTGTGATGACGCA
GTCTCCTAGTTCAGTATCAGCGTCTGTTGGGGATCGAGTCACGATTACATGCCAGGCGAGCCAAAATATTAGGACGT
ATCTGTCTTGGTATCAACAAAAGCCTGGTAAGGCTCCAAAACTCCTCATTTACGCCGCTGCCAACTTGGCTAGTGGA
GTACCTTCACGCTTTAGTGGGTCAGGTTCTGGAACAGATTTTACGTTGACGATCTCCGATTTGGAACCAGGGGACGC
TGCCACGTATTATTGCCAGTCCACCTACCTCGGGACTGACTACGTTGGTGGGGCGTTTGGCGGAGGAACAAAACTCA
CTGTACTTGGTGGTGGTGGTTCAGGCGGGGGGGGTTCCGGCGGAGGCGGTTCTGGGGGTGGAGGTTCCGAGGTTCAG
TTGGTTGAAAGTGGGGGGGGACTCGTTCAGCCTGGAGGCTCTTTGCGACTTTCTTGTACCGCTTCTGGGTTCACTAT
AAGTTCATATCATATGCAATGGGTAAGACAGGCGCCTGGTAAGGGACTTGAATACATAGGAACAATAAGCTCTGGTG
GTAACGTCTATTACGCCTCATCCGCGCGGGGGAGGTTTACAATTTCCAGGCCTTCTAGCAAAAACACGGTAGATCTG
CAAATGAACTCTCTTCGCGCTGAAGATACAGCAGTCTACTACTGCGCCAGGGATTCAGGGTATTCTGACCCCATGTG
GGGGCAGGGCACATTGGTTACCGTGTCCTCTTGA
>Sequence ID 31: SI-75P9 Chain B Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSSGGGGSGGGGSQSALTQPASVSGSPGQSITISCTGTSSDVGGYNFVSWYQQHPGKAP
KLMIYDVSDRPSGVSDRFSGSKSGNTASLIISGLQADDEADYYCSSYGSSSTHVIFGGGTKVTVLRTPKANPTVTLF
PPSSEELQANKATLVCLISDFYPGAVTVAWKADGSPVKAGVETTKPSKQSNNKYAASSYLSLTPEQWKSHRSYSCQV
THEGSTVEKTVAPTECSGGDKTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCAVSNKALPAPIEKTISKAKGQPREPQVYTLPPSR
DELTKNQVSLWCLVKGFYPSDIAVEWESNGQPENNYKTTPPVLDSDGSFFLYSKLTVDKSRWQQGNVFSCSVMHEAL
HNRFTQKSLSLSPGGGGGSGGGGSDIQMTQSPSTLSASVGDRVTITCQASQSISSHLNWYQQKPGKAPKLLIYKAST
LASGVPSRFSGSGSGTEFTLTISSLQPDDFATYYCQQGYSWGNVDNVFGGGTKVTVLGGGGSGGGGSGGGGSGGGGS
EVQLLESGGGLVQPGGSLSLSCAASGFSFSSGYDMCWVRQAPGKGLEWIACIAAGSAGITYDANWAKGRFTISRDNS
KNTLYLQMNSLRAEDTAVYYCARSAFSFDYAMDLWGQGTLVTVSS
>Sequence ID 32: SI-75P9 Chain B Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCCTCAGGCGGTGGAGGGTC
CGGCGGTGGTGGATCACAGAGTGCCCTGACCCAGCCAGCCTCTGTCTCTGGCAGCCCAGGCCAGTCCATCACCATCT
CCTGCACAGGCACCTCCAGTGATGTGGGAGGCTACAACTTTGTGTCCTGGTACCAGCAGCACCCAGGCAAGGCCCCC
AAGCTCATGATCTATGATGTGTCTGACAGACCCTCTGGAGTCTCTGACAGATTCTCTGGCTCCAAGAGTGGCAACAC
AGCCTCCCTCATCATCTCTGGCCTGCAGGCTGATGATGAAGCTGACTACTACTGCTCCTCCTATGGCTCCTCCTCCA
CCCATGTCATCTTTGGAGGAGGCACCAAGGTCACTGTGCTGCGTACGCCCAAGGCCAACCCCACCGTGACCCTGTTC
CCCCCAAGCAGCGAGGAACTGCAGGCCAACAAGGCCACCCTCGTGTGCCTGATCAGCGACTTCTACCCTGGCGCCGT
GACCGTGGCCTGGAAGGCCGATGGATCTCCCGTGAAGGCCGGCGTGGAAACCACCAAGCCCAGCAAGCAGAGCAACA
ACAAATACGCCGCCAGCTCCTACCTGAGCCTGACCCCCGAGCAGTGGAAGTCCCACCGGTCCTACAGCTGCCAGGTC
ACACACGAAGGCAGCACCGTGGAAAAGACAGTGGCCCCCACCGAGTGCAGCGGAGGCGACAAAACTCACACATGCCC
ACCGTGCCCAGCACCTGAAGCCGCGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCGCGGTCTCCAACAAAGCCCTCCCAG
CCCCCATCGAGAAAACCATCTCCAAAGCCAAAGGGCAGCCCCGAGAACCACAGGTGTACACCCTGCCCCCAAGCCGG
GATGAGCTGACCAAGAACCAGGTCAGCCTGTGGTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACAGATTCACGCAGAAGAGCCTCTCCCTGTCTCCGGGTGGCGGTGGAGGGTCCGGCGGTGGTGGATCCGACAT
TCAGATGACACAGTCACCGTCTACGCTCTCAGCCTCAGTAGGCGATAGGGTGACTATCACCTGCCAAGCCTCCCAAT
CTATAAGTTCACACCTCAACTGGTATCAACAGAAACCCGGTAAAGCACCGAAGCTGCTCATCTACAAGGCATCAACT
CTTGCCTCAGGTGTGCCTAGTCGCTTCTCCGGGAGCGGCTCCGGTACTGAGTTTACTTTGACGATATCTTCCCTCCA
ACCCGACGACTTCGCCACATACTATTGTCAGCAGGGTTATAGTTGGGGAAATGTTGACAATGTGTTTGGAGGGGGAA
CGAAAGTGACTGTGTTGGGAGGGGGAGGAAGTGGCGGCGGCGGAAGCGGAGGTGGTGGCTCCGGCGGGGGTGGAAGC
GAAGTGCAATTGCTCGAAAGTGGTGGCGGACTTGTGCAACCTGGTGGAAGCCTCTCACTCTCTTGCGCGGCGAGTGG
TTTTAGCTTCAGTAGCGGCTACGATATGTGCTGGGTAAGACAGGCTCCTGGTAAAGGCCTTGAGTGGATTGCGTGTA
TCGCCGCCGGGTCTGCTGGTATAACATACGATGCAAACTGGGCGAAAGGGCGCTTCACAATCTCCAGGGACAACTCA
AAAAATACATTGTACCTCCAGATGAATAGCCTGAGAGCCGAAGACACCGCAGTCTACTATTGCGCAAGGTCAGCATT
TAGCTTCGATTATGCAATGGATCTTTGGGGCCAGGGGACGCTCGTCACCGTCTCATCT
>Sequence ID 33: SI-75E1 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSDVVMTQSPSSVSASVGDRVTITCQASQNIRTYLSWYQQKPGKAPKLLIYAAANLA
SGVPSRFSGSGSGTDFTLTISDLEPGDAATYYCQSTYLGTDYVGGAFGGGTKLTVLGGGGSGGGGSGGGGSGGGGSE
VQLVESGGGLVQPGGSLRLSCTASGFTISSYHMQWVRQAPGKGLEYIGTISSGGNVYYASSARGRFTISRPSSKNTV
DLQMNSLRAEDTAVYYCARDSGYSDPMWGQGTLVTVSS
>Sequence ID 34: SI-75E1 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTATACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGCGGCGGTTCAGGTGGTGGGGGATCCGACGTTGTGAT
GACGCAGTCTCCTAGTTCAGTATCAGCGTCTGTTGGGGATCGAGTCACGATTACATGCCAGGCGAGCCAAAATATTA
GGACGTATCTGTCTTGGTATCAACAAAAGCCTGGTAAGGCTCCAAAACTCCTCATTTACGCCGCTGCCAACTTGGCT
AGTGGAGTACCTTCACGCTTTAGTGGGTCAGGTTCTGGAACAGATTTTACGTTGACGATCTCCGATTTGGAACCAGG
GGACGCTGCCACGTATTATTGCCAGTCCACCTACCTCGGGACTGACTACGTTGGTGGGGCGTTTGGCGGAGGAACAA
AACTCACTGTACTTGGTGGTGGTGGTTCAGGCGGGGGGGGTTCCGGCGGAGGCGGTTCTGGGGGTGGAGGTTCCGAG
GTTCAGTTGGTTGAAAGTGGGGGGGGACTCGTTCAGCCTGGAGGCTCTTTGCGACTTTCTTGTACCGCTTCTGGGTT
CACTATAAGTTCATATCATATGCAATGGGTAAGACAGGCGCCTGGTAAGGGACTTGAATACATAGGAACAATAAGCT
CTGGTGGTAACGTCTATTACGCCTCATCCGCGCGGGGGAGGTTTACAATTTCCAGGCCTTCTAGCAAAAACACGGTA
GATCTGCAAATGAACTCTCTTCGCGCTGAAGATACAGCAGTCTACTACTGCGCCAGGGATTCAGGGTATTCTGACCC
CATGTGGGGGCAGGGCACATTGGTTACCGTGTCCTCTTGA
>Sequence ID 35: SI-75E1 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPG
>Sequence ID 36: SI-75E1 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCCCCCTCTGTGTTCATCTTCCCACCCAGT
GATGAGCAGCTGAAGAGTGGCACAGCCTCTGTGGTGTGCCTGCTCAACAACTTCTACCCCAGAGAAGCCAAGGTGCA
GTGGAAGGTGGACAATGCCCTGCAGAGTGGCAACAGCCAGGAGAGTGTGACAGAGCAGGACAGCAAGGACAGCACCT
ACAGCCTGTCCTCCACCCTGACCCTGTCCAAGGCTGACTATGAGAAGCACAAGGTGTATGCCTGTGAGGTGACCCAC
CAGGGCCTGAGCAGCCCTGTGACCAAGAGCTTCAACCGAGGAGAGTGTGGAGGAGACAAGACCCACACCTGCCCTCC
CTGCCCAGCCCCAGAAGCTGCTGGAGGCCCCTCTGTGTTCCTCTTCCCTCCCAAGCCCAAGGACACCCTGATGATCT
CCAGGACCCCAGAAGTCACCTGTGTGGTGGTGGATGTGTCCCATGAAGACCCAGAAGTGAAGTTCAACTGGTATGTG
GATGGAGTGGAAGTGCACAATGCCAAGACCAAGCCCAGAGAAGAGCAGTACAACAGCACCTACCGAGTGGTGTCTGT
GCTGACTGTGCTGCACCAGGACTGGCTGAATGGGAAGGAGTACAAGTGTGCTGTGTCCAACAAGGCCCTGCCTGCCC
CCATTGAGAAGACCATCTCCAAGGCCAAGGGCCAGCCCAGAGAGCCCCAGGTCTACACCCTGCCTCCCTCCAGAGAT
GAGCTGACCAAGAACCAGGTGTCCCTGTGGTGCCTGGTGAAAGGCTTCTACCCCAGTGACATTGCTGTGGAGTGGGA
GAGCAATGGCCAGCCAGAGAACAACTACAAGACCACCCCTCCTGTGCTGGACTCTGATGGCTCCTTCTTCCTGTACA
GCAAGCTGACTGTGGACAAGAGCCGCTGGCAGCAGGGCAATGTGTTCTCCTGCTCTGTCATGCATGAGGCCCTGCAC
AACCGCTTCACCCAGAAGAGCCTGAGCCTGAGCCCTGGATGA
>Sequence ID 37: SI-75E2 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPG
>Sequence ID 38: SI-75E2 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAAGGCCCCTCTGTGTTC
CCTCTGGCCCCCTCCTCCAAGAGCACCTCTGGAGGCACTGCTGCCCTGGGCTGCCTGGTGAAGGACTACTTCCCAGA
GCCTGTGACTGTGTCCTGGAACTCTGGAGCCCTGACCTCTGGAGTGCACACCTTCCCTGCTGTGCTGCAGAGCTCTG
GCCTCTACAGCCTGTCCTCTGTGGTCACTGTGCCCTCCTCCAGCCTGGGCACCCAGACCTACATCTGCAATGTGAAC
CACAAGCCCTCCAACACCAAGGTGGACAAGAGAGTGGAGCCCAAGTCCTGTGACAAGACCCACACCTGCCCTCCCTG
CCCAGCCCCAGAGGCTGCTGGAGGCCCCTCTGTGTTCCTCTTCCCTCCCAAGCCCAAGGACACCCTGATGATCTCCA
GGACCCCAGAAGTCACCTGTGTGGTGGTGGATGTGTCCCATGAAGACCCAGAAGTGAAGTTCAACTGGTATGTGGAT
GGAGTGGAGGTGCACAATGCCAAGACCAAGCCCAGAGAAGAGCAGTACAACTCCACCTACCGAGTGGTGTCTGTGCT
GACTGTGCTGCACCAGGACTGGCTGAATGGGAAGGAGTACAAGTGTGCTGTGTCCAACAAGGCCCTGCCTGCCCCCA
TTGAGAAGACCATCTCCAAGGCCAAGGGCCAGCCCAGAGAGCCCCAGGTCTACACCCTGCCTCCCTCCAGAGATGAG
CTGACCAAGAACCAGGTGTCCCTGTCCTGTGCTGTGAAAGGCTTCTACCCCAGTGACATTGCTGTGGAGTGGGAGAG
CAATGGCCAGCCAGAGAACAACTACAAGACCACCCCTCCTGTGCTGGACTCTGATGGCTCCTTCTTCCTGGTCTCCA
AGCTGACTGTGGACAAGAGCCGCTGGCAGCAGGGCAATGTGTTCTCCTGCTCTGTCATGCATGAGGCCCTGCACAAC
CACTACACCCAGAAGAGCCTGAGCCTGAGCCCTGGATGA
>Sequence ID 39: SI-75E2 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPGGGGGSGGGGSDIQMTQSPSTLSASVGDRVTITCQASQSISSHLNWYQQKPGKAPKLLIYKASTL
ASGVPSRFSGSGSGTEFTLTISSLQPDDFATYYCQQGYSWGNVDNVFGGGTKVTVLGGGGSGGGGSGGGGSGGGGSE
VQLLESGGGLVQPGGSLSLSCAASGFSFSSGYDMCWVRQAPGKGLEWIACIAAGSAGITYDANWAKGRFTISRDNSK
NTLYLQMNSLRAEDTAVYYCARSAFSFDYAMDLWGQGTLVTVSS
>Sequence ID 40: SI-75E2 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCAAGCCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACAGATTCACGCAGAAGAGCCTCTCCCTGTCTCCGGGTGGCGGTGGAGGGTCCGGCGGTGGTGGATCCGACATTCA
GATGACACAGTCACCGTCTACGCTCTCAGCCTCAGTAGGCGATAGGGTGACTATCACCTGCCAAGCCTCCCAATCTA
TAAGTTCACACCTCAACTGGTATCAACAGAAACCCGGTAAAGCACCGAAGCTGCTCATCTACAAGGCATCAACTCTT
GCCTCAGGTGTGCCTAGTCGCTTCTCCGGGAGCGGCTCCGGTACTGAGTTTACTTTGACGATATCTTCCCTCCAACC
CGACGACTTCGCCACATACTATTGTCAGCAGGGTTATAGTTGGGGAAATGTTGACAATGTGTTTGGAGGGGGAACGA
AAGTGACTGTGTTGGGAGGGGGAGGAAGTGGCGGCGGCGGAAGCGGAGGTGGTGGCTCCGGCGGGGGTGGAAGCGAA
GTGCAATTGCTCGAAAGTGGTGGCGGACTTGTGCAACCTGGTGGAAGCCTCTCACTCTCTTGCGCGGCGAGTGGTTT
TAGCTTCAGTAGCGGCTACGATATGTGCTGGGTAAGACAGGCTCCTGGTAAAGGCCTTGAGTGGATTGCGTGTATCG
CCGCCGGGTCTGCTGGTATAACATACGATGCAAACTGGGCGAAAGGGCGCTTCACAATCTCCAGGGACAACTCAAAA
AATACATTGTACCTCCAGATGAATAGCCTGAGAGCCGAAGACACCGCAGTCTACTATTGCGCAAGGTCAGCATTTAG
CTTCGATTATGCAATGGATCTTTGGGGCCAGGGGACGCTCGTCACCGTCTCATCTTGA
>Sequence ID 41: SI-75x3 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRETISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPG
>Sequence ID 42: SI-75X3 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAAGGCCCCTCTGTGTTC
CCTCTGGCCCCCTCCTCCAAGAGCACCTCTGGAGGCACTGCTGCCCTGGGCTGCCTGGTGAAGGACTACTTCCCAGA
GCCTGTGACTGTGTCCTGGAACTCTGGAGCCCTGACCTCTGGAGTGCACACCTTCCCTGCTGTGCTGCAGAGCTCTG
GCCTCTACAGCCTGTCCTCTGTGGTCACTGTGCCCTCCTCCAGCCTGGGCACCCAGACCTACATCTGCAATGTGAAC
CACAAGCCCTCCAACACCAAGGTGGACAAGAGAGTGGAGCCCAAGTCCTGTGACAAGACCCACACCTGCCCTCCCTG
CCCAGCCCCAGAGGCTGCTGGAGGCCCCTCTGTGTTCCTCTTCCCTCCCAAGCCCAAGGACACCCTGATGATCTCCA
GGACCCCAGAAGTCACCTGTGTGGTGGTGGATGTGTCCCATGAAGACCCAGAAGTGAAGTTCAACTGGTATGTGGAT
GGAGTGGAGGTGCACAATGCCAAGACCAAGCCCAGAGAAGAGCAGTACAACTCCACCTACCGAGTGGTGTCTGTGCT
GACTGTGCTGCACCAGGACTGGCTGAATGGGAAGGAGTACAAGTGTGCTGTGTCCAACAAGGCCCTGCCTGCCCCCA
TTGAGAAGACCATCTCCAAGGCCAAGGGCCAGCCCAGAGAGCCCCAGGTCTACACCCTGCCTCCCTCCAGAGATGAG
CTGACCAAGAACCAGGTGTCCCTGTCCTGTGCTGTGAAAGGCTTCTACCCCAGTGACATTGCTGTGGAGTGGGAGAG
CAATGGCCAGCCAGAGAACAACTACAAGACCACCCCTCCTGTGCTGGACTCTGATGGCTCCTTCTTCCTGGTCTCCA
AGCTGACTGTGGACAAGAGCCGCTGGCAGCAGGGCAATGTGTTCTCCTGCTCTGTCATGCATGAGGCCCTGCACAAC
CACTACACCCAGAAGAGCCTGAGCCTGAGCCCTGGATGA
>Sequence ID 43: SI-75X3 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPG
>Sequence ID 44: SI-75X3 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCCCCCTCTGTGTTCATCTTCCCACCCAGT
GATGAGCAGCTGAAGAGTGGCACAGCCTCTGTGGTGTGCCTGCTCAACAACTTCTACCCCAGAGAAGCCAAGGTGCA
GTGGAAGGTGGACAATGCCCTGCAGAGTGGCAACAGCCAGGAGAGTGTGACAGAGCAGGACAGCAAGGACAGCACCT
ACAGCCTGTCCTCCACCCTGACCCTGTCCAAGGCTGACTATGAGAAGCACAAGGTGTATGCCTGTGAGGTGACCCAC
CAGGGCCTGAGCAGCCCTGTGACCAAGAGCTTCAACCGAGGAGAGTGTGGAGGAGACAAGACCCACACCTGCCCTCC
CTGCCCAGCCCCAGAAGCTGCTGGAGGCCCCTCTGTGTTCCTCTTCCCTCCCAAGCCCAAGGACACCCTGATGATCT
CCAGGACCCCAGAAGTCACCTGTGTGGTGGTGGATGTGTCCCATGAAGACCCAGAAGTGAAGTTCAACTGGTATGTG
GATGGAGTGGAAGTGCACAATGCCAAGACCAAGCCCAGAGAAGAGCAGTACAACAGCACCTACCGAGTGGTGTCTGT
GCTGACTGTGCTGCACCAGGACTGGCTGAATGGGAAGGAGTACAAGTGTGCTGTGTCCAACAAGGCCCTGCCTGCCC
CCATTGAGAAGACCATCTCCAAGGCCAAGGGCCAGCCCAGAGAGCCCCAGGTCTACACCCTGCCTCCCTCCAGAGAT
GAGCTGACCAAGAACCAGGTGTCCCTGTGGTGCCTGGTGAAAGGCTTCTACCCCAGTGACATTGCTGTGGAGTGGGA
GAGCAATGGCCAGCCAGAGAACAACTACAAGACCACCCCTCCTGTGCTGGACTCTGATGGCTCCTTCTTCCTGTACA
GCAAGCTGACTGTGGACAAGAGCCGCTGGCAGCAGGGCAATGTGTTCTCCTGCTCTGTCATGCATGAGGCCCTGCAC
AACCGCTTCACCCAGAAGAGCCTGAGCCTGAGCCCTGGATGA
>Sequence ID 45: SI-75X16 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSQVQLQESGGRLVQPGEPLSLTCKTSGIDLSSNAIGWVRQAPGKGLEWIGVIF
GSGNTYYASWAKGRFTISRSTSTVYLKMNSLRSEDTAIYYCARGGYSSDIWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVNHKPSNTK
VDKRVEPKSCDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LSCAVKGFYPSDIAVEWESNGQPENNYKTTPPVLDSDGSFFLVSKLTVDKSRWQQGNVFSCSVMHEALHNHYTQKSL
SLSPG
>Sequence ID 46: SI-75X16 Chain A Nucleotide Sequence
GAGATCGTGCTGACCCAGTCTCCTTCCACACTGTCTGTGTCTCCCGGCGAGAGAGCCACCTTCAGCTGTAGAGCCTC
TCAGTCCATCGGCACCAACATCCACTGGTATCAGCAGAAGCCCGGCAAGCCTCCTCGGCTGCTGATTAAGTACGCCT
CCGAGTCCATCAGCGGCATCCCTGACAGATTCTCCGGCTCTGGCTCTGGCACCGAGTTTACCCTGACCATCTCCTCC
GTGCAGTCCGAGGATTTCGCCGTGTACTACTGCCAGCAGAACAACAACTGGCCCACCACCTTTGGACCCGGCACCAA
GCTGACAGTTCTTGGCGGCGGAGGATCTGGCGGAGGTGGAAGCGGAGGCGGAGGAAGCGGTGGCGGCGGATCTCAAG
TTCAGTTGCAGCAGTCTGGCCCTGGCCTGGTCAAGCCTTCTGAGACACTGTCCATCACCTGTACCGTGTCCGGCTTC
TCCCTGACCAATTACGGCGTGCACTGGATCAGACAGGCCCCTGGCAAAGGACTGGAATGGCTGGGAGTGATTTGGAG
CGGCGGCAACACCGACTACAACACCCCTTTCACCAGCCGGTTCACCATCACCAAGGACAACTCCAAGAACCAGGTGT
ACTTCAAGCTGCGGAGCGTGCGGGCTGATGACACCGCCATCTACTACTGTGCTCGGGCCCTGACCTACTACGACTAC
GAGTTTGCTTACTGGGGCCAGGGCACCCTGGTCACAGTCTCGAGCGGCGGTGGAGGGTCCGGCGGTGGTGGATCACA
AGTGCAGTTGCAAGAAAGTGGTGGTAGACTGGTTCAGCCTGGTGAACCCTTGTCACTGACGTGTAAAACAAGCGGCA
TTGATCTGTCCTCTAACGCCATCGGATGGGTCCGACAGGCCCCAGGAAAAGGTCTGGAGTGGATCGGAGTTATCTTC
GGGAGCGGCAATACATACTACGCAAGCTGGGCAAAAGGGCGATTTACGATATCACGGAGCACCTCTACAGTTTATTT
GAAAATGAACTCCCTCCGGTCCGAGGATACCGCGATATATTACTGTGCCAGAGGGGGGTACTCCTCTGATATCTGGG
GGCAGGGTACACTGGTTACAGTTTCATCCGCTAGCACCAAAGGCCCCTCTGTGTTCCCTCTGGCCCCCTCCTCCAAG
AGCACCTCTGGAGGCACTGCTGCCCTGGGCTGCCTGGTGAAGGACTACTTCCCAGAGCCTGTGACTGTGTCCTGGAA
CTCTGGAGCCCTGACCTCTGGAGTGCACACCTTCCCTGCTGTGCTGCAGAGCTCTGGCCTCTACAGCCTGTCCTCTG
TGGTCACTGTGCCCTCCTCCAGCCTGGGCACCCAGACCTACATCTGCAATGTGAACCACAAGCCCTCCAACACCAAG
GTGGACAAGAGAGTGGAGCCCAAGTCCTGTGACAAGACCCACACCTGCCCTCCCTGCCCAGCCCCAGAGGCTGCTGG
AGGCCCCTCTGTGTTCCTCTTCCCTCCCAAGCCCAAGGACACCCTGATGATCTCCAGGACCCCAGAAGTCACCTGTG
TGGTGGTGGATGTGTCCCATGAAGACCCAGAAGTGAAGTTCAACTGGTATGTGGATGGAGTGGAGGTGCACAATGCC
AAGACCAAGCCCAGAGAAGAGCAGTACAACTCCACCTACCGAGTGGTGTCTGTGCTGACTGTGCTGCACCAGGACTG
GCTGAATGGGAAGGAGTACAAGTGTGCTGTGTCCAACAAGGCCCTGCCTGCCCCCATTGAGAAGACCATCTCCAAGG
CCAAGGGCCAGCCCAGAGAGCCCCAGGTCTACACCCTGCCTCCCTCCAGAGATGAGCTGACCAAGAACCAGGTGTCC
CTGTCCTGTGCTGTGAAAGGCTTCTACCCCAGTGACATTGCTGTGGAGTGGGAGAGCAATGGCCAGCCAGAGAACAA
CTACAAGACCACCCCTCCTGTGCTGGACTCTGATGGCTCCTTCTTCCTGGTCTCCAAGCTGACTGTGGACAAGAGCC
GCTGGCAGCAGGGCAATGTGTTCTCCTGCTCTGTCATGCATGAGGCCCTGCACAACCACTACACCCAGAAGAGCCTG
AGCCTGAGCCCTGGATGA
>Sequence ID 47: SI-75X16 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLWCLVKGFYPSDIAVEWESNGQPENNYKTTPPVLDSDGSFFLYSKLTVDKSRWQQGNVFSCSVMHEALHNR
FTQKSLSLSPG
>Sequence ID 48: SI-75X16 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCCCCCTCTGTGTTCATCTTCCCACCCAGTGATGAG
CAGCTGAAGAGTGGCACAGCCTCTGTGGTGTGCCTGCTCAACAACTTCTACCCCAGAGAAGCCAAGGTGCAGTGGAA
GGTGGACAATGCCCTGCAGAGTGGCAACAGCCAGGAGAGTGTGACAGAGCAGGACAGCAAGGACAGCACCTACAGCC
TGTCCTCCACCCTGACCCTGTCCAAGGCTGACTATGAGAAGCACAAGGTGTATGCCTGTGAGGTGACCCACCAGGGC
CTGAGCAGCCCTGTGACCAAGAGCTTCAACCGAGGAGAGTGTGGAGGAGACAAGACCCACACCTGCCCTCCCTGCCC
AGCCCCAGAAGCTGCTGGAGGCCCCTCTGTGTTCCTCTTCCCTCCCAAGCCCAAGGACACCCTGATGATCTCCAGGA
CCCCAGAAGTCACCTGTGTGGTGGTGGATGTGTCCCATGAAGACCCAGAAGTGAAGTTCAACTGGTATGTGGATGGA
GTGGAAGTGCACAATGCCAAGACCAAGCCCAGAGAAGAGCAGTACAACAGCACCTACCGAGTGGTGTCTGTGCTGAC
TGTGCTGCACCAGGACTGGCTGAATGGGAAGGAGTACAAGTGTGCTGTGTCCAACAAGGCCCTGCCTGCCCCCATTG
AGAAGACCATCTCCAAGGCCAAGGGCCAGCCCAGAGAGCCCCAGGTCTACACCCTGCCTCCCTCCAGAGATGAGCTG
ACCAAGAACCAGGTGTCCCTGTGGTGCCTGGTGAAAGGCTTCTACCCCAGTGACATTGCTGTGGAGTGGGAGAGCAA
TGGCCAGCCAGAGAACAACTACAAGACCACCCCTCCTGTGCTGGACTCTGATGGCTCCTTCTTCCTGTACAGCAAGC
TGACTGTGGACAAGAGCCGCTGGCAGCAGGGCAATGTGTTCTCCTGCTCTGTCATGCATGAGGCCCTGCACAACCGC
TTCACCCAGAAGAGCCTGAGCCTGAGCCCTGGA
>Sequence ID 49: SI-75X18 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPGGGGGSGGGGSQVQLQQSGPG
LVKPSETLSITCTVSGFSLTNYGVHWIRQAPGKGLEWLGVIWSGGNTDYNTPFTSRFTITKDNSKNQVYFKLRSVRA
DDTAIYYCARALTYYDYEFAYWGQGTLVTVSSGGGGSGGGGSGGGGSGGGGSEIVLTQSPSTLSVSPGERATFSCRA
SQSIGTNIHWYQQKPGKPPRLLIKYASESISGIPDRFSGSGSGTEFTLTISSVQSEDFAVYYCQQNNNWPTTFGPGT
KLTVL
>Sequence ID 50: SI-75X18 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGAGGAGGAGGAGGCTCTGGAGGAGGAGGATCCCAAGTTCAGTTGCAGCAGTCTGGCCCTGGC
CTGGTCAAGCCTTCTGAGACACTGTCCATCACCTGTACCGTGTCCGGCTTCTCCCTGACCAATTACGGCGTGCACTG
GATCAGACAGGCCCCTGGCAAAGGACTGGAATGGCTGGGAGTGATTTGGAGCGGCGGCAACACCGACTACAACACCC
CTTTCACCAGCCGGTTCACCATCACCAAGGACAACTCCAAGAACCAGGTGTACTTCAAGCTGCGGAGCGTGCGGGCT
GATGACACCGCCATCTACTACTGTGCTCGGGCCCTGACCTACTACGACTACGAGTTTGCTTACTGGGGCCAGGGCAC
CCTGGTCACAGTTTCTTCTGGTGGTGGTGGTTCAGGCGGGGGGGGTTCCGGCGGAGGCGGTTCTGGGGGTGGAGGTT
CCGAGATCGTGCTGACCCAGTCTCCTTCCACACTGTCTGTGTCTCCCGGCGAGAGAGCCACCTTCAGCTGTAGAGCC
TCTCAGTCCATCGGCACCAACATCCACTGGTATCAGCAGAAGCCCGGCAAGCCTCCTCGGCTGCTGATTAAGTACGC
CTCCGAGTCCATCAGCGGCATCCCTGACAGATTCTCCGGCTCTGGCTCTGGCACCGAGTTTACCCTGACCATCTCCT
CCGTGCAGTCCGAGGATTTCGCCGTGTACTACTGCCAGCAGAACAACAACTGGCCCACCACCTTTGGACCCGGCACC
AAGCTGACCGTGCTGTGA
>Sequence ID 51: SI-75X18 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPGGGGGSGGGGSQVQLQES
GGGLVKPGGSLSLSCAASGFTFSSYWMSWVRQAPGKGLEWVANINRDGSASYYVDSVKGRFTISRDDAKNSLYLQMN
SLRAEDTAVYYCARDRGVGYFDLWGRGTLVTVSSGGGGSGGGGSGGGGSQSALTQPASVSGSPGQSITISCTGTSSD
VGGYNFVSWYQQHPGKAPKLMIYDVSDRPSGVSDRFSGSKSGNTASLIISGLQADDEADYYCSSYGSSSTHVIFGGG
TKVTVL
>Sequence ID 52: SI-75X18 Chain B Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGAGGAGGAGGAGGCTCTGGAGGAGGAGGATCCCAGGTGCAGCTGCAGGAGTCG
GGGGGAGGCCTGGTCAAGCCTGGAGGGTCCCTGAGCCTCTCCTGTGCAGCCTCTGGATTCACCTTTAGTAGTTATTG
GATGAGCTGGGTCCGCCAGGCTCCAGGGAAGGGGCTGGAGTGGGTGGCCAACATAAACCGCGATGGAAGTGCGAGTT
ACTATGTGGACTCTGTGAAGGGCCGATTCACCATCTCCAGAGACGACGCCAAGAACTCACTGTATCTGCAAATGAAC
AGCCTGAGAGCTGAGGACACGGCTGTGTATTACTGTGCGAGAGATCGTGGGGTGGGCTACTTCGATCTCTGGGGCCG
TGGCACCCTGGTCACCGTGTCTAGCGGTGGAGGCGGTTCAGGCGGAGGTGGTTCCGGCGGTGGCGGCTCCCAGTCTG
CCCTGACTCAGCCTGCCTCCGTGTCTGGGTCTCCTGGACAGTCGATCACCATCTCCTGCACTGGAACCAGCAGTGAC
GTTGGTGGTTATAACTTTGTCTCCTGGTACCAACAACACCCAGGCAAAGCCCCCAAACTCATGATCTATGATGTCAG
TGATCGGCCCTCAGGGGTGTCTGATCGCTTCTCCGGCTCCAAGTCTGGCAACACGGCCTCCCTGATCATCTCTGGCC
TCCAGGCTGACGACGAGGCTGATTATTACTGCAGCTCATATGGGAGCAGCAGCACTCATGTGATTTTCGGCGGAGGG
ACCAAGGTGACCGTCCTATAA
>Sequence ID 53: SI-75X1 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPG
>Sequence ID 54: SI-75X1 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAAGGCCCCTCTGTGTTC
CCTCTGGCCCCCTCCTCCAAGAGCACCTCTGGAGGCACTGCTGCCCTGGGCTGCCTGGTGAAGGACTACTTCCCAGA
GCCTGTGACTGTGTCCTGGAACTCTGGAGCCCTGACCTCTGGAGTGCACACCTTCCCTGCTGTGCTGCAGAGCTCTG
GCCTCTACAGCCTGTCCTCTGTGGTCACTGTGCCCTCCTCCAGCCTGGGCACCCAGACCTACATCTGCAATGTGAAC
CACAAGCCCTCCAACACCAAGGTGGACAAGAGAGTGGAGCCCAAGTCCTGTGACAAGACCCACACCTGCCCTCCCTG
CCCAGCCCCAGAGGCTGCTGGAGGCCCCTCTGTGTTCCTCTTCCCTCCCAAGCCCAAGGACACCCTGATGATCTCCA
GGACCCCAGAAGTCACCTGTGTGGTGGTGGATGTGTCCCATGAAGACCCAGAAGTGAAGTTCAACTGGTATGTGGAT
GGAGTGGAGGTGCACAATGCCAAGACCAAGCCCAGAGAAGAGCAGTACAACTCCACCTACCGAGTGGTGTCTGTGCT
GACTGTGCTGCACCAGGACTGGCTGAATGGGAAGGAGTACAAGTGTGCTGTGTCCAACAAGGCCCTGCCTGCCCCCA
TTGAGAAGACCATCTCCAAGGCCAAGGGCCAGCCCAGAGAGCCCCAGGTCTACACCCTGCCTCCCTCCAGAGATGAG
CTGACCAAGAACCAGGTGTCCCTGTCCTGTGCTGTGAAAGGCTTCTACCCCAGTGACATTGCTGTGGAGTGGGAGAG
CAATGGCCAGCCAGAGAACAACTACAAGACCACCCCTCCTGTGCTGGACTCTGATGGCTCCTTCTTCCTGGTCTCCA
AGCTGACTGTGGACAAGAGCCGCTGGCAGCAGGGCAATGTGTTCTCCTGCTCTGTCATGCATGAGGCCCTGCACAAC
CACTACACCCAGAAGAGCCTGAGCCTGAGCCCTGGATGA
>Sequence ID 55: SI-75X1 Chain B Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGDKTHTCPPCP
APEAAGGPSVFLFPPKPKDTLMISRTPEVTCVVVDVSHEDPEVKFNWYVDGVEVHNAKTKPREEQYNSTYRVVSVLT
VLHQDWLNGKEYKCAVSNKALPAPIEKTISKAKGQPREPQVYTLPPSRDELTKNQVSLWCLVKGFYPSDIAVEWESN
GQPENNYKTTPPVLDSDGSFFLYSKLTVDKSRWQQGNVFSCSVMHEALHNRFTQKSLSLSPG
>Sequence ID 56: SI-75X1 Chain B Nucleotide Sequence
GACGTGGTCATGACACAGAGCCCTTCCACACTGTCTGCCTCCGTGGGCGATAGAGTGACCATCAACTGTCAGGCCTC
CGAGTCCATCTCCTCTTGGCTGGCTTGGTATCAGCAGAAGCCTGGCAAGGCCCCTAAGCTGCTGATCTACGAGGCTT
CCAAGCTGGCCTCTGGCGTGCCCTCTAGATTTTCCGGCTCTGGCTCTGGCACCGAGTTTACCCTGACAATCTCCAGC
CTGCAGCCTGACGACTTCGCCACCTACTACTGCCAAGGCTACTTCTACTTCATCTCCCGGACCTACGTGAACTCCTT
CGGCGGAGGCACCAAGGTGGAGATCAAGCGTACGGTGGCTGCCCCCTCTGTGTTCATCTTCCCACCCAGTGATGAGC
AGCTGAAGAGTGGCACAGCCTCTGTGGTGTGCCTGCTCAACAACTTCTACCCCAGAGAAGCCAAGGTGCAGTGGAAG
GTGGACAATGCCCTGCAGAGTGGCAACAGCCAGGAGAGTGTGACAGAGCAGGACAGCAAGGACAGCACCTACAGCCT
GTCCTCCACCCTGACCCTGTCCAAGGCTGACTATGAGAAGCACAAGGTGTATGCCTGTGAGGTGACCCACCAGGGCC
TGAGCAGCCCTGTGACCAAGAGCTTCAACCGAGGAGAGTGTGGAGGAGACAAGACCCACACCTGCCCTCCCTGCCCA
GCCCCAGAAGCTGCTGGAGGCCCCTCTGTGTTCCTCTTCCCTCCCAAGCCCAAGGACACCCTGATGATCTCCAGGAC
CCCAGAAGTCACCTGTGTGGTGGTGGATGTGTCCCATGAAGACCCAGAAGTGAAGTTCAACTGGTATGTGGATGGAG
TGGAAGTGCACAATGCCAAGACCAAGCCCAGAGAAGAGCAGTACAACAGCACCTACCGAGTGGTGTCTGTGCTGACT
GTGCTGCACCAGGACTGGCTGAATGGGAAGGAGTACAAGTGTGCTGTGTCCAACAAGGCCCTGCCTGCCCCCATTGA
GAAGACCATCTCCAAGGCCAAGGGCCAGCCCAGAGAGCCCCAGGTCTACACCCTGCCTCCCTCCAGAGATGAGCTGA
CCAAGAACCAGGTGTCCCTGTGGTGCCTGGTGAAAGGCTTCTACCCCAGTGACATTGCTGTGGAGTGGGAGAGCAAT
GGCCAGCCAGAGAACAACTACAAGACCACCCCTCCTGTGCTGGACTCTGATGGCTCCTTCTTCCTGTACAGCAAGCT
GACTGTGGACAAGAGCCGCTGGCAGCAGGGCAATGTGTTCTCCTGCTCTGTCATGCATGAGGCCCTGCACAACCGCT
TCACCCAGAAGAGCCTGAGCCTGAGCCCTGGATGA
>Sequence ID 57: SI-75X2 Chain A Amino Acid Sequence
EVQLVESGGGLVQPGGSLRLSCAASGETISTNAMSWVRQAPGKGLEWIGVITGRDITYYASWAKGRFTISRDNSKNT
LYLQMNSLRAEDTAVYYCARDGGSSAITSNNIWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVHNAKTKPREEQYNSTYRVVSV
LTVLHQDWLNGKEYKCAVSNKALPAPIEKTISKAKGQPREPQVYTLPPSRDELTKNQVSLSCAVKGFYPSDIAVEWE
SNGQPENNYKTTPPVLDSDGSFFLVSKLTVDKSRWQQGNVFSCSVMHEALHNHYTQKSLSLSPG
>Sequence ID 58: SI-75X2 Chain A Nucleotide Sequence
GAGGTGCAGCTGGTTGAATCTGGCGGAGGATTGGTTCAGCCTGGCGGCTCTCTGAGACTGTCTTGTGCCGCTTCTGG
CTTCACCATCTCCACCAACGCCATGTCCTGGGTCCGACAGGCTCCTGGCAAAGGCCTGGAATGGATCGGCGTGATCA
CCGGCAGAGATATTACCTACTACGCCTCCTGGGCCAAGGGCAGATTCACCATCAGCCGGGACAACTCCAAGAACACC
CTGTACCTGCAGATGAACTCCCTGAGAGCCGAGGACACCGCCGTGTACTACTGTGCTAGAGATGGCGGCAGCAGCGC
CATCACCTCCAACAATATTTGGGGCCAGGGCACCCTGGTCACTGTGTCCTCTGCTAGCACCAAAGGCCCCTCTGTGT
TCCCTCTGGCCCCCTCCTCCAAGAGCACCTCTGGAGGCACTGCTGCCCTGGGCTGCCTGGTGAAGGACTACTTCCCA
GAGCCTGTGACTGTGTCCTGGAACTCTGGAGCCCTGACCTCTGGAGTGCACACCTTCCCTGCTGTGCTGCAGAGCTC
TGGCCTCTACAGCCTGTCCTCTGTGGTCACTGTGCCCTCCTCCAGCCTGGGCACCCAGACCTACATCTGCAATGTGA
ACCACAAGCCCTCCAACACCAAGGTGGACAAGAGAGTGGAGCCCAAGTCCTGTGACAAGACCCACACCTGCCCTCCC
TGCCCAGCCCCAGAGGCTGCTGGAGGCCCCTCTGTGTTCCTCTTCCCTCCCAAGCCCAAGGACACCCTGATGATCTC
CAGGACCCCAGAAGTCACCTGTGTGGTGGTGGATGTGTCCCATGAAGACCCAGAAGTGAAGTTCAACTGGTATGTGG
ATGGAGTGGAGGTGCACAATGCCAAGACCAAGCCCAGAGAAGAGCAGTACAACTCCACCTACCGAGTGGTGTCTGTG
CTGACTGTGCTGCACCAGGACTGGCTGAATGGGAAGGAGTACAAGTGTGCTGTGTCCAACAAGGCCCTGCCTGCCCC
CATTGAGAAGACCATCTCCAAGGCCAAGGGCCAGCCCAGAGAGCCCCAGGTCTACACCCTGCCTCCCTCCAGAGATG
AGCTGACCAAGAACCAGGTGTCCCTGTCCTGTGCTGTGAAAGGCTTCTACCCCAGTGACATTGCTGTGGAGTGGGAG
AGCAATGGCCAGCCAGAGAACAACTACAAGACCACCCCTCCTGTGCTGGACTCTGATGGCTCCTTCTTCCTGGTCTC
CAAGCTGACTGTGGACAAGAGCCGCTGGCAGCAGGGCAATGTGTTCTCCTGCTCTGTCATGCATGAGGCCCTGCACA
ACCACTACACCCAGAAGAGCCTGAGCCTGAGCCCTGGATGA
>Sequence ID 59: SI-75X2 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLWCLVKGFYPSDIAVEWESNGQPENNYKTTPPVLDSDGSFFLYSKLTVDKSRWQQGNVFSCSVMHEALH
NRFTQKSLSLSPG
>Sequence ID 60: SI-75X2 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCCCCCTCTGTGTTCATCTTCCCACCCAGT
GATGAGCAGCTGAAGAGTGGCACAGCCTCTGTGGTGTGCCTGCTCAACAACTTCTACCCCAGAGAAGCCAAGGTGCA
GTGGAAGGTGGACAATGCCCTGCAGAGTGGCAACAGCCAGGAGAGTGTGACAGAGCAGGACAGCAAGGACAGCACCT
ACAGCCTGTCCTCCACCCTGACCCTGTCCAAGGCTGACTATGAGAAGCACAAGGTGTATGCCTGTGAGGTGACCCAC
CAGGGCCTGAGCAGCCCTGTGACCAAGAGCTTCAACCGAGGAGAGTGTGGAGGAGACAAGACCCACACCTGCCCTCC
CTGCCCAGCCCCAGAAGCTGCTGGAGGCCCCTCTGTGTTCCTCTTCCCTCCCAAGCCCAAGGACACCCTGATGATCT
CCAGGACCCCAGAAGTCACCTGTGTGGTGGTGGATGTGTCCCATGAAGACCCAGAAGTGAAGTTCAACTGGTATGTG
GATGGAGTGGAAGTGCACAATGCCAAGACCAAGCCCAGAGAAGAGCAGTACAACAGCACCTACCGAGTGGTGTCTGT
GCTGACTGTGCTGCACCAGGACTGGCTGAATGGGAAGGAGTACAAGTGTGCTGTGTCCAACAAGGCCCTGCCTGCCC
CCATTGAGAAGACCATCTCCAAGGCCAAGGGCCAGCCCAGAGAGCCCCAGGTCTACACCCTGCCTCCCTCCAGAGAT
GAGCTGACCAAGAACCAGGTGTCCCTGTGGTGCCTGGTGAAAGGCTTCTACCCCAGTGACATTGCTGTGGAGTGGGA
GAGCAATGGCCAGCCAGAGAACAACTACAAGACCACCCCTCCTGTGCTGGACTCTGATGGCTCCTTCTTCCTGTACA
GCAAGCTGACTGTGGACAAGAGCCGCTGGCAGCAGGGCAATGTGTTCTCCTGCTCTGTCATGCATGAGGCCCTGCAC
AACCGCTTCACCCAGAAGAGCCTGAGCCTGAGCCCTGGATGA
>Sequence ID 61: SI-75O2 Chain A Amino Acid Sequence
EVQLVESGGGLVQPGGSLRLSCAASGFTISTNAMSWVRQAPGKGLEWIGVITGRDITYYASWAKGRFTISRDNSKNT
LYLQMNSLRAEDTAVYYCARDGGSSAITSNNIWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVHNAKTKPREEQYNSTYRVVSV
LTVLHQDWLNGKEYKCAVSNKALPAPIEKTISKAKGQPREPQVYTLPPSRDELTKNQVSLSCAVKGFYPSDIAVEWE
SNGQPENNYKTTPPVLDSDGSFFLVSKLTVDKSRWQQGNVFSCSVMHEALHNHYTQKSLSLSPG
>Sequence ID 62: SI-75O2 Chain A Nucleotide Sequence
GAGGTGCAGCTGGTTGAATCTGGCGGAGGATTGGTTCAGCCTGGCGGCTCTCTGAGACTGTCTTGTGCCGCTTCTGG
CTTCACCATCTCCACCAACGCCATGTCCTGGGTCCGACAGGCTCCTGGCAAAGGCCTGGAATGGATCGGCGTGATCA
CCGGCAGAGATATTACCTACTACGCCTCCTGGGCCAAGGGCAGATTCACCATCAGCCGGGACAACTCCAAGAACACC
CTGTACCTGCAGATGAACTCCCTGAGAGCCGAGGACACCGCCGTGTACTACTGTGCTAGAGATGGCGGCAGCAGCGC
CATCACCTCCAACAATATTTGGGGCCAGGGCACCCTGGTCACTGTGTCCTCTGCTAGCACCAAAGGCCCCTCTGTGT
TCCCTCTGGCCCCCTCCTCCAAGAGCACCTCTGGAGGCACTGCTGCCCTGGGCTGCCTGGTGAAGGACTACTTCCCA
GAGCCTGTGACTGTGTCCTGGAACTCTGGAGCCCTGACCTCTGGAGTGCACACCTTCCCTGCTGTGCTGCAGAGCTC
TGGCCTCTACAGCCTGTCCTCTGTGGTCACTGTGCCCTCCTCCAGCCTGGGCACCCAGACCTACATCTGCAATGTGA
ACCACAAGCCCTCCAACACCAAGGTGGACAAGAGAGTGGAGCCCAAGTCCTGTGACAAGACCCACACCTGCCCTCCC
TGCCCAGCCCCAGAGGCTGCTGGAGGCCCCTCTGTGTTCCTCTTCCCTCCCAAGCCCAAGGACACCCTGATGATCTC
CAGGACCCCAGAAGTCACCTGTGTGGTGGTGGATGTGTCCCATGAAGACCCAGAAGTGAAGTTCAACTGGTATGTGG
ATGGAGTGGAGGTGCACAATGCCAAGACCAAGCCCAGAGAAGAGCAGTACAACTCCACCTACCGAGTGGTGTCTGTG
CTGACTGTGCTGCACCAGGACTGGCTGAATGGGAAGGAGTACAAGTGTGCTGTGTCCAACAAGGCCCTGCCTGCCCC
CATTGAGAAGACCATCTCCAAGGCCAAGGGCCAGCCCAGAGAGCCCCAGGTCTACACCCTGCCTCCCTCCAGAGATG
AGCTGACCAAGAACCAGGTGTCCCTGTCCTGTGCTGTGAAAGGCTTCTACCCCAGTGACATTGCTGTGGAGTGGGAG
AGCAATGGCCAGCCAGAGAACAACTACAAGACCACCCCTCCTGTGCTGGACTCTGATGGCTCCTTCTTCCTGGTCTC
CAAGCTGACTGTGGACAAGAGCCGCTGGCAGCAGGGCAATGTGTTCTCCTGCTCTGTCATGCATGAGGCCCTGCACA
ACCACTACACCCAGAAGAGCCTGAGCCTGAGCCCTGGATGA
>Sequence ID 63: SI-75O2 Chain B Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGDKTHTCPPCP
APEAAGGPSVFLFPPKPKDTLMISRTPEVTCVVVDVSHEDPEVKFNWYVDGVEVHNAKTKPREEQYNSTYRVVSVLT
VLHQDWLNGKEYKCAVSNKALPAPIEKTISKAKGQPREPQVYTLPPSRDELTKNQVSLWCLVKGFYPSDIAVEWESN
GQPENNYKTTPPVLDSDGSFFLYSKLTVDKSRWQQGNVFSCSVMHEALHNRFTQKSLSLSPG
>Sequence ID 64: SI-75O2 Chain B Nucleotide Sequence
GACGTGGTCATGACACAGAGCCCTTCCACACTGTCTGCCTCCGTGGGCGATAGAGTGACCATCAACTGTCAGGCCTC
CGAGTCCATCTCCTCTTGGCTGGCTTGGTATCAGCAGAAGCCTGGCAAGGCCCCTAAGCTGCTGATCTACGAGGCTT
CCAAGCTGGCCTCTGGCGTGCCCTCTAGATTTTCCGGCTCTGGCTCTGGCACCGAGTTTACCCTGACAATCTCCAGC
CTGCAGCCTGACGACTTCGCCACCTACTACTGCCAAGGCTACTTCTACTTCATCTCCCGGACCTACGTGAACTCCTT
CGGCGGAGGCACCAAGGTGGAGATCAAGCGTACGGTGGCTGCCCCCTCTGTGTTCATCTTCCCACCCAGTGATGAGC
AGCTGAAGAGTGGCACAGCCTCTGTGGTGTGCCTGCTCAACAACTTCTACCCCAGAGAAGCCAAGGTGCAGTGGAAG
GTGGACAATGCCCTGCAGAGTGGCAACAGCCAGGAGAGTGTGACAGAGCAGGACAGCAAGGACAGCACCTACAGCCT
GTCCTCCACCCTGACCCTGTCCAAGGCTGACTATGAGAAGCACAAGGTGTATGCCTGTGAGGTGACCCACCAGGGCC
TGAGCAGCCCTGTGACCAAGAGCTTCAACCGAGGAGAGTGTGGAGGAGACAAGACCCACACCTGCCCTCCCTGCCCA
GCCCCAGAAGCTGCTGGAGGCCCCTCTGTGTTCCTCTTCCCTCCCAAGCCCAAGGACACCCTGATGATCTCCAGGAC
CCCAGAAGTCACCTGTGTGGTGGTGGATGTGTCCCATGAAGACCCAGAAGTGAAGTTCAACTGGTATGTGGATGGAG
TGGAAGTGCACAATGCCAAGACCAAGCCCAGAGAAGAGCAGTACAACAGCACCTACCGAGTGGTGTCTGTGCTGACT
GTGCTGCACCAGGACTGGCTGAATGGGAAGGAGTACAAGTGTGCTGTGTCCAACAAGGCCCTGCCTGCCCCCATTGA
GAAGACCATCTCCAAGGCCAAGGGCCAGCCCAGAGAGCCCCAGGTCTACACCCTGCCTCCCTCCAGAGATGAGCTGA
CCAAGAACCAGGTGTCCCTGTGGTGCCTGGTGAAAGGCTTCTACCCCAGTGACATTGCTGTGGAGTGGGAGAGCAAT
GGCCAGCCAGAGAACAACTACAAGACCACCCCTCCTGTGCTGGACTCTGATGGCTCCTTCTTCCTGTACAGCAAGCT
GACTGTGGACAAGAGCCGCTGGCAGCAGGGCAATGTGTTCTCCTGCTCTGTCATGCATGAGGCCCTGCACAACCGCT
TCACCCAGAAGAGCCTGAGCCTGAGCCCTGGATGA
>Sequence ID 65: SI-7507 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 66: SI-7507 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGA
>Sequence ID 67: SI-7507 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPG
>Sequence ID 68: SI-7507 Chain B Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGATGA
>Sequence ID 69: SI-68P13 Chain A Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSDVVMTQSPSSVSASVGDRVTITCQASQNIRTYLSWYQQKPGKAPKLLIYAAANLA
SGVPSRFSGSGSGTDFTLTISDLEPGDAATYYCQSTYLGTDYVGGAFGGGTKLTVLGGGGSGGGGSGGGGSGGGGSE
VQLVESGGGLVQPGGSLRLSCTASGFTISSYHMQWVRQAPGKGLEYIGTISSGGNVYYASSARGRFTISRPSSKNTV
DLQMNSLRAEDTAVYYCARDSGYSDPMWGQGTLVTVSS
>Sequence ID 70: SI-68P13 Chain A Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTATACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGCGGCGGTTCAGGTGGTGGGGGATCCGACGTTGTGAT
GACGCAGTCTCCTAGTTCAGTATCAGCGTCTGTTGGGGATCGAGTCACGATTACATGCCAGGCGAGCCAAAATATTA
GGACGTATCTGTCTTGGTATCAACAAAAGCCTGGTAAGGCTCCAAAACTCCTCATTTACGCCGCTGCCAACTTGGCT
AGTGGAGTACCTTCACGCTTTAGTGGGTCAGGTTCTGGAACAGATTTTACGTTGACGATCTCCGATTTGGAACCAGG
GGACGCTGCCACGTATTATTGCCAGTCCACCTACCTCGGGACTGACTACGTTGGTGGGGCGTTTGGCGGAGGAACAA
AACTCACTGTACTTGGTGGTGGTGGTTCAGGCGGGGGGGGTTCCGGCGGAGGCGGTTCTGGGGGTGGAGGTTCCGAG
GTTCAGTTGGTTGAAAGTGGGGGGGGACTCGTTCAGCCTGGAGGCTCTTTGCGACTTTCTTGTACCGCTTCTGGGTT
CACTATAAGTTCATATCATATGCAATGGGTAAGACAGGCGCCTGGTAAGGGACTTGAATACATAGGAACAATAAGCT
CTGGTGGTAACGTCTATTACGCCTCATCCGCGCGGGGGAGGTTTACAATTTCCAGGCCTTCTAGCAAAAACACGGTA
GATCTGCAAATGAACTCTCTTCGCGCTGAAGATACAGCAGTCTACTACTGCGCCAGGGATTCAGGGTATTCTGACCC
CATGTGGGGGCAGGGCACATTGGTTACCGTGTCCTCTTGA
>Sequence ID 71: SI-68P13 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFSCSVMHEALHNRF
TQKSLSLSPGGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVTVLGGGGSGGGGSGGGGSGGGGSEVQL
LESGGGLVQPGGSLSLSCAASGFSFSSGYDMCWVRQAPGKGLEWIACIAAGSAGITYDANWAKGRFTISRDNSKNTL
YLQMNSLRAEDTAVYYCARSAFSFDYAMDLWGQGTLVTVSS
>Sequence ID 72: SI-68P13 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGCGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCAAGCCGGGATGAGCTGACC
AAGAACCAGGTCAGCCTGTGGTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCTCTGCACAACAGATTC
ACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCAC
ACCTCAACTGGTATCAACAGAAACCCGGTAAAGCACCGAAGCTGCTCATCTACAAGGCATCAACTCTTGCCTCAGGT
GTGCCTAGTCGCTTCTCCGGGAGCGGCTCCGGTACTGAGTTTACTTTGACGATATCTTCCCTCCAACCCGACGACTT
CGCCACATACTATTGTCAGCAGGGTTATAGTTGGGGAAATGTTGACAATGTGTTTGGAGGGGGAACGAAAGTGACTG
TGTTGGGAGGGGGAGGAAGTGGCGGCGGCGGAAGCGGAGGTGGTGGCTCCGGCGGGGGTGGAAGCGAAGTGCAATTG
CTCGAAAGTGGTGGCGGACTTGTGCAACCTGGTGGAAGCCTCTCACTCTCTTGCGCGGCGAGTGGTTTTAGCTTCAG
TAGCGGCTACGATATGTGCTGGGTAAGACAGGCTCCTGGTAAAGGCCTTGAGTGGATTGCGTGTATCGCCGCCGGGT
CTGCTGGTATAACATACGATGCAAACTGGGCGAAAGGGCGCTTCACAATCTCCAGGGACAACTCAAAAAATACATTG
TACCTCCAGATGAATAGCCTGAGAGCCGAAGACACCGCAGTCTACTATTGCGCAAGGTCAGCATTTAGCTTCGATTA
TGCAATGGATCTTTGGGGCCAGGGGACGCTCGTCACCGTCTCATCTTGA
>Sequence ID 73: SI-68P17 Chain A Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSSGGGGSGGGGSQVQLQQSGPGLVKPSETLSITCTVSGFSLTNYGVHWIRQAPGKGLE
WLGVIWSGGNTDYNTPFTSRFTITKDNSKNQVYFKLRSVRADDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRWQQGNVFSCSVMH
EALHNHYTQKSLSLSPGGGGGSGGGGSDVVMTQSPSSVSASVGDRVTITCQASQNIRTYLSWYQQKPGKAPKLLIYA
AANLASGVPSRFSGSGSGTDFTLTISDLEPGDAATYYCQSTYLGTDYVGGAFGGGTKLTVLGGGGSGGGGSGGGGSG
GGGSEVQLVESGGGLVQPGGSLRLSCTASGFTISSYHMQWVRQAPGKGLEYIGTISSGGNVYYASSARGRFTISRPS
SKNTVDLQMNSLRAEDTAVYYCARDSGYSDPMWGQGTLVTVSS
>Sequence ID 74: SI-68P17 Chain A Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTCTCGAGTGGCGGTGGAGGGTC
CGGCGGTGGTGGATCACAAGTACAGTTGCAGCAATCCGGTCCCGGTCTCGTCAAACCGAGTGAGACGCTTAGTATAA
CGTGTACTGTTTCAGGCTTTAGCCTTACGAACTATGGAGTTCACTGGATTCGGCAGGCACCCGGCAAAGGTTTGGAA
TGGCTGGGTGTTATTTGGTCAGGTGGAAATACAGACTATAACACCCCCTTTACAAGTCGGTTCACAATTACGAAAGA
TAATTCCAAAAATCAAGTTTATTTCAAGTTGAGATCCGTCCGCGCGGACGACACTGCGATCTACTATTGTGCGAGGG
CACTGACCTACTACGATTACGAATTTGCGTATTGGGGGCAAGGGACTCTTGTAACAGTCTCCAGTGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTATACCCTGCCC
CCATCCCGGGATGAGCTGACCAAGAACCAGGTCAGCCTGTCCTGCGCCGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCGTGAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGCGGCGGTTCAGGTGGTGGGGG
ATCCGACGTTGTGATGACGCAGTCTCCTAGTTCAGTATCAGCGTCTGTTGGGGATCGAGTCACGATTACATGCCAGG
CGAGCCAAAATATTAGGACGTATCTGTCTTGGTATCAACAAAAGCCTGGTAAGGCTCCAAAACTCCTCATTTACGCC
GCTGCCAACTTGGCTAGTGGAGTACCTTCACGCTTTAGTGGGTCAGGTTCTGGAACAGATTTTACGTTGACGATCTC
CGATTTGGAACCAGGGGACGCTGCCACGTATTATTGCCAGTCCACCTACCTCGGGACTGACTACGTTGGTGGGGCGT
TTGGCGGAGGAACAAAACTCACTGTACTTGGTGGTGGTGGTTCAGGCGGGGGGGGTTCCGGCGGAGGCGGTTCTGGG
GGTGGAGGTTCCGAGGTTCAGTTGGTTGAAAGTGGGGGGGGACTCGTTCAGCCTGGAGGCTCTTTGCGACTTTCTTG
TACCGCTTCTGGGTTCACTATAAGTTCATATCATATGCAATGGGTAAGACAGGCGCCTGGTAAGGGACTTGAATACA
TAGGAACAATAAGCTCTGGTGGTAACGTCTATTACGCCTCATCCGCGCGGGGGAGGTTTACAATTTCCAGGCCTTCT
AGCAAAAACACGGTAGATCTGCAAATGAACTCTCTTCGCGCTGAAGATACAGCAGTCTACTACTGCGCCAGGGATTC
AGGGTATTCTGACCCCATGTGGGGGCAGGGCACATTGGTTACCGTGTCCTCTTGA
>Sequence ID 75: SI-68P17 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGPGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LWCLVKGFYPSDIAVEWESNGQPENNYKTTPPVLDSDGSFFLYSKLTVDKSRWQQGNVFSCSVMHEALHNRFTQKSL
SLSPGGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVTVLGGGGSGGGGSGGGGSGGGGSEVQLLESGG
GLVQPGGSLSLSCAASGFSFSSGYDMCWVRQAPGKGLEWIACIAAGSAGITYDANWAKGRFTISRDNSKNTLYLQMN
SLRAEDTAVYYCARSAFSFDYAMDLWGQGTLVTVSS
>Sequence ID 76: SI-68P17 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGACCCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGAGGCGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCAAGCCGGGATGAGCTGACCAAGAACCAGGTCAGC
CTGTGGTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACCGTGGACAAGAGCA
GGTGGCAGCAGGGGAACGTCTTCTCATGCTCCGTGATGCATGAGGCTCTGCACAACAGATTCACGCAGAAGAGCCTC
TCCCTGTCTCCGGGTGGCGGTGGAGGGTCCGGCGGTGGTGGATCCGACATTCAGATGACACAGTCACCGTCTACGCT
CTCAGCCTCAGTAGGCGATAGGGTGACTATCACCTGCCAAGCCTCCCAATCTATAAGTTCACACCTCAACTGGTATC
AACAGAAACCCGGTAAAGCACCGAAGCTGCTCATCTACAAGGCATCAACTCTTGCCTCAGGTGTGCCTAGTCGCTTC
TCCGGGAGCGGCTCCGGTACTGAGTTTACTTTGACGATATCTTCCCTCCAACCCGACGACTTCGCCACATACTATTG
TCAGCAGGGTTATAGTTGGGGAAATGTTGACAATGTGTTTGGAGGGGGAACGAAAGTGACTGTGTTGGGAGGGGGAG
GAAGTGGCGGCGGCGGAAGCGGAGGTGGTGGCTCCGGCGGGGGTGGAAGCGAAGTGCAATTGCTCGAAAGTGGTGGC
GGACTTGTGCAACCTGGTGGAAGCCTCTCACTCTCTTGCGCGGCGAGTGGTTTTAGCTTCAGTAGCGGCTACGATAT
GTGCTGGGTAAGACAGGCTCCTGGTAAAGGCCTTGAGTGGATTGCGTGTATCGCCGCCGGGTCTGCTGGTATAACAT
ACGATGCAAACTGGGCGAAAGGGCGCTTCACAATCTCCAGGGACAACTCAAAAAATACATTGTACCTCCAGATGAAT
AGCCTGAGAGCCGAAGACACCGCAGTCTACTATTGCGCAAGGTCAGCATTTAGCTTCGATTATGCAATGGATCTTTG
GGGCCAGGGGACGCTCGTCACCGTCTCATCTTGA
>Sequence ID 77: SI-68P15 Chain A Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVLGGGGSGGGGSGGGGSGGGGSEVQLVESGGGLVQPGGSLRLSC
AASGFTISTNAMSWVRQAPGKGLEWVGVITGRDITYYASWAKGRFTISRDTSKNTVYLQMNSLRAEDTAVYYCARDG
GSSAITSNNIWGQGTLVTVSSGGGGSGGGGSQVQLQQSGPGLVKPSETLSITCTVSGFSLTNYGVHWIRQAPGKGLE
WLGVIWSGGNTDYNTPFTSRFTITKDNSKNQVYFKLRSVRADDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRWQQGNVFSCSVMH
EALHNHYTQKSLSLSPGGGGGSGGGGSDVVMTQSPSSVSASVGDRVTITCQASQNIRTYLSWYQQKPGKAPKLLIYA
AANLASGVPSRFSGSGSGTDFTLTISDLEPGDAATYYCQSTYLGTDYVGGAFGGGTKLTVLGGGGSGGGGSGGGGSG
GGGSEVQLVESGGGLVQPGGSLRLSCTASGFTISSYHMQWVRQAPGKGLEYIGTISSGGNVYYASSARGRFTISRPS
SKNTVDLQMNSLRAEDTAVYYCARDSGYSDPMWGQGTLVTVSS
>Sequence ID 78: SI-68P15 Chain A Nucleotide Sequence
GAAATCGTTATGACGCAGAGTCCCTCCACGCTCTCCGCTAGTGTCGGGGATCGCGTCATTATCACATGCCAGGCCTC
CGAGTCAATCAGCAGCTGGCTTGCATGGTATCAACAGAAGCCGGGAAAAGCTCCTAAATTGCTGATCTATGAAGCGT
CAAAATTGGCGTCTGGTGTCCCATCTAGGTTCTCCGGCTCTGGGTCTGGTGCGGAATTTACTTTGACAATCTCCAGT
CTTCAACCAGACGATTTCGCTACCTACTACTGCCAAGGGTATTTCTATTTTATAAGCCGGACATATGTAAACTCCTT
CGGCCAAGGAACAAAGTTGACTGTTCTTGGTGGCGGAGGCAGTGGTGGCGGGGGCAGCGGAGGTGGTGGTTCAGGGG
GTGGTGGGAGCGAAGTCCAATTGGTAGAAAGTGGCGGTGGTCTGGTGCAACCTGGTGGATCTCTTCGCCTCTCATGC
GCCGCTAGTGGCTTTACTATTTCAACTAATGCGATGAGCTGGGTTCGCCAGGCCCCCGGCAAAGGACTTGAGTGGGT
CGGCGTCATCACCGGCAGGGACATTACATACTATGCGAGTTGGGCAAAGGGCAGGTTCACGATTAGCCGCGATACTT
CAAAGAATACCGTTTACCTTCAAATGAATAGCTTGAGGGCGGAAGACACAGCTGTGTATTACTGCGCGAGGGATGGA
GGTAGTTCCGCCATAACTTCCAACAACATATGGGGACAAGGCACGCTGGTTACTGTCTCGAGTGGCGGTGGAGGGTC
CGGCGGTGGTGGATCACAAGTACAGTTGCAGCAATCCGGTCCCGGTCTCGTCAAACCGAGTGAGACGCTTAGTATAA
CGTGTACTGTTTCAGGCTTTAGCCTTACGAACTATGGAGTTCACTGGATTCGGCAGGCACCCGGCAAAGGTTTGGAA
TGGCTGGGTGTTATTTGGTCAGGTGGAAATACAGACTATAACACCCCCTTTACAAGTCGGTTCACAATTACGAAAGA
TAATTCCAAAAATCAAGTTTATTTCAAGTTGAGATCCGTCCGCGCGGACGACACTGCGATCTACTATTGTGCGAGGG
CACTGACCTACTACGATTACGAATTTGCGTATTGGGGGCAAGGGACTCTTGTAACAGTCTCCAGTGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTATACCCTGCCC
CCATCCCGGGATGAGCTGACCAAGAACCAGGTCAGCCTGTCCTGCGCCGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCGTGAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGCGGCGGTTCAGGTGGTGGGGG
ATCCGACGTTGTGATGACGCAGTCTCCTAGTTCAGTATCAGCGTCTGTTGGGGATCGAGTCACGATTACATGCCAGG
CGAGCCAAAATATTAGGACGTATCTGTCTTGGTATCAACAAAAGCCTGGTAAGGCTCCAAAACTCCTCATTTACGCC
GCTGCCAACTTGGCTAGTGGAGTACCTTCACGCTTTAGTGGGTCAGGTTCTGGAACAGATTTTACGTTGACGATCTC
CGATTTGGAACCAGGGGACGCTGCCACGTATTATTGCCAGTCCACCTACCTCGGGACTGACTACGTTGGTGGGGCGT
TTGGCGGAGGAACAAAACTCACTGTACTTGGTGGTGGTGGTTCAGGCGGGGGGGGTTCCGGCGGAGGCGGTTCTGGG
GGTGGAGGTTCCGAGGTTCAGTTGGTTGAAAGTGGGGGGGGACTCGTTCAGCCTGGAGGCTCTTTGCGACTTTCTTG
TACCGCTTCTGGGTTCACTATAAGTTCATATCATATGCAATGGGTAAGACAGGCGCCTGGTAAGGGACTTGAATACA
TAGGAACAATAAGCTCTGGTGGTAACGTCTATTACGCCTCATCCGCGCGGGGGAGGTTTACAATTTCCAGGCCTTCT
AGCAAAAACACGGTAGATCTGCAAATGAACTCTCTTCGCGCTGAAGATACAGCAGTCTACTACTGCGCCAGGGATTC
AGGGTATTCTGACCCCATGTGGGGGCAGGGCACATTGGTTACCGTGTCCTCTTGA
>Sequence ID 79: SI-68P15 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGPGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LWCLVKGFYPSDIAVEWESNGQPENNYKTTPPVLDSDGSFFLYSKLTVDKSRWQQGNVFSCSVMHEALHNRFTQKSL
SLSPGGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVTVLGGGGSGGGGSGGGGSGGGGSEVQLLESGG
GLVQPGGSLSLSCAASGFSFSSGYDMCWVRQAPGKGLEWIACIAAGSAGITYDANWAKGRFTISRDNSKNTLYLQMN
SLRAEDTAVYYCARSAFSFDYAMDLWGQGTLVTVSS
>Sequence ID 80: SI-68P15 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGACCCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGAGGCGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCAAGCCGGGATGAGCTGACCAAGAACCAGGTCAGC
CTGTGGTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACCGTGGACAAGAGCA
GGTGGCAGCAGGGGAACGTCTTCTCATGCTCCGTGATGCATGAGGCTCTGCACAACAGATTCACGCAGAAGAGCCTC
TCCCTGTCTCCGGGTGGCGGTGGAGGGTCCGGCGGTGGTGGATCCGACATTCAGATGACACAGTCACCGTCTACGCT
CTCAGCCTCAGTAGGCGATAGGGTGACTATCACCTGCCAAGCCTCCCAATCTATAAGTTCACACCTCAACTGGTATC
AACAGAAACCCGGTAAAGCACCGAAGCTGCTCATCTACAAGGCATCAACTCTTGCCTCAGGTGTGCCTAGTCGCTTC
TCCGGGAGCGGCTCCGGTACTGAGTTTACTTTGACGATATCTTCCCTCCAACCCGACGACTTCGCCACATACTATTG
TCAGCAGGGTTATAGTTGGGGAAATGTTGACAATGTGTTTGGAGGGGGAACGAAAGTGACTGTGTTGGGAGGGGGAG
GAAGTGGCGGCGGCGGAAGCGGAGGTGGTGGCTCCGGCGGGGGTGGAAGCGAAGTGCAATTGCTCGAAAGTGGTGGC
GGACTTGTGCAACCTGGTGGAAGCCTCTCACTCTCTTGCGCGGCGAGTGGTTTTAGCTTCAGTAGCGGCTACGATAT
GTGCTGGGTAAGACAGGCTCCTGGTAAAGGCCTTGAGTGGATTGCGTGTATCGCCGCCGGGTCTGCTGGTATAACAT
ACGATGCAAACTGGGCGAAAGGGCGCTTCACAATCTCCAGGGACAACTCAAAAAATACATTGTACCTCCAGATGAAT
AGCCTGAGAGCCGAAGACACCGCAGTCTACTATTGCGCAAGGTCAGCATTTAGCTTCGATTATGCAATGGATCTTTG
GGGCCAGGGGACGCTCGTCACCGTCTCATCTTGA
>Sequence ID 81: SI-68P18 Chain A Amino Acid Sequence
DVQMTQSPSTLSATVGQRVTINCQASESISSWLAWYQQKPGQPPKLLIYEASKLASGVPSRFSGSGSGTEFTLTISS
LQSDDFATYYCQGYFYFISRTYVNSFGGGTRVEIKGGGGSGGGGSGGGGSGGGGSQQLLESGGRLIKPGATLRLTCK
TSGFTISTNAMSWVRQPPGKGLEWIGVITGRDITYYASWAKGRFTISKTSINTVFLQMRSLRAEDTAVYYCARDGGS
SAITSNNIWGRGTLVTVSSGGGGSGGGGSQVQLQQSGPGLVKPSETLSITCTVSGFSLTNYGVHWIRQAPGKGLEWL
GVIWSGGNTDYNTPFTSRFTITKDNSKNQVYFKLRSVRADDTAIYYCARALTYYDYEFAYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCAVSNKALPAPIEKTISKAKGQPREPQVYTLPPS
RDELTKNQVSLSCAVKGFYPSDIAVEWESNGQPENNYKTTPPVLDSDGSFFLVSKLTVDKSRWQQGNVFSCSVMHEA
LHNHYTQKSLSLSPGGGGGSGGGGSDVVMTQSPSSVSASVGDRVTITCQASQNIRTYLSWYQQKPGKAPKLLIYAAA
NLASGVPSRFSGSGSGTDFTLTISDLEPGDAATYYCQSTYLGTDYVGGAFGGGTKLTVLGGGGSGGGGSGGGGSGGG
GSEVQLVESGGGLVQPGGSLRLSCTASGFTISSYHMQWVRQAPGKGLEYIGTISSGGNVYYASSARGRFTISRPSSK
NTVDLQMNSLRAEDTAVYYCARDSGYSDPMWGQGTLVTVSS
>Sequence ID 82: SI-68P18 Chain A Nucleotide Sequence
GATGTGCAGATGACCCAGAGCCCGAGCACCCTGAGCGCGACCGTGGGCCAGCGCGTGACCATTAACTGCCAGGCGAG
CGAAAGCATTAGCAGCTGGCTGGCGTGGTATCAGCAGAAACCGGGCCAGCCGCCGAAACTGCTGATTTATGAAGCGA
GCAAACTGGCGAGCGGCGTGCCGAGCCGCTTTAGCGGCAGCGGCAGCGGCACCGAATTTACCCTGACCATTAGCAGC
CTGCAGAGCGATGATTTTGCGACCTATTATTGCCAGGGCTATTTTTATTTTATTAGCCGCACCTATGTGAACAGCTT
TGGCGGCGGCACCCGCGTGGAAATTAAAGGCGGTGGCGGTAGTGGGGGAGGCGGTTCTGGCGGCGGAGGGTCCGGCG
GTGGAGGATCACAGCAGCTGCTGGAAAGCGGCGGCCGCCTGATTAAACCGGGCGCGACCCTGCGCCTGACCTGCAAA
ACCAGCGGCTTTACCATTAGCACCAACGCGATGAGCTGGGTGCGCCAGCCGCCGGGCAAAGGCCTGGAATGGATTGG
CGTGATTACCGGCCGCGATATTACCTATTATGCGAGCTGGGCGAAAGGCCGCTTTACCATTAGCAAAACCAGCATTA
ACACCGTGTTTCTGCAGATGCGCAGCCTGCGCGCGGAAGATACCGCGGTGTATTATTGCGCGCGCGATGGCGGCAGC
AGCGCGATTACCAGCAACAACATTTGGGGCCGCGGCACCCTGGTGACCGTGAGCAGCGGCGGTGGAGGGTCCGGCGG
TGGTGGATCACAAGTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGGTTTGGAATGGCTG
GGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGA
CCTACTACGATTACGAATTTGCGTATTGGGGGCAAGGGACTCTTGTAACAGTCTCCAGTGCTAGCACCAAGGGCCCA
TCGGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTGC
AACGTGAATCACAAGCCCAGCAACACCAAGGTGGACAAGAGAGTTGAGCCCAAATCTTGTGACAAAACTCACACATG
CCCACCGTGCCCAGCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTATACCCTGCCCCCATCC
CGGGATGAGCTGACCAAGAACCAGGTCAGCCTGTCCTGCGCCGTCAAAGGCTTCTATCCCAGCGACATCGCCGTGGA
GTGGGAGAGCAATGGGCAGCCGGAGAACAACTACAAGACCACGCCTCCCGTGCTGGACTCCGACGGCTCCTTCTTCC
TCGTGAGCAAGCTCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTGGCGGCGGCGGTTCAGGTGGTGGGGGATCCGA
CGTTGTGATGACGCAGTCTCCTAGTTCAGTATCAGCGTCTGTTGGGGATCGAGTCACGATTACATGCCAGGCGAGCC
AAAATATTAGGACGTATCTGTCTTGGTATCAACAAAAGCCTGGTAAGGCTCCAAAACTCCTCATTTACGCCGCTGCC
AACTTGGCTAGTGGAGTACCTTCACGCTTTAGTGGGTCAGGTTCTGGAACAGATTTTACGTTGACGATCTCCGATTT
GGAACCAGGGGACGCTGCCACGTATTATTGCCAGTCCACCTACCTCGGGACTGACTACGTTGGTGGGGCGTTTGGCG
GAGGAACAAAACTCACTGTACTTGGTGGTGGTGGTTCAGGCGGGGGGGGTTCCGGCGGAGGCGGTTCTGGGGGTGGA
GGTTCCGAGGTTCAGTTGGTTGAAAGTGGGGGGGGACTCGTTCAGCCTGGAGGCTCTTTGCGACTTTCTTGTACCGC
TTCTGGGTTCACTATAAGTTCATATCATATGCAATGGGTAAGACAGGCGCCTGGTAAGGGACTTGAATACATAGGAA
CAATAAGCTCTGGTGGTAACGTCTATTACGCCTCATCCGCGCGGGGGAGGTTTACAATTTCCAGGCCTTCTAGCAAA
AACACGGTAGATCTGCAAATGAACTCTCTTCGCGCTGAAGATACAGCAGTCTACTACTGCGCCAGGGATTCAGGGTA
TTCTGACCCCATGTGGGGGCAGGGCACATTGGTTACCGTGTCCTCTTGA
>Sequence ID 83: SI-68P18 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGPGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LWCLVKGFYPSDIAVEWESNGQPENNYKTTPPVLDSDGSFFLYSKLTVDKSRWQQGNVFSCSVMHEALHNRFTQKSL
SLSPGGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVTVLGGGGSGGGGSGGGGSGGGGSEVQLLESGG
GLVQPGGSLSLSCAASGFSFSSGYDMCWVRQAPGKGLEWIACIAAGSAGITYDANWAKGRFTISRDNSKNTLYLQMN
SLRAEDTAVYYCARSAFSFDYAMDLWGQGTLVTVSS
>Sequence ID 84: SI-68P18 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGACCCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGAGGCGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCAAGCCGGGATGAGCTGACCAAGAACCAGGTCAGC
CTGTGGTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACCGTGGACAAGAGCA
GGTGGCAGCAGGGGAACGTCTTCTCATGCTCCGTGATGCATGAGGCTCTGCACAACAGATTCACGCAGAAGAGCCTC
TCCCTGTCTCCGGGTGGCGGTGGAGGGTCCGGCGGTGGTGGATCCGACATTCAGATGACACAGTCACCGTCTACGCT
CTCAGCCTCAGTAGGCGATAGGGTGACTATCACCTGCCAAGCCTCCCAATCTATAAGTTCACACCTCAACTGGTATC
AACAGAAACCCGGTAAAGCACCGAAGCTGCTCATCTACAAGGCATCAACTCTTGCCTCAGGTGTGCCTAGTCGCTTC
TCCGGGAGCGGCTCCGGTACTGAGTTTACTTTGACGATATCTTCCCTCCAACCCGACGACTTCGCCACATACTATTG
TCAGCAGGGTTATAGTTGGGGAAATGTTGACAATGTGTTTGGAGGGGGAACGAAAGTGACTGTGTTGGGAGGGGGAG
GAAGTGGCGGCGGCGGAAGCGGAGGTGGTGGCTCCGGCGGGGGTGGAAGCGAAGTGCAATTGCTCGAAAGTGGTGGC
GGACTTGTGCAACCTGGTGGAAGCCTCTCACTCTCTTGCGCGGCGAGTGGTTTTAGCTTCAGTAGCGGCTACGATAT
GTGCTGGGTAAGACAGGCTCCTGGTAAAGGCCTTGAGTGGATTGCGTGTATCGCCGCCGGGTCTGCTGGTATAACAT
ACGATGCAAACTGGGCGAAAGGGCGCTTCACAATCTCCAGGGACAACTCAAAAAATACATTGTACCTCCAGATGAAT
AGCCTGAGAGCCGAAGACACCGCAGTCTACTATTGCGCAAGGTCAGCATTTAGCTTCGATTATGCAATGGATCTTTG
GGGCCAGGGGACGCTCGTCACCGTCTCATCTTGA
>Sequence ID 85: SI-68P19 Chain A Amino Acid Sequence
DVVMTQSPSSLSASVGDRVTISCQASESISSWLAWYQQKPGQAPKLLIYEASKLASGVPSRFSGSGSGTEFTLTISS
LEPEDFATYYCQGYFYFISRTYVNSFGGGTKVEIKGGGGSGGGGSGGGGSGGGGSQQLVESGGGLVKPGGSLRLSCA
ASGFTISTNAMSWVRQAPGKGLEWVGVITGRDITYYASWAKGRFTISKTSKNTVYLQMNSLRAEDTAVYYCARDGGS
SAITSNNIWGQGTLVTVSSGGGGSGGGGSQVQLQQSGPGLVKPSETLSITCTVSGFSLTNYGVHWIRQAPGKGLEWL
GVIWSGGNTDYNTPFTSRFTITKDNSKNQVYFKLRSVRADDTAIYYCARALTYYDYEFAYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCAVSNKALPAPIEKTISKAKGQPREPQVYTLPPS
RDELTKNQVSLSCAVKGFYPSDIAVEWESNGQPENNYKTTPPVLDSDGSFFLVSKLTVDKSRWQQGNVFSCSVMHEA
LHNHYTQKSLSLSPGGGGGSGGGGSDVVMTQSPSSVSASVGDRVTITCQASQNIRTYLSWYQQKPGKAPKLLIYAAA
NLASGVPSRFSGSGSGTDFTLTISDLEPGDAATYYCQSTYLGTDYVGGAFGGGTKLTVLGGGGSGGGGSGGGGSGGG
GSEVQLVESGGGLVQPGGSLRLSCTASGFTISSYHMQWVRQAPGKGLEYIGTISSGGNVYYASSARGRFTISRPSSK
NTVDLQMNSLRAEDTAVYYCARDSGYSDPMWGQGTLVTVSS
>Sequence ID 86: SI-68P19 Chain A Nucleotide Sequence
GATGTGGTGATGACCCAGAGCCCGAGCAGCCTGAGCGCGAGCGTGGGCGATCGCGTGACCATTAGCTGCCAGGCGAG
CGAAAGCATTAGCAGCTGGCTGGCGTGGTATCAGCAGAAACCGGGCCAGGCGCCGAAACTGCTGATTTATGAAGCGA
GCAAACTGGCGAGCGGCGTGCCGAGCCGCTTTAGCGGCAGCGGCAGCGGCACCGAATTTACCCTGACCATTAGCAGC
CTGGAACCGGAAGATTTTGCGACCTATTATTGCCAGGGCTATTTTTATTTTATTAGCCGCACCTATGTGAACAGCTT
TGGCGGCGGCACCAAAGTGGAAATTAAAGGCGGTGGCGGTAGTGGGGGAGGCGGTTCTGGCGGCGGAGGGTCCGGCG
GTGGAGGATCACAGCAGCTGGTGGAAAGCGGCGGCGGCCTGGTGAAACCGGGCGGCAGCCTGCGCCTGAGCTGCGCG
GCGAGCGGCTTTACCATTAGCACCAACGCGATGAGCTGGGTGCGCCAGGCGCCGGGCAAAGGCCTGGAATGGGTGGG
CGTGATTACCGGCCGCGATATTACCTATTATGCGAGCTGGGCGAAAGGCCGCTTTACCATTAGCAAAACCAGCAAAA
ACACCGTGTATCTGCAGATGAACAGCCTGCGCGCGGAAGATACCGCGGTGTATTATTGCGCGCGCGATGGCGGCAGC
AGCGCGATTACCAGCAACAACATTTGGGGCCAGGGCACCCTGGTGACCGTGAGCAGCGGCGGTGGAGGGTCCGGCGG
TGGTGGATCACAAGTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGGTTTGGAATGGCTG
GGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGA
CCTACTACGATTACGAATTTGCGTATTGGGGGCAAGGGACTCTTGTAACAGTCTCCAGTGCTAGCACCAAGGGCCCA
TCGGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTGC
AACGTGAATCACAAGCCCAGCAACACCAAGGTGGACAAGAGAGTTGAGCCCAAATCTTGTGACAAAACTCACACATG
CCCACCGTGCCCAGCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTATACCCTGCCCCCATCC
CGGGATGAGCTGACCAAGAACCAGGTCAGCCTGTCCTGCGCCGTCAAAGGCTTCTATCCCAGCGACATCGCCGTGGA
GTGGGAGAGCAATGGGCAGCCGGAGAACAACTACAAGACCACGCCTCCCGTGCTGGACTCCGACGGCTCCTTCTTCC
TCGTGAGCAAGCTCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTGGCGGCGGCGGTTCAGGTGGTGGGGGATCCGA
CGTTGTGATGACGCAGTCTCCTAGTTCAGTATCAGCGTCTGTTGGGGATCGAGTCACGATTACATGCCAGGCGAGCC
AAAATATTAGGACGTATCTGTCTTGGTATCAACAAAAGCCTGGTAAGGCTCCAAAACTCCTCATTTACGCCGCTGCC
AACTTGGCTAGTGGAGTACCTTCACGCTTTAGTGGGTCAGGTTCTGGAACAGATTTTACGTTGACGATCTCCGATTT
GGAACCAGGGGACGCTGCCACGTATTATTGCCAGTCCACCTACCTCGGGACTGACTACGTTGGTGGGGCGTTTGGCG
GAGGAACAAAACTCACTGTACTTGGTGGTGGTGGTTCAGGCGGGGGGGGTTCCGGCGGAGGCGGTTCTGGGGGTGGA
GGTTCCGAGGTTCAGTTGGTTGAAAGTGGGGGGGGACTCGTTCAGCCTGGAGGCTCTTTGCGACTTTCTTGTACCGC
TTCTGGGTTCACTATAAGTTCATATCATATGCAATGGGTAAGACAGGCGCCTGGTAAGGGACTTGAATACATAGGAA
CAATAAGCTCTGGTGGTAACGTCTATTACGCCTCATCCGCGCGGGGGAGGTTTACAATTTCCAGGCCTTCTAGCAAA
AACACGGTAGATCTGCAAATGAACTCTCTTCGCGCTGAAGATACAGCAGTCTACTACTGCGCCAGGGATTCAGGGTA
TTCTGACCCCATGTGGGGGCAGGGCACATTGGTTACCGTGTCCTCTTGA
>Sequence ID 87: SI-68P19 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGPGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LWCLVKGFYPSDIAVEWESNGQPENNYKTTPPVLDSDGSFFLYSKLTVDKSRWQQGNVFSCSVMHEALHNRFTQKSL
SLSPGGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVTVLGGGGSGGGGSGGGGSGGGGSEVQLLESGG
GLVQPGGSLSLSCAASGFSFSSGYDMCWVRQAPGKGLEWIACIAAGSAGITYDANWAKGRFTISRDNSKNTLYLQMN
SLRAEDTAVYYCARSAFSFDYAMDLWGQGTLVTVSS
>Sequence ID 88: SI-68P19 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGACCCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGAGGCGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCAAGCCGGGATGAGCTGACCAAGAACCAGGTCAGC
CTGTGGTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACCGTGGACAAGAGCA
GGTGGCAGCAGGGGAACGTCTTCTCATGCTCCGTGATGCATGAGGCTCTGCACAACAGATTCACGCAGAAGAGCCTC
TCCCTGTCTCCGGGTGGCGGTGGAGGGTCCGGCGGTGGTGGATCCGACATTCAGATGACACAGTCACCGTCTACGCT
CTCAGCCTCAGTAGGCGATAGGGTGACTATCACCTGCCAAGCCTCCCAATCTATAAGTTCACACCTCAACTGGTATC
AACAGAAACCCGGTAAAGCACCGAAGCTGCTCATCTACAAGGCATCAACTCTTGCCTCAGGTGTGCCTAGTCGCTTC
TCCGGGAGCGGCTCCGGTACTGAGTTTACTTTGACGATATCTTCCCTCCAACCCGACGACTTCGCCACATACTATTG
TCAGCAGGGTTATAGTTGGGGAAATGTTGACAATGTGTTTGGAGGGGGAACGAAAGTGACTGTGTTGGGAGGGGGAG
GAAGTGGCGGCGGCGGAAGCGGAGGTGGTGGCTCCGGCGGGGGTGGAAGCGAAGTGCAATTGCTCGAAAGTGGTGGC
GGACTTGTGCAACCTGGTGGAAGCCTCTCACTCTCTTGCGCGGCGAGTGGTTTTAGCTTCAGTAGCGGCTACGATAT
GTGCTGGGTAAGACAGGCTCCTGGTAAAGGCCTTGAGTGGATTGCGTGTATCGCCGCCGGGTCTGCTGGTATAACAT
ACGATGCAAACTGGGCGAAAGGGCGCTTCACAATCTCCAGGGACAACTCAAAAAATACATTGTACCTCCAGATGAAT
AGCCTGAGAGCCGAAGACACCGCAGTCTACTATTGCGCAAGGTCAGCATTTAGCTTCGATTATGCAATGGATCTTTG
GGGCCAGGGGACGCTCGTCACCGTCTCATCTTGA
>Sequence ID 89: SI-68P16 Chain A Amino Acid Sequence
DPVLTQSPSSLSASVGDRVTISCQSSQSVAKNNNLAWFQQKPGQAPKLLIYSASTLAAGVPSRFSGSGSGTDFTLTI
SSVQPEDFATYYCSARDSGNIQSFGGGTKVEIKGGGGSGGGGSGGGGSGGGGSQQLVESGGGLVKPGGSLRLSCAAS
GIDLSSNAIGWVRQAPGKGLEWVGVIFGSGNTYYASWAKGRFTISRSKNTVYLQMNSLRAEDTAVYYCARGGYSSDI
WGQGTLVTVSSGGGGSGGGGSQVQLQQSGPGLVKPSETLSITCTVSGFSLTNYGVHWIRQAPGKGLEWLGVIWSGGN
TDYNTPFTSRFTITKDNSKNQVYFKLRSVRADDTAIYYCARALTYYDYEFAYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCAVSNKALPAPIEKTISKAKGQPREPQVYTLPPSRDELTKNQ
VSLSCAVKGFYPSDIAVEWESNGQPENNYKTTPPVLDSDGSFFLVSKLTVDKSRWQQGNVFSCSVMHEALHNHYTQK
SLSLSPGGGGGSGGGGSDVVMTQSPSSVSASVGDRVTITCQASQNIRTYLSWYQQKPGKAPKLLIYAAANLASGVPS
RFSGSGSGTDFTLTISDLEPGDAATYYCQSTYLGTDYVGGAFGGGTKLTVLGGGGSGGGGSGGGGSGGGGSEVQLVE
SGGGLVQPGGSLRLSCTASGFTISSYHMQWVRQAPGKGLEYIGTISSGGNVYYASSARGRFTISRPSSKNTVDLQMN
SLRAEDTAVYYCARDSGYSDPMWGQGTLVTVSS
>Sequence ID 90: SI-68P16 Chain A Nucleotide Sequence
GATCCAGTTCTGACACAAAGTCCATCCAGCCTGTCTGCCTCAGTCGGCGACAGAGTGACCATCAGTTGCCAGAGCTC
ACAGTCTGTGGCTAAGAACAACAACTTGGCGTGGTTCCAACAGAAACCTGGACAGGCTCCGAAATTGCTGATCTATT
CTGCTTCCACGCTTGCTGCTGGTGTTCCTTCCCGCTTTTCAGGTAGTGGTAGCGGGACAGACTTCACTTTGACTATA
AGCAGCGTGCAGCCTGAAGATTTTGCGACCTACTATTGTTCTGCTAGAGACAGTGGAAATATTCAGTCCTTTGGGGG
GGGAACGAAGGTCGAAATAAAGGGCGGCGGAGGGTCTGGTGGGGGTGGCAGTGGTGGTGGAGGAAGTGGTGGCGGTG
GTTCCCAACAGTTGGTTGAGTCTGGTGGCGGTCTCGTTAAGCCTGGAGGGTCCCTTCGGCTTAGTTGTGCGGCCTCT
GGGATCGACCTGTCTAGCAACGCTATAGGATGGGTACGGCAAGCTCCGGGCAAAGGCCTTGAGTGGGTCGGCGTTAT
TTTTGGGAGCGGTAATACGTACTACGCCAGCTGGGCAAAAGGTAGGTTCACTATCAGTCGATCAAAGAACACGGTAT
ATCTTCAGATGAACAGTCTCCGGGCGGAGGACACCGCGGTTTATTATTGCGCAAGGGGGGGATACTCCTCAGACATA
TGGGGCCAAGGTACTTTGGTGACGGTCTCGAGCGGCGGTGGAGGGTCCGGCGGTGGTGGATCACAAGTACAGTTGCA
GCAATCCGGTCCCGGTCTCGTCAAACCGAGTGAGACGCTTAGTATAACGTGTACTGTTTCAGGCTTTAGCCTTACGA
ACTATGGAGTTCACTGGATTCGGCAGGCACCCGGCAAAGGTTTGGAATGGCTGGGTGTTATTTGGTCAGGTGGAAAT
ACAGACTATAACACCCCCTTTACAAGTCGGTTCACAATTACGAAAGATAATTCCAAAAATCAAGTTTATTTCAAGTT
GAGATCCGTCCGCGCGGACGACACTGCGATCTACTATTGTGCGAGGGCACTGACCTACTACGATTACGAATTTGCGT
ATTGGGGGCAAGGGACTCTTGTAACAGTCTCCAGTGCTAGCACCAAGGGCCCATCGGTCTTCCCCCTGGCACCCTCC
TCCAAGAGCACCTCTGGGGGCACAGCGGCCCTGGGCTGCCTGGTCAAGGACTACTTCCCCGAACCGGTGACGGTGTC
GTGGAACTCAGGCGCCCTGACCAGCGGCGTGCACACCTTCCCGGCTGTCCTACAGTCCTCAGGACTCTACTCCCTCA
GCAGCGTGGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCACCTGAAGC
CGCGGGGGGACCGTCAGTCTTCCTCTTCCCCCCAAAACCCAAGGACACCCTCATGATCTCCCGGACCCCTGAGGTCA
CATGCGTGGTGGTGGACGTGAGCCACGAAGACCCTGAGGTCAAGTTCAACTGGTACGTGGACGGCGTGGAGGTGCAT
AATGCCAAGACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCGCGGTCTCCAACAAAGCCCTCCCAGCCCCCATCGAGAAAACCATCT
CCAAAGCCAAAGGGCAGCCCCGAGAACCACAGGTGTATACCCTGCCCCCATCCCGGGATGAGCTGACCAAGAACCAG
GTCAGCCTGTCCTGCGCCGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCGTGAGCAAGCTCACCGTGGACA
AGAGCAGGTGGCAGCAGGGGAACGTCTTCTCATGCTCCGTGATGCATGAGGCTCTGCACAACCACTACACGCAGAAG
AGCCTCTCCCTGTCTCCGGGTGGCGGCGGCGGTTCAGGTGGTGGGGGATCCGACGTTGTGATGACGCAGTCTCCTAG
TTCAGTATCAGCGTCTGTTGGGGATCGAGTCACGATTACATGCCAGGCGAGCCAAAATATTAGGACGTATCTGTCTT
GGTATCAACAAAAGCCTGGTAAGGCTCCAAAACTCCTCATTTACGCCGCTGCCAACTTGGCTAGTGGAGTACCTTCA
CGCTTTAGTGGGTCAGGTTCTGGAACAGATTTTACGTTGACGATCTCCGATTTGGAACCAGGGGACGCTGCCACGTA
TTATTGCCAGTCCACCTACCTCGGGACTGACTACGTTGGTGGGGCGTTTGGCGGAGGAACAAAACTCACTGTACTTG
GTGGTGGTGGTTCAGGCGGGGGGGGTTCCGGCGGAGGCGGTTCTGGGGGTGGAGGTTCCGAGGTTCAGTTGGTTGAA
AGTGGGGGGGGACTCGTTCAGCCTGGAGGCTCTTTGCGACTTTCTTGTACCGCTTCTGGGTTCACTATAAGTTCATA
TCATATGCAATGGGTAAGACAGGCGCCTGGTAAGGGACTTGAATACATAGGAACAATAAGCTCTGGTGGTAACGTCT
ATTACGCCTCATCCGCGCGGGGGAGGTTTACAATTTCCAGGCCTTCTAGCAAAAACACGGTAGATCTGCAAATGAAC
TCTCTTCGCGCTGAAGATACAGCAGTCTACTACTGCGCCAGGGATTCAGGGTATTCTGACCCCATGTGGGGGCAGGG
CACATTGGTTACCGTGTCCTCTTGA
>Sequence ID 91: SI-68P16 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGPGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGDKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLPPSRDELTKNQVS
LWCLVKGFYPSDIAVEWESNGQPENNYKTTPPVLDSDGSFFLYSKLTVDKSRWQQGNVFSCSVMHEALHNRFTQKSL
SLSPGGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVTVLGGGGSGGGGSGGGGSGGGGSEVQLLESGG
GLVQPGGSLSLSCAASGFSFSSGYDMCWVRQAPGKGLEWIACIAAGSAGITYDANWAKGRFTISRDNSKNTLYLQMN
SLRAEDTAVYYCARSAFSFDYAMDLWGQGTLVTVSS
>Sequence ID 92: SI-68P16 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGACCCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGAGGCGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCAAGCCGGGATGAGCTGACCAAGAACCAGGTCAGC
CTGTGGTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACCGTGGACAAGAGCA
GGTGGCAGCAGGGGAACGTCTTCTCATGCTCCGTGATGCATGAGGCTCTGCACAACAGATTCACGCAGAAGAGCCTC
TCCCTGTCTCCGGGTGGCGGTGGAGGGTCCGGCGGTGGTGGATCCGACATTCAGATGACACAGTCACCGTCTACGCT
CTCAGCCTCAGTAGGCGATAGGGTGACTATCACCTGCCAAGCCTCCCAATCTATAAGTTCACACCTCAACTGGTATC
AACAGAAACCCGGTAAAGCACCGAAGCTGCTCATCTACAAGGCATCAACTCTTGCCTCAGGTGTGCCTAGTCGCTTC
TCCGGGAGCGGCTCCGGTACTGAGTTTACTTTGACGATATCTTCCCTCCAACCCGACGACTTCGCCACATACTATTG
TCAGCAGGGTTATAGTTGGGGAAATGTTGACAATGTGTTTGGAGGGGGAACGAAAGTGACTGTGTTGGGAGGGGGAG
GAAGTGGCGGCGGCGGAAGCGGAGGTGGTGGCTCCGGCGGGGGTGGAAGCGAAGTGCAATTGCTCGAAAGTGGTGGC
GGACTTGTGCAACCTGGTGGAAGCCTCTCACTCTCTTGCGCGGCGAGTGGTTTTAGCTTCAGTAGCGGCTACGATAT
GTGCTGGGTAAGACAGGCTCCTGGTAAAGGCCTTGAGTGGATTGCGTGTATCGCCGCCGGGTCTGCTGGTATAACAT
ACGATGCAAACTGGGCGAAAGGGCGCTTCACAATCTCCAGGGACAACTCAAAAAATACATTGTACCTCCAGATGAAT
AGCCTGAGAGCCGAAGACACCGCAGTCTACTATTGCGCAAGGTCAGCATTTAGCTTCGATTATGCAATGGATCTTTG
GGGCCAGGGGACGCTCGTCACCGTCTCATCTTGA
>Sequence ID 93: SI-75P5 Chain A Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSSGGGGSGGGGSEVQLVESGGGLVQPGGSLRL
SCAASGFTISTNAMSWVRQAPGKGLEWIGVITGRDITYYASWAKGRFTISRDNSKNTLYLQMNSLRAEDTAVYYCAR
DGGSSAITSNNIWGQGTLVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQDWLNGKEYKCAVSN
KALPAPIEKTISKAKGQPREPQVYTLPPSRDELTKNQVSLSCAVKGFYPSDIAVEWESNGQPENNYKTTPPVLDSDG
SFFLVSKLTVDKSRWQQGNVFSCSVMHEALHNHYTQKSLSLSPGGGGGSGGGGSDVVMTQSPSSVSASVGDRVTITC
QASQNIRTYLSWYQQKPGKAPKLLIYAAANLASGVPSRFSGSGSGTDFTLTISDLEPGDAATYYCQSTYLGTDYVGG
AFGGGTKLTVLGGGGSGGGGSGGGGSGGGGSEVQLVESGGGLVQPGGSLRLSCTASGFTISSYHMQWVRQAPGKGLE
YI GT1SSGGNVYYASSARGRFTISRPSSKNTVDLQMNSLRAEDTAVYYCARDSGYSDPMWGQGTLVTVSS
>Sequence ID 94: SI-75P5 Chain A Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGCGGCGGTGGAGGGT
CCGGCGGTGGTGGATCAGAGGTGCAGCTGGTGGAGTCTGGGGGAGGCTTGGTCCAGCCTGGGGGGTCCCTGAGACTC
TCCTGTGCAGCCTCTGGATTCACCATCAGTACCAATGCAATGAGCTGGGTCCGCCAGGCTCCAGGGAAGGGGCTGGA
GTGGATCGGAGTCATTACTGGTCGTGATATCACATACTACGCGAGCTGGGCGAAAGGCAGATTCACCATCTCCAGAG
ACAATTCCAAGAACACGCTGTATCTTCAAATGAACAGCCTGAGAGCCGAGGACACGGCTGTGTATTACTGTGCGAGA
GACGGTGGTTCTTCTGCTATTACTAGTAACAACATTTGGGGCCAGGGAACCCTGGTCACCGTGTCCTCAGCTAGCAC
CAAGGGCCCATCGGTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTGGGCACCCAGAC
CTACATCTGCAACGTGAATCACAAGCCCAGCAACACCAAGGTGGACAAGAGAGTTGAGCCCAAATCTTGTGACAAAA
CTCACACATGCCCACCGTGCCCAGCACCTGAAGCCGCGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCGCGGTCTCCAAC
AAAGCCCTCCCAGCCCCCATCGAGAAAACCATCTCCAAAGCCAAAGGGCAGCCCCGAGAACCACAGGTGTATACCCT
GCCCCCATCCCGGGATGAGCTGACCAAGAACCAGGTCAGCCTGTCCTGCGCCGTCAAAGGCTTCTATCCCAGCGACA
TCGCCGTGGAGTGGGAGAGCAATGGGCAGCCGGAGAACAACTACAAGACCACGCCTCCCGTGCTGGACTCCGACGGC
TCCTTCTTCCTCGTGAGCAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTGGCGGCGGCGGTTCAGGTGGTG
GGGGATCCGACGTTGTGATGACGCAGTCTCCTAGTTCAGTATCAGCGTCTGTTGGGGATCGAGTCACGATTACATGC
CAGGCGAGCCAAAATATTAGGACGTATCTGTCTTGGTATCAACAAAAGCCTGGTAAGGCTCCAAAACTCCTCATTTA
CGCCGCTGCCAACTTGGCTAGTGGAGTACCTTCACGCTTTAGTGGGTCAGGTTCTGGAACAGATTTTACGTTGACGA
TCTCCGATTTGGAACCAGGGGACGCTGCCACGTATTATTGCCAGTCCACCTACCTCGGGACTGACTACGTTGGTGGG
GCGTTTGGCGGAGGAACAAAACTCACTGTACTTGGTGGTGGTGGTTCAGGCGGGGGGGGTTCCGGCGGAGGCGGTTC
TGGGGGTGGAGGTTCCGAGGTTCAGTTGGTTGAAAGTGGGGGGGGACTCGTTCAGCCTGGAGGCTCTTTGCGACTTT
CTTGTACCGCTTCTGGGTTCACTATAAGTTCATATCATATGCAATGGGTAAGACAGGCGCCTGGTAAGGGACTTGAA
TACATAGGAACAATAAGCTCTGGTGGTAACGTCTATTACGCCTCATCCGCGCGGGGGAGGTTTACAATTTCCAGGCC
TTCTAGCAAAAACACGGTAGATCTGCAAATGAACTCTCTTCGCGCTGAAGATACAGCAGTCTACTACTGCGCCAGGG
ATTCAGGGTATTCTGACCCCATGTGGGGGCAGGGCACATTGGTTACCGTGTCCTCTTGA
>Sequence ID 95: SI-75P5 Chain B Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSSGGGGSGGGGSDVVMTQSPSTLSASVGD
RVTINCQASESISSWLAWYQQKPGKAPKLLIYEASKLASGVPSRFSGSGSGTEFTLTISSLQPDDFATYYCQGYFYF
ISRTYVNSFGGGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLWCLVKGFYPSDIAVEWESNGQPENNYKTTPPVLDSD
GSFFLYSKLTVDKSRWQQGNVFSCSVMHEALHNRFTQKSLSLSPGGGGGSGGGGSDIQMTQSPSTLSASVGDRVTIT
CQASQSISSHLNWYQQKPGKAPKLLIYKASTLASGVPSRFSGSGSGTEFTLTISSLQPDDFATYYCQQGYSWGNVDN
VFGGGTKVTVLGGGGSGGGGSGGGGSGGGGSEVQLLESGGGLVQPGGSLSLSCAASGFSFSSGYDMCWVRQAPGKGL
EWIACIAAGSAGITYDANWAKGRFTISRDNSKNTLYLQMNSLRAEDTAVYYCARSAFSFDYAMDLWGQGTLVTVSS
>Sequence ID 96: SI-75P5 Chain B Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGCGGCG
GTGGAGGGTCCGGCGGTGGTGGATCAGACGTCGTGATGACCCAGTCTCCTTCCACCCTGTCTGCATCTGTAGGAGAC
AGAGTCACCATCAATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGCAGTGGATCTGGGA
CAGAATTCACTCTCACCATCAGCAGCCTGCAGCCTGATGATTTTGCAACTTATTACTGCCAAGGCTATTTTTATTTT
ATTAGTCGTACTTATGTAAATTCTTTCGGCGGAGGGACCAAGGTGGAGATCAAACGTACGGTGGCTGCACCATCTGT
CTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGAGAAACACAAAGTCTA
CGCCTGCGAAGTCACCCATCAGGGCCTGAGCTCGCCCGTCACAAAGAGCTTCAACAGGGGAGAGTGTGGAGGCGACA
AAACTCACACATGCCCACCGTGCCCAGCACCTGAAGCCGCGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAACCACAGGTGTACAC
CCTGCCCCCAAGCCGGGATGAGCTGACCAAGAACCAGGTCAGCCTGTGGTGCCTGGTCAAAGGCTTCTATCCCAGCG
ACATCGCCGTGGAGTGGGAGAGCAATGGGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCAGCAGGGGAACGTCTTCTCATGCTCCGT
GATGCATGAGGCTCTGCACAACAGATTCACGCAGAAGAGCCTCTCCCTGTCTCCGGGTGGCGGTGGAGGGTCCGGCG
GTGGTGGATCCGACATTCAGATGACACAGTCACCGTCTACGCTCTCAGCCTCAGTAGGCGATAGGGTGACTATCACC
TGCCAAGCCTCCCAATCTATAAGTTCACACCTCAACTGGTATCAACAGAAACCCGGTAAAGCACCGAAGCTGCTCAT
CTACAAGGCATCAACTCTTGCCTCAGGTGTGCCTAGTCGCTTCTCCGGGAGCGGCTCCGGTACTGAGTTTACTTTGA
CGATATCTTCCCTCCAACCCGACGACTTCGCCACATACTATTGTCAGCAGGGTTATAGTTGGGGAAATGTTGACAAT
GTGTTTGGAGGGGGAACGAAAGTGACTGTGTTGGGAGGGGGAGGAAGTGGCGGCGGCGGAAGCGGAGGTGGTGGCTC
CGGCGGGGGTGGAAGCGAAGTGCAATTGCTCGAAAGTGGTGGCGGACTTGTGCAACCTGGTGGAAGCCTCTCACTCT
CTTGCGCGGCGAGTGGTTTTAGCTTCAGTAGCGGCTACGATATGTGCTGGGTAAGACAGGCTCCTGGTAAAGGCCTT
GAGTGGATTGCGTGTATCGCCGCCGGGTCTGCTGGTATAACATACGATGCAAACTGGGCGAAAGGGCGCTTCACAAT
CTCCAGGGACAACTCAAAAAATACATTGTACCTCCAGATGAATAGCCTGAGAGCCGAAGACACCGCAGTCTACTATT
GCGCAAGGTCAGCATTTAGCTTCGATTATGCAATGGATCTTTGGGGCCAGGGGACGCTCGTCACCGTCTCATCTTGA
>Sequence ID 97: SI-75P8 Chain A Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSSGGGGSGGGGSQVQLQESGGRLVQPGEPLSL
TCKTSGIDLSSNAIGWVRQAPGKGLEWIGVIFGSGNTYYASWAKGRFTISRSTSTVYLKMNSLRSEDTAIYYCARGG
YSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPSVFLFPPKPKDTLMISR
TPEVTCVVVDVSHEDPEVKFNWYVDGVEVHNAKTKPREEQYNSTYRVVSVLTVLHQDWLNGKEYKCAVSNKALPAPI
EKTISKAKGQPREPQVYTLPPSRDELTKNQVSLSCAVKGFYPSDIAVEWESNGQPENNYKTTPPVLDSDGSFFLVSK
LTVDKSRWQQGNVFSCSVMHEALHNHYTQKSLSLSPGGGGGSGGGGSQVQLQQSGPGLVKPSETLSITCTVSGFSLT
NYGVHWIRQAPGKGLEWLGVIWSGGNTDYNTPFTSRFTITKDNSKNQVYFKLRSVRADDTAIYYCARALTYYDYEFA
YWGQGTLVTVSSGGGGSGGGGSGGGGSGGGGSEIVLTQSPSTLSVSPGERATFSCRASQSIGTNIHWYQQKPGKPPR
LLIKYASESISGIPDRFSGSGSGTEFTLTISSVQSEDFAVYYCQQNNNWPTTFGPGTKLTVL
>Sequence ID 98: SI-75P8 Chain A Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGCGGCGGTGGAGGGT
CCGGCGGTGGTGGATCACAAGTGCAGTTGCAAGAAAGTGGTGGTAGACTGGTTCAGCCTGGTGAACCCTTGTCACTG
ACGTGTAAAACAAGCGGCATTGATCTGTCCTCTAACGCCATCGGATGGGTCCGACAGGCCCCAGGAAAAGGTCTGGA
GTGGATCGGAGTTATCTTCGGGAGCGGCAATACATACTACGCAAGCTGGGCAAAAGGGCGATTTACGATATCACGGA
GCACCTCTACAGTTTATTTGAAAATGAACTCCCTCCGGTCCGAGGATACCGCGATATATTACTGTGCCAGAGGGGGG
TACTCCTCTGATATCTGGGGGCAGGGTACACTGGTTACAGTTTCATCCGCTAGCACCAAAGGCCCCTCTGTGTTCCC
TCTGGCCCCCTCCTCCAAGAGCACCTCTGGAGGCACTGCTGCCCTGGGCTGCCTGGTGAAGGACTACTTCCCAGAGC
CTGTGACTGTGTCCTGGAACTCTGGAGCCCTGACCTCTGGAGTGCACACCTTCCCTGCTGTGCTGCAGAGCTCTGGC
CTCTACAGCCTGTCCTCTGTGGTCACTGTGCCCTCCTCCAGCCTGGGCACCCAGACCTACATCTGCAATGTGAACCA
CAAGCCCTCCAACACCAAGGTGGACAAGAGAGTGGAGCCCAAGTCCTGTGACAAGACCCACACCTGCCCTCCCTGCC
CAGCCCCAGAGGCTGCTGGAGGCCCCTCTGTGTTCCTCTTCCCTCCCAAGCCCAAGGACACCCTGATGATCTCCAGG
ACCCCAGAAGTCACCTGTGTGGTGGTGGATGTGTCCCATGAAGACCCAGAAGTGAAGTTCAACTGGTATGTGGATGG
AGTGGAGGTGCACAATGCCAAGACCAAGCCCAGAGAAGAGCAGTACAACTCCACCTACCGAGTGGTGTCTGTGCTGA
CTGTGCTGCACCAGGACTGGCTGAATGGGAAGGAGTACAAGTGTGCTGTGTCCAACAAGGCCCTGCCTGCCCCCATT
GAGAAGACCATCTCCAAGGCCAAGGGCCAGCCCAGAGAGCCCCAGGTCTACACCCTGCCTCCCTCCAGAGATGAGCT
GACCAAGAACCAGGTGTCCCTGTCCTGTGCTGTGAAAGGCTTCTACCCCAGTGACATTGCTGTGGAGTGGGAGAGCA
ATGGCCAGCCAGAGAACAACTACAAGACCACCCCTCCTGTGCTGGACTCTGATGGCTCCTTCTTCCTGGTCTCCAAG
CTGACTGTGGACAAGAGCCGCTGGCAGCAGGGCAATGTGTTCTCCTGCTCTGTCATGCATGAGGCCCTGCACAACCA
CTACACCCAGAAGAGCCTGAGCCTGAGCCCTGGAGGAGGAGGAGGCTCTGGAGGAGGAGGATCCCAAGTTCAGTTGC
AGCAGTCTGGCCCTGGCCTGGTCAAGCCTTCTGAGACACTGTCCATCACCTGTACCGTGTCCGGCTTCTCCCTGACC
AATTACGGCGTGCACTGGATCAGACAGGCCCCTGGCAAAGGACTGGAATGGCTGGGAGTGATTTGGAGCGGCGGCAA
CACCGACTACAACACCCCTTTCACCAGCCGGTTCACCATCACCAAGGACAACTCCAAGAACCAGGTGTACTTCAAGC
TGCGGAGCGTGCGGGCTGATGACACCGCCATCTACTACTGTGCTCGGGCCCTGACCTACTACGACTACGAGTTTGCT
TACTGGGGCCAGGGCACCCTGGTCACAGTTTCTTCTGGTGGTGGTGGTTCAGGCGGGGGGGGTTCCGGCGGAGGCGG
TTCTGGGGGTGGAGGTTCCGAGATCGTGCTGACCCAGTCTCCTTCCACACTGTCTGTGTCTCCCGGCGAGAGAGCCA
CCTTCAGCTGTAGAGCCTCTCAGTCCATCGGCACCAACATCCACTGGTATCAGCAGAAGCCCGGCAAGCCTCCTCGG
CTGCTGATTAAGTACGCCTCCGAGTCCATCAGCGGCATCCCTGACAGATTCTCCGGCTCTGGCTCTGGCACCGAGTT
TACCCTGACCATCTCCTCCGTGCAGTCCGAGGATTTCGCCGTGTACTACTGCCAGCAGAACAACAACTGGCCCACCA
CCTTTGGACCCGGCACCAAGCTGACCGTGCTGTGA
>Sequence ID 99: SI-75P8 Chain B Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSSGGGGSGGGGSDPQLTQSPSSLSATVGQ
RVTINCQSSQSVAKNNNLAWFQQKPGKPPKLLIYSASTLAAGVPSRFSGSGSGTQFTLTITRVQSEDFATYYCSARD
SGNIQSFGGGTRVEIKRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECGGDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQDWLNGKEYKCAVSNK
ALPAPIEKTISKAKGQPREPQVYTLPPSRDELTKNQVSLWCLVKGFYPSDIAVEWESNGQPENNYKTTPPVLDSDGS
FFLYSKLTVDKSRWQQGNVFSCSVMHEALHNRFTQKSLSLSPGGGGGSGGGGSQVQLQESGGGLVKPGGSLSLSCAA
SGFTFSSYWMSWVRQAPGKGLEWVANINRDGSASYYVDSVKGRFTISRDDAKNSLYLQMNSLRAEDTAVYYCARDRG
VGYFDLWGRGTLVTVSSGGGGSGGGGSGGGGSQSALTQPASVSGSPGQSITISCTGTSSDVGGYNFVSWYQQHPGKA
PKLMIYDVSDRPSGVSDRFSGSKSGNTASLIISGLQADDEADYYCSSYGSSSTHVIFGGGTKVTVL
>Sequence ID 100: SI-75P8 Chain B Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGTGGCG
GTGGAGGGTCCGGCGGTGGTGGATCAGATCCTCAATTGACCCAGTCCCCTAGTAGCCTTTCTGCAACCGTGGGCCAG
AGGGTAACAATCAATTGCCAGTCAAGCCAGTCCGTAGCGAAGAATAATAATTTGGCATGGTTCCAACAAAAACCGGG
TAAACCTCCTAAACTCCTTATCTACTCAGCTTCAACCCTTGCCGCGGGTGTGCCCAGCCGGTTCTCAGGCTCAGGTT
CCGGGACTCAATTTACACTTACCATCACTCGCGTCCAAAGTGAGGATTTTGCAACCTACTATTGTTCTGCACGGGAT
TCCGGGAACATACAGTCCTTTGGGGGTGGAACTCGGGTGGAGATAAAACGTACGGTGGCTGCCCCCTCTGTGTTCAT
CTTCCCACCCAGTGATGAGCAGCTGAAGAGTGGCACAGCCTCTGTGGTGTGCCTGCTCAACAACTTCTACCCCAGAG
AAGCCAAGGTGCAGTGGAAGGTGGACAATGCCCTGCAGAGTGGCAACAGCCAGGAGAGTGTGACAGAGCAGGACAGC
AAGGACAGCACCTACAGCCTGTCCTCCACCCTGACCCTGTCCAAGGCTGACTATGAGAAGCACAAGGTGTATGCCTG
TGAGGTGACCCACCAGGGCCTGAGCAGCCCTGTGACCAAGAGCTTCAACCGAGGAGAGTGTGGAGGAGACAAGACCC
ACACCTGCCCTCCCTGCCCAGCCCCAGAAGCTGCTGGAGGCCCCTCTGTGTTCCTCTTCCCTCCCAAGCCCAAGGAC
ACCCTGATGATCTCCAGGACCCCAGAAGTCACCTGTGTGGTGGTGGATGTGTCCCATGAAGACCCAGAAGTGAAGTT
CAACTGGTATGTGGATGGAGTGGAAGTGCACAATGCCAAGACCAAGCCCAGAGAAGAGCAGTACAACAGCACCTACC
GAGTGGTGTCTGTGCTGACTGTGCTGCACCAGGACTGGCTGAATGGGAAGGAGTACAAGTGTGCTGTGTCCAACAAG
GCCCTGCCTGCCCCCATTGAGAAGACCATCTCCAAGGCCAAGGGCCAGCCCAGAGAGCCCCAGGTCTACACCCTGCC
TCCCTCCAGAGATGAGCTGACCAAGAACCAGGTGTCCCTGTGGTGCCTGGTGAAAGGCTTCTACCCCAGTGACATTG
CTGTGGAGTGGGAGAGCAATGGCCAGCCAGAGAACAACTACAAGACCACCCCTCCTGTGCTGGACTCTGATGGCTCC
TTCTTCCTGTACAGCAAGCTGACTGTGGACAAGAGCCGCTGGCAGCAGGGCAATGTGTTCTCCTGCTCTGTCATGCA
TGAGGCCCTGCACAACCGCTTCACCCAGAAGAGCCTGAGCCTGAGCCCTGGAGGAGGAGGAGGCTCTGGAGGAGGAG
GATCCCAGGTGCAGCTGCAGGAGTCGGGGGGAGGCCTGGTCAAGCCTGGAGGGTCCCTGAGCCTCTCCTGTGCAGCC
TCTGGATTCACCTTTAGTAGTTATTGGATGAGCTGGGTCCGCCAGGCTCCAGGGAAGGGGCTGGAGTGGGTGGCCAA
CATAAACCGCGATGGAAGTGCGAGTTACTATGTGGACTCTGTGAAGGGCCGATTCACCATCTCCAGAGACGACGCCA
AGAACTCACTGTATCTGCAAATGAACAGCCTGAGAGCTGAGGACACGGCTGTGTATTACTGTGCGAGAGATCGTGGG
GTGGGCTACTTCGATCTCTGGGGCCGTGGCACCCTGGTCACCGTGTCTAGCGGTGGAGGCGGTTCAGGCGGAGGTGG
TTCCGGCGGTGGCGGCTCCCAGTCTGCCCTGACTCAGCCTGCCTCCGTGTCTGGGTCTCCTGGACAGTCGATCACCA
TCTCCTGCACTGGAACCAGCAGTGACGTTGGTGGTTATAACTTTGTCTCCTGGTACCAACAACACCCAGGCAAAGCC
CCCAAACTCATGATCTATGATGTCAGTGATCGGCCCTCAGGGGTGTCTGATCGCTTCTCCGGCTCCAAGTCTGGCAA
CACGGCCTCCCTGATCATCTCTGGCCTCCAGGCTGACGACGAGGCTGATTATTACTGCAGCTCATATGGGAGCAGCA
GCACTCATGTGATTTTCGGCGGAGGGACCAAGGTGACCGTCCTATAA
>Sequence ID 101: SI-75X4 Chain A Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSSGGGGSGGGGSEVQLVESGGGLVQPGGSLRL
SCAASGFTISTNAMSWVRQAPGKGLEWIGVITGRDITYYASWAKGRFTISRDNSKNTLYLQMSSLRSEDTAVYYCAR
DGGSSAITSNNIWGQGTLVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQDWLNGKEYKCAVSN
KALPAPIEKTISKAKGQPREPQVYTLPPSRDELTKNQVSLSCAVKGFYPSDIAVEWESNGQPENNYKTTPPVLDSDG
SFFLVSKLTVDKSRWQQGNVFSCSVMHEALHNHYTQKSLSLSPG
>Sequence ID 102: SI-75X4 Chain A Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGCGGAGGAGGAGGCT
CTGGAGGAGGAGGCAGTGAGGTGCAGCTGGTGGAGAGTGGAGGAGGCCTGGTGCAGCCTGGAGGCAGCCTGCGGCTG
TCCTGTGCTGCCTCTGGCTTCACCATCTCCACCAATGCCATGTCCTGGGTGCGCCAGGCCCCTGGCAAGGGCCTGGA
GTGGATTGGAGTCATCACTGGCAGAGACATCACCTACTATGCCTCCTGGGCCAAAGGCCGCTTCACCATCTCCAGAG
ACAACAGCAAGAACACCCTGTACCTGCAGATGAGCAGCCTGCGCAGTGAGGACACAGCTGTGTACTACTGTGCCCGA
GATGGAGGCAGCAGTGCCATCACCTCCAACAACATCTGGGGCCAGGGCACCCTGGTCACTGTGTCCTCTGCTAGCAC
CAAAGGCCCCTCTGTGTTCCCTCTGGCCCCCTCCTCCAAGAGCACCTCTGGAGGCACTGCTGCCCTGGGCTGCCTGG
TGAAGGACTACTTCCCAGAGCCTGTGACTGTGTCCTGGAACTCTGGAGCCCTGACCTCTGGAGTGCACACCTTCCCT
GCTGTGCTGCAGAGCTCTGGCCTCTACAGCCTGTCCTCTGTGGTCACTGTGCCCTCCTCCAGCCTGGGCACCCAGAC
CTACATCTGCAATGTGAACCACAAGCCCTCCAACACCAAGGTGGACAAGAGAGTGGAGCCCAAGTCCTGTGACAAGA
CCCACACCTGCCCTCCCTGCCCAGCCCCAGAGGCTGCTGGAGGCCCCTCTGTGTTCCTCTTCCCTCCCAAGCCCAAG
GACACCCTGATGATCTCCAGGACCCCAGAAGTCACCTGTGTGGTGGTGGATGTGTCCCATGAAGACCCAGAAGTGAA
GTTCAACTGGTATGTGGATGGAGTGGAGGTGCACAATGCCAAGACCAAGCCCAGAGAAGAGCAGTACAACTCCACCT
ACCGAGTGGTGTCTGTGCTGACTGTGCTGCACCAGGACTGGCTGAATGGGAAGGAGTACAAGTGTGCTGTGTCCAAC
AAGGCCCTGCCTGCCCCCATTGAGAAGACCATCTCCAAGGCCAAGGGCCAGCCCAGAGAGCCCCAGGTCTACACCCT
GCCTCCCTCCAGAGATGAGCTGACCAAGAACCAGGTGTCCCTGTCCTGTGCTGTGAAAGGCTTCTACCCCAGTGACA
TTGCTGTGGAGTGGGAGAGCAATGGCCAGCCAGAGAACAACTACAAGACCACCCCTCCTGTGCTGGACTCTGATGGC
TCCTTCTTCCTGGTCTCCAAGCTGACTGTGGACAAGAGCCGCTGGCAGCAGGGCAATGTGTTCTCCTGCTCTGTCAT
GCATGAGGCCCTGCACAACCACTACACCCAGAAGAGCCTGAGCCTGAGCCCTGGATGA
>Sequence ID 103: SI-75X4 Chain B Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSSGGGGSGGGGSDVVMTQSPSTLSASVGD
RVTINCQASESISSWLAWYQQKPGKAPKLLIYEASKLASGVPSRFSGSGSGTEFTLTISSLQPDDFATYYCQGYFYF
ISRTYVNSFGGGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLWCLVKGFYPSDIAVEWESNGQPENNYKTTPPVLDSD
GSFFLYSKLTVDKSRWQQGNVFSCSVMHEALHNRFTQKSLSLSPG
>Sequence ID 104: SI-75X4 Chain B Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGCGGAG
GAGGAGGCTCTGGAGGAGGAGGCAGTGATGTGGTGATGACCCAGAGCCCCTCCACCCTGTCTGCCTCTGTGGGAGAC
AGAGTGACCATCAACTGCCAGGCCAGTGAGAGCATCTCCTCCTGGCTGGCCTGGTACCAGCAGAAGCCAGGCAAGGC
CCCCAAGCTGCTCATCTATGAAGCCTCCAAGCTGGCCTCTGGAGTGCCCTCCCGCTTCTCTGGCTCTGGCTCTGGCA
CAGAGTTCACCCTGACCATCTCCAGCCTGCAGCCAGATGACTTTGCCACCTACTACTGCCAGGGCTACTTCTACTTC
ATCTCCAGGACCTATGTGAACAGCTTTGGAGGAGGCACCAAGGTGGAGATCAAGCGTACGGTGGCTGCCCCCTCTGT
GTTCATCTTCCCACCCAGTGATGAGCAGCTGAAGAGTGGCACAGCCTCTGTGGTGTGCCTGCTCAACAACTTCTACC
CCAGAGAAGCCAAGGTGCAGTGGAAGGTGGACAATGCCCTGCAGAGTGGCAACAGCCAGGAGAGTGTGACAGAGCAG
GACAGCAAGGACAGCACCTACAGCCTGTCCTCCACCCTGACCCTGTCCAAGGCTGACTATGAGAAGCACAAGGTGTA
TGCCTGTGAGGTGACCCACCAGGGCCTGAGCAGCCCTGTGACCAAGAGCTTCAACCGAGGAGAGTGTGGAGGAGACA
AGACCCACACCTGCCCTCCCTGCCCAGCCCCAGAAGCTGCTGGAGGCCCCTCTGTGTTCCTCTTCCCTCCCAAGCCC
AAGGACACCCTGATGATCTCCAGGACCCCAGAAGTCACCTGTGTGGTGGTGGATGTGTCCCATGAAGACCCAGAAGT
GAAGTTCAACTGGTATGTGGATGGAGTGGAAGTGCACAATGCCAAGACCAAGCCCAGAGAAGAGCAGTACAACAGCA
CCTACCGAGTGGTGTCTGTGCTGACTGTGCTGCACCAGGACTGGCTGAATGGGAAGGAGTACAAGTGTGCTGTGTCC
AACAAGGCCCTGCCTGCCCCCATTGAGAAGACCATCTCCAAGGCCAAGGGCCAGCCCAGAGAGCCCCAGGTCTACAC
CCTGCCTCCCTCCAGAGATGAGCTGACCAAGAACCAGGTGTCCCTGTGGTGCCTGGTGAAAGGCTTCTACCCCAGTG
ACATTGCTGTGGAGTGGGAGAGCAATGGCCAGCCAGAGAACAACTACAAGACCACCCCTCCTGTGCTGGACTCTGAT
GGCTCCTTCTTCCTGTACAGCAAGCTGACTGTGGACAAGAGCCGCTGGCAGCAGGGCAATGTGTTCTCCTGCTCTGT
CATGCATGAGGCCCTGCACAACCGCTTCACCCAGAAGAGCCTGAGCCTGAGCCCTGGATGA
>Sequence ID 105: SI-75X17 Chain A Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSSGGGGSGGGGSQVQLQESGGRLVQPGEPLSL
TCKTSGIDLSSNAIGWVRQAPGKGLEWIGVIFGSGNTYYASWAKGRFTISRSTSTVYLKMNSLRSEDTAIYYCARGG
YSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPSVFLFPPKPKDTLMISR
TPEVTCVVVDVSHEDPEVKFNWYVDGVEVHNAKTKPREEQYNSTYRVVSVLTVLHQDWLNGKEYKCAVSNKALPAPI
EKTISKAKGQPREPQVYTLPPSRDELTKNQVSLSCAVKGFYPSDIAVEWESNGQPENNYKTTPPVLDSDGSFFLVSK
LTVDKSRWQQGNVFSCSVMHEALHNHYTQKSLSLSPG
>Sequence ID 106: SI-75X17 Chain A Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGCGGCGGTGGAGGGT
CCGGCGGTGGTGGATCACAAGTGCAGTTGCAAGAAAGTGGTGGTAGACTGGTTCAGCCTGGTGAACCCTTGTCACTG
ACGTGTAAAACAAGCGGCATTGATCTGTCCTCTAACGCCATCGGATGGGTCCGACAGGCCCCAGGAAAAGGTCTGGA
GTGGATCGGAGTTATCTTCGGGAGCGGCAATACATACTACGCAAGCTGGGCAAAAGGGCGATTTACGATATCACGGA
GCACCTCTACAGTTTATTTGAAAATGAACTCCCTCCGGTCCGAGGATACCGCGATATATTACTGTGCCAGAGGGGGG
TACTCCTCTGATATCTGGGGGCAGGGTACACTGGTTACAGTTTCATCCGCTAGCACCAAAGGCCCCTCTGTGTTCCC
TCTGGCCCCCTCCTCCAAGAGCACCTCTGGAGGCACTGCTGCCCTGGGCTGCCTGGTGAAGGACTACTTCCCAGAGC
CTGTGACTGTGTCCTGGAACTCTGGAGCCCTGACCTCTGGAGTGCACACCTTCCCTGCTGTGCTGCAGAGCTCTGGC
CTCTACAGCCTGTCCTCTGTGGTCACTGTGCCCTCCTCCAGCCTGGGCACCCAGACCTACATCTGCAATGTGAACCA
CAAGCCCTCCAACACCAAGGTGGACAAGAGAGTGGAGCCCAAGTCCTGTGACAAGACCCACACCTGCCCTCCCTGCC
CAGCCCCAGAGGCTGCTGGAGGCCCCTCTGTGTTCCTCTTCCCTCCCAAGCCCAAGGACACCCTGATGATCTCCAGG
ACCCCAGAAGTCACCTGTGTGGTGGTGGATGTGTCCCATGAAGACCCAGAAGTGAAGTTCAACTGGTATGTGGATGG
AGTGGAGGTGCACAATGCCAAGACCAAGCCCAGAGAAGAGCAGTACAACTCCACCTACCGAGTGGTGTCTGTGCTGA
CTGTGCTGCACCAGGACTGGCTGAATGGGAAGGAGTACAAGTGTGCTGTGTCCAACAAGGCCCTGCCTGCCCCCATT
GAGAAGACCATCTCCAAGGCCAAGGGCCAGCCCAGAGAGCCCCAGGTCTACACCCTGCCTCCCTCCAGAGATGAGCT
GACCAAGAACCAGGTGTCCCTGTCCTGTGCTGTGAAAGGCTTCTACCCCAGTGACATTGCTGTGGAGTGGGAGAGCA
ATGGCCAGCCAGAGAACAACTACAAGACCACCCCTCCTGTGCTGGACTCTGATGGCTCCTTCTTCCTGGTCTCCAAG
CTGACTGTGGACAAGAGCCGCTGGCAGCAGGGCAATGTGTTCTCCTGCTCTGTCATGCATGAGGCCCTGCACAACCA
CTACACCCAGAAGAGCCTGAGCCTGAGCCCTGGATGA
>Sequence ID 107: SI-75X17 Chain B Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSSGGGGSGGGGSDPQLTQSPSSLSATVGQ
RVTINCQSSQSVAKNNNLAWFQQKPGKPPKLLIYSASTLAAGVPSRFSGSGSGTQFTLTITRVQSEDFATYYCSARD
SGNIQSFGGGTRVEIKRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECGGDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQDWLNGKEYKCAVSNK
ALPAPIEKTISKAKGQPREPQVYTLPPSRDELTKNQVSLWCLVKGFYPSDIAVEWESNGQPENNYKTTPPVLDSDGS
FFLYSKLTVDKSRWQQGNVFSCSVMHEALHNRFTQKSLSLSPG
>Sequence ID 108: SI-75X17 Chain B Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGTGGCG
GTGGAGGGTCCGGCGGTGGTGGATCAGATCCTCAATTGACCCAGTCCCCTAGTAGCCTTTCTGCAACCGTGGGCCAG
AGGGTAACAATCAATTGCCAGTCAAGCCAGTCCGTAGCGAAGAATAATAATTTGGCATGGTTCCAACAAAAACCGGG
TAAACCTCCTAAACTCCTTATCTACTCAGCTTCAACCCTTGCCGCGGGTGTGCCCAGCCGGTTCTCAGGCTCAGGTT
CCGGGACTCAATTTACACTTACCATCACTCGCGTCCAAAGTGAGGATTTTGCAACCTACTATTGTTCTGCACGGGAT
TCCGGGAACATACAGTCCTTTGGGGGTGGAACTCGGGTGGAGATAAAACGTACGGTGGCTGCCCCCTCTGTGTTCAT
CTTCCCACCCAGTGATGAGCAGCTGAAGAGTGGCACAGCCTCTGTGGTGTGCCTGCTCAACAACTTCTACCCCAGAG
AAGCCAAGGTGCAGTGGAAGGTGGACAATGCCCTGCAGAGTGGCAACAGCCAGGAGAGTGTGACAGAGCAGGACAGC
AAGGACAGCACCTACAGCCTGTCCTCCACCCTGACCCTGTCCAAGGCTGACTATGAGAAGCACAAGGTGTATGCCTG
TGAGGTGACCCACCAGGGCCTGAGCAGCCCTGTGACCAAGAGCTTCAACCGAGGAGAGTGTGGAGGAGACAAGACCC
ACACCTGCCCTCCCTGCCCAGCCCCAGAAGCTGCTGGAGGCCCCTCTGTGTTCCTCTTCCCTCCCAAGCCCAAGGAC
ACCCTGATGATCTCCAGGACCCCAGAAGTCACCTGTGTGGTGGTGGATGTGTCCCATGAAGACCCAGAAGTGAAGTT
CAACTGGTATGTGGATGGAGTGGAAGTGCACAATGCCAAGACCAAGCCCAGAGAAGAGCAGTACAACAGCACCTACC
GAGTGGTGTCTGTGCTGACTGTGCTGCACCAGGACTGGCTGAATGGGAAGGAGTACAAGTGTGCTGTGTCCAACAAG
GCCCTGCCTGCCCCCATTGAGAAGACCATCTCCAAGGCCAAGGGCCAGCCCAGAGAGCCCCAGGTCTACACCCTGCC
TCCCTCCAGAGATGAGCTGACCAAGAACCAGGTGTCCCTGTGGTGCCTGGTGAAAGGCTTCTACCCCAGTGACATTG
CTGTGGAGTGGGAGAGCAATGGCCAGCCAGAGAACAACTACAAGACCACCCCTCCTGTGCTGGACTCTGATGGCTCC
TTCTTCCTGTACAGCAAGCTGACTGTGGACAAGAGCCGCTGGCAGCAGGGCAATGTGTTCTCCTGCTCTGTCATGCA
TGAGGCCCTGCACAACCGCTTCACCCAGAAGAGCCTGAGCCTGAGCCCTGGATGA
>Sequence ID 109: SI-75X19 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPGGGGGSGGGGSEVQLQESGGG
LVQAGDSLSLSCLVSGRSFNSYTMGWFRQAPGKEREFVAAILWSGPTTYYADSVKGRFTISRDNAKNTVYLQMNSLK
PEDTAVYYCAAALGVLVLAPGNVYSYWGQGTQVTVSS
>Sequence ID 110: SI-75X19 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGAGGAGGAGGAGGCTCTGGAGGAGGAGGATCCGAGGTGCAGCTGCAGGAGTCTGGCGGCGGC
CTGGTGCAGGCTGGGGACAGCCTGAGCCTGTCCTGCCTGGTGTCTGGCCGCTCCTTCAACTCCTACACCATGGGCTG
GTTCCGCCAGGCCCCTGGGAAGGAGCGGGAGTTTGTGGCTGCCATCCTGTGGAGTGGCCCCACCACCTACTATGCTG
ACTCTGTGAAGGGCCGCTTCACCATCTCCAGAGACAATGCCAAGAACACTGTGTACCTGCAGATGAACAGCCTGAAG
CCAGAGGACACAGCTGTGTACTACTGTGCTGCTGCCCTGGGAGTGCTGGTGCTGGCCCCTGGCAATGTGTACAGCTA
CTGGGGCCAGGGCACCCAGGTGACTGTGTCTAGCTGA
>Sequence ID 111: SI-75X19 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPGGGGGSGGGGSQVQLVQS
GGGLVQAGGSLSLSCAFSGRTFSMYTMGWFRQAPGKEREFVAANRGRGLSPDIADSVNGRFTISRDNAKNTLYLQMD
SLKPEDTAVYYCAADLQYGSSWPQRSSAEYDYWGQGTTVTVSS
>Sequence ID 112: SI-75X19 Chain B Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGAGGAGGAGGAGGCTCTGGAGGAGGAGGATCCCAGGTGCAGCTGGTGCAGTCT
GGCGGAGGCCTGGTGCAGGCTGGGGGCAGCCTGAGCCTGAGCTGTGCCTTCTCTGGCCGCACCTTCTCCATGTACAC
CATGGGCTGGTTCCGCCAGGCCCCAGGGAAGGAGAGAGAGTTTGTGGCTGCCAACAGAGGCAGAGGCCTGAGCCCAG
ACATTGCTGACTCTGTGAATGGCCGCTTCACCATCTCCAGAGACAATGCCAAGAACACCCTGTACCTGCAGATGGAC
AGCCTGAAGCCAGAGGACACAGCTGTGTACTACTGTGCTGCTGACCTGCAGTATGGCAGCAGCTGGCCTCAGAGGAG
CTCTGCTGAGTATGACTACTGGGGCCAGGGCACCACTGTGACTGTGTCTAGCTGA
>Sequence ID 113: SI-75X9 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 114: SI-75X9 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGATGA
>Sequence ID 115: SI-75X9 Chain B Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSSGGGGSGGGGSDPQLTQSPSSLSATVGQ
RVTINCQSSQSVAKNNNLAWFQQKPGKPPKLLIYSASTLAAGVPSRFSGSGSGTQFTLTITRVQSEDFATYYCSARD
SGNIQSFGGGTRVEIKRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECGGDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQDWLNGKEYKCAVSNK
ALPAPIEKTISKAKGQPREPQVYTLPPSRDELTKNQVSLWCLVKGFYPSDIAVEWESNGQPENNYKTTPPVLDSDGS
FFLYSKLTVDKSRWQQGNVFSCSVMHEALHNRFTQKSLSLSPG
>Sequence ID 116: SI-75X9 Chain B Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGTGGCG
GTGGAGGGTCCGGCGGTGGTGGATCAGATCCTCAATTGACCCAGTCCCCTAGTAGCCTTTCTGCAACCGTGGGCCAG
AGGGTAACAATCAATTGCCAGTCAAGCCAGTCCGTAGCGAAGAATAATAATTTGGCATGGTTCCAACAAAAACCGGG
TAAACCTCCTAAACTCCTTATCTACTCAGCTTCAACCCTTGCCGCGGGTGTGCCCAGCCGGTTCTCAGGCTCAGGTT
CCGGGACTCAATTTACACTTACCATCACTCGCGTCCAAAGTGAGGATTTTGCAACCTACTATTGTTCTGCACGGGAT
TCCGGGAACATACAGTCCTTTGGGGGTGGAACTCGGGTGGAGATAAAACGTACGGTGGCTGCCCCCTCTGTGTTCAT
CTTCCCACCCAGTGATGAGCAGCTGAAGAGTGGCACAGCCTCTGTGGTGTGCCTGCTCAACAACTTCTACCCCAGAG
AAGCCAAGGTGCAGTGGAAGGTGGACAATGCCCTGCAGAGTGGCAACAGCCAGGAGAGTGTGACAGAGCAGGACAGC
AAGGACAGCACCTACAGCCTGTCCTCCACCCTGACCCTGTCCAAGGCTGACTATGAGAAGCACAAGGTGTATGCCTG
TGAGGTGACCCACCAGGGCCTGAGCAGCCCTGTGACCAAGAGCTTCAACCGAGGAGAGTGTGGAGGAGACAAGACCC
ACACCTGCCCTCCCTGCCCAGCCCCAGAAGCTGCTGGAGGCCCCTCTGTGTTCCTCTTCCCTCCCAAGCCCAAGGAC
ACCCTGATGATCTCCAGGACCCCAGAAGTCACCTGTGTGGTGGTGGATGTGTCCCATGAAGACCCAGAAGTGAAGTT
CAACTGGTATGTGGATGGAGTGGAAGTGCACAATGCCAAGACCAAGCCCAGAGAAGAGCAGTACAACAGCACCTACC
GAGTGGTGTCTGTGCTGACTGTGCTGCACCAGGACTGGCTGAATGGGAAGGAGTACAAGTGTGCTGTGTCCAACAAG
GCCCTGCCTGCCCCCATTGAGAAGACCATCTCCAAGGCCAAGGGCCAGCCCAGAGAGCCCCAGGTCTACACCCTGCC
TCCCTCCAGAGATGAGCTGACCAAGAACCAGGTGTCCCTGTGGTGCCTGGTGAAAGGCTTCTACCCCAGTGACATTG
CTGTGGAGTGGGAGAGCAATGGCCAGCCAGAGAACAACTACAAGACCACCCCTCCTGTGCTGGACTCTGATGGCTCC
TTCTTCCTGTACAGCAAGCTGACTGTGGACAAGAGCCGCTGGCAGCAGGGCAATGTGTTCTCCTGCTCTGTCATGCA
TGAGGCCCTGCACAACCGCTTCACCCAGAAGAGCCTGAGCCTGAGCCCTGGATGA
>Sequence ID 117: SI-75X11 Chain A Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSSGGGGSGGGGSQVQLQESGGRLVQPGEPLSL
TCKTSGIDLSSNAIGWVRQAPGKGLEWIGVIFGSGNTYYASWAKGRFTISRSTSTVYLKMNSLRSEDTAIYYCARGG
YSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPSVFLFPPKPKDTLMISR
TPEVTCVVVDVSHEDPEVKFNWYVDGVEVHNAKTKPREEQYNSTYRVVSVLTVLHQDWLNGKEYKCAVSNKALPAPI
EKTISKAKGQPREPQVYTLPPSRDELTKNQVSLSCAVKGFYPSDIAVEWESNGQPENNYKTTPPVLDSDGSFFLVSK
LTVDKSRWQQGNVFSCSVMHEALHNHYTQKSLSLSPG
>Sequence ID 118: SI-75X11 Chain A Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGCGGCGGTGGAGGGT
CCGGCGGTGGTGGATCACAAGTGCAGTTGCAAGAAAGTGGTGGTAGACTGGTTCAGCCTGGTGAACCCTTGTCACTG
ACGTGTAAAACAAGCGGCATTGATCTGTCCTCTAACGCCATCGGATGGGTCCGACAGGCCCCAGGAAAAGGTCTGGA
GTGGATCGGAGTTATCTTCGGGAGCGGCAATACATACTACGCAAGCTGGGCAAAAGGGCGATTTACGATATCACGGA
GCACCTCTACAGTTTATTTGAAAATGAACTCCCTCCGGTCCGAGGATACCGCGATATATTACTGTGCCAGAGGGGGG
TACTCCTCTGATATCTGGGGGCAGGGTACACTGGTTACAGTTTCATCCGCTAGCACCAAAGGCCCCTCTGTGTTCCC
TCTGGCCCCCTCCTCCAAGAGCACCTCTGGAGGCACTGCTGCCCTGGGCTGCCTGGTGAAGGACTACTTCCCAGAGC
CTGTGACTGTGTCCTGGAACTCTGGAGCCCTGACCTCTGGAGTGCACACCTTCCCTGCTGTGCTGCAGAGCTCTGGC
CTCTACAGCCTGTCCTCTGTGGTCACTGTGCCCTCCTCCAGCCTGGGCACCCAGACCTACATCTGCAATGTGAACCA
CAAGCCCTCCAACACCAAGGTGGACAAGAGAGTGGAGCCCAAGTCCTGTGACAAGACCCACACCTGCCCTCCCTGCC
CAGCCCCAGAGGCTGCTGGAGGCCCCTCTGTGTTCCTCTTCCCTCCCAAGCCCAAGGACACCCTGATGATCTCCAGG
ACCCCAGAAGTCACCTGTGTGGTGGTGGATGTGTCCCATGAAGACCCAGAAGTGAAGTTCAACTGGTATGTGGATGG
AGTGGAGGTGCACAATGCCAAGACCAAGCCCAGAGAAGAGCAGTACAACTCCACCTACCGAGTGGTGTCTGTGCTGA
CTGTGCTGCACCAGGACTGGCTGAATGGGAAGGAGTACAAGTGTGCTGTGTCCAACAAGGCCCTGCCTGCCCCCATT
GAGAAGACCATCTCCAAGGCCAAGGGCCAGCCCAGAGAGCCCCAGGTCTACACCCTGCCTCCCTCCAGAGATGAGCT
GACCAAGAACCAGGTGTCCCTGTCCTGTGCTGTGAAAGGCTTCTACCCCAGTGACATTGCTGTGGAGTGGGAGAGCA
ATGGCCAGCCAGAGAACAACTACAAGACCACCCCTCCTGTGCTGGACTCTGATGGCTCCTTCTTCCTGGTCTCCAAG
CTGACTGTGGACAAGAGCCGCTGGCAGCAGGGCAATGTGTTCTCCTGCTCTGTCATGCATGAGGCCCTGCACAACCA
CTACACCCAGAAGAGCCTGAGCCTGAGCCCTGGATGA
>Sequence ID 119: SI-75X11 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPG
>Sequence ID 120: SI-75X11 Chain B Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGATGA
>Sequence ID 121: SI-75X15 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPG
>Sequence ID 122: SI-75X15 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGATGA
>Sequence ID 123: SI-75X15 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPGGGGGSGGGGSQVQLVQS
GGGLVQAGGSLSLSCAFSGRTFSMYTMGWFRQAPGKEREFVAANRGRGLSPDIADSVNGRFTISRDNAKNTLYLQMD
SLKPEDTAVYYCAADLQYGSSWPQRSSAEYDYWGQGTTVTVSS
>Sequence ID 124: SI-75X15 Chain B Amino Acid Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGAGGAGGAGGAGGCTCTGGAGGAGGAGGATCCCAGGTGCAGCTGGTGCAGTCT
GGCGGAGGCCTGGTGCAGGCTGGGGGCAGCCTGAGCCTGAGCTGTGCCTTCTCTGGCCGCACCTTCTCCATGTACAC
CATGGGCTGGTTCCGCCAGGCCCCAGGGAAGGAGAGAGAGTTTGTGGCTGCCAACAGAGGCAGAGGCCTGAGCCCAG
ACATTGCTGACTCTGTGAATGGCCGCTTCACCATCTCCAGAGACAATGCCAAGAACACCCTGTACCTGCAGATGGAC
AGCCTGAAGCCAGAGGACACAGCTGTGTACTACTGTGCTGCTGACCTGCAGTATGGCAGCAGCTGGCCTCAGAGGAG
CTCTGCTGAGTATGACTACTGGGGCCAGGGCACCACTGTGACTGTGTCTAGCTGA
>Sequence ID 125: SI-75X13 Chain A Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAA
GGPSVFLFPPKPKDTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSPGGGGGSGGGGSEVQLQESGGG
LVQAGDSLSLSCLVSGRSFNSYTMGWFRQAPGKEREFVAAILWSGPTTYYADSVKGRFTISRDNAKNTVYLQMNSLK
PEDTAVYYCAAALGVLVLAPGNVYSYWGQGTQVTVSS
>Sequence ID 126: SI-75X13 Chain A Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAAGGCCCCTCTGTGTTCCCTCTGGCCCCCTCCTCCA
AGAGCACCTCTGGAGGCACTGCTGCCCTGGGCTGCCTGGTGAAGGACTACTTCCCAGAGCCTGTGACTGTGTCCTGG
AACTCTGGAGCCCTGACCTCTGGAGTGCACACCTTCCCTGCTGTGCTGCAGAGCTCTGGCCTCTACAGCCTGTCCTC
TGTGGTCACTGTGCCCTCCTCCAGCCTGGGCACCCAGACCTACATCTGCAATGTGAACCACAAGCCCTCCAACACCA
AGGTGGACAAGAGAGTGGAGCCCAAGTCCTGTGACAAGACCCACACCTGCCCTCCCTGCCCAGCCCCAGAGGCTGCT
GGAGGCCCCTCTGTGTTCCTCTTCCCTCCCAAGCCCAAGGACACCCTGATGATCTCCAGGACCCCAGAAGTCACCTG
TGTGGTGGTGGATGTGTCCCATGAAGACCCAGAAGTGAAGTTCAACTGGTATGTGGATGGAGTGGAGGTGCACAATG
CCAAGACCAAGCCCAGAGAAGAGCAGTACAACTCCACCTACCGAGTGGTGTCTGTGCTGACTGTGCTGCACCAGGAC
TGGCTGAATGGGAAGGAGTACAAGTGTGCTGTGTCCAACAAGGCCCTGCCTGCCCCCATTGAGAAGACCATCTCCAA
GGCCAAGGGCCAGCCCAGAGAGCCCCAGGTCTACACCCTGCCTCCCTCCAGAGATGAGCTGACCAAGAACCAGGTGT
CCCTGTCCTGTGCTGTGAAAGGCTTCTACCCCAGTGACATTGCTGTGGAGTGGGAGAGCAATGGCCAGCCAGAGAAC
AACTACAAGACCACCCCTCCTGTGCTGGACTCTGATGGCTCCTTCTTCCTGGTCTCCAAGCTGACTGTGGACAAGAG
CCGCTGGCAGCAGGGCAATGTGTTCTCCTGCTCTGTCATGCATGAGGCCCTGCACAACCACTACACCCAGAAGAGCC
TGAGCCTGAGCCCTGGAGGAGGAGGAGGCTCTGGAGGAGGAGGATCCGAGGTGCAGCTGCAGGAGTCTGGCGGCGGC
CTGGTGCAGGCTGGGGACAGCCTGAGCCTGTCCTGCCTGGTGTCTGGCCGCTCCTTCAACTCCTACACCATGGGCTG
GTTCCGCCAGGCCCCTGGGAAGGAGCGGGAGTTTGTGGCTGCCATCCTGTGGAGTGGCCCCACCACCTACTATGCTG
ACTCTGTGAAGGGCCGCTTCACCATCTCCAGAGACAATGCCAAGAACACTGTGTACCTGCAGATGAACAGCCTGAAG
CCAGAGGACACAGCTGTGTACTACTGTGCTGCTGCCCTGGGAGTGCTGGTGCTGGCCCCTGGCAATGTGTACAGCTA
CTGGGGCCAGGGCACCCAGGTGACTGTGTCTAGCTGA
>Sequence ID 127: SI-75X13 Chain B Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGECGGDKTHTCPPCPAP
EAAGGPSVFLFPPKPKDTLMISRTPEVTCVVVDVSHEDPEVKFNWYVDGVEVHNAKTKPREEQYNSTYRVVSVLTVL
HQDWLNGKEYKCAVSNKALPAPIEKTISKAKGQPREPQVYTLPPSRDELTKNQVSLWCLVKGFYPSDIAVEWESNGQ
PENNYKTTPPVLDSDGSFFLYSKLTVDKSRWQQGNVFSCSVMHEALHNRFTQKSLSLSPG
>Sequence ID 128: SI-75X13 Chain B Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAACGTACGGTGGCTGCCCCCTCTGTGTTCATCTTCCCACCCAGTGATGAGCAGCTGA
AGAGTGGCACAGCCTCTGTGGTGTGCCTGCTCAACAACTTCTACCCCAGAGAAGCCAAGGTGCAGTGGAAGGTGGAC
AATGCCCTGCAGAGTGGCAACAGCCAGGAGAGTGTGACAGAGCAGGACAGCAAGGACAGCACCTACAGCCTGTCCTC
CACCCTGACCCTGTCCAAGGCTGACTATGAGAAGCACAAGGTGTATGCCTGTGAGGTGACCCACCAGGGCCTGAGCA
GCCCTGTGACCAAGAGCTTCAACCGAGGAGAGTGTGGAGGAGACAAGACCCACACCTGCCCTCCCTGCCCAGCCCCA
GAAGCTGCTGGAGGCCCCTCTGTGTTCCTCTTCCCTCCCAAGCCCAAGGACACCCTGATGATCTCCAGGACCCCAGA
AGTCACCTGTGTGGTGGTGGATGTGTCCCATGAAGACCCAGAAGTGAAGTTCAACTGGTATGTGGATGGAGTGGAAG
TGCACAATGCCAAGACCAAGCCCAGAGAAGAGCAGTACAACAGCACCTACCGAGTGGTGTCTGTGCTGACTGTGCTG
CACCAGGACTGGCTGAATGGGAAGGAGTACAAGTGTGCTGTGTCCAACAAGGCCCTGCCTGCCCCCATTGAGAAGAC
CATCTCCAAGGCCAAGGGCCAGCCCAGAGAGCCCCAGGTCTACACCCTGCCTCCCTCCAGAGATGAGCTGACCAAGA
ACCAGGTGTCCCTGTGGTGCCTGGTGAAAGGCTTCTACCCCAGTGACATTGCTGTGGAGTGGGAGAGCAATGGCCAG
CCAGAGAACAACTACAAGACCACCCCTCCTGTGCTGGACTCTGATGGCTCCTTCTTCCTGTACAGCAAGCTGACTGT
GGACAAGAGCCGCTGGCAGCAGGGCAATGTGTTCTCCTGCTCTGTCATGCATGAGGCCCTGCACAACCGCTTCACCC
AGAAGAGCCTGAGCCTGAGCCCTGGATGA
>Sequence ID 129: SI-38P11 Chain A Amino Acid Sequence
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLTVLGGGGSGGGGSGGGGSGGGGSQVQLQQPGAELVKPGASVKMSCKASGYT
FTSYNMHWVKQTPGRGLEWIGAIYPGNGDTSYNQKFKGKATLTADKSSSTAYMQLSSLTSEDSAVYYCARSTYYGGD
WYFNVWGAGTTVTVSSGGGGSGGGGSEVQLVESGGGLVQPGGSLRLSCAASGFTISTNAMSWVRQAPGKGLEWIGVI
TGRDITYYASWAKGRFTISRDNSKNTLYLQMNSLRAEDTAVYYCARDGGSSAITSNNIWGQGTLVTVSSASTKGPSV
FPLAPSSKSTSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLSCAVKGFYPSDIAVEWESNGQPENNYKTTPPVLDSDGSFFLVSKLTVDKSRWQQGNVFSCSVMHEALH
NHYTQKSLSLSPGGGGGSGGGGSDVVMTQSPSSVSASVGDRVTITCQASQNIRTYLSWYQQKPGKAPKLLIYAAANL
ASGVPSRFSGSGSGTDFTLTISDLEPGDAATYYCQSTYLGTDYVGGAFGGGTKLTVLGGGGSGGGGSGGGGSGGGGS
EVQLVESGGGLVQPGGSLRLSCTASGFTISSYHMQWVRQAPGKGLEYIGTISSGGNVYYASSARGRFTISRPSSKNT
VDLQMNSLRAEDTAVYYCARDSGYSDPMWGQGTLVTVSS
>Sequence ID 130: SI-38P11 Chain A Nucleotide Sequence
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GACCGTGCTGGGCGGTGGCGGTAGTGGGGGAGGCGGTTCTGGCGGCGGAGGGTCCGGCGGTGGAGGATCACAGGTGC
AGCTGCAGCAGCCCGGCGCCGAGCTGGTGAAGCCCGGCGCCAGCGTGAAGATGAGCTGCAAGGCCAGCGGCTACACC
TTCACCAGCTACAACATGCACTGGGTGAAGCAGACCCCCGGCCGGGGCCTGGAGTGGATCGGCGCCATCTACCCCGG
CAACGGCGACACCAGCTACAACCAGAAGTTCAAGGGCAAGGCCACCCTGACCGCCGACAAGAGCAGCAGCACCGCCT
ACATGCAGCTGAGCAGCCTGACCAGCGAGGACAGCGCCGTGTACTACTGCGCCCGGAGCACCTACTACGGCGGCGAC
TGGTACTTCAACGTGTGGGGCGCCGGCACCACCGTGACCGTCTCGAGCGGCGGTGGAGGGTCCGGCGGTGGTGGATC
AGAGGTGCAGCTGGTGGAGTCTGGGGGAGGCTTGGTCCAGCCTGGGGGGTCCCTGAGACTCTCCTGTGCAGCCTCTG
GATTCACCATCAGTACCAATGCAATGAGCTGGGTCCGCCAGGCTCCAGGGAAGGGGCTGGAGTGGATCGGAGTCATT
ACTGGTCGTGATATCACATACTACGCGAGCTGGGCGAAAGGCAGATTCACCATCTCCAGAGACAATTCCAAGAACAC
GCTGTATCTTCAAATGAACAGCCTGAGAGCCGAGGACACGGCTGTGTATTACTGTGCGAGAGACGGTGGTTCTTCTG
CTATTACTAGTAACAACATTTGGGGCCAGGGAACCCTGGTCACCGTGTCCTCAGCTAGCACCAAGGGCCCATCGGTC
TTCCCCCTGGCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTATACCCTGCCCCCATCCCGGGAT
GAGCTGACCAAGAACCAGGTCAGCCTGTCCTGCGCCGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCGTGA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTGGCGGCGGCGGTTCAGGTGGTGGGGGATCCGACGTTGT
GATGACGCAGTCTCCTAGTTCAGTATCAGCGTCTGTTGGGGATCGAGTCACGATTACATGCCAGGCGAGCCAAAATA
TTAGGACGTATCTGTCTTGGTATCAACAAAAGCCTGGTAAGGCTCCAAAACTCCTCATTTACGCCGCTGCCAACTTG
GCTAGTGGAGTACCTTCACGCTTTAGTGGGTCAGGTTCTGGAACAGATTTTACGTTGACGATCTCCGATTTGGAACC
AGGGGACGCTGCCACGTATTATTGCCAGTCCACCTACCTCGGGACTGACTACGTTGGTGGGGCGTTTGGCGGAGGAA
CAAAACTCACTGTACTTGGTGGTGGTGGTTCAGGCGGGGGGGGTTCCGGCGGAGGCGGTTCTGGGGGTGGAGGTTCC
GAGGTTCAGTTGGTTGAAAGTGGGGGGGGACTCGTTCAGCCTGGAGGCTCTTTGCGACTTTCTTGTACCGCTTCTGG
GTTCACTATAAGTTCATATCATATGCAATGGGTAAGACAGGCGCCTGGTAAGGGACTTGAATACATAGGAACAATAA
GCTCTGGTGGTAACGTCTATTACGCCTCATCCGCGCGGGGGAGGTTTACAATTTCCAGGCCTTCTAGCAAAAACACG
GTAGATCTGCAAATGAACTCTCTTCGCGCTGAAGATACAGCAGTCTACTACTGCGCCAGGGATTCAGGGTATTCTGA
CCCCATGTGGGGGCAGGGCACATTGGTTACCGTGTCCTCTTGA
>Sequence ID 131: SI-38P11 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFSCSVMHEALHNRF
TQKSLSLSPGGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVTVLGGGGSGGGGSGGGGSGGGGSEVQL
LESGGGLVQPGGSLSLSCAASGFSFSSGYDMCWVRQAPGKGLEWIACIAAGSAGITYDANWAKGRFTISRDNSKNTL
YLQMNSLRAEDTAVYYCARSAFSFDYAMDLWGQGTLVTVSS
>Sequence ID 132: SI-38P11 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGCGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCAAGCCGGGATGAGCTGACC
AAGAACCAGGTCAGCCTGTGGTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCTCTGCACAACAGATTC
ACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCAC
ACCTCAACTGGTATCAACAGAAACCCGGTAAAGCACCGAAGCTGCTCATCTACAAGGCATCAACTCTTGCCTCAGGT
GTGCCTAGTCGCTTCTCCGGGAGCGGCTCCGGTACTGAGTTTACTTTGACGATATCTTCCCTCCAACCCGACGACTT
CGCCACATACTATTGTCAGCAGGGTTATAGTTGGGGAAATGTTGACAATGTGTTTGGAGGGGGAACGAAAGTGACTG
TGTTGGGAGGGGGAGGAAGTGGCGGCGGCGGAAGCGGAGGTGGTGGCTCCGGCGGGGGTGGAAGCGAAGTGCAATTG
CTCGAAAGTGGTGGCGGACTTGTGCAACCTGGTGGAAGCCTCTCACTCTCTTGCGCGGCGAGTGGTTTTAGCTTCAG
TAGCGGCTACGATATGTGCTGGGTAAGACAGGCTCCTGGTAAAGGCCTTGAGTGGATTGCGTGTATCGCCGCCGGGT
CTGCTGGTATAACATACGATGCAAACTGGGCGAAAGGGCGCTTCACAATCTCCAGGGACAACTCAAAAAATACATTG
TACCTCCAGATGAATAGCCTGAGAGCCGAAGACACCGCAGTCTACTATTGCGCAAGGTCAGCATTTAGCTTCGATTA
TGCAATGGATCTTTGGGGCCAGGGGACGCTCGTCACCGTCTCATCTTGA
>Sequence ID 133: SI-76PM1 Chain A Amino Acid Sequence
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLTVLGGGGSGGGGSGGGGSGGGGSQVQLQQPGAELVKPGASVKMSCKASGYT
FTSYNMHWVKQTPGRGLEWIGAIYPGNGDTSYNQKFKGKATLTADKSSSTAYMQLSSLTSEDSAVYYCARSTYYGGD
WYFNVWGAGTTVTVSSGGGGSGGGGSEVQLVESGGGLVQPGGSLRLSCAASGFTISTNAMSWVRQAPGKCLEWIGVI
TGRDITYYASWAKGRFTISRDNSKNTLYLQMNSLRAEDTAVYYCARDGGSSAITSNNIWGQGTLVTVSSASTKGPSV
FPLAPSSKSTSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLSCAVKGFYPSDIAVEWESNGQPENNYKTTPPVLDSDGSFFLVSKLTVDKSRWQQGNVFSCSVMHEALH
NHYTQKSLSLSPGGGGGSGGGGSDVVMTQSPSSVSASVGDRVTITCQASQNIRTYLSWYQQKPGKAPKLLIYAAANL
ASGVPSRFSGSGSGTDFTLTISDLEPGDAATYYCQSTYLGTDYVGGAFGGGTKLTVLGGGGSGGGGSGGGGSGGGGS
EVQLVESGGGLVQPGGSLRLSCTASGFTISSYHMQWVRQAPGKGLEYIGTISSGGNVYYASSARGRFTISRPSSKNT
VDLQMNSLRAEDTAVYYCARDSGYSDPMWGQGTLVTVSS
>Sequence ID 134: SI-76PM1 Chain A Nucleotide Sequence
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GACCGTGCTGGGCGGTGGCGGTAGTGGGGGAGGCGGTTCTGGCGGCGGAGGGTCCGGCGGTGGAGGATCACAGGTGC
AGCTGCAGCAGCCCGGCGCCGAGCTGGTGAAGCCCGGCGCCAGCGTGAAGATGAGCTGCAAGGCCAGCGGCTACACC
TTCACCAGCTACAACATGCACTGGGTGAAGCAGACCCCCGGCCGGGGCCTGGAGTGGATCGGCGCCATCTACCCCGG
CAACGGCGACACCAGCTACAACCAGAAGTTCAAGGGCAAGGCCACCCTGACCGCCGACAAGAGCAGCAGCACCGCCT
ACATGCAGCTGAGCAGCCTGACCAGCGAGGACAGCGCCGTGTACTACTGCGCCCGGAGCACCTACTACGGCGGCGAC
TGGTACTTCAACGTGTGGGGCGCCGGCACCACCGTGACCGTCTCGAGCGGCGGTGGAGGGTCCGGCGGTGGTGGATC
AGAGGTGCAGCTGGTGGAGTCTGGGGGAGGCTTGGTCCAGCCTGGGGGGTCCCTGAGACTCTCCTGTGCAGCCTCTG
GATTCACCATCAGTACCAATGCAATGAGCTGGGTCCGCCAGGCTCCAGGGAAGTGTCTGGAGTGGATCGGAGTCATT
ACTGGTCGTGATATCACATACTACGCGAGCTGGGCGAAAGGCAGATTCACCATCTCCAGAGACAATTCCAAGAACAC
GCTGTATCTTCAAATGAACAGCCTGAGAGCCGAGGACACGGCTGTGTATTACTGTGCGAGAGACGGTGGTTCTTCTG
CTATTACTAGTAACAACATTTGGGGCCAGGGAACCCTGGTCACCGTGTCCTCAGCTAGCACCAAGGGCCCATCGGTC
TTCCCCCTGGCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTATACCCTGCCCCCATCCCGGGAT
GAGCTGACCAAGAACCAGGTCAGCCTGTCCTGCGCCGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCGTGA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTGGCGGCGGCGGTTCAGGTGGTGGGGGATCCGACGTTGT
GATGACGCAGTCTCCTAGTTCAGTATCAGCGTCTGTTGGGGATCGAGTCACGATTACATGCCAGGCGAGCCAAAATA
TTAGGACGTATCTGTCTTGGTATCAACAAAAGCCTGGTAAGGCTCCAAAACTCCTCATTTACGCCGCTGCCAACTTG
GCTAGTGGAGTACCTTCACGCTTTAGTGGGTCAGGTTCTGGAACAGATTTTACGTTGACGATCTCCGATTTGGAACC
AGGGGACGCTGCCACGTATTATTGCCAGTCCACCTACCTCGGGACTGACTACGTTGGTGGGGCGTTTGGCGGAGGAA
CAAAACTCACTGTACTTGGTGGTGGTGGTTCAGGCGGGGGGGGTTCCGGCGGAGGCGGTTCTGGGGGTGGAGGTTCC
GAGGTTCAGTTGGTTGAAAGTGGGGGGGGACTCGTTCAGCCTGGAGGCTCTTTGCGACTTTCTTGTACCGCTTCTGG
GTTCACTATAAGTTCATATCATATGCAATGGGTAAGACAGGCGCCTGGTAAGGGACTTGAATACATAGGAACAATAA
GCTCTGGTGGTAACGTCTATTACGCCTCATCCGCGCGGGGGAGGTTTACAATTTCCAGGCCTTCTAGCAAAAACACG
GTAGATCTGCAAATGAACTCTCTTCGCGCTGAAGATACAGCAGTCTACTACTGCGCCAGGGATTCAGGGTATTCTGA
CCCCATGTGGGGGCAGGGCACATTGGTTACCGTGTCCTCTTGA
>Sequence ID 135: SI-76PM1 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGCGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFSCSVMHEALHNRF
TQKSLSLSPGGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVTVLGGGGSGGGGSGGGGSGGGGSEVQL
LESGGGLVQPGGSLSLSCAASGFSFSSGYDMCWVRQAPGKGLEWIACIAAGSAGITYDANWAKGRETISRDNSKNTL
YLQMNSLRAEDTAVYYCARSAFSFDYAMDLWGQGTLVTVSS
>Sequence ID 136: SI-76PM1 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCTGTGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGCGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCAAGCCGGGATGAGCTGACC
AAGAACCAGGTCAGCCTGTGGTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCTCTGCACAACAGATTC
ACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCAC
ACCTCAACTGGTATCAACAGAAACCCGGTAAAGCACCGAAGCTGCTCATCTACAAGGCATCAACTCTTGCCTCAGGT
GTGCCTAGTCGCTTCTCCGGGAGCGGCTCCGGTACTGAGTTTACTTTGACGATATCTTCCCTCCAACCCGACGACTT
CGCCACATACTATTGTCAGCAGGGTTATAGTTGGGGAAATGTTGACAATGTGTTTGGAGGGGGAACGAAAGTGACTG
TGTTGGGAGGGGGAGGAAGTGGCGGCGGCGGAAGCGGAGGTGGTGGCTCCGGCGGGGGTGGAAGCGAAGTGCAATTG
CTCGAAAGTGGTGGCGGACTTGTGCAACCTGGTGGAAGCCTCTCACTCTCTTGCGCGGCGAGTGGTTTTAGCTTCAG
TAGCGGCTACGATATGTGCTGGGTAAGACAGGCTCCTGGTAAAGGCCTTGAGTGGATTGCGTGTATCGCCGCCGGGT
CTGCTGGTATAACATACGATGCAAACTGGGCGAAAGGGCGCTTCACAATCTCCAGGGACAACTCAAAAAATACATTG
TACCTCCAGATGAATAGCCTGAGAGCCGAAGACACCGCAGTCTACTATTGCGCAAGGTCAGCATTTAGCTTCGATTA
TGCAATGGATCTTTGGGGCCAGGGGACGCTCGTCACCGTCTCATCTTGA
>Sequence ID 137: SI-68X2 Chain A Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGCGTKVEIKGGGGSGGGGSGGGGSGGGGSQVQLQESGPGLVKPSETLSLTCTVSGG
SVSSGDYYWTWIRQSPGKCLEWIGHIYYSGNTNYNPSLKSRLTISIDTSKTQFSLKLSSVTAADTAIYYCVRDRVTG
AFDIWGQGTMVTVSTGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGK
>Sequence ID 138: SI-68X2 Chain A Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAAGGCGGTGGCGGTAGTGGGGGAGGCGGTTCTGGCGGCGGAGGGTCCGGCGGTGGAGGATCACAGG
TGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGTGCCTGGAGTGGATTGGACACAT
CTATTACAGTGGGAACACCAATTATAACCCCTCCCTCAAGAGCCGACTCACCATATCAATTGACACGTCCAAGACTC
AGTTCTCCCTGAAGCTGAGTTCTGTGACCGCTGCGGACACGGCCATTTATTACTGTGTGCGAGATCGAGTGACTGGT
GCTTTTGATATCTGGGGCCAAGGGACAATGGTCACCGTGTCGACAGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTGTACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTAAATAG
>Sequence ID 139: SI-68X2 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGCGTKVTVLGGGGSGGGGSGGGGSGGGGSQVQLQESGGGLVKPGGSLRLSCA
ASGFTFSSYWMSWVRQAPGKCLEWVANINRDGSASYYVDSVKGRFTISRDDAKNSLYLQMNSLRAEDTAVYYCARDR
GVGYFDLWGRGTLVTVSTGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVYTLPPCRD
ELTKNQVSLWCLVKGFYPSDIAVEWESNGQPENNYKTTPPVLDSDGSFFLYSKLTVDKSRWQQGNVFSCSVMHEALH
NHYTQKSLSLSPGK
>Sequence ID 140: SI-68X2 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CTGTGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGTGCCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTGTCGACAGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACGCCAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCATGTCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAATAG
>Sequence ID 141: SI-68E1 Chain A Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGCGTKVEIKGGGGSGGGGSGGGGSGGGGSQVQLQESGPGLVKPSETLSLTCTVSGG
SVSSGDYYWTWIRQSPGKCLEWIGHIYYSGNTNYNPSLKSRLTISIDTSKTQFSLKLSSVTAADTAIYYCVRDRVTG
AFDIWGQGTMVTVSTGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSRSLVESGGGLVQPGGSLRLSCTASGFTISSYHMQWVRQAPGKCLEYIGTISSGGN
VYYASSARGRFTISRPSSKNTVDLQMNSLRAEDTAVYYCARDSGYSDPMWGQGTLVTVSSGGGGSGGGGSGGGGSGG
GGSDVVMTQSPSSVSASVGDRVTITCQASQNIRTYLSWYQQKPGKAPKLLIYAAANLASGVPSRFSGSGSGTDFTLT
ISDLEPGDAATYYCQSTYLGTDYVGGAFGCGTKVEIK
>Sequence ID 142: SI-68E1 Chain A Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAAGGCGGTGGCGGTAGTGGGGGAGGCGGTTCTGGCGGCGGAGGGTCCGGCGGTGGAGGATCACAGG
TGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGTGCCTGGAGTGGATTGGACACAT
CTATTACAGTGGGAACACCAATTATAACCCCTCCCTCAAGAGCCGACTCACCATATCAATTGACACGTCCAAGACTC
AGTTCTCCCTGAAGCTGAGTTCTGTGACCGCTGCGGACACGGCCATTTATTACTGTGTGCGAGATCGAGTGACTGGT
GCTTTTGATATCTGGGGCCAAGGGACAATGGTCACCGTGTCGACAGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTGTACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGTGGAGGGTCCGGCGGTGGTGGATCCCGGTCGCTGGT
GGAGTCTGGGGGAGGCTTGGTCCAGCCTGGGGGGTCCCTGAGACTCTCCTGTACTGCCTCTGGATTCACCATCAGTA
GCTACCACATGCAGTGGGTCCGGCAGGCACCTGGGAAGTGCCTGGAGTACATCGGAACCATTAGTAGTGGTGGTAAT
GTATACTACGCAAGCTCCGCTAGAGGCAGATTCACCATCTCCAGACCCTCGTCCAAGAACACGGTGGATCTTCAAAT
GAACAGCCTGAGAGCCGAGGACACGGCTGTGTATTACTGTGCGAGAGACTCTGGTTATAGTGATCCTATGTGGGGCC
AGGGAACCCTGGTCACCGTCTCTTCAGGCGGTGGCGGTAGTGGGGGAGGCGGTTCTGGCGGCGGAGGGTCCGGCGGT
GGAGGATCAGACGTTGTGATGACCCAGTCTCCATCTTCCGTGTCTGCATCTGTAGGAGACAGAGTCACCATCACCTG
TCAGGCCAGTCAGAACATTAGGACTTACTTATCCTGGTATCAGCAGAAACCAGGGAAAGCCCCTAAGCTCCTGATCT
ATGCTGCAGCCAATCTGGCATCTGGGGTCCCATCAAGGTTCAGCGGCAGTGGATCTGGGACAGATTTCACTCTCACC
ATCAGCGACTTGGAACCTGGCGATGCTGCAACTTACTATTGTCAGTCTACCTATCTTGGTACTGATTATGTTGGCGG
TGCTTTCGGCTGTGGGACCAAGGTGGAGATCAAATGA
>Sequence ID 143: SI-68E1 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGCGTKVTVLGGGGSGGGGSGGGGSGGGGSQVQLQESGGGLVKPGGSLRLSCA
ASGFTFSSYWMSWVRQAPGKCLEWVANINRDGSASYYVDSVKGRFTISRDDAKNSLYLQMNSLRAEDTAVYYCARDR
GVGYFDLWGRGTLVTVSTGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVYTLPPCRD
ELTKNQVSLWCLVKGFYPSDIAVEWESNGQPENNYKTTPPVLDSDGSFFLYSKLTVDKSRWQQGNVFSCSVMHEALH
NHYTQKSLSLSPGK
>Sequence ID 144: SI-68E1 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CTGTGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGTGCCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTGTCGACAGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACGCCAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCATGTCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAATAG
>Sequence ID 145: SI-68E2 Chain A Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGCGTKVEIKGGGGSGGGGSGGGGSGGGGSQVQLQESGPGLVKPSETLSLTCTVSGG
SVSSGDYYWTWIRQSPGKCLEWIGHIYYSGNTNYNPSLKSRLTISIDTSKTQFSLKLSSVTAADTAIYYCVRDRVTG
AFDIWGQGTMVTVSTGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGK
>Sequence ID 146: SI-68E2 Chain A Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAAGGCGGTGGCGGTAGTGGGGGAGGCGGTTCTGGCGGCGGAGGGTCCGGCGGTGGAGGATCACAGG
TGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGTGCCTGGAGTGGATTGGACACAT
CTATTACAGTGGGAACACCAATTATAACCCCTCCCTCAAGAGCCGACTCACCATATCAATTGACACGTCCAAGACTC
AGTTCTCCCTGAAGCTGAGTTCTGTGACCGCTGCGGACACGGCCATTTATTACTGTGTGCGAGATCGAGTGACTGGT
GCTTTTGATATCTGGGGCCAAGGGACAATGGTCACCGTGTCGACAGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTGTACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTAAATAG
>Sequence ID 147: SI-68E2 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGCGTKVTVLGGGGSGGGGSGGGGSGGGGSQVQLQESGGGLVKPGGSLRLSCA
ASGFTFSSYWMSWVRQAPGKCLEWVANINRDGSASYYVDSVKGRFTISRDDAKNSLYLQMNSLRAEDTAVYYCARDR
GVGYFDLWGRGTLVTVSTGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVYTLPPCRD
ELTKNQVSLWCLVKGFYPSDIAVEWESNGQPENNYKTTPPVLDSDGSFFLYSKLTVDKSRWQQGNVFSCSVMHEALH
NHYTQKSLSLSPGGGGGSGGGGSEVQLLESGGGLVQPGGSLRLSCAASGFSFSSGYDMCWVRQAPGKCLEWIACIAA
GSAGITYDANWAKGRFTISRDNSKNTLYLQMNSLRAEDTAVYYCARSAFSFDYAMDLWGQGTLVTVSSGGGGSGGGG
SGGGGSGGGGSDIQMTQSPSTLSASVGDRVTITCQASQSISSHLNWYQQKPGKAPKLLIYKASTLASGVPSRFSGSG
SGTEFTLTISSLQPDDFATYYCQQGYSWGNVDNVFGCGTKVEIK
>Sequence ID 148: SI-68E2 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CTGTGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGTGCCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTGTCGACAGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACGCCAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCATGTCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTGGCGGTGGAGGGTCCGGCGGTGGTGGATCCGAGGTGCA
GCTGTTGGAGTCTGGGGGAGGCTTGGTACAGCCTGGGGGGTCCCTGAGACTCTCCTGTGCAGCCTCTGGATTCTCCT
TCAGTAGCGGGTACGACATGTGCTGGGTCCGCCAGGCTCCAGGGAAGTGCCTGGAGTGGATCGCATGCATTGCTGCT
GGTAGTGCTGGTATCACTTACGACGCGAACTGGGCGAAAGGCCGGTTCACCATCTCCAGAGACAATTCCAAGAACAC
GCTGTATCTGCAAATGAACAGCCTGAGAGCCGAGGACACGGCCGTATATTACTGTGCGAGATCGGCGTTTTCGTTCG
ACTACGCCATGGACCTCTGGGGCCAGGGAACCCTGGTCACCGTCTCGTCTGGTGGAGGCGGTTCAGGCGGAGGTGGA
AGTGGTGGTGGCGGCTCTGGAGGCGGCGGATCTGACATCCAGATGACCCAGTCTCCTTCCACCCTGTCTGCATCTGT
AGGAGACAGAGTCACCATCACTTGCCAGGCCAGTCAGAGCATTAGTTCCCACTTAAACTGGTATCAGCAGAAACCAG
GGAAAGCCCCTAAGCTCCTGATCTATAAGGCATCCACTCTGGCATCTGGGGTCCCATCAAGGTTCAGCGGCAGTGGA
TCTGGGACAGAATTTACTCTCACCATCAGCAGCCTGCAGCCTGATGATTTTGCAACTTATTACTGCCAACAGGGTTA
TAGTTGGGGTAATGTTGATAATGTTTTCGGCTGCGGGACCAAGGTGGAGATCAAATAG
>Sequence ID 149: SI-68P1 Chain A Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGCGTKVEIKGGGGSGGGGSGGGGSGGGGSQVQLQESGPGLVKPSETLSLTCTVSGG
SVSSGDYYWTWIRQSPGKCLEWIGHIYYSGNTNYNPSLKSRLTISIDTSKTQFSLKLSSVTAADTAIYYCVRDRVTG
AFDIWGQGTMVTVSTGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALHN
HYTQKSLSLSPGGGGGSGGGGSRSLVESGGGLVQPGGSLRLSCTASGFTISSYHMQWVRQAPGKCLEYIGTISSGGN
VYYASSARGRFTISRPSSKNTVDLQMNSLRAEDTAVYYCARDSGYSDPMWGQGTLVTVSSGGGGSGGGGSGGGGSGG
GGSDVVMTQSPSSVSASVGDRVTITCQASQNIRTYLSWYQQKPGKAPKLLIYAAANLASGVPSRFSGSGSGTDFTLT
ISDLEPGDAATYYCQSTYLGTDYVGGAFGCGTKVEIK
>Sequence ID 150: SI-68P1 Chain A Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAAGGCGGTGGCGGTAGTGGGGGAGGCGGTTCTGGCGGCGGAGGGTCCGGCGGTGGAGGATCACAGG
TGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGTGCCTGGAGTGGATTGGACACAT
CTATTACAGTGGGAACACCAATTATAACCCCTCCCTCAAGAGCCGACTCACCATATCAATTGACACGTCCAAGACTC
AGTTCTCCCTGAAGCTGAGTTCTGTGACCGCTGCGGACACGGCCATTTATTACTGTGTGCGAGATCGAGTGACTGGT
GCTTTTGATATCTGGGGCCAAGGGACAATGGTCACCGTGTCGACAGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTGTACCCTGCCCCCATCCCGGGATGAG
CTGACCAAGAACCAGGTCAGCCTGTCCTGCGCCGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCGTGAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGTGGAGGGTCCGGCGGTGGTGGATCCCGGTCGCTGGT
GGAGTCTGGGGGAGGCTTGGTCCAGCCTGGGGGGTCCCTGAGACTCTCCTGTACTGCCTCTGGATTCACCATCAGTA
GCTACCACATGCAGTGGGTCCGGCAGGCACCTGGGAAGTGCCTGGAGTACATCGGAACCATTAGTAGTGGTGGTAAT
GTATACTACGCAAGCTCCGCTAGAGGCAGATTCACCATCTCCAGACCCTCGTCCAAGAACACGGTGGATCTTCAAAT
GAACAGCCTGAGAGCCGAGGACACGGCTGTGTATTACTGTGCGAGAGACTCTGGTTATAGTGATCCTATGTGGGGCC
AGGGAACCCTGGTCACCGTCTCTTCAGGCGGTGGCGGTAGTGGGGGAGGCGGTTCTGGCGGCGGAGGGTCCGGCGGT
GGAGGATCAGACGTTGTGATGACCCAGTCTCCATCTTCCGTGTCTGCATCTGTAGGAGACAGAGTCACCATCACCTG
TCAGGCCAGTCAGAACATTAGGACTTACTTATCCTGGTATCAGCAGAAACCAGGGAAAGCCCCTAAGCTCCTGATCT
ATGCTGCAGCCAATCTGGCATCTGGGGTCCCATCAAGGTTCAGCGGCAGTGGATCTGGGACAGATTTCACTCTCACC
ATCAGCGACTTGGAACCTGGCGATGCTGCAACTTACTATTGTCAGTCTACCTATCTTGGTACTGATTATGTTGGCGG
TGCTTTCGGCTGTGGGACCAAGGTGGAGATCAAATGA
>Sequence ID 151: SI-68P1 Chain B Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGCGTKVTVLGGGGSGGGGSGGGGSGGGGSQVQLQESGGGLVKPGGSLRLSCA
ASGFTFSSYWMSWVRQAPGKCLEWVANINRDGSASYYVDSVKGRFTISRDDAKNSLYLQMNSLRAEDTAVYYCARDR
GVGYFDLWGRGTLVTVSTGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGDKTHTCPPCPAPEAAGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVYTLPPCRD
ELTKNQVSLWCLVKGFYPSDIAVEWESNGQPENNYKTTPPVLDSDGSFFLYSKLTVDKSRWQQGNVFSCSVMHEALH
NHYTQKSLSLSPGGGGGSGGGGSEVQLLESGGGLVQPGGSLRLSCAASGFSFSSGYDMCWVRQAPGKCLEWIACIAA
GSAGITYDANWAKGRFTISRDNSKNTLYLQMNSLRAEDTAVYYCARSAFSFDYAMDLWGQGTLVTVSSGGGGSGGGG
SGGGGSGGGGSDIQMTQSPSTLSASVGDRVTITCQASQSISSHLNWYQQKPGKAPKLLIYKASTLASGVPSRFSGSG
SGTEFTLTISSLQPDDFATYYCQQGYSWGNVDNVFGCGTKVEIK
>Sequence ID 152: SI-68P1 Chain B Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CTGTGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGTGCCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTGTCGACAGGCGGTGGAGGGTCCGGCGGTGG
TGGATCAGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTCACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGAGGCGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACGCCAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCATGTCGGGAT
GAGCTGACCAAGAACCAGGTCAGCCTGTGGTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTGGCGGTGGAGGGTCCGGCGGTGGTGGATCCGAGGTGCA
GCTGTTGGAGTCTGGGGGAGGCTTGGTACAGCCTGGGGGGTCCCTGAGACTCTCCTGTGCAGCCTCTGGATTCTCCT
TCAGTAGCGGGTACGACATGTGCTGGGTCCGCCAGGCTCCAGGGAAGTGCCTGGAGTGGATCGCATGCATTGCTGCT
GGTAGTGCTGGTATCACTTACGACGCGAACTGGGCGAAAGGCCGGTTCACCATCTCCAGAGACAATTCCAAGAACAC
GCTGTATCTGCAAATGAACAGCCTGAGAGCCGAGGACACGGCCGTATATTACTGTGCGAGATCGGCGTTTTCGTTCG
ACTACGCCATGGACCTCTGGGGCCAGGGAACCCTGGTCACCGTCTCGTCTGGTGGAGGCGGTTCAGGCGGAGGTGGA
AGTGGTGGTGGCGGCTCTGGAGGCGGCGGATCTGACATCCAGATGACCCAGTCTCCTTCCACCCTGTCTGCATCTGT
AGGAGACAGAGTCACCATCACTTGCCAGGCCAGTCAGAGCATTAGTTCCCACTTAAACTGGTATCAGCAGAAACCAG
GGAAAGCCCCTAAGCTCCTGATCTATAAGGCATCCACTCTGGCATCTGGGGTCCCATCAAGGTTCAGCGGCAGTGGA
TCTGGGACAGAATTTACTCTCACCATCAGCAGCCTGCAGCCTGATGATTTTGCAACTTATTACTGCCAACAGGGTTA
TAGTTGGGGTAATGTTGATAATGTTTTCGGCTGCGGGACCAAGGTGGAGATCAAATAG
>Sequence ID 153: SI-49R26 Chain A Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASTKGPSVFPLAPSS
KSTSGGTAALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGGPSVFLFPPKPKDTLMISRTPEVTCVVVDVSHEDPEVKFNWYVDGVEVHN
AKTKPREEQYASTYRVVSVLTVLHQDWLNGKEYKCAVSNKALPAPIEKTISKAKGQPREPQVCTLPPSRDELTKNQV
SLSCAVKGFYPSDIAVEWESNGQPENNYKTTPPVLDSDGSFFLVSKLTVDKSRWQQGNVFSCSVMHEALHNHYTQKS
LSLSPG
>Sequence ID 154: SI-49R26 Chain A Nucleotide Sequence
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTGAAGCCGC
GGGGGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACGCCAGCACGTACCGTGTGGTCAGCGTCCTCACCGTCCTGCACCAGGA
CTGGCTGAATGGCAAGGAGTACAAGTGCGCGGTCTCCAACAAAGCCCTCCCAGCCCCCATCGAGAAAACCATCTCCA
AAGCCAAAGGGCAGCCCCGAGAACCACAGGTGTGTACCCTGCCCCCATCCCGGGATGAGCTGACCAAGAACCAGGTC
AGCCTGTCCTGCGCCGTCAAAGGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCGTGAGCAAGCTCACCGTGGACAAGA
GCAGGTGGCAGCAGGGGAACGTCTTCTCATGCTCCGTGATGCATGAGGCTCTGCACAACCACTACACGCAGAAGAGC
CTCTCCCTGTCTCCGGGTTAA
>Sequence ID 155: SI-49R26 Chain B Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGECGGDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFSCSVMHEALHN
HYTQKSLSLSPG
>Sequence ID 156: SI-49R26 Chain B Nucleotide Sequence
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTGGAGGCGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTACACCCTGCCCCCATGTCGGGATGAG
CTGACCAAGAACCAGGTCAGCCTGTGGTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTTAA
>Sequence ID 157: SI-49R27 Chain A Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVCTLPPSRDELTKNQVSLSCAVKGFYPSDIAVEWESNGQPENNYKTTPPVLDSDGSFFLVSKLT
VDKSRWQQGNVFSCSVMHEALHNHYTQKSLSLSPG
>Sequence ID 158: SI-49R27 Chain A Nucleotide Sequence
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGCGGTGGAGGGTCCGGCGG
TGGTGGATCATTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTGTACCCTGCCCCCATCCCGGGATGAGCTGACCAA
GAACCAGGTCAGCCTGTCCTGCGCCGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCGTGAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTTAA
>Sequence ID 159: SI-49R27 Chain B Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGECGGDKTHTCPPCPAPEAAGGPSVFLFPPKPKDTLMISRTPEVTCVVVDVSHEDPEVKFNWYVD
GVEVHNAKTKPREEQYASTYRVVSVLTVLHQDWLNGKEYKCAVSNKALPAPIEKTISKAKGQPREPQVYTLPPCRDE
LTKNQVSLWCLVKGEYPSDIAVEWESNGQPENNYKTTPPVLDSDGSFFLYSKLTVDKSRWQQGNVFSCSVMHEALHN
HYTQKSLSLSPG
>Sequence ID 160: SI-49R27 Chain B Nucleotide Sequences
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTGGAGGCGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACGCCAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTACACCCTGCCCCCATGTCGGGATGAG
CTGACCAAGAACCAGGTCAGCCTGTGGTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTTAA
>Sequence ID 161: SI-49R25 Chain A Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVCTLPPSRDELTKNQVSLSCAVKGFYPSDIAVEWESNGQPENNYKTTPPVLDSDGSFFLVSKLT
VDKSRWQQGNVFSCSVMHEALHNHYTQKSLSLSPG
>Sequence ID 162: SI-49R25 Chain A Nucleotide Sequence
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGCGGTGGAGGGTCCGGCGG
TGGTGGATCATTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTGTACCCTGCCCCCATCCCGGGATGAGCTGACCAA
GAACCAGGTCAGCCTGTCCTGCGCCGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCGTGAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTTAA
>Sequence ID 163: SI-49R25 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSTGGGGSGGGGSFLNSLFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTIIEMQKGDCALYASSFKGYIENCSTPNT
YICMQRTVRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECGGDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPCRDELTKNQVSLWCLVKGFYPSDIAVEWESNGQPENNYKTTPPVLDSDGSFFLYSKLT
VDKSRWQQGNVFSCSVMHEALHNHYTQKSLSLSPG
>Sequence ID 164: SI-49R25 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCGACTGGCGGTGGAGGGTCCGGCGGTGGTGGATCATTCCTAAA
CTCATTATTCAACCAAGAAGTTCAAATTCCCTTGACCGAAAGTTACTGTGGCCCATGTCCTAAAAACTGGATATGTT
ACAAAAATAACTGCTACCAATTTTTTGATGAGAGTAAAAACTGGTATGAGAGCCAGGCTTCTTGTATGTCTCAAAAT
GCCAGCCTTCTGAAAGTATACAGCAAAGAGGACCAGGATTTACTTAAACTGGTGAAGTCATATCATTGGATGGGACT
AGTACACATTCCAACAAATGGATCTTGGCAGTGGGAAGATGGCTCCATTCTCTCACCCAACCTACTAACAATAATTG
AAATGCAGAAGGGAGACTGTGCACTCTATGCCTCGAGCTTTAAAGGCTATATAGAAAACTGTTCAACTCCAAATACG
TACATCTGCATGCAAAGGACTGTGCGTACGGTGGCTGCACCATCTGTCTTCATCTTCCCGCCATCTGATGAGCAGTT
GAAATCTGGAACTGCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTGGAGGCGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATGTCGGGATGAGCTGACCAA
GAACCAGGTCAGCCTGTGGTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTTAA
>Sequence ID 165: SI-49P_x Chain A Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVCTLPPSRDELTKNQVSLSCAVKGFYPSDIAVEWESNGQPENNYKTTPPVLDSDGSFFLVSKLT
VDKSRWQQGNVFSCSVMHEALHNHYTQKSLSLSPG
>Sequence ID 166: SI-49P_x Chain A Nucleotide Sequence
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGCGGTGGAGGGTCCGGCGG
TGGTGGATCATTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTGTACCCTGCCCCCATCCCGGGATGAGCTGACCAA
GAACCAGGTCAGCCTGTCCTGCGCCGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCGTGAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTTAA
>Sequence ID 167: SI-49P_x Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSTGGGGSGGGGSFLNSLFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTIIEMQKGDCALYASSFKGYIENCSTPNT
YICMQRTVRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECGGDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPCRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIK
>Sequence ID 168: SI-49P_x Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCGACTGGCGGTGGAGGGTCCGGCGGTGGTGGATCATTCCTAAA
CTCATTATTCAACCAAGAAGTTCAAATTCCCTTGACCGAAAGTTACTGTGGCCCATGTCCTAAAAACTGGATATGTT
ACAAAAATAACTGCTACCAATTTTTTGATGAGAGTAAAAACTGGTATGAGAGCCAGGCTTCTTGTATGTCTCAAAAT
GCCAGCCTTCTGAAAGTATACAGCAAAGAGGACCAGGATTTACTTAAACTGGTGAAGTCATATCATTGGATGGGACT
AGTACACATTCCAACAAATGGATCTTGGCAGTGGGAAGATGGCTCCATTCTCTCACCCAACCTACTAACAATAATTG
AAATGCAGAAGGGAGACTGTGCACTCTATGCCTCGAGCTTTAAAGGCTATATAGAAAACTGTTCAACTCCAAATACG
TACATCTGCATGCAAAGGACTGTGCGTACGGTGGCTGCACCATCTGTCTTCATCTTCCCGCCATCTGATGAGCAGTT
GAAATCTGGAACTGCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTGGAGGCGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATGTCGGGATGAGCTGACCAA
GAACCAGGTCAGCCTGTGGTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGTCTGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAATAG
>Sequence ID 169: SI-49P8 Chain A Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVCTLPPSRDELTKNQVSLSCAVKGFYPSDIAVEWESNGQPENNYKTTPPVLDSDGSFFLVSKLT
VDKSRWQQGNVFSCSVMHEALHNHYTQKSLSLSPGGGGGSGGGGSRSLVESGGGLVQPGGSLRLSCTASGFTISSYH
MQWVRQAPGKCLEYIGTISSGGNVYYASSARGRFTISRPSSKNTVDLQMNSLRAEDTAVYYCARDSGYSDPMWGQGT
LVTVSSGGGGSGGGGSGGGGSGGGGSDVVMTQSPSSVSASVGDRVTITCQASQNIRTYLSWYQQKPGKAPKLLIYAA
ANLASGVPSRFSGSGSGTDFTLTISDLEPGDAATYYCQSTYLGTDYVGGAFGCGTKVEIK
>Sequence ID 170: SI-49P8 Chain A Nucleotide Sequence
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGCGGTGGAGGGTCCGGCGG
TGGTGGATCATTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTGTACCCTGCCCCCATCCCGGGATGAGCTGACCAA
GAACCAGGTCAGCCTGTCCTGCGCCGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCGTGAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCCGGTCGCTGGTGGAGTCTG
GGGGAGGCTTGGTCCAGCCTGGGGGGTCCCTGAGACTCTCCTGTACTGCCTCTGGATTCACCATCAGTAGCTACCAC
ATGCAGTGGGTCCGGCAGGCACCTGGGAAGTGCCTGGAGTACATCGGAACCATTAGTAGTGGTGGTAATGTATACTA
CGCAAGCTCCGCTAGAGGCAGATTCACCATCTCCAGACCCTCGTCCAAGAACACGGTGGATCTTCAAATGAACAGCC
TGAGAGCCGAGGACACGGCTGTGTATTACTGTGCGAGAGACTCTGGTTATAGTGATCCTATGTGGGGCCAGGGAACC
CTGGTCACCGTCTCTTCAGGCGGTGGCGGTAGTGGGGGAGGCGGTTCTGGCGGCGGAGGGTCCGGCGGTGGAGGATC
AGACGTTGTGATGACCCAGTCTCCATCTTCCGTGTCTGCATCTGTAGGAGACAGAGTCACCATCACCTGTCAGGCCA
GTCAGAACATTAGGACTTACTTATCCTGGTATCAGCAGAAACCAGGGAAAGCCCCTAAGCTCCTGATCTATGCTGCA
GCCAATCTGGCATCTGGGGTCCCATCAAGGTTCAGCGGCAGTGGATCTGGGACAGATTTCACTCTCACCATCAGCGA
CTTGGAACCTGGCGATGCTGCAACTTACTATTGTCAGTCTACCTATCTTGGTACTGATTATGTTGGCGGTGCTTTCG
GCTGTGGGACCAAGGTGGAGATCAAATGA
>Sequence ID 171: SI-49P8 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSTGGGGSGGGGSFLNSLFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTIIEMQKGDCALYASSFKGYIENCSTPNT
YICMQRTVRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECGGDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPCRDELTKNQVSLWCLVKGEYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIK
>Sequence ID 172: SI-49P8 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCGACTGGCGGTGGAGGGTCCGGCGGTGGTGGATCATTCCTAAA
CTCATTATTCAACCAAGAAGTTCAAATTCCCTTGACCGAAAGTTACTGTGGCCCATGTCCTAAAAACTGGATATGTT
ACAAAAATAACTGCTACCAATTTTTTGATGAGAGTAAAAACTGGTATGAGAGCCAGGCTTCTTGTATGTCTCAAAAT
GCCAGCCTTCTGAAAGTATACAGCAAAGAGGACCAGGATTTACTTAAACTGGTGAAGTCATATCATTGGATGGGACT
AGTACACATTCCAACAAATGGATCTTGGCAGTGGGAAGATGGCTCCATTCTCTCACCCAACCTACTAACAATAATTG
AAATGCAGAAGGGAGACTGTGCACTCTATGCCTCGAGCTTTAAAGGCTATATAGAAAACTGTTCAACTCCAAATACG
TACATCTGCATGCAAAGGACTGTGCGTACGGTGGCTGCACCATCTGTCTTCATCTTCCCGCCATCTGATGAGCAGTT
GAAATCTGGAACTGCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTGGAGGCGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATGTCGGGATGAGCTGACCAA
GAACCAGGTCAGCCTGTGGTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGTCTGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAATAG
>Sequence ID 173: SI-49PM1 Chain A Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTVVDGGGGSGGG
GSEVQLVESGGGLVQPGGSLRLSCAASGFTISTNAMSWVRQAPGKGLEWIGVITGRDITYYASWAKGRFTISRDNSK
NTLYLQMNSLRAEDTAVYYCARDGGSSAITSNNIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKENWYVDGVEVHNAKTKPREEQYASTYRVV
SVLTVLHQDWLNGKEYKCAVSNKALPAPIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHEALHNHYTQKSLSLSPGGGGGSGGGGSR
SLVESGGGLVQPGGSLRLSCTASGFTISSYHMQWVRQAPGKCLEYIGTISSGGNVYYASSARGRFTISRPSSKNTVD
LQMNSLRAEDTAVYYCARDSGYSDPMWGQGTLVTVSSGGGGSGGGGSGGGGSGGGGSDVVMTQSPSSVSASVGDRVT
ITCQASQNIRTYLSWYQQKPGKAPKLLIYAAANLASGVPSRFSGSGSGTDFTLTISDLEPGDAATYYCQSTYLGTDY
VGGAFGCGTKVEIK
>Sequence ID 174: SI-49PM1 Chain A Nucleotide Sequence
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTGGTCGACGGCGGTGGAGGGTCCGGCGGTGGT
GGATCAGAGGTGCAGCTGGTGGAGTCTGGGGGAGGCTTGGTCCAGCCTGGGGGGTCCCTGAGACTCTCCTGTGCAGC
CTCTGGATTCACCATCAGTACCAATGCAATGAGCTGGGTCCGCCAGGCTCCAGGGAAGGGGCTGGAGTGGATCGGAG
TCATTACTGGTCGTGATATCACATACTACGCGAGCTGGGCGAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGACGGTGGTTC
TTCTGCTATTACTAGTAACAACATTTGGGGCCAGGGAACCCTGGTCACCGTGTCCTCAGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACGCCAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTGTACCCTGCCCCCATCCC
GGGATGAGCTGACCAAGAACCAGGTCAGCCTGTCCTGCGCCGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CGTGAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTGGCGGTGGAGGGTCCGGCGGTGGTGGATCCCGG
TCGCTGGTGGAGTCTGGGGGAGGCTTGGTCCAGCCTGGGGGGTCCCTGAGACTCTCCTGTACTGCCTCTGGATTCAC
CATCAGTAGCTACCACATGCAGTGGGTCCGGCAGGCACCTGGGAAGTGCCTGGAGTACATCGGAACCATTAGTAGTG
GTGGTAATGTATACTACGCAAGCTCCGCTAGAGGCAGATTCACCATCTCCAGACCCTCGTCCAAGAACACGGTGGAT
CTTCAAATGAACAGCCTGAGAGCCGAGGACACGGCTGTGTATTACTGTGCGAGAGACTCTGGTTATAGTGATCCTAT
GTGGGGCCAGGGAACCCTGGTCACCGTCTCTTCAGGCGGTGGCGGTAGTGGGGGAGGCGGTTCTGGCGGCGGAGGGT
CCGGCGGTGGAGGATCAGACGTTGTGATGACCCAGTCTCCATCTTCCGTGTCTGCATCTGTAGGAGACAGAGTCACC
ATCACCTGTCAGGCCAGTCAGAACATTAGGACTTACTTATCCTGGTATCAGCAGAAACCAGGGAAAGCCCCTAAGCT
CCTGATCTATGCTGCAGCCAATCTGGCATCTGGGGTCCCATCAAGGTTCAGCGGCAGTGGATCTGGGACAGATTTCA
CTCTCACCATCAGCGACTTGGAACCTGGCGATGCTGCAACTTACTATTGTCAGTCTACCTATCTTGGTACTGATTAT
GTTGGCGGTGCTTTCGGCTGTGGGACCAAGGTGGAGATCAAATGA
>Sequence ID 175: SI-49PM1 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFSCSVMHEALHNRF
TQKSLSLSPGGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVTVLGGGGSGGGGSGGGGSGGGGSEVQL
LESGGGLVQPGGSLSLSCAASGFSFSSGYDMCWVRQAPGKGLEWIACIAAGSAGITYDANWAKGRFTISRDNSKNTL
YLQMNSLRAEDTAVYYCARSAFSFDYAMDLWGQGTLVTVSS
>Sequence ID 176: SI-49PM1 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGCGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCAAGCCGGGATGAGCTGACC
AAGAACCAGGTCAGCCTGTGGTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCTCTGCACAACAGATTC
ACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCAC
ACCTCAACTGGTATCAACAGAAACCCGGTAAAGCACCGAAGCTGCTCATCTACAAGGCATCAACTCTTGCCTCAGGT
GTGCCTAGTCGCTTCTCCGGGAGCGGCTCCGGTACTGAGTTTACTTTGACGATATCTTCCCTCCAACCCGACGACTT
CGCCACATACTATTGTCAGCAGGGTTATAGTTGGGGAAATGTTGACAATGTGTTTGGAGGGGGAACGAAAGTGACTG
TGTTGGGAGGGGGAGGAAGTGGCGGCGGCGGAAGCGGAGGTGGTGGCTCCGGCGGGGGTGGAAGCGAAGTGCAATTG
CTCGAAAGTGGTGGCGGACTTGTGCAACCTGGTGGAAGCCTCTCACTCTCTTGCGCGGCGAGTGGTTTTAGCTTCAG
TAGCGGCTACGATATGTGCTGGGTAAGACAGGCTCCTGGTAAAGGCCTTGAGTGGATTGCGTGTATCGCCGCCGGGT
CTGCTGGTATAACATACGATGCAAACTGGGCGAAAGGGCGCTTCACAATCTCCAGGGACAACTCAAAAAATACATTG
TACCTCCAGATGAATAGCCTGAGAGCCGAAGACACCGCAGTCTACTATTGCGCAAGGTCAGCATTTAGCTTCGATTA
TGCAATGGATCTTTGGGGCCAGGGGACGCTCGTCACCGTCTCATCTTGA
>Sequence ID 177: SI-49P9 Chain A Amino Acid
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVCTLPPSRDELTKNQVSLSCAVKGFYPSDIAVEWESNGQPENNYKTTPPVLDSDGSFFLVSKLT
VDKSRWQQGNVFSCSVMHEALHNHYTQKSLSLSPGGGGGSGGGGSREGPELSPDDPAGLLDLRQGMFAQLVAQNVLL
IDGPLSWYSDPGLAGVSLTGGLSYKEDTKELVVAKAGVYYVFFQLELRRVVAGEGSGSVSLALHLQPLRSAAGAAAL
ALTVDLPPASSEARNSAFGFQGRLLHLSAGQRLGVHLHTEARARHAWQLTQGATVLGLFRVTPEIPAGLGSTGSGSK
PGSGEGSTKGREGPELSPDDPAGLLDLRQGMFAQLVAQNVLLIDGPLSWYSDPGLAGVSLTGGLSYKEDTKELVVAK
AGVYYVFFQLELRRVVAGEGSGSVSLALHLQPLRSAAGAAALALTVDLPPASSEARNSAFGFQGRLLHLSAGQRLGV
HLHTEARARHAWQLTQGATVLGLFRVTPEIPAGLGGGGSGGGGSREGPELSPDDPAGLLDLRQGMFAQLVAQNVLLI
DGPLSWYSDPGLAGVSLTGGLSYKEDTKELVVAKAGVYYVFFQLELRRVVAGEGSGSVSLALHLQPLRSAAGAAALA
LTVDLPPASSEARNSAFGFQGRLLHLSAGQRLGVHLHTEARARHAWQLTQGATVLGLFRVTPEIPAGL
>Sequence ID 178: SI-49P9 Chain A Nucleotide Sequence
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGCGGTGGAGGGTCCGGCGG
TGGTGGATCATTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTGTACCCTGCCCCCATCCCGGGATGAGCTGACCAA
GAACCAGGTCAGCCTGTCCTGCGCCGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCGTGAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCCGAGAGGGCCCCGAGCTGT
CTCCTGATGACCCAGCAGGCCTCTTGGACTTGCGGCAGGGTATGTTCGCTCAACTTGTGGCTCAGAATGTTCTGCTC
ATTGATGGACCACTCTCTTGGTATAGTGACCCCGGTCTGGCCGGGGTGAGTCTGACCGGCGGGCTCTCTTATAAAGA
GGATACTAAGGAACTGGTCGTAGCAAAAGCGGGCGTTTATTACGTTTTTTTTCAGCTGGAGCTCAGGCGCGTGGTGG
CCGGCGAGGGCAGTGGCTCTGTGTCCCTGGCCCTGCACTTACAGCCCTTGAGAAGCGCTGCAGGTGCTGCCGCACTG
GCTTTAACTGTTGACCTCCCTCCGGCCTCTTCTGAAGCTAGAAACAGCGCTTTCGGCTTCCAAGGGCGCCTGCTGCA
CCTGAGCGCAGGCCAGCGCTTAGGTGTGCACCTTCATACAGAGGCCAGGGCCCGACACGCTTGGCAGCTCACACAGG
GTGCCACGGTTCTCGGACTTTTCCGCGTTACTCCCGAGATCCCCGCTGGCCTCGGAAGTACTGGTTCTGGGTCTAAA
CCCGGTTCCGGCGAAGGTAGTACTAAAGGACGAGAAGGGCCAGAGTTAAGTCCAGATGACCCTGCTGGGCTTTTGGA
CCTGCGGCAGGGCATGTTCGCTCAACTGGTGGCTCAGAACGTGCTGCTGATCGATGGCCCCCTGAGTTGGTACAGCG
ATCCCGGGCTGGCAGGCGTGTCACTTACAGGGGGCCTCTCTTACAAGGAAGACACCAAGGAGTTAGTGGTCGCTAAG
GCTGGCGTGTATTACGTGTTCTTCCAACTGGAGCTGAGAAGGGTTGTGGCAGGAGAGGGTAGCGGCAGCGTGTCTTT
AGCCCTTCACTTGCAGCCCCTGAGGTCTGCTGCAGGTGCAGCCGCTCTCGCGCTCACCGTGGATCTCCCCCCAGCCT
CATCTGAAGCTAGGAACAGTGCATTTGGCTTTCAGGGACGCTTGCTGCACCTCTCCGCTGGACAGAGGCTGGGCGTG
CACCTTCACACAGAGGCCCGTGCCAGGCATGCATGGCAGCTCACTCAGGGGGCAACAGTGCTGGGTCTCTTCCGCGT
GACTCCTGAAATACCAGCTGGACTTGGCGGTGGAGGCAGCGGCGGAGGAGGATCTCGTGAGGGGCCAGAACTGTCCC
CCGATGACCCAGCCGGACTGCTCGATCTCAGACAGGGCATGTTCGCTCAGCTTGTAGCCCAAAATGTCCTCCTGATT
GACGGCCCTTTGAGCTGGTATAGTGATCCCGGCTTGGCCGGGGTATCTCTGACCGGAGGCCTCTCCTACAAGGAAGA
CACCAAAGAGCTGGTGGTGGCAAAAGCGGGGGTGTATTATGTGTTCTTTCAGCTCGAGCTGCGGAGAGTTGTGGCCG
GGGAAGGGTCTGGGAGCGTATCTCTTGCACTTCACCTGCAGCCCCTGCGCAGCGCCGCTGGAGCCGCCGCCCTTGCT
CTTACTGTGGATCTGCCTCCTGCTTCCTCAGAAGCACGCAACAGCGCCTTCGGCTTTCAAGGACGTCTCCTGCACTT
GTCCGCAGGACAGAGGTTGGGCGTCCATTTACACACTGAGGCACGGGCACGGCACGCTTGGCAGCTTACCCAGGGAG
CCACCGTGCTGGGACTCTTTAGAGTGACACCCGAGATCCCCGCTGGCTTGTAA
>Sequence ID 179: SI-49P9 Chain B Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSTGGGGSGGGGSFLNSLFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTIIEMQKGDCALYASSFKGYIENCSTPNT
YICMQRTVRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGECGGDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPCRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIK
>Sequence ID 180: SI-49P9 Chain B Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCGACTGGCGGTGGAGGGTCCGGCGGTGGTGGATCATTCCTAAA
CTCATTATTCAACCAAGAAGTTCAAATTCCCTTGACCGAAAGTTACTGTGGCCCATGTCCTAAAAACTGGATATGTT
ACAAAAATAACTGCTACCAATTTTTTGATGAGAGTAAAAACTGGTATGAGAGCCAGGCTTCTTGTATGTCTCAAAAT
GCCAGCCTTCTGAAAGTATACAGCAAAGAGGACCAGGATTTACTTAAACTGGTGAAGTCATATCATTGGATGGGACT
AGTACACATTCCAACAAATGGATCTTGGCAGTGGGAAGATGGCTCCATTCTCTCACCCAACCTACTAACAATAATTG
AAATGCAGAAGGGAGACTGTGCACTCTATGCCTCGAGCTTTAAAGGCTATATAGAAAACTGTTCAACTCCAAATACG
TACATCTGCATGCAAAGGACTGTGCGTACGGTGGCTGCACCATCTGTCTTCATCTTCCCGCCATCTGATGAGCAGTT
GAAATCTGGAACTGCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGTGGAGGCGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATGTCGGGATGAGCTGACCAA
GAACCAGGTCAGCCTGTGGTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGTCTGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAATAG
>Sequence ID 181: SI-1C3 Heavy Chain Amino Acid Sequence
QVQLQESGPGLVKPSETLSLTCTVSGGSVSSGDYYWTWIRQSPGKGLEWIGHIYYSGNTNYNPSLKSRLTISIDTSK
TQFSLKLSSVTAADTAIYYCVRDRVTGAFDIWGQGTMVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
>Sequence ID 182: SI-1C3 Heavy Chain Nucleotide Sequence
CAGGTGCAGCTGCAGGAGTCGGGCCCAGGACTGGTGAAGCCTTCGGAGACCCTGTCCCTCACCTGCACTGTCTCTGG
TGGCTCCGTCAGCAGTGGTGATTACTACTGGACCTGGATCCGGCAGTCCCCAGGGAAGGGACTGGAGTGGATTGGAC
ACATCTATTACAGTGGGAACACCAATTATAACCCCTCCCTCAAGAGCCGACTCACCATATCAATTGACACGTCCAAG
ACTCAGTTCTCCCTGAAGCTGAGTTCTGTGACCGCTGCGGACACGGCCATTTATTACTGTGTGCGAGATCGAGTGAC
TGGTGCTTTTGATATCTGGGGCCAAGGGACAATGGTCACCGTCTCGAGCGCTAGCACCAAGGGCCCATCGGTCTTCC
CCCTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGTACACCCTGCCCCCATCCCGGGAGGAGA
TGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTATAGCAA
GCTCACCGTGGACAAGAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAATGA
>Sequence ID 183: SI-1C3 Light Chain Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>Sequence ID 184: SI-1C3 Light Chain Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>Sequence ID 185: SI-1C7 Amino Acid Sequence
EPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLSLSPG
GGGGSGGGGSQVQLQESGGGLVKPGGSLRLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSSGGGGSGGGGSGGGGSQSALTQPASVS
GSPGQSITISCTGTSSDVGGYNFVSWYQQHPGKAPKLMIYDVSDRPSGVSDRFSGSKSGNTASLIISGLQADDEADY
YCSSYGSSSTHVIFGGGTKVTVL
>Sequence ID 186: SI-1C7 Nucleotide Sequence
GAGCCCAAATCTTCCGACAAAACTCACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTCTCCGGGT
GGCGGTGGAGGGTCCGGCGGTGGTGGATCCCAGGTGCAGCTGCAGGAGTCGGGGGGAGGCCTGGTCAAGCCTGGAGG
GTCCCTGAGACTCTCCTGTGCAGCCTCTGGATTCACCTTTAGTAGTTATTGGATGAGCTGGGTCCGCCAGGCTCCAG
GGAAGGGGCTGGAGTGGGTGGCCAACATAAACCGCGATGGAAGTGCGAGTTACTATGTGGACTCTGTGAAGGGCCGA
TTCACCATCTCCAGAGACGACGCCAAGAACTCACTGTATCTGCAAATGAACAGCCTGAGAGCTGAGGACACGGCTGT
GTATTACTGTGCGAGAGATCGTGGGGTGGGCTACTTCGATCTCTGGGGCCGTGGCACCCTGGTCACCGTCTCGAGCG
GTGGAGGCGGTTCAGGCGGAGGTGGTTCCGGCGGTGGCGGCTCCCAGTCTGCCCTGACTCAGCCTGCCTCCGTGTCT
GGGTCTCCTGGACAGTCGATCACCATCTCCTGCACTGGAACCAGCAGTGACGTTGGTGGTTATAACTTTGTCTCCTG
GTACCAACAACACCCAGGCAAAGCCCCCAAACTCATGATCTATGATGTCAGTGATCGGCCCTCAGGGGTGTCTGATC
GCTTCTCCGGCTCCAAGTCTGGCAACACGGCCTCCCTGATCATCTCTGGCCTCCAGGCTGACGACGAGGCTGATTAT
TACTGCAGCTCATATGGGAGCAGCAGCACTCATGTGATTTTCGGCGGAGGGACCAAGGTGACCGTCCTATAA
>Sequence ID 187: SI-9C21 Heavy Chain Amino Acid Sequence
EVQLVESGGGLVQPGGSLRLSCTASGFTISTNAMSWVRQAPGKGLEWVGVITGRDITYYASWAKGRFTISRDTSKNT
VYLQMNSLRAEDTAVYYCARDGGSSAITSNNIWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKRVEPKSCDKTHTCPP
CPAPEAAGAPSVFLFPPKPKDTLMISRTPEVTCVVVDVSHEDPEVKFNWYVDGVEVHNAKTKPREEQYNSTYRVVSV
LTVLHQDWLNGKEYKCAVSNKALPAPIEKTISKAKGQPREPQVYTLPPSRDELTKNQVSLTCLVKGFYPSDIAVEWE
SNGQPENNYKTTPPVLDSDGSFLLYSKLTVDKSRWQQGNVFSCSVMHEALHNHYTQKSLSLSPG
>Sequence ID 188: SI-9C21 Heavy Chain Nucleotide Sequence
GAAGTCCAATTGGTAGAAAGTGGCGGTGGTCTGGTGCAACCTGGTGGATCTCTTCGCCTCTCATGCACGGCTAGTGG
CTTTACTATTTCAACTAATGCGATGAGCTGGGTTCGCCAGGCCCCCGGCAAAGGACTTGAGTGGGTCGGCGTCATCA
CCGGCAGGGACATTACATACTATGCGAGTTGGGCAAAGGGCAGGTTCACGATTAGCCGCGATACTTCAAAGAATACC
GTTTACCTTCAAATGAATAGCTTGCGCGCGGAAGACACAGCTGTGTATTACTGCGCGCGGGATGGAGGTAGTTCCGC
CATAACTTCCAACAACATATGGGGACAAGGCACGCTGGTTACTGTGTCGTCAGCTAGCACCAAGGGCCCATCGGTCT
TCCCCCTGGCACCCTCCTCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCACCG
TGCCCAGCACCTGAAGCCGCGGGGGCACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGATG
AGCTGACCAAGAACCAGGTCAGCCTGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGACGGCTCCTTCCTGCTCTATAG
CAAGCTCACCGTGGACAAGAGCAGGTGGCAGCAGGGGAACGTCTTCTCATGCTCCGTGATGCATGAGGCTCTGCACA
ACCACTACACGCAGAAGAGCCTCTCCCTGTCTCCGGGTTGA
>Sequence ID 189: SI-9C21 Light Chain Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVLRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>Sequence ID 190: SI-9C21 Light Chain Nucleotide Sequence
GAAATCGTTATGACGCAGAGTCCCTCCACGCTCTCCGCTAGTGTCGGGGATCGCGTCATTATCACATGCCAGGCCTC
CGAGTCAATCAGCAGCTGGCTTGCATGGTATCAACAGAAGCCGGGAAAAGCTCCTAAATTGCTGATCTATGAAGCGT
CAAAATTGGCGTCTGGTGTCCCATCTAGGTTCTCCGGCTCTGGGTCTGGTGCGGAATTTACTTTGACAATCTCCAGT
CTTCAACCAGACGATTTCGCTACCTACTACTGCCAAGGGTATTTCTATTTTATAAGCCGGACATATGTAAACTCCTT
CGGCCAAGGAACAAAGTTGACTGTTCTTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAG
>Sequence ID 191: SI-35FS11 Amino Acid Sequence
EPKSSDKTHTCPPCPAPEAAGAPSVFLFPPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLSLSPG
GGGGSGGGGSRSLVESGGGLVQPGGSLRLSCTASGFTISSYHMQWVRQAPGKGLEYIGTISSGGNVYYASSARGRFT
ISRPSSKNTVDLQMNSLRAEDTAVYYCARDSGYSDPMWGQGTLVTVSSGGGGSGGGGSGGGGSGGGGSDVVMTQSPS
SVSASVGDRVTITCQASQNIRTYLSWYQQKPGKAPKLLIYAAANLASGVPSRFSGSGSGTDFTLTISDLEPGDAATY
YCQSTYLGTDYVGGAFGGGTKVEIK
>Sequence ID 192: SI-35FS11 Nucleotide Sequence
GAGCCCAAATCTTCCGACAAAACTCACACATGCCCACCGTGCCCAGCACCTGAAGCCGCGGGGGCACCGTCAGTCTT
CCTCTTCCCCCCAAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGGCTGAATGGCAAGGA
GTACAAGTGCGCGGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTCTCCGGGT
GGCGGTGGAGGGTCCGGCGGTGGTGGATCCCGGTCGCTGGTGGAGTCTGGGGGAGGCTTGGTCCAGCCTGGGGGGTC
CCTGAGACTCTCCTGTACTGCCTCTGGATTCACCATCAGTAGCTACCACATGCAGTGGGTCCGCCAGGCTCCAGGGA
AGGGGCTGGAGTACATCGGAACCATTAGTAGTGGTGGTAATGTATACTACGCGAGCTCCGCGAGAGGCAGATTCACC
ATCTCCAGACCCTCGTCCAAGAACACGGTGGATCTTCAAATGAACAGCCTGAGAGCCGAGGACACGGCTGTGTATTA
CTGTGCGAGAGACTCTGGTTATAGTGATCCTATGTGGGGCCAGGGAACCCTGGTCACCGTCTCTTCAGGCGGTGGCG
GTAGTGGGGGAGGCGGTTCTGGCGGCGGAGGGTCCGGCGGTGGAGGATCAGACGTTGTGATGACCCAGTCTCCATCT
TCCGTGTCTGCATCTGTAGGAGACAGAGTCACCATCACCTGTCAGGCCAGTCAGAACATTAGGACTTACTTATCCTG
GTATCAGCAGAAACCAGGGAAAGCCCCTAAGCTCCTGATCTATGCTGCAGCCAATCTGGCATCTGGGGTCCCATCAA
GGTTCAGCGGCAGTGGATCTGGGACAGATTTCACTCTCACCATCAGCGACCTGGAGCCTGGCGATGCTGCAACTTAC
TATTGTCAGTCTACCTATCTTGGTACTGATTATGTTGGCGGTGCTTTCGGCGGAGGGACCAAGGTGGAGATCAAATG
A
>Sequence ID 193: SI-3FS11 Amino Acid Sequence
EPKSSDKTHTCPPCPAPEAAGAPSVFLFPPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLSLSPG
GGGGSGGGGSEVQLLESGGGLVQPGGSLRLSCAASGFSFSSGYDMCWVRQAPGKGLEWIACIAAGSAGITYDANWAK
GRFTISRDNSKNTLYLQMNSLRAEDTAVYYCARSAFSFDYAMDLWGQGTLVTVSSGGGGSGGGGSGGGGSGGGGSDI
QMTQSPSTLSASVGDRVTITCQASQSISSHLNWYQQKPGKAPKLLIYKASTLASGVPSRFSGSGSGTEFTLTISSLQ
PDDFATYYCQQGYSWGNVDNVFGGGTKVEIK
>Sequence ID 194: SI-3FS11 Nucleotide Sequence
GAGCCCAAATCTTCCGACAAAACTCACACATGCCCACCGTGCCCAGCACCTGAAGCCGCGGGGGCACCGTCAGTCTT
CCTCTTCCCCCCAAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGGCTGAATGGCAAGGA
GTACAAGTGCGCGGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTCTCCGGGT
GGCGGTGGAGGGTCCGGCGGTGGTGGATCCGAGGTGCAGCTGTTGGAGTCTGGGGGAGGCTTGGTACAGCCTGGGGG
GTCCCTGAGACTCTCCTGTGCAGCCTCTGGATTCTCCTTCAGTAGCGGGTACGACATGTGCTGGGTCCGCCAGGCTC
CAGGGAAGGGGCTGGAGTGGATCGCATGCATTGCTGCTGGTAGTGCTGGTATCACTTACGACGCGAACTGGGCGAAA
GGCCGGTTCACCATCTCCAGAGACAATTCCAAGAACACGCTGTATCTGCAAATGAACAGCCTGAGAGCCGAGGACAC
GGCCGTATATTACTGTGCGAGATCGGCGTTTTCGTTCGACTACGCCATGGACCTCTGGGGCCAGGGAACCCTGGTCA
CCGTCTCGAGCGGTGGAGGCGGATCTGGCGGAGGTGGTTCCGGCGGTGGCGGCTCCGGTGGAGGCGGCTCTGACATC
CAGATGACCCAGTCTCCTTCCACCCTGTCTGCATCTGTAGGAGACAGAGTCACCATCACTTGCCAGGCCAGTCAGAG
CATTAGTTCCCACTTAAACTGGTATCAGCAGAAACCAGGGAAAGCCCCTAAGCTCCTGATCTATAAGGCATCCACTC
TGGCATCTGGGGTCCCATCAAGGTTCAGCGGCAGTGGATCTGGGACAGAATTTACTCTCACCATCAGCAGCCTGCAG
CCTGATGATTTTGCAACTTATTACTGCCAACAGGGTTATAGTTGGGGTAATGTTGATAATGTTTTCGGCGGAGGGAC
CAAGGTGGAGATCAAATAA
>Sequence ID 195: SI-68X1 Chain A Amino Acid Sequence
DIQMTQSPSTLSASVGDRVTITCQASENIYSNLAWYQQKPGKAPKLLIYDASTLASGVPSRFSGSGSGTEFTLTISS
LQPDDFATYYCQSTYYNNIIYGAAFGCGTKVEIKGGGGSGGGGSGGGGSGGGGSEVQLVESGGGLVQPGGSLRLSCA
ASGFSFSGTQYVCWVRQAPGKCLEWIGCIYVDVANTYYATSVKGRFTISRDNSKNTLYLQMNSLRAEDTAVYYCASH
DTVCAGCGLDLWGQGTLVTVSTGGGGSGGGGSEVQLVESGGGLVQPGGSLRLSCAASGFTISTNAMSWVRQAPGKGL
EWIGVITGRDITYYASWAKGRFTISRDNSKNTLYLQMNSLRAEDTAVYYCARDGGSSAITSNNIWGQGTLVTVSSAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGGPSVFLFPPKPKDTLMISRTPEVTCVVVDVSHEDPEV
KFNWYVDGVEVHNAKTKPREEQYASTYRVVSVLTVLHQDWLNGKEYKCAVSNKALPAPIEKTISKAKGQPREPQVCT
LPPSRDELTKNQVSLSCAVKGFYPSDIAVEWESNGQPENNYKTTPPVLDSDGSFFLVSKLTVDKSRWQQGNVFSCSV
MHEALHNHYTQKSLSLSPGK
>Sequence ID 196: SI-68X1 Chain A Nucleotide Sequence
GACATCCAGATGACCCAGTCTCCTTCCACCCTGTCTGCATCTGTAGGAGACAGAGTCACCATCACTTGCCAGGCCAG
TGAGAACATTTACAGCAATTTAGCCTGGTATCAGCAGAAACCAGGGAAAGCCCCTAAGCTCCTGATCTATGATGCTT
CCACTCTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAATCTACTTATTATAATAATATCATTTATGGGGCTGCTTTCGG
CTGTGGGACCAAGGTGGAGATCAAAGGCGGTGGCGGTAGTGGGGGAGGCGGTTCTGGCGGCGGAGGGTCCGGCGGTG
GAGGATCAGAGGTGCAGCTGGTGGAGTCTGGGGGAGGCTTGGTCCAGCCTGGGGGGTCCCTGAGACTCTCCTGTGCA
GCCTCTGGATTCTCCTTCAGTGGCACCCAGTACGTGTGCTGGGTCCGCCAGGCTCCAGGGAAGTGCCTGGAGTGGAT
CGGATGCATTTATGTGGATGTGGCTAACACTTACTACGCGACCTCCGTGAAGGGCAGATTCACCATCTCCAGAGACA
ATTCCAAGAACACGCTGTATCTTCAAATGAACAGCCTGAGAGCCGAGGACACGGCTGTGTATTACTGTGCGAGTCAT
GATACTGTTTGTGCTGGTTGTGGTTTGGACTTGTGGGGCCAGGGAACCCTGGTCACCGTGTCGACAGGCGGTGGAGG
GTCCGGCGGTGGTGGATCAGAGGTGCAGCTGGTGGAGTCTGGGGGAGGCTTGGTCCAGCCTGGGGGGTCCCTGAGAC
TCTCCTGTGCAGCCTCTGGATTCACCATCAGTACCAATGCAATGAGCTGGGTCCGCCAGGCTCCAGGGAAGGGGCTG
GAGTGGATCGGAGTCATTACTGGTCGTGATATCACATACTACGCGAGCTGGGCGAAAGGCAGATTCACCATCTCCAG
AGACAATTCCAAGAACACGCTGTATCTTCAAATGAACAGCCTGAGAGCCGAGGACACGGCTGTGTATTACTGTGCGA
GAGACGGTGGTTCTTCTGCTATTACTAGTAACAACATTTGGGGCCAGGGAACCCTGGTCACCGTGTCCTCAGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACGCCAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTGTACC
CTGCCCCCATCCCGGGATGAGCTGACCAAGAACCAGGTCAGCCTGTCCTGCGCCGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCGTGAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTAAA
>Sequence ID 197: SI-68X1 Chain B Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGCGTKVEIKGGGGSGGGGSGGGGSGGGGSQVQLQESGPGLVKPSETLSLTCTVSGG
SVSSGDYYWTWIRQSPGKCLEWIGHIYYSGNTNYNPSLKSRLTISIDTSKTQFSLKLSSVTAADTAIYYCVRDRVTG
AFDIWGQGTMVTVSTGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGDKTHTCPPCPAPEAAGGPSVFLFPPKPKDTLMISRTPEVTCVVVDVSHEDPEVKFNWYVDGV
EVHNAKTKPREEQYASTYRVVSVLTVLHQDWLNGKEYKCAVSNKALPAPIEKTISKAKGQPREPQVYTLPPCRDELT
KNQVSLWCLVKGFYPSDIAVEWESNGQPENNYKTTPPVLDSDGSFFLYSKLTVDKSRWQQGNVFSCSVMHEALHNHY
TQKSLSLSPGK
>Sequence ID 198: SI-68X1 Chain B Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAAGGCGGTGGCGGTAGTGGGGGAGGCGGTTCTGGCGGCGGAGGGTCCGGCGGTGGAGGATCACAGG
TGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGTGCCTGGAGTGGATTGGACACAT
CTATTACAGTGGGAACACCAATTATAACCCCTCCCTCAAGAGCCGACTCACCATATCAATTGACACGTCCAAGACTC
AGTTCTCCCTGAAGCTGAGTTCTGTGACCGCTGCGGACACGGCCATTTATTACTGTGTGCGAGATCGAGTGACTGGT
GCTTTTGATATCTGGGGCCAAGGGACAATGGTCACCGTGTCGACAGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGCGACAAAACTCACACATGCCCACCGTGCCCAGC
ACCTGAAGCCGCGGGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACGCCAGCACGTACCGTGTGGTCAGCGTCCTCACCGT
CCTGCACCAGGACTGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATGTCGGGATGAGCTGACC
AAGAACCAGGTCAGCCTGTGGTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTAAA
>Sequence ID 199: SI-68X3 Chain A
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIKGGGGSGGGGSGGGGSGGGGSEVQLVESGGGLVQPGGSLRLSCAASGF
NIKDTYIHWVRQAPGKGLEWVARIYPTNGYTRYADSVKGRFTISADTSKNTAYLQMNSLRAEDTAVYYCSRWGGDGF
YAMDYWGQGTLVTVSTGGGGSGGGGSEVQLVESGGGLVQPGGSLRLSCAASGFTISTNAMSWVRQAPGKGLEWIGVI
TGRDITYYASWAKGRFTISRDNSKNTLYLQMNSLRAEDTAVYYCARDGGSSAITSNNIWGQGTLVTVSSASTKGPSV
FPLAPSSKSTSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPEAAGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVCTLPPSRD
ELTKNQVSLSCAVKGFYPSDIAVEWESNGQPENNYKTTPPVLDSDGSFFLVSKLTVDKSRWQQGNVFSCSVMHEALH
NHYTQKSLSLSPGK
>Sequence ID 200: SI-68X3 Chain A Nucleotide Sequence
GACATCCAAATGACGCAGTCTCCCTCCAGTTTGAGTGCAAGTGTGGGAGACAGAGTTACGATCACATGTAGGGCAAG
TCAGGACGTCAATACAGCCGTCGCATGGTACCAGCAGAAGCCTGGAAAGGCCCCGAAACTTCTGATTTACTCTGCAA
GTTTCCTCTACAGTGGTGTTCCTAGTCGCTTCAGTGGGTCTCGGTCAGGCACCGATTTTACCCTGACAATTAGCAGC
TTGCAACCGGAAGATTTTGCGACCTATTATTGCCAGCAACATTATACCACCCCTCCCACATTTGGACAAGGCACGAA
GGTAGAGATTAAAGGCGGTGGAGGGAGCGGTGGAGGCGGTTCCGGTGGAGGTGGTTCTGGTGGAGGTGGCAGTGAAG
TACAGCTTGTTGAGAGTGGTGGTGGACTTGTCCAACCAGGGGGAAGTCTGCGGCTGTCATGCGCAGCCTCTGGCTTC
AACATTAAGGACACCTACATTCACTGGGTTCGGCAAGCGCCGGGGAAGGGGCTGGAATGGGTCGCCAGGATATACCC
TACCAACGGGTACACCAGGTATGCAGATAGCGTTAAGGGTAGATTCACTATTTCAGCTGACACTAGCAAGAACACCG
CGTATCTCCAGATGAACTCCCTTCGCGCCGAGGATACGGCTGTGTACTACTGCTCTAGGTGGGGAGGAGATGGGTTT
TACGCAATGGATTACTGGGGACAGGGAACACTCGTTACGGTGTCGACAGGCGGTGGAGGGTCCGGCGGTGGTGGATC
AGAGGTGCAGCTGGTGGAGTCTGGGGGAGGCTTGGTCCAGCCTGGGGGGTCCCTGAGACTCTCCTGTGCAGCCTCTG
GATTCACCATCAGTACCAATGCAATGAGCTGGGTCCGCCAGGCTCCAGGGAAGGGGCTGGAGTGGATCGGAGTCATT
ACTGGTCGTGATATCACATACTACGCGAGCTGGGCGAAAGGCAGATTCACCATCTCCAGAGACAATTCCAAGAACAC
GCTGTATCTTCAAATGAACAGCCTGAGAGCCGAGGACACGGCTGTGTATTACTGTGCGAGAGACGGTGGTTCTTCTG
CTATTACTAGTAACAACATTTGGGGCCAGGGAACCCTGGTCACCGTGTCCTCAGCTAGCACCAAGGGCCCATCGGTC
TTCCCCCTGGCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCACC
GTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACGCCAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTGTACCCTGCCCCCATCCCGGGAT
GAGCTGACCAAGAACCAGGTCAGCCTGTCCTGCGCCGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCGTGA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAA
>Sequence ID 201: SI-68X3 Chain B Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGDKTHTCPPCP
APEAAGGPSVFLFPPKPKDTLMISRTPEVTCVVVDVSHEDPEVKFNWYVDGVEVHNAKTKPREEQYASTYRVVSVLT
VLHQDWLNGKEYKCAVSNKALPAPIEKTISKAKGQPREPQVYTLPPCRDELTKNQVSLWCLVKGFYPSDIAVEWESN
GQPENNYKTTPPVLDSDGSFFLYSKLTVDKSRWQQGNVFSCSVMHEALHNHYTQKSLSLSPGK
>Sequence ID 202: SI-68X3 Chain B Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTGGAGGCGACAAAACTCACACATGCCCACCGTGCCCA
GCACCTGAAGCCGCGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACGCCAGCACGTACCGTGTGGTCAGCGTCCTCACC
GTCCTGCACCAGGACTGGCTGAATGGCAAGGAGTACAAGTGCGCGGTCTCCAACAAAGCCCTCCCAGCCCCCATCGA
GAAAACCATCTCCAAAGCCAAAGGGCAGCCCCGAGAACCACAGGTGTACACCCTGCCCCCATGTCGGGATGAGCTGA
CCAAGAACCAGGTCAGCCTGTGGTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCGTGATGCATGAGGCTCTGCACAACCACT
ACACGCAGAAGAGCCTCTCCCTGTCTCCGGGTAAA
>Sequence ID 203: αEGFR VH Amino Acid Sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYYDYEFAYWGQGTLVTVSS
>Sequence ID 204: αEGFR VH Nucleotide Sequence
CAAGTACAGTTGCAGCAATCCGGTCCCGGTCTCGTCAAACCGAGTGAGACGCTTAGTATAACGTGTACTGTTTCAGG
CTTTAGCCTTACGAACTATGGAGTTCACTGGATTCGGCAGGCACCCGGCAAAGGTTTGGAATGGCTGGGTGTTATTT
GGTCAGGTGGAAATACAGACTATAACACCCCCTTTACAAGTCGGTTCACAATTACGAAAGATAATTCCAAAAATCAA
GTTTATTTCAAGTTGAGATCCGTCCGCGCGGACGACACTGCGATCTACTATTGTGCGAGGGCACTGACCTACTACGA
TTACGAATTTGCGTATTGGGGGCAAGGGACTCTTGTAACAGTCTCGAGC
>Sequence ID 205: αEGFR VL Amino Acid Sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVL
>Sequence ID 206: αEGFR VL Nucleotide Sequence
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTA
>Sequence ID 207: αEGFR* VH Amino Acid Sequence
QVQLQESGPGLVKPSETLSLTCTVSGGSVSSGDYYWTWIRQSPGKGLEWIGHIYYSGNTNYNPSLKSRLTISIDTSK
TQFSLKLSSVTAADTAIYYCVRDRVTGAFDIWGQGTMVTVSS
>Sequence ID 208: αEGFR* VH Nucleotide Sequence
CAAGTGCAACTCCAGGAGAGTGGGCCGGGATTGGTTAAACCGAGCGAGACCCTGAGTCTCACGTGTACAGTTTCAGG
CGGGAGCGTGAGCTCAGGCGACTATTACTGGACATGGATAAGACAGAGTCCTGGAAAGGGCCTGGAGTGGATCGGTC
ATATTTATTATTCAGGGAACACTAACTATAATCCATCTTTGAAATCCCGATTGACAATCTCTATCGATACCAGTAAA
ACCCAGTTTAGCCTCAAGCTGTCCAGCGTTACTGCTGCAGACACAGCCATATACTATTGCGTACGCGATCGCGTGAC
TGGAGCCTTCGACATATGGGGGCAGGGAACGATGGTAACAGTCTCGAGC
>Sequence ID 209: αEGFR* VL Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIK
>Sequence ID 210: αEGFR* VL Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAA
>Sequence ID 211: αEGFR* VH Stapled Amino Acid Sequence
QVQLQESGPGLVKPSETLSLTCTVSGGSVSSGDYYWTWIRQSPGKCLEWIGHIYYSGNTNYNPSLKSRLTISIDTSK
TQFSLKLSSVTAADTAIYYCVRDRVTGAFDIWGQGTMVTVST
>Sequence ID 212: αEGFR* VH Stapled Nucleotide Sequence
CAGGTGCAGCTGCAGGAGTCGGGCCCAGGACTGGTGAAGCCTTCGGAGACCCTGTCCCTCACCTGCACTGTCTCTGG
TGGCTCCGTCAGCAGTGGTGATTACTACTGGACCTGGATCCGGCAGTCCCCAGGGAAGTGCCTGGAGTGGATTGGAC
ACATCTATTACAGTGGGAACACCAATTATAACCCCTCCCTCAAGAGCCGACTCACCATATCAATTGACACGTCCAAG
ACTCAGTTCTCCCTGAAGCTGAGTTCTGTGACCGCTGCGGACACGGCCATTTATTACTGTGTGCGAGATCGAGTGAC
TGGTGCTTTTGATATCTGGGGCCAAGGGACAATGGTCACCGTGTCGACA
>Sequence ID 213: αEGFR* VL Stapled Amino Acid Sequence
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIK
>Sequence ID 214: αEGFR* VL Stapled Nucleotide Sequence
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCTGTGGGACCAA
GGTGGAAATTAAA
>Sequence ID 215: αCD19 VH Amino Acid Sequence
QVTLKESGPGLVQPGQTLSLTCAFSGFSLSTSGMGVGWIRQPPGKGLEWLAHIWWDDDKRYNPALKSRLTISKDTSK
NQVYLQMNSLDAEDTAVYYCARMELWSYYFDYWGQGTLVTVSS
>Sequence ID 216: αCD19 VH Nucleotide Sequence
CAGGTCACATTGAAGGAATCTGGCCCCGGCCTTGTTCAGCCAGGACAGACCCTTAGCCTCACCTGTGCCTTCAGTGG
TTTTTCTCTTAGCACTAGCGGTATGGGGGTCGGCTGGATTCGGCAGCCTCCCGGCAAAGGTCTTGAGTGGTTGGCTC
ACATTTGGTGGGACGACGACAAACGGTATAATCCTGCCTTGAAAAGTCGGCTGACCATTAGTAAGGATACCTCAAAA
AATCAAGTGTACTTGCAAATGAATAGCCTTGACGCCGAGGATACGGCTGTATATTATTGCGCGCGGATGGAACTCTG
GTCTTACTACTTTGATTATTGGGGGCAGGGGACTCTCGTCACGGTCTCGAGC
>Sequence ID 217: αCD19 VL Amino Acid Sequence
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVL
>Sequence ID 218: αCD19 VL Nucleotide Sequence
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTA
>Sequence ID 219: αCD3 VH Amino Acid Sequence
EVQLVESGGGLVQPGGSLRLSCAASGFTISTNAMSWVRQAPGKGLEWIGVITGRDITYYASWAKGRFTISRDNSKNT
LYLQMNSLRAEDTAVYYCARDGGSSAITSNNIWGQGTLVTVSS
>Sequence ID 220: αCD3 VH Nucleotide Sequence
GAGGTGCAGCTGGTGGAGTCTGGGGGAGGCTTGGTCCAGCCTGGGGGGTCCCTGAGACTCTCCTGTGCAGCCTCTGG
ATTCACCATCAGTACCAATGCAATGAGCTGGGTCCGCCAGGCTCCAGGGAAGGGGCTGGAGTGGATCGGAGTCATTA
CTGGTCGTGATATCACATACTACGCGAGCTGGGCGAAAGGCAGATTCACCATCTCCAGAGACAATTCCAAGAACACG
CTGTATCTTCAAATGAACAGCCTGAGAGCCGAGGACACGGCTGTGTATTACTGTGCGAGAGACGGTGGTTCTTCTGC
TATTACTAGTAACAACATTTGGGGCCAGGGAACCCTGGTCACCGTGTCCTCA
>Sequence ID 221: αCD3 VL Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIK
>Sequence ID 222: αCD3 VL Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAA
>Sequence ID 223: αCD3 VH stapled Amino Acid Sequence
EVQLVESGGGLVQPGGSLRLSCAASGFTISTNAMSWVRQAPGKCLEWIGVITGRDITYYASWAKGRFTISRDNSKNT
LYLQMNSLRAEDTAVYYCARDGGSSAITSNNIWGQGTLVTVSS
>Sequence ID 224: αCD3 VH stapled Nucleotide Sequence
GAGGTGCAGCTGGTGGAGTCTGGGGGAGGCTTGGTCCAGCCTGGGGGGTCCCTGAGACTCTCCTGTGCAGCCTCTGG
ATTCACCATCAGTACCAATGCAATGAGCTGGGTCCGCCAGGCTCCAGGGAAGTGTCTGGAGTGGATCGGAGTCATTA
CTGGTCGTGATATCACATACTACGCGAGCTGGGCGAAAGGCAGATTCACCATCTCCAGAGACAATTCCAAGAACACG
CTGTATCTTCAAATGAACAGCCTGAGAGCCGAGGACACGGCTGTGTATTACTGTGCGAGAGACGGTGGTTCTTCTGC
TATTACTAGTAACAACATTTGGGGCCAGGGAACCCTGGTCACCGTGTCCTCA
>Sequence ID 225: αCD3 VL stapled Amino Acid Sequence
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIK
>Sequence ID 226: αCD3 VL stapled Nucleotide Sequence
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAA
>Sequence ID 227: αCD3* VH Amino Acid Sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSS
>Sequence ID 228: αCD3* VH Nucleotide Sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCC
>Sequence ID 229: αCD3* VL Amino Acid Sequence
DPQLTQSPSSLSATVGQRVTINCQSSQSVAKNNNLAWFQQKPGKPPKLLIYSASTLAAGVPSRFSGSGSGTQFTLTI
TRVQSEDFATYYCSARDSGNIQSFGGGTRVEIK
>Sequence ID 230: αCD3* VL Nucleotide Sequence
GATCCTCAATTGACCCAGTCCCCTAGTAGCCTTTCTGCAACCGTGGGCCAGAGGGTAACAATCAATTGCCAGTCAAG
CCAGTCCGTAGCGAAGAATAATAATTTGGCATGGTTCCAACAAAAACCGGGTAAACCTCCTAAACTCCTTATCTACT
CAGCTTCAACCCTTGCCGCGGGTGTGCCCAGCCGGTTCTCAGGCTCAGGTTCCGGGACTCAATTTACACTTACCATC
ACTCGCGTCCAAAGTGAGGATTTTGCAACCTACTATTGTTCTGCACGGGATTCCGGGAACATACAGTCCTTTGGGGG
TGGAACTCGGGTGGAGATAAAA
>Sequence ID 231: αCD3** VH Amino Acid Sequence
EVQLVESGGGLVQPGGSLRLSCAASGFTISTNAMSWVRQAPGKGLEWVGVITGRDITYYASWAKGRFTISRDTSKNT
VYLQMNSLRAEDTAVYYCARDGGSSAITSNNIWGQGTLVTVSS
>Sequence ID 232: αCD3** VH Nucleotide Sequence
GAAGTCCAATTGGTAGAAAGTGGCGGTGGTCTGGTGCAACCTGGTGGATCTCTTCGCCTCTCATGCGCCGCTAGTGG
CTTTACTATTTCAACTAATGCGATGAGCTGGGTTCGCCAGGCCCCCGGCAAAGGACTTGAGTGGGTCGGCGTCATCA
CCGGCAGGGACATTACATACTATGCGAGTTGGGCAAAGGGCAGGTTCACGATTAGCCGCGATACTTCAAAGAATACC
GTTTACCTTCAAATGAATAGCTTGAGGGCGGAAGACACAGCTGTGTATTACTGCGCGAGGGATGGAGGTAGTTCCGC
CATAACTTCCAACAACATATGGGGACAAGGCACGCTGGTTACTGTCTCGAGT
>Sequence ID 233: αCD3** VL Amino Acid Sequence
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVL
>Sequence ID 234: αCD3** VL Nucleotide Sequence
GAAATCGTTATGACGCAGAGTCCCTCCACGCTCTCCGCTAGTGTCGGGGATCGCGTCATTATCACATGCCAGGCCTC
CGAGTCAATCAGCAGCTGGCTTGCATGGTATCAACAGAAGCCGGGAAAAGCTCCTAAATTGCTGATCTATGAAGCGT
CAAAATTGGCGTCTGGTGTCCCATCTAGGTTCTCCGGCTCTGGGTCTGGTGCGGAATTTACTTTGACAATCTCCAGT
CTTCAACCAGACGATTTCGCTACCTACTACTGCCAAGGGTATTTCTATTTTATAAGCCGGACATATGTAAACTCCTT
CGGCCAAGGAACAAAGTTGACTGTTCTT
>Sequence ID 235: αCD3*** VH Amino Acid Sequence
QQLLESGGRLIKPGATLRLTCKTSGFTISTNAMSWVRQPPGKGLEWIGVITGRDITYYASWAKGRFTISKTSINTVF
LQMRSLRAEDTAVYYCARDGGSSAITSNNIWGRGTLVTVSS
>Sequence ID 236: αCD3*** VH Nucleotide Sequence
CAGCAGCTGCTGGAAAGCGGCGGCCGCCTGATTAAACCGGGCGCGACCCTGCGCCTGACCTGCAAAACCAGCGGCTT
TACCATTAGCACCAACGCGATGAGCTGGGTGCGCCAGCCGCCGGGCAAAGGCCTGGAATGGATTGGCGTGATTACCG
GCCGCGATATTACCTATTATGCGAGCTGGGCGAAAGGCCGCTTTACCATTAGCAAAACCAGCATTAACACCGTGTTT
CTGCAGATGCGCAGCCTGCGCGCGGAAGATACCGCGGTGTATTATTGCGCGCGCGATGGCGGCAGCAGCGCGATTAC
CAGCAACAACATTTGGGGCCGCGGCACCCTGGTGACCGTGAGCAGC
>Sequence ID 237: αCD3*** VL Amino Acid Sequence
DVQMTQSPSTLSATVGQRVTINCQASESISSWLAWYQQKPGQPPKLLIYEASKLASGVPSRFSGSGSGTEFTLTISS
LQSDDFATYYCQGYFYFISRTYVNSFGGGTRVEIK
>Sequence ID 238: αCD3*** VL Nucleotide Sequence
GATGTGCAGATGACCCAGAGCCCGAGCACCCTGAGCGCGACCGTGGGCCAGCGCGTGACCATTAACTGCCAGGCGAG
CGAAAGCATTAGCAGCTGGCTGGCGTGGTATCAGCAGAAACCGGGCCAGCCGCCGAAACTGCTGATTTATGAAGCGA
GCAAACTGGCGAGCGGCGTGCCGAGCCGCTTTAGCGGCAGCGGCAGCGGCACCGAATTTACCCTGACCATTAGCAGC
CTGCAGAGCGATGATTTTGCGACCTATTATTGCCAGGGCTATTTTTATTTTATTAGCCGCACCTATGTGAACAGCTT
TGGCGGCGGCACCCGCGTGGAAATTAAA
>Sequence ID 239: αCD3**** VH Amino Acid Sequence
QQLVESGGGLVKPGGSLRLSCAASGFTISTNAMSWVRQAPGKGLEWVGVITGRDITYYASWAKGRFTISKTSKNTVY
LQMNSLRAEDTAVYYCARDGGSSAITSNNIWGQGTLVTVSS
>Sequence ID 240: αCD3**** VH Nucleotide Sequence
CAGCAGCTGGTGGAAAGCGGCGGCGGCCTGGTGAAACCGGGCGGCAGCCTGCGCCTGAGCTGCGCGGCGAGCGGCTT
TACCATTAGCACCAACGCGATGAGCTGGGTGCGCCAGGCGCCGGGCAAAGGCCTGGAATGGGTGGGCGTGATTACCG
GCCGCGATATTACCTATTATGCGAGCTGGGCGAAAGGCCGCTTTACCATTAGCAAAACCAGCAAAAACACCGTGTAT
CTGCAGATGAACAGCCTGCGCGCGGAAGATACCGCGGTGTATTATTGCGCGCGCGATGGCGGCAGCAGCGCGATTAC
CAGCAACAACATTTGGGGCCAGGGCACCCTGGTGACCGTGAGCAGC
>Sequence ID 241: αCD3**** VL Amino Acid Sequence
DVVMTQSPSSLSASVGDRVTISCQASESISSWLAWYQQKPGQAPKLLIYEASKLASGVPSRFSGSGSGTEFTLTISS
LEPEDFATYYCQGYFYFISRTYVNSFGGGTKVEIK
>Sequence ID 242: αCD3**** VL Nucleotide Sequence
GATGTGGTGATGACCCAGAGCCCGAGCAGCCTGAGCGCGAGCGTGGGCGATCGCGTGACCATTAGCTGCCAGGCGAG
CGAAAGCATTAGCAGCTGGCTGGCGTGGTATCAGCAGAAACCGGGCCAGGCGCCGAAACTGCTGATTTATGAAGCGA
GCAAACTGGCGAGCGGCGTGCCGAGCCGCTTTAGCGGCAGCGGCAGCGGCACCGAATTTACCCTGACCATTAGCAGC
CTGGAACCGGAAGATTTTGCGACCTATTATTGCCAGGGCTATTTTTATTTTATTAGCCGCACCTATGTGAACAGCTT
TGGCGGCGGCACCAAAGTGGAAATTAAA
>Sequence ID 243: αPD-L1 VH Amino Acid Sequence
EVQLLESGGGLVQPGGSLSLSCAASGFSFSSGYDMCWVRQAPGKGLEWIACIAAGSAGITYDANWAKGRFTISRDNS
KNTLYLQMNSLRAEDTAVYYCARSAFSFDYAMDLWGQGTLVTVSS
>Sequence ID 244: αPD-L1 VH Nucleotide Sequence
GAAGTGCAATTGCTCGAAAGTGGTGGCGGACTTGTGCAACCTGGTGGAAGCCTCTCACTCTCTTGCGCGGCGAGTGG
TTTTAGCTTCAGTAGCGGCTACGATATGTGCTGGGTAAGACAGGCTCCTGGTAAAGGCCTTGAGTGGATTGCGTGTA
TCGCCGCCGGGTCTGCTGGTATAACATACGATGCAAACTGGGCGAAAGGGCGCTTCACAATCTCCAGGGACAACTCA
AAAAATACATTGTACCTCCAGATGAATAGCCTGAGAGCCGAAGACACCGCAGTCTACTATTGCGCAAGGTCAGCATT
TAGCTTCGATTATGCAATGGATCTTTGGGGCCAGGGGACGCTCGTCACCGTCTCATCTTGA
>Sequence ID 245: αPD-L1 VL Amino Acid Sequence
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVTVL
>Sequence ID 246: αPD-L1 VL Nucleotide Sequence
GACATTCAGATGACACAGTCACCGTCTACGCTCTCAGCCTCAGTAGGCGATAGGGTGACTATCACCTGCCAAGCCTC
CCAATCTATAAGTTCACACCTCAACTGGTATCAACAGAAACCCGGTAAAGCACCGAAGCTGCTCATCTACAAGGCAT
CAACTCTTGCCTCAGGTGTGCCTAGTCGCTTCTCCGGGAGCGGCTCCGGTACTGAGTTTACTTTGACGATATCTTCC
CTCCAACCCGACGACTTCGCCACATACTATTGTCAGCAGGGTTATAGTTGGGGAAATGTTGACAATGTGTTTGGAGG
GGGAACGAAAGTGACTGTGTTG
>Sequence ID 247: α4-1BB VH Amino Acid Sequence
EVQLVESGGGLVQPGGSLRLSCTASGFTISSYHMQWVRQAPGKGLEYIGTISSGGNVYYASSARGRFTISRPSSKNT
VDLQMNSLRAEDTAVYYCARDSGYSDPMWGQGTLVTVSS
>Sequence ID 248: α4-1BB VH Nucleotide Sequence
GAGGTTCAGTTGGTTGAAAGTGGGGGGGGACTCGTTCAGCCTGGAGGCTCTTTGCGACTTTCTTGTACCGCTTCTGG
GTTCACTATAAGTTCATATCATATGCAATGGGTAAGACAGGCGCCTGGTAAGGGACTTGAATACATAGGAACAATAA
GCTCTGGTGGTAACGTCTATTACGCCTCATCCGCGCGGGGGAGGTTTACAATTTCCAGGCCTTCTAGCAAAAACACG
GTAGATCTGCAAATGAACTCTCTTCGCGCTGAAGATACAGCAGTCTACTACTGCGCCAGGGATTCAGGGTATTCTGA
CCCCATGTGGGGGCAGGGCACATTGGTTACCGTGTCCTCT
>Sequence ID 249: α4-1BB VL Amino Acid Sequence
DVVMTQSPSSVSASVGDRVTITCQASQNIRTYLSWYQQKPGKAPKLLIYAAANLASGVPSRFSGSGSGTDFTLTISD
LEPGDAATYYCQSTYLGTDYVGGAFGGGTKLTVL
>Sequence ID 250: α4-1BB VL Nucleotide Sequence
GACGTTGTGATGACGCAGTCTCCTAGTTCAGTATCAGCGTCTGTTGGGGATCGAGTCACGATTACATGCCAGGCGAG
CCAAAATATTAGGACGTATCTGTCTTGGTATCAACAAAAGCCTGGTAAGGCTCCAAAACTCCTCATTTACGCCGCTG
CCAACTTGGCTAGTGGAGTACCTTCACGCTTTAGTGGGTCAGGTTCTGGAACAGATTTTACGTTGACGATCTCCGAT
TTGGAACCAGGGGACGCTGCCACGTATTATTGCCAGTCCACCTACCTCGGGACTGACTACGTTGGTGGGGCGTTTGG
CGGAGGAACAAAACTCACTGTACTT
>Sequence ID 251: 41BBL Amino Acid Sequence
REGPELSPDDPAGLLDLRQGMFAQLVAQNVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSAFGFQGRLLHLSAGQRLGVHLHTEARARH
AWQLTQGATVLGLFRVTPEIPAGLGSTGSGSKPGSGEGSTKGREGPELSPDDPAGLLDLRQGMFAQLVAQNVLLIDG
PLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGLGGGGSGGGGSR
EGPELSPDDPAGLLDLRQGMFAQLVAQNVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFGFQGRLLHLSAGQRLGVHLHTEARARHA
WQLTQGATVLGLFRVTPEIPAGL
>Sequence ID 252: 41BBL Nucleotide Sequence
CGAGAGGGCCCCGAGCTGTCTCCTGATGACCCAGCAGGCCTCTTGGACTTGCGGCAGGGTATGTTCGCTCAACTTGT
GGCTCAGAATGTTCTGCTCATTGATGGACCACTCTCTTGGTATAGTGACCCCGGTCTGGCCGGGGTGAGTCTGACCG
GCGGGCTCTCTTATAAAGAGGATACTAAGGAACTGGTCGTAGCAAAAGCGGGCGTTTATTACGTTTTTTTTCAGCTG
GAGCTCAGGCGCGTGGTGGCCGGCGAGGGCAGTGGCTCTGTGTCCCTGGCCCTGCACTTACAGCCCTTGAGAAGCGC
TGCAGGTGCTGCCGCACTGGCTTTAACTGTTGACCTCCCTCCGGCCTCTTCTGAAGCTAGAAACAGCGCTTTCGGCT
TCCAAGGGCGCCTGCTGCACCTGAGCGCAGGCCAGCGCTTAGGTGTGCACCTTCATACAGAGGCCAGGGCCCGACAC
GCTTGGCAGCTCACACAGGGTGCCACGGTTCTCGGACTTTTCCGCGTTACTCCCGAGATCCCCGCTGGCCTCGGAAG
TACTGGTTCTGGGTCTAAACCCGGTTCCGGCGAAGGTAGTACTAAAGGACGAGAAGGGCCAGAGTTAAGTCCAGATG
ACCCTGCTGGGCTTTTGGACCTGCGGCAGGGCATGTTCGCTCAACTGGTGGCTCAGAACGTGCTGCTGATCGATGGC
CCCCTGAGTTGGTACAGCGATCCCGGGCTGGCAGGCGTGTCACTTACAGGGGGCCTCTCTTACAAGGAAGACACCAA
GGAGTTAGTGGTCGCTAAGGCTGGCGTGTATTACGTGTTCTTCCAACTGGAGCTGAGAAGGGTTGTGGCAGGAGAGG
GTAGCGGCAGCGTGTCTTTAGCCCTTCACTTGCAGCCCCTGAGGTCTGCTGCAGGTGCAGCCGCTCTCGCGCTCACC
GTGGATCTCCCCCCAGCCTCATCTGAAGCTAGGAACAGTGCATTTGGCTTTCAGGGACGCTTGCTGCACCTCTCCGC
TGGACAGAGGCTGGGCGTGCACCTTCACACAGAGGCCCGTGCCAGGCATGCATGGCAGCTCACTCAGGGGGCAACAG
TGCTGGGTCTCTTCCGCGTGACTCCTGAAATACCAGCTGGACTTGGCGGTGGAGGCAGCGGCGGAGGAGGATCTCGT
GAGGGGCCAGAACTGTCCCCCGATGACCCAGCCGGACTGCTCGATCTCAGACAGGGCATGTTCGCTCAGCTTGTAGC
CCAAAATGTCCTCCTGATTGACGGCCCTTTGAGCTGGTATAGTGATCCCGGCTTGGCCGGGGTATCTCTGACCGGAG
GCCTCTCCTACAAGGAAGACACCAAAGAGCTGGTGGTGGCAAAAGCGGGGGTGTATTATGTGTTCTTTCAGCTCGAG
CTGCGGAGAGTTGTGGCCGGGGAAGGGTCTGGGAGCGTATCTCTTGCACTTCACCTGCAGCCCCTGCGCAGCGCCGC
TGGAGCCGCCGCCCTTGCTCTTACTGTGGATCTGCCTCCTGCTTCCTCAGAAGCACGCAACAGCGCCTTCGGCTTTC
AAGGACGTCTCCTGCACTTGTCCGCAGGACAGAGGTTGGGCGTCCATTTACACACTGAGGCACGGGCACGGCACGCT
TGGCAGCTTACCCAGGGAGCCACCGTGCTGGGACTCTTTAGAGTGACACCCGAGATCCCCGCTGGCTTGTGA
>Sequence ID 253: NKG2D dimer Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTV
>Sequence ID 254: NKG2D dimer Nucleotide Sequence
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTG
>Sequence ID 255: αHER3 VH Amino Acid Sequence
QVQLQESGGGLVKPGGSLSLSCAASGFTFSSYWMSWVRQAPGKGLEWVANINRDGSASYYVDSVKGRFTISRDDAKN
SLYLQMNSLRAEDTAVYYCARDRGVGYFDLWGRGTLVTVSS
>Sequence ID 256: αHER3 VH Nucleotide Sequence
CAGGTGCAATTGCAGGAGTCGGGGGGAGGCCTGGTCAAGCCTGGAGGGTCCCTGAGCCTCTCCTGTGCAGCCTCTGG
ATTCACCTTTAGTAGTTATTGGATGAGCTGGGTCCGCCAGGCTCCAGGGAAGGGGCTGGAGTGGGTGGCCAACATAA
ACCGCGATGGAAGTGCGAGTTACTATGTGGACTCTGTGAAGGGCCGATTCACCATCTCCAGAGACGACGCCAAGAAC
TCACTGTATCTGCAAATGAACAGCCTGAGAGCTGAGGACACGGCTGTGTATTACTGTGCGAGAGATCGTGGGGTGGG
CTACTTCGATCTCTGGGGCCGTGGCACCCTGGTCACCGTCTCGAGT
>Sequence ID 257: αHER3 VL Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVL
>Sequence ID 258: αHER3 VL Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTA
>Sequence ID 259: αCD20 VH Amino Acid Sequence
QVQLQQPGAELVKPGASVKMSCKASGYTFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSS
>Sequence ID 260: αCD20 VH Nucleotide Sequence
CAGGTGCAGCTGCAGCAGCCCGGCGCCGAGCTGGTGAAGCCCGGCGCCAGCGTGAAGATGAGCTGCAAGGCCAGCGG
CTACACCTTCACCAGCTACAACATGCACTGGGTGAAGCAGACCCCCGGCCGGGGCCTGGAGTGGATCGGCGCCATCT
ACCCCGGCAACGGCGACACCAGCTACAACCAGAAGTTCAAGGGCAAGGCCACCCTGACCGCCGACAAGAGCAGCAGC
ACCGCCTACATGCAGCTGAGCAGCCTGACCAGCGAGGACAGCGCCGTGTACTACTGCGCCCGGAGCACCTACTACGG
CGGCGACTGGTACTTCAACGTGTGGGGCGCCGGCACCACCGTGACCGTCTCGAGC
>Sequence ID 261: αCD20 VL Amino Acid Sequence
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLTVL
>Sequence ID 262: αCD20 VL Nucleotide Sequence
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GACCGTGCTG
>Sequence ID 263: EGFR VHH Amino Acid Sequence
EVQLQESGGGLVQAGDSLSLSCLVSGRSFNSYTMGWFRQAPGKEREFVAAILWSGPTTYYADSVKGRFTISRDNAKN
TVYLQMNSLKPEDTAVYYCAAALGVLVLAPGNVYSYWGQGTQVTVSS
>Sequence ID 264: EGFR VHH Nucleotide Sequence
GAGGTGCAGCTGCAGGAGTCTGGCGGCGGCCTGGTGCAGGCTGGGGACAGCCTGAGCCTGTCCTGCCTGGTGTCTGG
CCGCTCCTTCAACTCCTACACCATGGGCTGGTTCCGCCAGGCCCCTGGGAAGGAGCGGGAGTTTGTGGCTGCCATCC
TGTGGAGTGGCCCCACCACCTACTATGCTGACTCTGTGAAGGGCCGCTTCACCATCTCCAGAGACAATGCCAAGAAC
ACTGTGTACCTGCAGATGAACAGCCTGAAGCCAGAGGACACAGCTGTGTACTACTGTGCTGCTGCCCTGGGAGTGCT
GGTGCTGGCCCCTGGCAATGTGTACAGCTACTGGGGCCAGGGCACCCAGGTGACTGTCTCGAGC
>Sequence ID 265: HER3 VHH Amino Acid Sequence
QVQLVQSGGGLVQAGGSLSLSCAFSGRTFSMYTMGWFRQAPGKEREFVAANRGRGLSPDIADSVNGRFTISRDNAKN
TLYLQMDSLKPEDTAVYYCAADLQYGSSWPQRSSAEYDYWGQGTTVTVSS
>Sequence ID 266: HER3 VHH Nucleotide Sequence
CAGGTGCAGCTGGTGCAGTCTGGCGGAGGCCTGGTGCAGGCTGGGGGCAGCCTGAGCCTGAGCTGTGCCTTCTCTGG
CCGCACCTTCTCCATGTACACCATGGGCTGGTTCCGCCAGGCCCCAGGGAAGGAGAGAGAGTTTGTGGCTGCCAACA
GAGGCAGAGGCCTGAGCCCAGACATTGCTGACTCTGTGAATGGCCGCTTCACCATCTCCAGAGACAATGCCAAGAAC
ACCCTGTACCTGCAGATGGACAGCCTGAAGCCAGAGGACACAGCTGTGTACTACTGTGCTGCTGACCTGCAGTATGG
CAGCAGCTGGCCTCAGAGGAGCTCTGCTGAGTATGACTACTGGGGCCAGGGCACCACTGTGACTGTCTCGAGC
>Sequence ID 267: αPD-L1 Stapled VH Amino Acid Sequence
EVQLLESGGGLVQPGGSLRLSCAASGFSFSSGYDMCWVRQAPGKCLEWIACIAAGSAGITYDANWAKGRFTISRDNS
KNTLYLQMNSLRAEDTAVYYCARSAFSFDYAMDLWGQGTLVTVSS
>Sequence ID 268: αPD-L1 Stapled VH Nucleotide Sequence
GAGGTGCAGCTGTTGGAGTCTGGGGGAGGCTTGGTACAGCCTGGGGGGTCCCTGAGACTCTCCTGTGCAGCCTCTGG
ATTCTCCTTCAGTAGCGGGTACGACATGTGCTGGGTCCGCCAGGCTCCAGGGAAGTGCCTGGAGTGGATCGCATGCA
TTGCTGCTGGTAGTGCTGGTATCACTTACGACGCGAACTGGGCGAAAGGCCGGTTCACCATCTCCAGAGACAATTCC
AAGAACACGCTGTATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTCTCGTCT
>Sequence ID 269: αPD-L1 Stapled VL Amino Acid Sequence
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGCGTKVEIK
>Sequence ID 270: αPD-L1 Stapled VL Nucleotide Sequence
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCTG
CGGGACCAAGGTGGAGATCAAATAG
>Sequence ID 271: α4-1BB VH Stapled Amino Acid Sequence
RSLVESGGGLVQPGGSLRLSCTASGFTISSYHMQWVRQAPGKCLEYIGTISSGGNVYYASSARGRFTISRPSSKNTV
DLQMNSLRAEDTAVYYCARDSGYSDPMWGQGTLVTVSS
>Sequence ID 272: α4-1BB VH Stapled Nucleotide Sequence
CGGTCGCTGGTGGAGTCTGGGGGAGGCTTGGTCCAGCCTGGGGGGTCCCTGAGACTCTCCTGTACTGCCTCTGGATT
CACCATCAGTAGCTACCACATGCAGTGGGTCCGGCAGGCACCTGGGAAGTGCCTGGAGTACATCGGAACCATTAGTA
GTGGTGGTAATGTATACTACGCAAGCTCCGCTAGAGGCAGATTCACCATCTCCAGACCCTCGTCCAAGAACACGGTG
GATCTTCAAATGAACAGCCTGAGAGCCGAGGACACGGCTGTGTATTACTGTGCGAGAGACTCTGGTTATAGTGATCC
TATGTGGGGCCAGGGAACCCTGGTCACCGTCTCTTCA
>Sequence ID 273: α4-1BB VL Stapled Amino Acid Sequence
DVVMTQSPSSVSASVGDRVTITCQASQNIRTYLSWYQQKPGKAPKLLIYAAANLASGVPSRFSGSGSGTDFTLTISD
LEPGDAATYYCQSTYLGTDYVGGAFGCGTKVEIK
>Sequence ID 274: α4-1BB VL Stapled Nucleotide Sequence
GACGTTGTGATGACCCAGTCTCCATCTTCCGTGTCTGCATCTGTAGGAGACAGAGTCACCATCACCTGTCAGGCCAG
TCAGAACATTAGGACTTACTTATCCTGGTATCAGCAGAAACCAGGGAAAGCCCCTAAGCTCCTGATCTATGCTGCAG
CCAATCTGGCATCTGGGGTCCCATCAAGGTTCAGCGGCAGTGGATCTGGGACAGATTTCACTCTCACCATCAGCGAC
TTGGAACCTGGCGATGCTGCAACTTACTATTGTCAGTCTACCTATCTTGGTACTGATTATGTTGGCGGTGCTTTCGG
CTGTGGGACCAAGGTGGAGATCAAATGA
>Sequence ID 275: αHer3 VH Stapled Amino Acid Sequence
QVQLQESGGGLVKPGGSLRLSCAASGFTFSSYWMSWVRQAPGKCLEWVANINRDGSASYYVDSVKGRFTISRDDAKN
SLYLQMNSLRAEDTAVYYCARDRGVGYFDLWGRGTLVTVST
>Sequence ID 276: αHer3 VH Stapled Nucleotide Sequence
CAGGTGCAATTGCAGGAGTCGGGGGGAGGCCTGGTCAAGCCTGGAGGGTCCCTGAGACTCTCCTGTGCAGCCTCTGG
ATTCACCTTTAGTAGTTATTGGATGAGCTGGGTCCGCCAGGCTCCAGGGAAGTGCCTGGAGTGGGTGGCCAACATAA
ACCGCGATGGAAGTGCGAGTTACTATGTGGACTCTGTGAAGGGCCGATTCACCATCTCCAGAGACGACGCCAAGAAC
TCACTGTATCTGCAAATGAACAGCCTGAGAGCTGAGGACACGGCTGTGTATTACTGTGCGAGAGATCGTGGGGTGGG
CTACTTCGATCTCTGGGGCCGTGGCACCCTGGTCACCGTGTCGACA
>Sequence ID 277: αHer3 VL Stapled Amino Acid Sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGCGTKVTVL
>Sequence ID 278: αHer3 VL Stapled Nucleotide Sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CTGTGGGACCAAGGTGACCGTCCTA
>Sequence ID 279: αCEA 656E11 VH Amino Acid Sequence
EVQLVESGGGLVQPGGSLRLSCAASGFSFSGTQYVCWVRQAPGKGLEWIGCIYVDVANTYYATSVKGRFTISRDNSK
NTLYLQMNSLRAEDTAVYYCASHDTVCAGCGLDLWGQGTLVTVSS
>Sequence ID 280: αCEA 656E11 VH Nucleotide Sequence
GAGGTGCAGCTGGTGGAGTCTGGGGGAGGCTTGGTCCAGCCTGGGGGGTCCCTGAGACTCTCCTGTGCAGCCTCTGG
ATTCTCCTTCAGTGGCACCCAGTACGTGTGCTGGGTCCGCCAGGCTCCAGGGAAGGGGCTGGAGTGGATCGGATGCA
TTTATGTGGATGTGGCTAACACTTACTACGCGACCTCCGTGAAGGGCAGATTCACCATCTCCAGAGACAATTCCAAG
AACACGCTGTATCTTCAAATGAACAGCCTGAGAGCCGAGGACACGGCTGTGTATTACTGTGCGAGTCATGATACTGT
TTGTGCTGGTTGTGGTTTGGACTTGTGGGGCCAGGGAACCCTGGTCACCGTCTCGAGC
>Sequence ID 281: αCEA 656E11 VL Amino Acid Sequence
DIQMTQSPSTLSASVGDRVTITCQASENIYSNLAWYQQKPGKAPKLLIYDASTLASGVPSRFSGSGSGTEFTLTISS
LQPDDFATYYCQSTYYNNIIYGAAFGGGTKVEIK
>Sequence ID 282: αCEA 656E11 VL Nucleotide Sequence
GACATCCAGATGACCCAGTCTCCTTCCACCCTGTCTGCATCTGTAGGAGACAGAGTCACCATCACTTGCCAGGCCAG
TGAGAACATTTACAGCAATTTAGCCTGGTATCAGCAGAAACCAGGGAAAGCCCCTAAGCTCCTGATCTATGATGCTT
CCACTCTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAATCTACTTATTATAATAATATCATTTATGGGGCTGCTTTCGG
CGGAGGGACCAAGGTGGAGATCAAA
>Sequence ID 283: αHER2 VH Amino Acid Sequence
EVQLVESGGGLVQPGGSLRLSCAASGFNIKDTYIHWVRQAPGKGLEWVARIYPTNGYTRYADSVKGRFTISADTSKN
TAYLQMNSLRAEDTAVYYCSRWGGDGFYAMDYWGQGTLVTVST
>Sequence ID 284: αHER2 VH Nucleotide Sequence
GAAGTACAGCTTGTTGAGAGTGGTGGTGGACTTGTCCAACCAGGGGGAAGTCTGCGGCTGTCATGCGCAGCCTCTGG
CTTCAACATTAAGGACACCTACATTCACTGGGTTCGGCAAGCGCCGGGGAAGGGGCTGGAATGGGTCGCCAGGATAT
ACCCTACCAACGGGTACACCAGGTATGCAGATAGCGTTAAGGGTAGATTCACTATTTCAGCTGACACTAGCAAGAAC
ACCGCGTATCTCCAGATGAACTCCCTTCGCGCCGAGGATACGGCTGTGTACTACTGCTCTAGGTGGGGAGGAGATGG
GTTTTACGCAATGGATTACTGGGGACAGGGAACACTCGTTACGGTGTCGACA
>Sequence ID 285: αHER2 VL Amino Acid Sequence
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIK
>Sequence ID 286: αHER2 VL Nucleotide Sequence
GACATCCAAATGACGCAGTCTCCCTCCAGTTTGAGTGCAAGTGTGGGAGACAGAGTTACGATCACATGTAGGGCAAG
TCAGGACGTCAATACAGCCGTCGCATGGTACCAGCAGAAGCCTGGAAAGGCCCCGAAACTTCTGATTTACTCTGCAA
GTTTCCTCTACAGTGGTGTTCCTAGTCGCTTCAGTGGGTCTCGGTCAGGCACCGATTTTACCCTGACAATTAGCAGC
TTGCAACCGGAAGATTTTGCGACCTATTATTGCCAGCAACATTATACCACCCCTCCCACATTTGGACAAGGCACGAA
GGTAGAGATTAAA
>Sequence ID 287: SI-20C14 (anti-CEA 656E11) Heavy Chain Amino Acid Sequence
EVQLVESGGGLVQPGGSLRLSCAASGFSFSGTQYVCWVRQAPGKGLEWIGCIYVDVANTYYATSVKGRFTISRDNSK
NTLYLQMNSLRAEDTAVYYCASHDTVCAGCGLDLWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGAPSVFLFPPKPKDTLMISRTPEVTCVVVDVSHEDPEVKFNWYVDGVEVHNAKTKPREEQYNSTYRVV
SVLTVLHQDWLNGKEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPG
>Sequence ID 288: SI-20C14 (anti-CEA 656E11) Heavy Chain Nucleotide Sequence
GAGGTGCAGCTGGTGGAGTCTGGGGGAGGCTTGGTCCAGCCTGGGGGGTCCCTGAGACTCTCCTGTGCAGCCTCTGG
ATTCTCCTTCAGTGGCACCCAGTACGTGTGCTGGGTCCGCCAGGCTCCAGGGAAGGGGCTGGAGTGGATCGGATGCA
TTTATGTGGATGTGGCTAACACTTACTACGCGACCTCCGTGAAGGGCAGATTCACCATCTCCAGAGACAATTCCAAG
AACACGCTGTATCTTCAAATGAACAGCCTGAGAGCCGAGGACACGGCTGTGTATTACTGTGCGAGTCATGATACTGT
TTGTGCTGGTTGTGGTTTGGACTTGTGGGGCCAGGGAACCCTGGTCACCGTCTCGAGCGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTTAA
>Sequence ID 289: SI-20C14 (anti-CEA 656E11) Light Chain Amino Acid Sequence
DIQMTQSPSTLSASVGDRVTITCQASENIYSNLAWYQQKPGKAPKLLIYDASTLASGVPSRFSGSGSGTEFTLTISS
LQPDDFATYYCQSTYYNNIIYGAAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
>Sequence ID 290: SI-20C14 (anti-CEA 656E11) Light Chain Nucleotide Sequence
GACATCCAGATGACCCAGTCTCCTTCCACCCTGTCTGCATCTGTAGGAGACAGAGTCACCATCACTTGCCAGGCCAG
TGAGAACATTTACAGCAATTTAGCCTGGTATCAGCAGAAACCAGGGAAAGCCCCTAAGCTCCTGATCTATGATGCTT
CCACTCTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAATCTACTTATTATAATAATATCATTTATGGGGCTGCTTTCGG
CGGAGGGACCAAGGTGGAGATCAAACGTACGGTGGCTGCACCATCTGTCTTCATCTTCCCGCCATCTGATGAGCAGT
TGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGTTAG
>Sequence ID 291: αCD3***** VH Amino Acid Sequence
QQLVESGGGLVKPGGSLRLSCAASGIDLSSNAIGWVRQAPGKGLEWVGVIFGSGNTYYASWAKGRFTISRSKNTVYL
QMNSLRAEDTAVYYCARGGYSSDIWGQGTLVTVSS
>Sequence ID 292: αCD3***** VH Nucleotide Sequence
CAACAGTTGGTTGAGTCTGGTGGCGGTCTCGTTAAGCCTGGAGGGTCCCTTCGGCTTAGTTGTGCGGCCTCTGGGAT
CGACCTGTCTAGCAACGCTATAGGATGGGTACGGCAAGCTCCGGGCAAAGGCCTTGAGTGGGTCGGCGTTATTTTTG
GGAGCGGTAATACGTACTACGCCAGCTGGGCAAAAGGTAGGTTCACTATCAGTCGATCAAAGAACACGGTATATCTT
CAGATGAACAGTCTCCGGGCGGAGGACACCGCGGTTTATTATTGCGCAAGGGGGGGATACTCCTCAGACATATGGGG
CCAAGGTACTTTGGTGACGGTCTCGAGC
>Sequence ID 293: αCD3***** VL Amino Acid Sequence
DPVLTQSPSSLSASVGDRVTISCQSSQSVAKNNNLAWFQQKPGQAPKLLIYSASTLAAGVPSRFSGSGSGTDFTLTI
SSVQPEDFATYYCSARDSGNIQSFGGGTKVEIK
>Sequence ID 294: αCD3***** VL Nucleotide Sequence
GATCCAGTTCTGACACAAAGTCCATCCAGCCTGTCTGCCTCAGTCGGCGACAGAGTGACCATCAGTTGCCAGAGCTC
ACAGTCTGTGGCTAAGAACAACAACTTGGCGTGGTTCCAACAGAAACCTGGACAGGCTCCGAAATTGCTGATCTATT
CTGCTTCCACGCTTGCTGCTGGTGTTCCTTCCCGCTTTTCAGGTAGTGGTAGCGGGACAGACTTCACTTTGACTATA
AGCAGCGTGCAGCCTGAAGATTTTGCGACCTACTATTGTTCTGCTAGAGACAGTGGAAATATTCAGTCCTTTGGGGG
GGGAACGAAGGTCGAAATAAAG
>Sequence ID 295: NKG2D Monomer Amino Acid Sequence
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTV
>Sequence ID 296: NKG2D Monomer Nucleotide Sequence
TTTCTGAATAGCCTGTTCAACCAGGAGGTGCAGATCCCACTGACCGAGAGCTATTGTGGACCTTGTCCTAAGAACTG
GATCTGTTACAAGAACAACTGCTACCAGTTCTTCGACGAGAGCAAGAATTGGTATGAAAGCCAGGCCAGCTGCATGA
GCCAAAACGCCTCTCTGCTGAAGGTGTACTCCAAAGAAGATCAGGACCTGCTGAAGCTGGTGAAGTCCTACCACTGG
ATGGGCCTGGTGCACATCCCAACCAACGGCAGCTGGCAGTGGGAAGACGGCAGCATCCTGAGCCCTAACCTGCTGAC
AATCATTGAGATGCAGAAAGGCGACTGCGCCCTGTACGCCAGTTCTTTTAAGGGCTACATCGAAAACTGTAGCACCC
CTAACACCTACATCTGCATGCAGAGAACAGTG
>Sequence ID 297: SI-75XM9 chain A amino acid sequence
QVQLQESGGRLVQPGEPLSLTCKTSGIDLSSNAIGWVRQAPGKGLEWIGVIFGSGNTYYASWAKGRFTISRSTSTVY
LKMNSLRSEDTAIYYCARGGYSSDIWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEAAGGP
SVFLFPPKPKDTLMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLSCAVKGFYPSDIAVEWESNGQPENNYK
TTPPVLDSDGSFFLVSRLTVDKSRWQEGNVFSCSVMHEALHNHYTQKSLSLSLG
>Sequence ID 298: SI-75XM9 chain A nucleotide sequence
CAAGTGCAGTTGCAAGAAAGTGGTGGTAGACTGGTTCAGCCTGGTGAACCCTTGTCACTGACGTGTAAAACAAGCGG
CATTGATCTGTCCTCTAACGCCATCGGATGGGTCCGACAGGCCCCAGGAAAAGGTCTGGAGTGGATCGGAGTTATCT
TCGGGAGCGGCAATACATACTACGCAAGCTGGGCAAAAGGGCGATTTACGATATCACGGAGCACCTCTACAGTTTAT
TTGAAAATGAACTCCCTCCGGTCCGAGGATACCGCGATATATTACTGTGCCAGAGGGGGGTACTCCTCTGATATCTG
GGGGCAGGGTACACTGGTTACAGTTTCATCCGCTAGCACCAAGGGACCCTCTGTGTTTCCTCTGGCTCCTTGCTCCA
GATCCACCTCCGAGTCTACAGCTGCTCTGGGCTGCCTGGTCAAGGACTACTTTCCTGAGCCTGTGACCGTGTCCTGG
AACTCTGGCGCTCTGACATCTGGCGTGCACACCTTTCCAGCTGTGCTGCAGTCCTCCGGCCTGTACTCTCTGTCCTC
TGTCGTGACCGTGCCTTCCTCTAGCCTGGGCACCAAGACCTACACCTGTAATGTGGACCACAAGCCTTCCAACACCA
AGGTGGACAAGCGCGTGGAATCTAAGTACGGCCCTCCTTGTCCTCCATGTCCTGCTCCAGAAGCTGCTGGCGGCCCA
TCCGTGTTTCTGTTCCCTCCAAAGCCTAAGGACACCCTGATGATCTCTCGGACCCCTGAAGTGACCTGCGTGGTGGT
GGATGTGTCCCAAGAGGATCCCGAGGTGCAGTTCAATTGGTACGTGGACGGCGTGGAAGTGCACAACGCCAAGACCA
AGCCTAGAGAGGAACAGTTCAACTCCACCTACAGAGTGGTGTCCGTGCTGACCGTGCTGCACCAGGATTGGCTGAAC
GGCAAAGAGTACAAGTGCAAGGTGTCCAACAAGGGCCTGCCTTCCAGCATCGAAAAGACCATCTCCAAGGCCAAGGG
CCAGCCTAGGGAACCCCAGGTTTACACCCTGCCTCCAAGCCAAGAGGAAATGACCAAGAACCAGGTGTCCCTGTCCT
GCGCCGTGAAGGGCTTCTACCCTTCCGATATCGCCGTGGAATGGGAGAGCAATGGCCAGCCTGAGAACAACTACAAG
ACAACCCCTCCTGTGCTGGACTCCGACGGCTCCTTCTTCCTGGTGTCCCGCCTGACCGTGGACAAGTCCAGATGGCA
AGAGGGCAACGTGTTCTCCTGCTCCGTGATGCACGAGGCCCTGCACAATCACTACACCCAGAAGTCCCTGTCTCTGT
CCCTGGGATGA
>Sequence ID 299: SI-75XM9 chain B amino acid sequence
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLSLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDPQLTQSPSSLSATVGQRVTINCQSSQSVAKNNNLAWFQQKPGKPPKLL
IYSASTLAAGVPSRFSGSGSGTQFTLTITRVQSEDFATYYCSARDSGNIQSFGGGTRVEIKRTVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECGGGPPCPPCPAPEAAGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQVYTLPPSQEEMTK
NQVSLWCLVKGFYPSDIAVEWESNGQPENNYKTTPPVLDSDGSFFLYSRLTVDKSRWQEGNVFSCSVMHEALHNRFT
QKSLSLSLG
>Sequence ID 300: SI-75XM9 chain B nucleotide sequence
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGCGGTGGCGGTAGTGGGGGAGGCGGTTCTGGCGGCGGAGGGTCCGGCGGTG
GAGGATCACAGGTGCAATTGCAGGAGTCGGGGGGAGGCCTGGTCAAGCCTGGAGGGTCCCTGAGCCTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGTGGCGGTGGAGGGTCCGGCGGTGG
TGGATCAGATCCTCAATTGACCCAGTCCCCTAGTAGCCTTTCTGCAACCGTGGGCCAGAGGGTAACAATCAATTGCC
AGTCAAGCCAGTCCGTAGCGAAGAATAATAATTTGGCATGGTTCCAACAAAAACCGGGTAAACCTCCTAAACTCCTT
ATCTACTCAGCTTCAACCCTTGCCGCGGGTGTGCCCAGCCGGTTCTCAGGCTCAGGTTCCGGGACTCAATTTACACT
TACCATCACTCGCGTCCAAAGTGAGGATTTTGCAACCTACTATTGTTCTGCACGGGATTCCGGGAACATACAGTCCT
TTGGGGGTGGAACTCGGGTGGAGATAAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTGGCGGCGGACCTCCTTGTCCTCCATGTCCTGCTCC
AGAAGCTGCTGGCGGCCCATCCGTGTTTCTGTTCCCTCCAAAGCCTAAGGACACCCTGATGATCTCTCGGACCCCTG
AAGTGACCTGCGTGGTGGTGGATGTGTCCCAAGAGGATCCCGAGGTGCAGTTCAATTGGTACGTGGACGGCGTGGAA
GTGCACAACGCCAAGACCAAGCCTAGAGAGGAACAGTTCAACTCCACCTACAGAGTGGTGTCCGTGCTGACCGTGCT
GCACCAGGATTGGCTGAACGGCAAAGAGTACAAGTGCAAGGTGTCCAACAAGGGCCTGCCTTCCAGCATCGAAAAGA
CCATCTCCAAGGCCAAGGGCCAGCCTAGGGAACCCCAGGTTTACACCCTGCCTCCAAGCCAAGAGGAAATGACCAAG
AACCAGGTGTCCCTGTGGTGCCTCGTGAAGGGCTTCTACCCTTCCGATATCGCCGTGGAATGGGAGAGCAATGGCCA
GCCTGAGAACAACTACAAGACAACCCCTCCTGTGCTGGACTCCGACGGCTCCTTCTTCCTGTATTCCCGCCTGACCG
TGGACAAGTCCAGATGGCAAGAGGGCAACGTGTTCTCCTGCTCCGTGATGCACGAGGCCCTGCACAATCGCTTCACC
CAGAAGTCCCTGTCTCTGTCCCTGGGATGA
>Sequence ID 301: anti-CEA 656E11 CDR-H1 amino acid sequence
GTQYVC
>Sequence ID 302: anti-CEA 656E11 CDR-H2 amino acid sequence
CIYVDVANTYYATSVKG
>Sequence ID 303: anti-CEA 656E11 CDR-H3 amino acid sequence
HDTVCAGCGLDL
>Sequence ID 304: anti-CEA 656E11 CDR-L1 amino acid sequence
QASENIYSNLA
>Sequence ID 305: anti-CEA 656E11 CDR-L2 amino acid sequence
DASTLAS
>Sequence ID 306: anti-CEA 656E11 CDR-L3 amino acid sequence
QSTYYNNIIYGAA
>Sequence ID 307: anti-CD3 283E3 (BSM/FRS) CDR-H1 amino acid sequence
SNAIG
>Sequence ID 308: anti-CD3 283E3 (BSM/FRS) CDR-H2 amino acid sequence
VIFGSGNTYYASWAKG
>Sequence ID 309: anti-CD3 283E3 (BSM/FRS) CDR-H3 amino acid sequence
GGYSSDI
>Sequence ID 310: anti-CD3 283E3 (BSM/FRS) CDR-L1 amino acid sequence
QSSQSVAKNNNLA
>Sequence ID 311: anti-CD3 283E3 (BSM/FRS) CDR-L2 amino acid sequence
SASTLAA
>Sequence ID 312: anti-CD3 283E3 (BSM/FRS) CDR-L3 amino acid sequence
SARDSGNIQS
>Sequence ID 313: human IgG1 amino acid sequence
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQQGNVFSC
SVMHEALHNHYTQKSLSLSPG
>Sequence ID 314: human IgG4 amino acid sequence
ASTKGPSVFPLAPCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLTVDKSRWQEGNVFSCSVM
HEALHNHYTQKSLSLSLG

What is claimed is:

1. A multi-specific antibody-like protein having a N-terminus and a C-terminus, comprising,
a first monomer, comprising, from the N-terminus to the C-terminus, a first binding monomer, a CH1 domain, a first hinge, a first CH2 domain, and a first CH3 domain, wherein the first monomer comprises optionally a first binding domain (D1) linked to the N-terminus, a fourth binding domain (D4) linked to the C-terminus, or both,
a second monomer, comprising, from the N-terminus to the C-terminus, a second binding monomer, a CL domain, a second hinge, a second CH2 domain, and a second CH3 domain, wherein the second monomer comprises optionally a second binding domain (D2) linked to the N-terminus, a fifth binding domain (D5) linked to the C-terminus, or both,
wherein the first binding monomer and a second binding monomer are configured to form a dimer,
wherein the first monomer and the second monomer are covalently paired through at least one disulfide bond between the CH1 domain and the CL domain and at least one disulfide bond between the first hinge and the second hinge, and
wherein the multi-specific antibody-like protein is at least bi-specific.

2. The multi-specific antibody-like protein of claim 1, wherein the first binding monomer comprises a VH domain, a second binding monomer comprises a VL domain, and wherein the VH, CH1, VL, CL domains form a Fab region as a third binding domain (D3).

3. The multi-specific antibody-like protein of claim 1, wherein the first binding monomer and the second binding monomer form a NKG2D receptor as a third binding domain (D3).

4. The multi-specific antibody-like protein of claim 1, wherein the first CH3 domain is configured to form a hole structure, wherein the second CH3 domain is configured to form a knob structure, and wherein the first CH2 and CH3 domains and the second CH2 and CH3 domains are configured to heterodimerize to form a complementary Fc domain.

5. The multi-specific antibody-like protein of claim 1, wherein the D1, D2, D4, and D5 is independently a scFv domain, a VHH domain, a receptor, or a ligand.

6. The multi-specific antibody-like protein of claim 1, wherein the D1, D2, D3, D4, and D5 each independently has a binding specificity against an antigen selected, EGFR, HER2, HER3, EGFRvIII, ROR1, CD3, CD28, CEA, LMP1, LMP2A, Mesothelin, PSMA, EpCAM, glypican-3, gpA33, GD2, TROP2, NKG2D, NKG2D ligand, BCMA, CD19, CD20, CD33, CD123, CD22, CD30, PD-L1, PD1, OX40, 4-1BB, GITR, TIGIT, TIM-3, LAG-3, CTLA4, CD40, CD40L, VISTA, ICOS, BTLA, LIGHT, HVEM, CSF1R, CD73, CD39, CLDN18.2, DLL3, HLA-G, FcRH5, GPRC5D, LIV-1, MUC1, CD138, CD70, CD16, uPAR, Siglec-15, CD47, CD38, NKp46, PD-L2, CD160, LOX-1, SIRPα, CD27 and wherein the Fc domain comprises a human IgG Fc domain.

7. The multi-specific antibody-like protein of claim 1, wherein D1 has a binding specificity to CD3, CD20, CEA, HER2, EGFR, or NKG2D ligand.

8. The multi-specific antibody-like protein of claim 1, wherein D2 has a binding specificity to HER3, EGFR, CD3, or CD19.

9. The multi-specific antibody-like protein of claim 1, wherein D3 has a binding specificity to HER3, EGFR, CD3, or NKG2D ligands.

10. The multi-specific antibody-like protein of claim 1, wherein D4 has a binding specificity to 4-1BB, or EGFR.

11. The multi-specific antibody-like protein of claim 1, wherein D5 has a binding specificity to PD-L1 or HER3.

12. The multi-specific antibody-like protein of claim 1, wherein the antibody is a tri-specific antibody having the binding specificity to EGFR, HER3, and CD3.

13. The multi-specific antibody-like protein of claim 1, wherein the antibody is a tetra-specific antibody having the binding specificity to EGFR, HER3, CD3, and 4-1BB.

14. The multi-specific antibody-like protein of claim 1, wherein the antibody is a tetra-specific antibody having the binding specificity to EGFR, HER3, CD3, and PD-L1.

15. The multi-specific antibody-like protein of claim 1, wherein the antibody is a penta-specific antibody having the binding specificity to EGFR, HER3, CD3, 4-1BB and PD-L1.

16. A complimentary determining region (CDR) having an affinity to CEA, comprising an amino acid sequence having sequence identity to SEQ ID NO. 301, 302, 303, 304, 305, or 306.

17. A protein having an affinity to CEA, comprising the CDR of claim 16.

18. A multi-specific antibody-like protein having a binding affinity to CEA, wherein the antibody-like protein comprises an amino acid sequence having sequence identity to an amino acid sequence selected from SEQ ID NO. 279, 280, 281 or 282.

19. A complimentary determining region (CDR) having a binding affinity to CD3, comprising an amino acid sequence having sequence identity to SEQ ID NO. 307, 308, 309, 310, 311 or 312.

20. A protein having an affinity to CD3, comprising CDR of claim 19.

21. A multi-specific antibody-like protein having a binding affinity to CD3, wherein the antibody-like protein comprises an amino acid sequence having sequence identity to an amino acid sequence selected from SEQ ID NO. 227-230, 231-234, 235-238, 239-242 and 291-294.

22. An isolated nucleic acid sequence encoding the multi-specific antibody-like protein of claim 1.

23. An expression vector comprising the isolated nucleic acid sequences of claim 22.

24. A host cell comprising the isolated nucleic acid sequence of claim 22.

25. An immunoconjugate comprising the multi-specific antibody-like protein of claim 1 and a cytotoxic agent.

26. A pharmaceutical composition, comprising the multi-specific antibody-like protein of claim 1 and a pharmaceutically acceptable carrier.

27. A pharmaceutical composition, comprising the immunoconjugate of claim 25 and a pharmaceutically acceptable carrier.

28. A method for treating or preventing a cancer, an autoimmune disease, or an infectious disease in a subject, said method comprising administering to the subject a pharmaceutical composition comprising a purified multi-specific antibody-like protein of claim 1.

29. A method for producing the multi-specific antibody-like protein of claim 1, comprising
culturing a host cell such that the DNA sequence encoding the multi-specific antibody-like protein of claim 1 is expressed, and
purifying said multi-specific antibody-like protein.

30. (canceled)
